(19) United States
(12) Patent Application Publication
(10) Pub. No.: US 2026/0103824 A1
(43) Pub. Date: Apr. 16, 2026

(54) Compositions and Methods for Selectively Synthesizing Triple-indexed cDNA Libraries

(71) Applicant: The Rockefeller University, New York, NY (US)

(72) Inventors: Junyue Cao, New York, NY (US); Wei Zhou, New York, NY (US); Jasper Lee, New York, NY (US); Ziyu Lu, New York, NY (US); Melissa Zhang, New York, NY (US); Andras Sziraki, New York, NY (US); Zihan Xu, New York, NY (US)

(21) Appl. No.: 19/114,973

(22) PCT Filed: Sep. 26, 2023
(86) PCT No.: PCT/US2023/075123

§ 371 Date: Mar. 25, 2025

Related U.S. Application Data
(60) Provisional application No. 63/377,061, filed on Sep. 26, 2022.
(60) Provisional application No. 63/385,479, filed on Nov. 30, 2022.

(51) Int. Cl. C40B 40/06; C12N 5/00; C12N 15/10; C12Q 1/25; C12Q 1/48; C12Q 1/6806; C12Q 1/686; C12Q 1/6876; C40B 50/00; C40B 70/00

(52) U.S. Cl. CPC C40B 40/06; C12N 5/0081; C12N 15/10; C12Q 1/25; C12Q 1/48; C12Q 1/6806; C12Q 1/686; C12Q 1/6876; C12Y 207/07049; C12Y 605/01001; C40B 50/00; C40B 70/00

(57) ABSTRACT

Provided herein are methods for preparing a sequencing library from a plurality of single cells that includes nucleic acids having three index sequences, as well as methods for generating an RNA sequencing library from single cells that can be used to dissect the critical regulators of gene-specific transcription, splicing, and degradation in a massive-parallel manner. Also provided herein are compositions, such as oligonucleotide sets for generating the sequencing libraries and kits for preparing the sequencing libraries.

### CROSS REFERENCE TO RELATED APPLICATIONS

This application claims priority to U.S. Provisional Application No. 63/377,061, filed Sep. 26, 2022 and to U.S. Provisional Application No. 63/385,479, filed Nov. 30, 2022, each of which is hereby incorporated by reference herein in its entirety.

### STATEMENT REGARDING FEDERALLY SPONSORED RESEARCH OR DEVELOPMENT

This invention was made with government support under Grant No. 1DP2HG012522, Grant No. 1R01AG076932 and Grant No. RM1HG011014 awarded by the National Institutes of Health (NIH). The government has certain rights in the invention.

### BACKGROUND OF THE INVENTION

New neurons and glia cells are continuously produced in the adult mammalian brains, a critical process associated with memory, learning, and stress (Lugert et al., Cell Stem Cell 6, 445-456 (2010); Spalding et al., Cell 153, 1219-1227 (2013)). There is a consensus that adult neurogenesis and oligodendrogenesis decline with advancing ages and in neuropathological conditions (Pollina et al., Oncogene 30, 3105-3126 (2011); Galvan et al., Clin. Interv. Aging 2, 605-610 (2007)), but to what extent is debated (Sorrells et al., Nature 555, 377-381 (2018); Mathews et al., Aging Cell 16, 1195-1199 (2017)). The ambiguity mainly stems from technical limitations-most studies rely upon the utilization of proxy markers and are unreliable in accurately quantifying the dynamics of rare progenitor cells. Therefore, novel approaches to precisely capturing newborn cells and tracking their dynamics are critical to understanding brain cell population dynamics in development, ageing, and diseases.

Cellular functions are determined by the expression of millions of RNA molecules, which are tightly regulated by their synthesis, splicing, and degradation. However, understanding how key regulators impact genome-wide RNA kinetics is constrained by existing tools, which provide only snapshots of the transcriptome (Jaitin et al., Cell 167, 1883-1896.e15 (2016); Adamson et al., Cell 167, 1867-1882.e21 (2016); Dixit et al., Cell 167, 1853-1866.e17 (2016); Xie et al., Mol. Cell 66, 285-299.e5 (2017); Datlinger et al., Nat. Methods 14, 297-301 (2017); Hill et al., Nat. Methods 15, 271-274 (2018); Replogle et al., Cell 185, 2559-2575.e28 (2022); Replogle et al., Nat. Biotechnol. 38, 954-961 (2020)).

The mammalian brain is a remarkably complex system made up of millions or billions of highly heterogeneous cells, comprising a myriad of different cell types and subtypes (Ero et al., Front. Neuroinform. 12, 84 (2018); Zeisel et al., Cell 174, 999-1014.e22 (2018)). Progressive changes in brain cell populations, which occurs during the normal aging process, may contribute to functional decline and increased risks for neurodegenerative diseases such as Alzheimer's disease (AD) (Mathys et al., Nature 570, 332-337 (2019); Xia et al., Aging Cell 17, el2802 (2018)). While the recent advances in single-cell genomics are creating unprecedented opportunities to explore the cell-type-specific dynamics across the entire mammalian brain in aging and AD models (Ximerakis et al., Nat. Neurosci. 22, 1696-1708 (2019); Morabito et al., Nature Genetics vol. 53 1143-1155 (2021); Tabula et al., Nature 583, 590-595 (2020); Wang et al., Nucleic Acids Res. (2022) doi:10.1093/nar/gkac633), most prior studies relied on a relatively shallow sampling of the brain cell populations, decreasing their abilities to investigate the dynamics of the global brain population and to identify rare aging or AD-associated cell types. While providing proof of key concepts, the prior studies were technically limited in several ways, including failing to recover isoform-level gene expression patterns for rare cell types, providing few insights into how the chromatin landscape regulates cell-type-specific alterations across aging stages, and often lacking integrative analyses with spatial visualization to explore the anatomic region-specific changes.

Single-cell RNA sequencing by combinatorial indexing has previously been developed, which provides a methodological framework involving split-pool barcoding of cells or nuclei for single-cell transcriptome profiling (Cao et al., Science 357, 661-667 (2017). While the method has been widely used to study embryonic and fetal tissues (Cao et al., Nature 566, 496-502 (2019); Cao et al., Science 370, (2020)), it remains restricted to gene quantification proximal to the 3′ end (i.e., full-length transcript isoform information is lost) and is limited in terms of efficiency and cell recovery (up to 95% cell loss rate) (Cao et al., Nature 566, 496-502 (2019)), which pose a challenge when dealing with aged tissues.

There is thus a need in the art for improved methods for single-cell RNA sequencing. The present invention addresses this unmet need in the art.

### SUMMARY OF THE INVENTION

In one embodiment, the invention relates to a method for preparing a sequencing library comprising nucleic acids from a plurality of single nuclei or cells, the method comprising:
(a) providing a plurality of nuclei or cells in a first plurality of compartments, wherein each compartment comprises a subset of nuclei or cells;
(b) labeling and processing RNA molecules in the subsets of cells or nuclei obtained from the cells; wherein the labeling comprises adding to RNA molecules present in each subset of nuclei or cells a first compartment specific index sequence to result in indexed DNA nucleic acids present in indexed nuclei or cells, wherein the method comprises the steps of contacting the RNA molecules with a reverse transcriptase, a reverse transcription primer from a set of indexed reverse transcription primers that anneals to a polyA tail of RNA molecules, an indexed random hexamer primer from a set of indexed random hexamer primers, or a combination thereof;
(d) combining the indexed nuclei or cells to generate pooled indexed nuclei or cells;
(e) providing the plurality of nuclei or cells in a second plurality of compartments, wherein each compartment comprises a subset of nuclei or cells;
(f) labeling the indexed DNA nucleic acids in the subsets of cells or nuclei obtained from the cells; wherein the process of labeling comprises adding to the indexed DNA nucleic acids present in each subset of nuclei or cells a second compartment a specific indexed ligation primer from a set of indexed ligation primers to result in double indexed DNA molecules present in double indexed nuclei or cells, wherein the labeling comprises the steps of: contacting the indexed DNA molecules with a chemically modified DNA ligation primer/adaptor complex and a DNA ligase, and ligating the compartment specific DNA ligation primer to the indexed DNA molecules to generate double indexed single stranded DNA (ssDNA) molecules;
(g) combining the double indexed nuclei or cells to generate pooled double indexed nuclei or cells;
(h) providing the plurality of double indexed nuclei or cells in a third plurality of compartments, wherein each compartment comprises a subset of nuclei or cells;
(i) generating double indexed double stranded DNA (dsDNA) molecules by contacting the ssDNA molecules with a second-strand synthesis enzyme mix and synthesizing a second complementary DNA strand;
(j) performing bead-based purification of the double indexed dsDNA molecules;
(k) performing tagmentation on the purified dsDNA molecules;
(l) labeling the double indexed DNA nucleic acids in the subsets of cells or nuclei obtained from the cells; wherein the process of labeling comprises adding to the double indexed DNA molecules present in each subset of nuclei or cells a third compartment specific index sequence to result in triple indexed DNA nucleic acids present in triple indexed nuclei or cells, wherein the labeling comprises contacting the double indexed DNA molecules with a compartment specific indexed PCR primer (referred to as P7), a universal PCR primer (referred to as P5), and a polymerase, and performing PCR amplification of the double indexed DNA molecules to generate triple indexed DNA molecules.

In one embodiment, the reverse transcriptase comprises Maxima Reverse Transcriptase.

In one embodiment, the set of oligo-dT primers comprises a set of primers comprising sequences selected from the sequences as set forth in Table 3.

In one embodiment, the set of indexed random hexamer primers comprises a set of primers comprising sequences selected from the sequences as set forth in Table 4.

In one embodiment, the set of indexed ligation primers comprises a set of primers comprising sequences selected from the sequences as set forth in Table 5.

In one embodiment, the adaptor comprises SEQ ID NO: 2445.

In one embodiment, the ligation is performed using T4 ligase.

In one embodiment, the method further includes one or more steps selected from the group consisting of:
a) nuclei extraction;
b) nuclei fixation; and
c) nuclei storage
which are performed prior to step a) of claim 1.

In one embodiment, the step of nuclei extraction is performed using a buffer comprising 1% DEPC and 0.1% SUPREase.

In one embodiment, the step of nuclei fixation is performed by contacting extracted nuclei with 0.1% formaldehyde for 10 minutes.

In one embodiment, the method of nuclei storage comprises contacting nuclei with 10% DMSO and then freezing.

In one embodiment, the compartment comprises a well or a droplet.

In one embodiment, the compartments of the first plurality of compartments comprise from 50 to 20,000 nuclei or cells.

In one embodiment, the compartments of the second plurality of compartments comprise from 50 to 20,000 nuclei or cells.

In one embodiment, the compartments of the third plurality of compartments comprise from 50 to 20,000 nuclei or cells.

In one embodiment, the method further comprises pooling and collecting the triple indexed nucleic acids, thereby producing a sequencing library from the plurality of nuclei or cells.

In one embodiment, the invention relates to a kit for use in preparing a sequencing library, the kit comprising at least one set of indexed oligonucleotides.

In one embodiment, the kit comprises a set of 192 indexed primers as set forth in Table 3.

In one embodiment, the kit comprises a set of 192 indexed primers as set forth in Table 4.

In one embodiment, the kit comprises a set of 382 indexed primers as set forth in Table 5.

In one embodiment, the invention relates to a method for preparing a sequencing library for determination of transcriptome kinetics, the method comprising:
a) providing a plurality of cells comprising an expression construct for expression of a catalytically dead Cas9 protein;
b) contacting the cells of a) with an sgRNA library;
c) culturing the cells of b) in the presence of a selection agent for selection of cells containing an sgRNA library molecule;
d) splitting the cells of c) into
   i) a first population of cells for generation of a first “bulk” sequencing library; and
   ii) a second population of cells for subsequent culturing;
e) culturing the cells of d) ii) in the presence of at least one of:
   i) an inducing agent to induce expression of the catalytically dead Cas9 protein;
   ii) at least one agent for perturbing cells; and
   iii) at least one agent for sensitizing cells to perturbations;
f) culturing at least a portion of the cells of e) in the presence of an RNA metabolic label to label nascent transcripts;
g) splitting the cells of f) into
   i) a first population of cells for generation of a second “bulk” sequencing library; and
   ii) a second population of cells for subsequent chemical conversion and indexing;
h) chemically converting the RNA metabolic label in the RNA molecules from the cells of g) ii);
i) generating one or more sequencing library from the DNA molecules, RNA molecules, or a combination thereof, from the cells of step d) i), step g) i) and step h).

In one embodiment, the catalytically dead Cas9 protein is under the control of an inducible promoter.

In one embodiment, the promoter is inducible by contacting the cell with doxycycline (Dox).

In one embodiment, the inducing agent of step e) i) comprises doxycycline.

In one embodiment, the catalytically dead Cas9 protein comprises Dox-inducible dCas9-KRAB-MeCP2.

In one embodiment, the method of step e) iii) comprises culturing the cells in L-glutamine+, sodium pyruvate−, high glucose DMEM.

In one embodiment, the cell culture medium further comprises doxycycline.

In one embodiment, the sgRNA library comprises a library of plasmids encoding at least 500 different sgRNA molecules.

In one embodiment, the RNA metabolic label comprises 4-thiouridine (4sU).

In one embodiment, the method of step i) includes the steps of:
a) providing a plurality of nuclei or cells in a first plurality of compartments, wherein each compartment comprises a subset of nuclei or cells;
b) labeling and processing RNA molecules obtained from the cells; wherein the labeling comprises adding to RNA molecules present in each subset of nuclei or cells a first compartment specific index sequence to result in indexed DNA nucleic acids present in indexed nuclei or cells, wherein the method comprises the steps of contacting the RNA molecules with a reverse transcriptase, a reverse transcription primer from a set of indexed reverse transcription primers that anneals to a polyA tail of RNA molecules, an indexed random hexamer primer from a set of indexed random hexamer primers, or a combination thereof;
c) combining the indexed nuclei or cells to generate pooled indexed nuclei or cells;
d) providing the plurality of nuclei or cells in a second plurality of compartments, wherein each compartment comprises a subset of nuclei or cells;
e) labeling the indexed DNA nucleic acids in the subsets of cells or nuclei obtained from the cells; wherein the process of labeling comprises adding to the indexed DNA nucleic acids present in each subset of nuclei or cells a second compartment specific indexed ligation primer sequence to result in double indexed DNA molecules present in double indexed nuclei or cells, wherein the labeling comprises the steps of: contacting the indexed DNA molecules with a chemically modified DNA ligation primer/adaptor complex and a DNA ligase, and ligating the compartment specific DNA ligation primer to the indexed DNA molecules to generate double indexed single stranded DNA (ssDNA) molecules;
f) combining the double indexed nuclei or cells to generate pooled double indexed nuclei or cells;
g) providing the plurality of double indexed nuclei or cells in a third plurality of compartments, wherein each compartment comprises a subset of nuclei or cells;
h) generating double indexed double stranded DNA (dsDNA) molecules by contacting the ssDNA molecules with a second-strand synthesis enzyme mix and synthesizing a second complementary DNA strand;
i) performing bead-based purification of the double indexed dsDNA molecules;
j) performing tagmentation on the purified dsDNA molecules; and
k) labeling the double indexed DNA nucleic acids in the subsets of cells or nuclei obtained from the cells; wherein the process of labeling comprises adding to the double indexed DNA molecules present in each subset of nuclei or cells a third compartment specific index sequence to result in triple indexed DNA nucleic acids present in triple indexed nuclei or cells, wherein the labeling comprises contacting the double indexed DNA molecules with a compartment specific indexed PCR primer (referred to as P7), a universal PCR primer (referred to as P5), and a polymerase, and performing PCR amplification of the double indexed DNA molecules to generate triple indexed DNA molecules.

In one embodiment, the set of oligo-dT primers comprises a set of primers comprising sequences selected from the sequences as set forth in Table 3.

In one embodiment, the set of indexed random hexamer primers comprises a set of primers comprising sequences selected from the sequences as set forth in Table 4.

In one embodiment, the set of indexed ligation primers comprises a set of primers comprising sequences selected from the sequences as set forth in Table 5.

In one embodiment, the adaptor comprises SEQ ID NO: 2445.

In one embodiment, the ligation is performed using T4 ligase.

In one embodiment, the method further includes one or more steps selected from the group consisting of:
a) nuclei extraction;
b) nuclei fixation; and
c) nuclei storage
which are performed prior to step a) of claim 2.

In one embodiment, the step of nuclei extraction is performed using a buffer comprising 1% DEPC and 0.1% SUPREase.

In one embodiment, the step of nuclei fixation is performed by contacting extracted nuclei with 0.1% formaldehyde for 10 minutes.

In one embodiment, the method of nuclei storage comprises contacting nuclei with 10% DMSO and then freezing.

In one embodiment, the compartment comprises a well or a droplet.

In one embodiment, the compartments of the first plurality of compartments comprise from 50 to 20,000 nuclei or cells.

In one embodiment, the compartments of the second plurality of compartments comprise from 50 to 20,000 nuclei or cells.

In one embodiment, the compartments of the third plurality of compartments comprise from 50 to 20,000 nuclei or cells.

In one embodiment, the method further comprising pooling and collecting the triple indexed nucleic acids, thereby producing a sequencing library from the plurality of nuclei or cells.

In one embodiment, the invention relates to a method for preparing a sequencing library comprising nucleic acids from a plurality of single nuclei or cells, the method comprising:
(a) contacting a plurality of nuclei or cells with 5-Ethynyl-2-deoxyuridine (EdU);
(b) contacting the plurality of nuclei or cells with reagents for Click chemistry ligation to an azide-containing fluorophore;
(c) sorting the nuclei in a first plurality of compartments, wherein each compartment comprises a subset of nuclei or cells, wherein the sorting enriches for EdU+ nuclei or cells;
(d) labeling and processing RNA molecules in the subsets of cells or nuclei obtained from the cells; wherein the labeling comprises adding to RNA molecules present in each subset of nuclei or cells a first compartment-specific index sequence to result in indexed DNA nucleic acids present in indexed nuclei or cells, wherein the method comprises the steps of contacting the RNA molecules with a reverse transcriptase, an Oligo-dT primer that anneals to a polyA tail of RNA molecules and an indexed random primer;
(e) combining the indexed nuclei or cells to generate pooled indexed nuclei or cells;
(f) sorting the plurality of nuclei or cells into a second plurality of compartments, wherein each compartment comprises a subset of nuclei or cells;
(g) generating double stranded DNA (dsDNA) molecules by contacting the ssDNA molecules with a second-strand synthesis enzyme mix and synthesizing a second complementary DNA strand;
(h) performing tagmentation on the dsDNA molecules; and
(i) labeling the DNA nucleic acids in the subsets of cells or nuclei obtained from the cells; wherein the process of labeling comprises adding to the indexed DNA molecules present in each subset of nuclei or cells an additional compartment specific-index sequence to result in multi-indexed DNA nucleic acids present in multi-indexed nuclei or cells, wherein the labeling comprises contacting the indexed DNA molecules with a compartment specific indexed PCR primer (referred to as P7), a universal PCR primer (referred to as P5), and a polymerase, and performing PCR amplification of the double indexed DNA molecules to generate multi-indexed DNA molecules.

In one embodiment, the sorting in steps (c) and (f) is performed using FACS sorting gated for fluorophore and DAPI positive nuclei.

In one embodiment, the oligo-dT primer comprises a 5′ end as set forth in SEQ ID NO:2447 and a 3′ end as set forth in SEQ ID NO:2448 flanking a barcode sequence, wherein the barcode sequence comprises any nucleotide sequence from 5 to 20 nucleotides in length.

In one embodiment, the compartments of the first plurality of compartments comprise from about 250 to 500 nuclei or cells.

In one embodiment, the compartments of the second plurality of compartments comprise about 25 nuclei or cells.

In one embodiment, the method further comprises pooling and collecting the multi-indexed nucleic acids, thereby producing a sequencing library from the plurality of nuclei or cells.

In one embodiment, the invention relates to a method for preparing a sequencing library comprising nucleic acids from a plurality of single nuclei or cells, the method comprising:
(a) contacting a plurality of nuclei or cells with 5-Ethynyl-2-deoxyuridine (EdU);
(b) contacting the plurality of nuclei or cells with reagents for Click chemistry ligation to an azide-containing fluorophore;
(c) permeabilizing the nuclei or cells;
(d) sorting the nuclei in a first plurality of compartments, wherein each compartment comprises a subset of nuclei or cells, wherein the sorting enriches for EdU+ nuclei or cells;
(e) performing tagmentation on the nucleic acid molecules using a barcoded transposase;
(f) combining the indexed nuclei or cells to generate pooled indexed nuclei or cells;
(g) sorting the plurality of nuclei or cells into a second plurality of compartments, wherein each compartment comprises a subset of nuclei or cells; and
(h) labeling the DNA nucleic acids in the subsets of cells or nuclei obtained from the cells; wherein the process of labeling comprises adding to the indexed DNA molecules present in each subset of nuclei or cells an additional compartment specific-index sequence to result in multi-indexed DNA nucleic acids present in multi-indexed nuclei or cells, wherein the labeling comprises contacting the indexed DNA molecules with a compartment specific indexed PCR primer (referred to as P7), a universal PCR primer (referred to as P5), and a polymerase, and performing PCR amplification of the double indexed DNA molecules to generate multi-indexed DNA molecules.

In one embodiment, the sorting in steps (d) and (g) is performed using FACS sorting gated for fluorophore and DAPI positive nuclei.

In one embodiment, the compartments of the first plurality of compartments comprise from about 250 to 500 nuclei or cells.

In one embodiment, the compartments of the second plurality of compartments comprise about 25 nuclei or cells.

In one embodiment, the method further comprises pooling and collecting the multi-indexed nucleic acids, thereby producing a sequencing library from the plurality of nuclei or cells.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1a through FIG. 1k depict data demonstrating that EasySci enables high-throughput and low-cost single-cell transcriptome and chromatin accessibility profiling across the entire mammalian brain. FIG. 1a-b: EasySci-RNA workflow. Key steps are outlined in the texts. FIG. 1b: Pie chart showing the estimated cost compositions of library preparation for profiling 1 million single-cell transcriptomes using EasySci-RNA. FIG. 1c: Density plot showing the gene body coverage comparing single-cell transcriptome profiling using 10× genomics and EasySci-RNA. Reads from indexed oligo-dT priming and random hexamers priming are plotted separately for EasySci-RNA. FIG. 1d: Barplot showing the number of unique transcripts detected per cell comparing 10× genomics and an EasySci-RNA library at similar sequencing depth (˜20,000 raw reads/cell). FIG. 1e: Experiment scheme to reconstruct a brain cell atlas of both gene expression and chromatin accessibility across different ages, sexes, and genotypes. FIG. 1f: Barplot showing the cell-type-specific proportion in the brain cell population profiled by EasySci-RNA. FIG. 1g: UMAP visualization of mouse brain cells from single-cell transcriptome (Top) and chromatin accessibility (Bottom) analysis, colored by main cell types in (FIG. 1f). FIG. 1h: Heatmap showing the aggregated gene expression (Top) and gene body accessibility (Bottom) of the top ten marker genes (columns) in each main cell type (rows). For both RNA-seq and ATAC-seq, unique reads overlapping with the gene bodies of cell-type-specific markers were aggregated, normalized first by library size and then by the maximum expression or accessibility across all cell types. FIG. 1i: Scatter plot showing the fraction of each cell type in the global brain population by single-cell transcriptome (x-axis) or chromatin accessibility analysis (y-axis) in EasySci. FIG. 1j-k: Mouse brain sagittal (FIG. 1j) and coronal (FIG. 1k) sections showing the H&E staining (Left) and the localizations of main neuron types through NNLS-based integration (Right), colored by main cell types in (FIG. 1f). The numbers correspond to cell-type-specific cluster-ID in (FIG. 1f).

FIG. 2 depicts a summary of key optimizations of EasySci-RNA compared to published single-cell RNA-seq by combinatorial indexing (sci-RNA-seq3 (Cao et al., Nature 566, 496-502 (2019)).

FIG. 3a through FIG. 3n depict representative examples showing the performance of optimized conditions of EasySci-RNA. FIG. 3a-b: Boxplots showing the number of unique transcripts detected per nucleus in different lysis conditions: 1% DEPC vs. no DEPC in lysis buffer (FIG. 3a); EZ lysis buffer vs. nuclei lysis buffer used in the published sci-RNA-seq3 (Cao et al., Nature 566, 496-502 (2019) (FIG. 3b). FIG. 3c-d: Boxplot showing the number of unique transcripts detected per nucleus across different fixation conditions: formaldehyde vs. paraformaldehyde (FIG. 3c); 0.1% formaldehyde vs. 1% formaldehyde (FIG. 3d). FIG. 3e-f: Two conditions were compared for preserving the fixed nuclei. The slow freezing condition (in 10% DMSO) outperformed the flash freezing condition in sci-RNA-seq3 (Cao et al., Nature 566, 496-502 (2019) by increasing the number of nuclei recovered in the experiment (FIG. 3e) and the number of unique transcripts detected per nucleus (FIG. 3f). FIG. 3g-h: Maxima reverse transcriptase greatly reduces the enzyme cost (FIG. 3g) without affecting the number of transcripts detected per nucleus (FIG. 3h). FIG. 3i-j: Both short oligo-dT and random primers were included in reverse transcription to increase the number of unique transcripts (FIG. 3i) and genes (FIG. 3j) detected per nucleus. FIG. 3k: EasySci-RNA used T4 ligase instead of quick ligase for a higher recovery rate of nuclei. FIG. 3l: Chemically modified ligation primers were used in EasySci, which greatly reduced primer dimers in the following PCR reaction and slightly increased the number of unique transcripts detected per nucleus. FIG. 3m: Additional cDNA purification step after second strand synthesis increased the number of unique transcripts per nucleus. FIG. 3n: The efficiency of the novel EasySci-RNA method was compared with the sci-RNA-seq3 using mouse brain nuclei. The raw data was subset to 4448 reads/cell to remove any potential bias from sequencing depth.

FIG. 4a through FIG. 4c depict representative examples showing the performance of optimized conditions of EasySci-ATAC. Two fixation conditions were compared: nuclei were either fixed with 1% formaldehyde for 10 minutes at room temperature or directly used for tagmentation without fixation. The unfixed condition outperformed the fixed condition by increasing cell recovery (FIG. 4a), the number of reads (FIG. 4b) and the ratio of reads in promoters (FIG. 4c) per nucleus.

FIG. 5a through FIG. 5f depict data demonstrating the performance of EasySci-RNA and EasySci-ATAC profiling of mouse brain samples. FIG. 5a-b: Scatter plots showing the number of single-cell transcriptomes (FIG. 5a) and single-cell chromatin accessibility (FIG. 5b) profiled in each mouse individual across five conditions, colored by sex. Of note, the number of cells recovered from two mouse individuals in the EOAD model (RNA) are very close and cannot be separated in the plot. FIG. 5c-d: Boxplots showing the number of unique transcripts (FIG. 5c) and genes (FIG. 5d) detected per nucleus in each condition profiled by EasySci-RNA. FIG. 5e-f: Boxplots showing the number of unique fragments (FIG. 5e) and the ratio of reads in promoters (FIG. 5f) per cell in each condition profiled by EasySci-ATAC.

FIG. 6a through FIG. 6b depict data demonstrating identification of main brain cell types and cell-type-specific markers by EasySci-RNA. FIG. 6a: Dot plot showing the number of single-cell transcriptomes recovered from each individual, colored by conditions. FIG. 6b: UMAP plots showing the gene expression of identified novel markers for Microglia (Arhgap45, Wdfy4), Astrocytes (Clerr, Adamts9), and Oligodendrocytes (Sec14l5, Galnt5). UMI counts for these genes are scaled by the library size, log-transformed, and then mapped to Z scores.

FIG. 7a through FIG. 7c depict data demonstrating identification of cell-type-specific isoforms in the mouse brain. FIG. 7a: RandomN primed EasySci-RNA reads from each main cell type were aggregated in every mouse individual, yielding 617 pseudocells. The tSNE plot showed the separation of main cell types by isoform expression. FIG. 7b: Violin plots showing the expression of gene App and isoform App-202 across main cell types. FIG. 7c: Violin plots showing the expression of gene Aplp2 and isoform Aplp2-209 across main cell types. White circles represent the normalized expression of genes and isoforms (log(1+TPM)). White bars represent standard deviation.

FIG. 8a through FIG. 8d depict data demonstrating the characterization of cell-type-specific chromatin accessibility and key TF regulators using EasySci-ATAC. FIG. 8a: UMAP plot of the EasySci-ATAC dataset subsampled to 5,000 cells per cell type (or all cells if the number of cells is less than 5,000), colored by main cell types in FIG. 1g. The analysis was performed using the peak-count matrix without integration with RNA-seq dataset. FIG. 8b: Barplot showing the number of cell-type-specific peaks for each main cell type (defined as differential accessible sites across main cell types with q-value<0.05 and TPM>20 in the target cell type). FIG. 8c: Heatmap showing the aggregated accessibility of top 100 DA peaks per cell type (ranked by fold change between the maximum and the second accessible cell type). Unique counts for cell-type-specific peaks are first aggregated, normalized by the library size, and then mapped to Z-scores. FIG. 8d: Scatter plots showing the correlation between gene expression and motif accessibility of cell-type specific TF regulators, together with a linear regression line. TF gene expressions are calculated by aggregating scRNA-seq gene counts for each main cluster, normalized by the library size, and then mapped to Z-scores. TF motif accessibilities are quantified by chromVar (Schep et al., Nat. Methods 14, 975-978 (2017)), then aggregated per main cell type and mapped to Z-scores.

FIG. 9a through FIG. 9j depict data demonstrating the identification and characterization of cell sub-clusters of the mouse brain. FIG. 9a: Schematic plot showing the computational framework for identifying and characterizing cell sub-clusters. Each main cell type was subjected to sub-clustering analysis based on both gene and exon expression. Genes were then clustered into gene modules based on their expression pattern across all sub-clusters. Further, the spatial location of rare cell types was mapped through spatial transcriptomic analysis. FIG. 9b: By sub-clustering analysis, a total of 362 sub-clusters across 31 main cell types was identified. The barplot (Left) shows the number of sub-clusters for each main cell type. The dot plot (Right) shows the number of cells from each sub-cluster. The two smallest sub-clusters (choroid plexus epithelial cells-7 and vascular leptomeningeal cells-2) are circled out. FIG. 9c: UMAP visualizations showing sub-clustering analysis for choroid plexus epithelial cells (Top) and vascular leptomeningeal cells (Bottom) colored by sub-cluster IDs, highlighting two rare sub-clusters shown in (FIG. 9b). FIG. 9d: Dot plot showing the expression of selected marker genes for choroid plexus epithelial cells_7 (Top) and vascular leptomeningeal cells_2 (Bottom), including both normal genes (Left five genes) and transcription factors (Right five genes). FIG. 9e: UMAP visualizations of genes colored by identified gene module IDs. FIG. 9f: Scatterplots showing examples of gene modules and their expression levels across sub-clusters (ordered by gene module expression): GM-11 is specific to ependymal cells; GM-9 is specific to pituitary cell-6 (corticotropic cells); GM-6 marks four proliferating sub-clusters from different main cell types. FIG. 9g: UMAP visualization showing four proliferating sub-clusters identified from OB neurons 1, astrocytes, oligodendrocyte progenitor cells, and microglia, colored by the normalized expression of canonical proliferating marker Mki67 (Top) and the aggregated expression of lncRNAs in GM-6 (Bottom). UMI counts are first normalized by library size, log-transformed, aggregated (for multiple genes), and then mapped to Z-scores. FIG. 9h-i: Plots showing the normalized expression of gene modules in spatial transcriptomic datasets profiling mouse sagittal (Left) and coronal (Right) sections: GM-11, specific to ependymal cells, was mapped along all brain ventricles (FIG. 9h); GM-6, specific to proliferating cells, was mapped to proliferation active areas including subventricular zone (FIG. 9i). FIG. 9J: Similar to (FIG. 9h), plots showing the normalized expression of gene modules in spatial transcriptomic dataset profiling a mouse coronal section. UMI counts for genes from each gene module are scaled for library size, log-transformed, aggregated, and then mapped to Z scores.

FIG. 10a through FIG. 10c depict data characterizing microglia subtypes incorporating both gene and exon level expression. FIG. 10a-b: UMAP analysis of microglia cells was performed based on gene expression alone (FIG. 10a), or both gene and exon level expression (FIG. 10b). Cells are colored by sub-cluster ID from Louvain clustering analysis with combined gene and exon level information. Several sub-clusters cannot be separated from each other in the UMAP space by gene expression alone. FIG. 10c: UMAP plots same as (FIG. 10a) and (FIG. 10b), showing the expression of an exonic marker Ttr-ENSMUSE00000477272.5 of microglia sub-cluster 13. Microglia-13 can be better separated when combining both gene and exon level information. FIG. 10d: UMAP plots same as (FIG. 10b), showing the specific expression of an example exon marker Map2-ENSMUSE00000443205.3 (left) of microglia sub-cluster 8 and the lack of specificity of its corresponding gene Map2 (right). Single-cell gene expression was normalized first by library size, log-transformed, and then scaled to Z-scores.

FIG. 11a through FIG. 11b depict exemplary characteristics of subclusters. FIG. 11a: Density plot showing the number of individuals per subcluster. The rug plot below the density plot represents the individual subclusters. FIG. 11b: Density plot of the number of marker exons per subcluster. The rug plot below the density plot represents the individual subclusters.

FIG. 12 depicts the characterization of cell types/subtypes by gene module expression. Scatter plot showing the expression of each gene module across 362 sub-clusters. The associated cell types were annotated on the plot. UMI counts for genes from each gene module are scaled for library size, log-transformed, aggregated, and then mapped to Z scores.

FIG. 13a through FIG. 13h depict data identifying brain cell population changes across the lifespan at sub-cluster resolution. FIG. 13a: Dot plots showing the cell-type-specific fraction changes (i.e., log-transformed fold change) of main cell types and sub-clusters in the early growth stage (adult vs. young, left plot) and the aging process (aged vs. adult, right plot) in EasySci-RNA data. Differential abundant sub-clusters were colored by the direction of changes. Representative sub-clusters were labeled along with top gene markers. FIG. 13b: Scatter plots showing the correlation of the sub-cluster specific fraction changes between males and females in the early growth stage (top) and the aging stage (bottom), with a linear regression line. The most significantly changed sub-clusters are annotated on the plots. FIG. 13c: Examples of development- or aging-associated subclusters are highlighted in (FIG. 13a) and their spatial positions. Left: scatterplots showing the aggregated expression of sub-cluster-specific marker genes across all sub-clusters. Right: plots showing the aggregated expression of sub-cluster-specific marker genes across a brain sagittal section in 10× Visium spatial transcriptomics data. UMI counts for gene markers are scaled for library size, log-transformed, aggregated, and then mapped to Z scores. FIG. 13d: Line plots showing the relative fractions of depleted subclusters across three age groups identified from EasySci-RNA (left) and EasySci-ATAC (right). FIG. 13e: Scatter plots showing the correlated gene expression and motif accessibility of transcription factors enriched in OB neurons 1-17 (Sox2 and E2F2, left and middle) and oligodendrocytes-7 (Stat3, right), together with a linear regression line. FIG. 13f: Box plots showing the fractions of the reactive microglia (left) and reactive oligodendrocytes (right) across three age groups profiled by EasySci-RNA (top) and EasySci-ATAC (bottom). FIG. 13g-h: Mouse brain coronal sections showing the expression level of C4b (FIG. 13g) and Serpina3 (FIG. 13h) in the adult (left) and aged (right) brains from spatial transcriptomics analysis.

FIG. 14a through FIG. 14d depict data demonstrating the identification of cell subtypes underlying olfactory bulb expansion from the young to adult stage in EasySci-RNA and EasySci-ATAC. FIG. 14a: Heatmaps showing the aggregated gene expression (top) and gene body accessibility (bottom) of sub-cluster specific gene markers (columns) in OB expansion-associated sub-clusters (rows) from OB neurons 1 (left), OB neurons 2 (middle), and OB neurons 3 (right). UMI counts for genes or reads overlapping with gene bodies were aggregated for each sub-cluster, normalized first by the total number of reads, column centered, and scaled across all cell sub-clusters. FIG. 14b-c: UMAP visualization showing astrocytes subtype 14 (FIG. 14b) and vascular leptomeningeal cells (VLC) subtype 14 (FIG. 14c), colored by subcluster ID in EasySci-RNA (top left) and EasySci-ATAC (bottom left), the aggregated gene expression (top right) and gene body accessibility (bottom right) of sub-cluster specific gene markers. FIG. 14d: For the OB expansion-related sub-clusters, their log 2-transformed fold changes were plotted between each age group and the young mice, profiled by EasySci-RNA (left) and EasySci-ATAC (right).

FIG. 15a through FIG. 15d depict data demonstrating identification of reduced endothelial cells in the aged brain by spatial transcriptomics. FIG. 15a: Boxplot showing the aggregated expression of endothelial marker genes across single cells recovered from adult and aged brains. The top ten gene markers of endothelial cells (FDR of 5%, ordered by q-value in differentiation gene analysis) were first selected. Next, three gene markers that significantly changed in aging (FDR of 5%) were filtered out. The remaining seven genes were combined as the gene module for marking endothelial cells in adult and aged brains: Rgs5, Nostrin, Ly6c1, Zfp366, Abcc9, Emen, Ptprb, Adgrl4, Flt1, Slc38a11. UMI counts for these genes are scaled for library size, log-transformed, aggregated, and then mapped to Z scores. FIG. 15b: UMAP visualization of all spatial spots from spatial transcriptomic analysis of adult, aged and 5×FAD brains, colored by conditions (left) or spatial clusters (right). FIG. 15c: Plots showing the mouse brain coronal sections (left) and the distribution of identified spatial clusters (right) in spatial transcriptomic datasets profiling adult (top) and aged (bottom) brains. FIG. 15d: Boxplots showing the expression of endothelial markers across all spatial spots (left) and across spatial spots within each spatial cluster (right) between adult and aged brains.

FIG. 16a through FIG. 16d depict data identifying aging-associated sub-clusters related to neurogenesis, oligodendrogenesis, and inflammation in EasySci-ATAC. FIG. 16a: UMAP visualization showing OB neurons 1-11 and OB neurons 1-17 identified from EasySci-RNA (top) and EasySci-ATAC (bottom), colored by subcluster id (left), aggregated gene expression or gene activity of OB neurons 1-11 gene markers (middle) and OB neurons 1-17 gene markers (right). FIG. 16b: UMAP visualization showing oligodendrocytes-6 and oligodendrocytes-7 identified from EasySci-RNA (top) and EasySci-ATAC (bottom), colored by subcluster id (left), aggregated gene expression or gene activity of oligodendrocytes-6 gene markers (middle) and oligodendrocytes-7 markers (right). FIG. 16 c: UMAP visualization showing microglia-9 identified from EasySci-RNA (top) and EasySci-ATAC (bottom), colored by subcluster id (left), aggregated gene expression or gene activity of microglia-9 gene markers (right). Subcluster marker genes were identified by differential expression analysis using scRNA-seq data. FIG. 16d: Heatmap showing the gene expression (top) and the promoter accessibility (bottom) of microglia-9 enriched genes across subclusters. The scRNA-seq data (UMI count matrix) and scATAC-seq data (read count matrix) were aggregated per sub-cluster, normalized by the total number of reads, column centered, and scaled.

FIG. 17a and FIG. 17b depict data demonstrating the identification of aging-associated gene expression changes across sub-clusters. FIG. 17a: Volcano plot showing the differentially expressed genes between aged and adult brains in all subclusters (left), colored by grey (not significant) or main cell types. FIG. 17b: The plots highlight several aging-associated gene markers, colored by main cell types.

FIG. 18a through FIG. 18l depict data identifying AD pathogenesis-associated gene expression signatures and cell subtypes. FIG. 18a: Volcano plots showing the differentially expressed (DE) genes between WT and EOAD model (top) or LOAD model (bottom) across all sub-clusters. Significantly changed genes are colored by the main cell type identity for the corresponding sub-cluster. FIG. 18b-c: Volcano plot same as (FIG. 18a), highlighting example DE genes with concordant changes across multiple sub-clusters comparing WT and EOAD (FIG. 18b) or LOAD (FIG. 18c) models, labeled with related biological pathways. FIG. 18d: Scatterplot showing the correlation of the number of DE genes identified in each sub-cluster between EOAD and LOAD, together with a linear regression line. FIG. 18e: 558 DE genes significantly changed within the same sub-cluster in both AD models (both compared with the wild-type). The scatterplot shows the correlation of the log 2-transformed fold changes of these 559 shared DE genes in EOAD model (x-axis) and LOAD model (y-axis). FIG. 18f: Dot plots showing the log-transformed fold changes of main cell types and sub-clusters comparing EOAD vs. WT (left) and LOAD vs. WT (right). Differential abundant sub-clusters were colored by the direction of changes. Representative sub-clusters were labeled along with top gene markers. FIG. 18g: Scatter plots showing the correlation of the log-transformed fold changes of sub-clusters (top: EOAD vs. WT, bottom: LOAD vs. WT) between male and female. FIG. 18h: Scatter plot showing the correlation of the log-transformed fold changes of sub-clusters in two AD models (both compared with the wild-type). Only sub-clusters showing significant changes in at least one AD model are included. FIG. 18i: Scatterplots showing the aggregated expression of gene markers of two cell subtypes (top: choroid plexus epithelial cells-4; bottom: the interbrain and midbrain neurons 1-4) across all sub-clusters from EasySci-RNA data. FIG. 18j: Brain coronal sections showing the spatial expression of subtype-specific gene markers of two subtypes (top: choroid plexus epithelial cells-4; bottom: the interbrain and midbrain neurons 1-4) in the WT and EOAD (5×FAD) brains in 10× Visium spatial transcriptomics data. FIG. 18k: Box plots showing the fraction of microglia-9 cells across different conditions profiled by EasySci-RNA (left) or EasySci-ATAC (right). FIG. 18l: Scatter plot showing the correlated gene expression and motif accessibility of four transcription factors (Nfe2l2, Nfkb1, Relb, and Srebf2) enriched in microglia-9, together with a linear regression line.

FIG. 19 depicts an agarose E-Gel quantification of the library concentration. Column M: 50 base pair ladder. Column 1: PCR product for the first 96-well plate, no purifications. Column 2: One 0.8× beads purification, plate one. Column 3: 0.8× purification and 0.9× purification, plate one. Column 4: PCR product for the second 96-well plate, no purifications. Column 5: One 0.8× beads purification, plate two. Column 6:0.8× purification and 0.9× purification, plate two.

FIG. 20a and FIG. 20f depict data demonstrating TrackerSci enables single-cell transcriptome and chromatin accessibility profiling of rare proliferating cells in the mammalian brain. FIG. 20a: TrackerSci workflow and experiment scheme. Key steps are outlined in the text. FIG. 20b-c: UMAP visualization of mouse brain cells, integrating the single-cell transcriptome and chromatin accessibility profiles of EdU+ cells and DAPI singlets (representing the global brain cell population). Cells are colored by sources (FIG. 20b, top), molecular layers (FIG. 20b, bottom), and main cell types (FIG. 20c). The identified neurogenesis and oligodendrogenesis trajectories are both annotated in (c). FIG. 20d: Pie plots showing the proportion of main cell types identified in the global cell population (left) and the enriched EdU+ cell population (right). FIG. 20e: Scatter plot showing the fraction of each cell type in the enriched EdU+ cell population by single-cell transcriptome (x-axis) or chromatin accessibility analysis (y-axis) in TrackerSci. FIG. 20f: The TrackerSci dataset, including both EdU+ cells and DAPI singlets, was integrated with a large-scale brain cell atlas comprising 1,469,111 cells. For the brain cell atlas, 5,000 cells of each cell type were sampled for the integration analysis. The UMAP plots show the integrated cells, colored by assay types (left, cell types from TrackerSci are annotated) or cell annotations from the brain cell atlas (right, cells from TrackerSci are colored in grey).

FIG. 21a and FIG. 21b depict data demonstrating that TrackerSci relies on two rounds of sorting to enrich and purify rare EdU+ proliferating cells in mammalian brains. FIG. 21a: Representative Fluorescent-activated cell sorting (FACS) scatter plots showing the percentage of EdU+ cells in mouse brains across different conditions during the first round of sorting. FIG. 21b: FACS scatter plot (left) and contour plot (right) showing the percentage of EdU+ cells during the second round of sorting in TrackerSci.

FIG. 22a through FIG. 22e depict the quality control of TrackerSci for single-cell transcriptome profiling. FIG. 22a: Boxplot showing the number of unique transcripts detected per cell (HEK293T nuclei) after different treatment conditions of click-chemistry (CC). The result indicated copper and reaction addictive in the conventional click-chemistry reaction decreased the scRNA-seq efficiency. FIG. 22b: Boxplot showing the number of unique transcripts detected per cell (mouse brain nuclei) across three conditions: no click-chemistry (No CC), conventional click-chemistry (CC), and click-chemistry plus condition (with picolyl azide dye and copper protectant, CC Plus). FIG. 22c: Scatter plots showing the number of unique human and mouse transcripts detected per cell across different conditions (with/without EdU labeling, with/without click chemistry plus reaction). FIG. 22d: Boxplot showing the number of unique transcripts (top) and genes (bottom) detected per cell in HEK293T and NIH/3T3 nuclei across the four conditions described in (FIG. 22c). FIG. 22e: Scatter plot showing the correlation between log-transformed aggregated gene expression profiled by TrackerSci and sci-RNA-seq in HEK293T cells (left) and mouse brain cells (right), together with the linear regression line (blue).

FIG. 23a through FIG. 23e depict the quality control of TrackerSci for single-cell chromatin accessibility profiling. FIG. 23a: Scatter plots showing the number of unique human and mouse ATAC-seq fragments detected per cell across different conditions (with/without EdU labeling, with/without click chemistry plus reaction). FIG. 23b: The aggregated fragment length distribution in ATAC-seq from TrackerSci of all cells across the four conditions described in FIG. 23a. FIG. 23c-d: Boxplots showing the number of unique ATAC-seq reads (Top) and the fraction of reads in promoters (Bottom) in HEK293T and NIH/3T3 nuclei (FIG. 23c) and mouse brain nuclei (FIG. 23d). FIG. 23e: Scatter plot showing the correlation between log-transformed aggregated ATAC-seq fragments (tags per million) profiled by TrackerSci and sci-ATAC-seq in HEK293T cells (top) and mouse brain cells (bottom), together with the linear regression line (blue). CC: click-chemistry. CC plus: click-chemistry plus condition (with picolyl azide dye and copper protectant).

FIG. 24 depicts data demonstrating increased expression of C4b in oligodendrocyte progenitor cells. Barplots showing the gene expression (left) and promoter accessibility (middle) of C4b from the TrackerSci dataset, and the gene expression of C4b from the EasySci dataset (right) in Oligodendrocytes progenitor cells (OPC) and committed oligodendrocyte precursors (COP), quantified by transcripts per million (TPM) for gene expression and reads per million for promoter accessibility. Error bars represent standard errors of the means.

FIG. 25a through FIG. 25e depict data demonstrating that TrackerSci recovered single-cell transcriptomes of rare newborn cells in the mammalian brain. FIG. 25a: Scatter plots showing the number of single-cell transcriptomes profiled in each mouse individual across four conditions, colored by sexes. Only mice from the main experiment group (EdU labeling for 5 days) are shown. FIG. 25b: Boxplot showing the log-transformed number of unique transcripts (left) and genes (right) detected per cell profiled by TrackerSci and the DAPI singlet (without enrichment of EdU+ cells, adult mouse brain). FIG. 25c-d: UMAP visualization of single-cell transcriptomes, including EdU+ cells (profiled by TrackerSci) and all brain cells (without enrichment of EdU+ cells), colored by experiments (FIG. 25c, top), conditions (FIG. 25c, bottom), and main cell types (FIG. 25d). FIG. 25e: Scatter plots showing the correlation of cell-type-specific fractions between two replicates (with relatively high numbers of cells recovered) in each condition profiled by single-cell RNA-seq analysis of TrackerSci.

FIG. 26a through FIG. 26e depict data demonstrating that TrackerSci recovered single-cell chromatin accessibility of rare newborn cells in the mammalian brain. FIG. 26a: Scatter plot showing the number of single-cell chromatin accessibility profiled in mouse individuals across four conditions, colored by sexes. Only mice from the main experiment group (EdU labeling for 5 days) are shown. FIG. 26b: Boxplot showing the fraction of reads in promoters and peaks (left) and the log-transformed number of unique ATAC-seq reads (right) detected per cell across different conditions in TrackerSci and the DAPI singlet (adult mouse brain, without enrichment of EdU+ cells). FIG. 26c-d: UMAP visualization of single-cell chromatin accessibility profiles, including EdU+ cells (profiled by TrackerSci) and all brain cells (without enrichment of EdU+ cells), colored by experiments (c, top), conditions (c, bottom), and main cell types (FIG. 26d). FIG. 26e: Scatter plots showing the correlation of cell-type-specific fractions between two replicates (with relatively high numbers of cells recovered) in each condition profiled by single-cell ATAC-seq analysis of TrackerSci.

FIG. 27 depicts data demonstrating that the cell population distributions are correlated between single-cell transcriptome and chromatin accessibility profiling of newborn cells in the mouse brain. Scatter plot showing the fraction of each cell type in the enriched EdU+ cell population by single-cell transcriptome (x-axis) or chromatin accessibility analysis (y-axis) in TrackerSci across different conditions.

FIG. 28 depicts a UMAP visualization of the full brain atlas dataset (˜1.5 million cells) with the same parameter settings as in FIG. 20f. Neurogenesis and oligodendrogenesis-related cell types are separated into distinct clusters, while the “bridge” cells in the intermediate stages are missing.

FIG. 29a through FIG. 29g depict data identifying epigenetic elements and transcription factors associated with heterogeneous cellular states of newborn cells in the mouse brain. FIG. 29a: Heatmap showing the relative expression (top) and chromatin accessibility (bottom) of cell-type-specific genes across cell types. The UMI count matrix (gene expression) and read count matrix (ATAC-seq) were normalized by the library size and then log-transformed, column centered, and scaled. The resulting values clamped to [−2, 2]. FIG. 29b: Density plot showing the distribution of Pearson correlation coefficients between gene expression and the accessibility of promoter (colored in red) or nearby accessible elements (within ±500 kb of the promoter, colored in blue) across pseudo-cells. In addition, the background distribution of the Pearson correlation coefficient was plotted after permuting the accessibility of peaks across pseudo-cells. FIG. 29c: Density plot showing the distribution of Pearson correlation coefficients between TF expression and their motif accessibility across pseudo-cells. The background distribution was calculated after permuting the motif accessibility of TFs across pseudo-cells. FIG. 29d: Genome browser plot showing links between distal regulatory sites and genes for a neurogenesis marker (Dlx2, top) and an oligodendrogenesis marker (Olig2, bottom). FIG. 29e: UMAP plots showing the cell-type-specific expression (left), the accessibility of promoter (middle), and linked distal site (right) for genes Dlx2 (top) and Olig2 (bottom). The single-cell expression data (UMI count) and ATAC-seq data (read count) were normalized first by library size and then log-transformed, column centered, and scaled. FIG. 29f: Scatter plots showing the correlation between the scaled gene expression and motif accessibility across cell types for Dlx2 (top) and Olig2 (bottom), together with a linear regression line. (ASC: astrocytes, CBGR: cerebellum granule neurons, COP: committed oligodendrocytes precursors, DGNB: dentate gyrus neuroblasts, ERY: erythroblasts, MFO: myelin-forming oligodendrocytes, MG: microglia, NPC: neuronal progenitor cells, OBNB: olfactory bulb neuroblasts, OBIN: olfactory bulb inhibitory neurons, OPC: oligodendrocytes progenitor cells, VEC: vascular endothelial cells). FIG. 29g: Scatter plots showing the correlation between the scaled gene expression and motif accessibility of less-characterized TF regulators, together with a linear regression line.

FIG. 30 depicts data identifying canonical and novel gene markers of neuronal progenitors and oligodendrocyte precursors. Each scatter plot shows the correlation between expression and promoter accessibility of known (left two columns) or novel (right two columns) cell-type-specific gene markers, together with a linear regression line.

FIG. 31 depicts data demonstrating the low cell-type-specificity of certain canonical neurogenesis markers. UMAP plots showing the expression of canonical neurogenesis markers (Sox2 and Dcx) across different cell types. The single-cell expression data (UMI count) were normalized first by the total number of reads for each cell and then log-transformed, column centered, and scaled.

FIG. 32a through FIG. 32e depict data demonstrating linking cis-regulatory elements and their regulated genes. FIG. 32a: UMAP visualization of EdU+ cells in FIG. 20b, colored by k-means clustering ID. FIG. 32b: The left histogram shows the number of accessible sites per gene. The right histogram shows the distance distribution of accessible sites within 500 kb of genes. Both plots include all nearby accessible sites (colored in black) and the linked accessible sites (colored in red). FIG. 32c: Heatmap showing the cell-type-specific peak accessibility of four Dlx2 linked sites. Cell types are ordered by hierarchical clustering. FIG. 32d: Heatmap showing the cell-type-specific peak accessibility of ten Olig2 linked sites. Cell types are ordered by hierarchical clustering. FIG. 32e: Barplots showing the average expression, the accessibility of promoter and linked distal sites for neurogenesis marker Dlx2 across different cell types. Gene expression values for each cell type were quantified by transcripts per million (TPM). Site accessibilities for each cell were quantified by the number of reads per million. Error bars represent standard errors of the means.

FIG. 33 depicts data identifying key transcription factor regulators of the newborn cells. Each scatter plot shows the correlation between cell-type-specific gene expression and motif accessibility for known TF regulators, together with a linear regression line.

FIG. 34a through FIG. 34h depict data deciphering the impact of ageing on the proliferation status and differentiation dynamics of different cell types in the mammalian brain. FIG. 34a: Boxplot showing the fraction of EdU+ cells in the mouse brain after five days of EdU labeling. The plot includes data from both single-cell transcriptome and chromatin accessibility analysis in TrackerSci. FIG. 34b: With the single-cell RNA-seq or ATAC-seq data of TrackerSci, the cell-type-specific fractions were first calculated in each condition (i.e., young, adult, aged, and 5×FAD), multiplied by the fraction of EdU+ cells in the entire brain. Then, the fold changes of normalized cell-type-specific fractions were quantified between the aged and adult brains. The scatter plot shows the correlation of the log-transformed fold changes (aged vs. adult) between single-cell transcriptome and chromatin accessibility analysis in TrackerSci. FIG. 34c: Similar to the analysis in (b), the dot plot shows the log-transformed cell-type-specific fold changes between each condition and the adult brain. FIG. 34d: Area plot showing the cell-type-specific proportions in EdU+ cells over time. FIG. 34e: Cells corresponding to OB neurogenesis (top), oligodendrogenesis (middle), and microglia (bottom) were integrated in TrackerSci and brain cell atlas; the left UMAP plot shows the integrated cells, colored by cell type annotations in TrackerSci or grey (brain cell atlas). The two UMAP plots on the right show cells from the brain cell atlas or the EdU+ cells recovered by TrackerSci, colored by the expression of the neuronal progenitor marker Mki67 (top), the committed oligodendrocyte precursor cells marker Bmp4 (middle) and the ageing/AD-associated microglia marker Csf1 (bottom). FIG. 34f: Box plots showing the cell-type-specific fractions of neuronal progenitor cells (top), committed oligodendrocyte precursors (middle) and ageing/AD-associated microglia (bottom) across different conditions in the brain cell atlas (left) or newborn cells from TrackerSci (right). FIG. 34g: Schematic showing how to calculate the self-renewal potential and differentiation potential of progenitor cells. FIG. 34h: Left: Line plot showing the estimated self-renewal potential of neuronal progenitor cells over time. Right: Line plot showing the estimated differentiation potential of the newly generated oligodendrocyte progenitor cells across three age groups.

FIG. 35a through FIG. 35e depict data characterizing the impact of ageing on the transcriptional and epigenetic regulations of neurogenesis and oligodendrogenesis. FIG. 35a: UMAP plots showing the differentiation trajectory of the neurogenesis trajectory (top) and the oligodendrogenesis trajectory (bottom), colored by main cell types (left) or pseudotime (right). The differentiation trajectories are inferred by RNA velocity analysis (left) and annotated on the right plot. FIG. 35b: Heatmap showing the dynamics of gene expression and motif accessibility of cell-type-specific TFs across the pseudotime of neurogenesis (left) and oligodendrogenesis (right) trajectories. FIG. 35c: Contour plots showing the distribution of EdU+ cells from TrackerSci-RNA in the neurogenesis trajectory (top) and oligodendrogenesis trajectory (bottom) across conditions. The arrows point to the significantly reduced cell states in each trajectory. FIG. 35d: A neighborhood graph from Milo differential abundance analysis on the neurogenesis trajectory (top) and oligodendrogenesis trajectory (bottom). The layout of the graph is determined by the position of the neighborhood index cell in FIG. 35a. Nodes represent cellular neighborhoods from the KNN graph. Differential abundance neighborhoods are colored by the log-transformed fold change across ages. Graph edges depict the number of cells shared between neighborhoods. FIG. 35e: The dot plots and heatmaps show the scaled gene expression and promoter accessibility of top differentially expressed genes in the neuronal progenitor cells (top) and oligodendrocyte progenitor cells (bottom).

FIG. 36 depicts data validating in vivo cell differentiation trajectory by a pulse-chase experiment. The mice brains were harvested one day, three days and nine days after EdU labeling (EdU was administered daily through i.p. injection during the first five days), followed by single-cell transcriptome analysis of EdU+ cells by TrackerSci. The contour plots show the distribution of EdU+ cells in the neurogenesis trajectory (left) and oligodendrogenesis trajectory (right) across conditions and the distribution of all brain cells without enrichment of EdU+ cells.

FIG. 37a through FIG. 37c depict data characterizing gene expression and chromatin accessibility dynamics along adult neurogenesis and oligodendrogenesis. FIG. 37a: Heatmap showing the dynamics of gene expression of 1,799 shared DE genes along DG neurogenesis (left) and OB neurogenesis (right). Genes are ordered and clustered by hierarchical clustering. Representative gene names (left) and enriched pathways (right) for each gene group are labeled. FIG. 37b: Heatmap showing examples TFs exhibiting trajectory-specific gene expression dynamics: Neurod1, Neurod2, Emx1, Stat3 and Rarb are uniquely upregulated in DG neurogenesis, while Dlx6, Ets1, Pbx1, Zfp711, Foxp2, Meis1 and Mef2c are uniquely upregulated in OB neurogenesis. FIG. 37c: Heatmap showing the dynamics of 8,443 DE genes (top) and 15,164 DA sites (bottom) along the oligodendrogenesis trajectory. Genes are ordered and clustered based on hierarchical clustering. Representative gene names (left) and enriched pathways (right) for each gene group are labeled. Peaks are ordered based on hierarchical clustering, and peaks corresponding to promoters of known and novel oligodendrogenesis markers are labeled.

FIG. 38 depicts an overview of ceramide/sphingomyelin metabolism. Sphingomyelin production from ceramide is catalyzed by sphingomyelin synthase and is hydrolyzed to ceramide by sphingomyelinase.

FIG. 39A through FIG. 39K depict data demonstrating that PerturbSci-Kinetics enables joint profiling of transcriptome dynamics and high-throughput gene perturbations by pooled CRISPR screens. FIG. 39A: Scheme of the experimental and computational strategy for PerturbSci-Kinetics. The dot plot on the upper right shows the number of cells profiled in this study compared to published single-cell metabolic profiling datasets. IAA, iodoacetamide. Asterisk, chemically modified 4sU. R, steady-state RNA level. α, mRNA synthesis rate. β, mRNA degradation rate. Exp, steady-state expression. Synth, synthesis rates. Deg, degradation rates. FIG. 39B: Barplot showing the estimated library preparation cost across different single-cell perturbation techniques. FIG. 39C: Scatter plot showing the number of unique sgRNA transcripts detected per cell in the experiment for profiling cells transduced with sgNTC or sgIGF1R. FIG. 39D: The left boxplot shows the normalized expression of dCas9-KRAB-MeCP2 in untreated and Dox-induced HEK293-idCas9 cells. The right boxplot shows the normalized expression of IGF1R in induced HEK293-idCas9 transduced with sgNTC/sgIGF1R. Gene counts of each single cell were normalized to a total of 1e4 to ease the batch effect caused by different sequencing depths across single cells, and were then log-transformed for visualization. FIG. 39E: Barplot showing normalized fractions of all possible single base mismatches in reads from sci-fate, PerturbSci-kinetics on unconverted cells, and PerturbSci-Kinetics on labeled converted cells. The single-base alignment information was retrieved from a subset of cells, and the strandness was considered. Then the normalized mismatch rates were calculated by dividing the counts of 12 mismatches by the total number of single bases aligned. FIG. 39F: Boxplot showing the fraction of recovered nascent reads in single-cell transcriptomes across conditions: no 4sU labeling+no chemical conversion, 4sU labeling+no chemical conversion, and 4sU labeling+chemical conversion. FIG. 39G: Boxplot comparing the ratio of reads mapped to exonic regions of the genome between nascent reads, pre-existing reads, and reads of whole transcriptomes of single cells. FIG. 39H-FIG. 39I: Barplots showing the significantly enriched Gene Ontology (GO) terms in analyzing the list of genes with low (FIG. 39H) or high (FIG. 39I) nascent reads ratio. FIG. 39J: Boxplot comparing the number of unique sgRNA transcripts detected per cell in cells with or without the chemical conversion. FIG. 39K: Stacked barplot showing the fraction of cells identified as sgNTC/sgIGF1R singlets, doublets, and cells without sgRNA detected in cells with or without chemical conversion.

FIG. 40A and FIG. 40B depict a scheme of plasmids and experiment procedures of PerturbSci. FIG. 40A: The vector system used in PerturbSci for sgRNA expression and CRISPRi. FIG. 40B: The library preparation scheme and the final library structures of PerturbSci.

FIG. 41A through FIG. 41L depict representative optimizations on sgRNA capture, sgRNA enrichment strategy, and fixation conditions. FIG. 41A: Multiple RT primers targeting different gRNA scaffold regions were included in the test experiment for targeted enrichment of gRNA. FIG. 41B: The enrichment efficiency of different RT primers was tested in PerturbSci with (Direct PCR) or without (sgRNA-only PCR) tagmentation (Scheme shown in FIG. 41B), analyzed by gel electrophoresis (FIG. 41C). As shown in c, gRNA primers 2 and 3 both yielded reasonable amplification signals following PCR, compared with other primers. FIG. 41D: Different purification conditions were tested for recovery of the gRNA library. Left lane: 0.7× Ampure beads purification post second strand synthesis+1.5× Ampure beads purification post PCR. Middle lane: 0.8× Ampure beads purification post second strand synthesis+1.2× Ampure beads purification post PCR. Right lane: 0.8× Ampure beads purification post second strand synthesis+gel purification post PCR. FIG. 41E: Gel Electrophoresis showing PCR products of the final libraries including sgRNA library (Lane 1) and the transcriptome library (Lane 2). FIG. 41F: Boxplot showing the number of unique sgRNA transcripts detected per cell with different sgRNA RT primer concentrations in both sgFto and sgNTC conditions. FIG. 41G: Boxplot showing the number of unique transcripts detected per cell with different sgRNA RT primer concentrations in both sgFto and sgNTC conditions. FIG. 41H: Boxplot showing normalized cell number with different sgRNA RT primer concentrations in both sgFto and sgNTC conditions. FIG. 41I: Boxplot showing sgRNA capture purity with different sgRNA RT primer concentrations. FIG. 41J: Boxplot showing the number of unique sgRNA transcripts detected with pooled or separated method in both sgFto and sgNTC conditions. FIG. 41K: Boxplot showing sgRNA capture purity with pooled or separated method. 1. Scatter plot showing the correlation between log-transformed aggregated gene expression profiled by PerturbSci and EasySci in a mouse 3T3-L1-CRISPRi cell line.

FIG. 42A through FIG. 42F depict representative optimizations on fixation conditions for chemical conversion and quality control on chemical conversion. FIG. 42A: Stacked barplot showing the fraction of cells identified as sgNTC, sgIGF1R, mixed, unmatched with different fixation conditions. FIG. 42B: Boxplot showing the number of unique sgRNA transcripts detected per cell with different fixation conditions. FIG. 42C: Boxplot showing the number of unique transcripts detected per cell with different fixation conditions. FIG. 42D: Dot plot showing the relative recovery rate of HEK293-idCas9 cells fixed in different fixation conditions after 0.05N HCl permeabilization step. FIG. 42E: Dot plot showing the relative recovery rate of HEK293-idCas9 cells fixed in different fixation conditions after chemical conversion. FIG. 42F: Boxplot showing the number of unique transcripts detected per cell in control and chemical conversion condition.

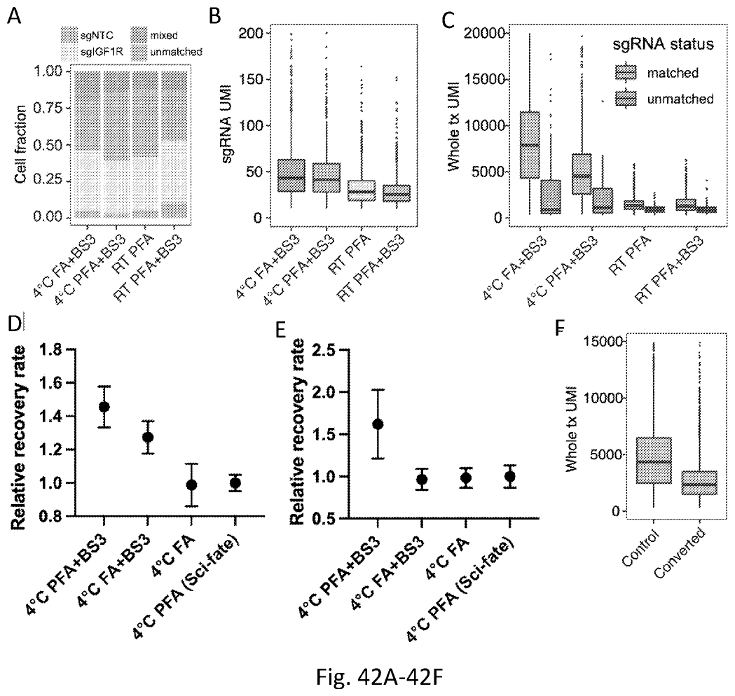

FIG. 43A and FIG. 43B depict data demonstrating strongly reduced IGF-1R mRNA and protein levels after Dox induction were further validated by FIG. 43A: RT-qPCR and FIG. 43B: flow cytometry.

FIG. 44A through FIG. 44Q depict data characterizing the impact of genetic perturbations on gene-specific transcriptional and degradation dynamics with PerturbSci-Kinetics. FIG. 44A: Scheme of the experimental design of the PerturbSci-Kinetics screen. The main steps are described in the text. FIG. 44B: UMAP visualization of genetic perturbations profiled by PerturbSci-Kinetics. Single-cell transcriptomes in each genetic perturbation were aggregated, followed by dimension reduction using PCA and UMAP. Population classes: the functional categories of genes targeted in different perturbations. FIG. 44C: The Scatter plot shows the correlation between perturbation-associated cell count (PerturbSci-Kinetics) and sgRNA read counts (bulk screen). FIG. 44D through FIG. 44F: Boxplot showing the log 2 transformed fold change of gene expression (FIG. 44D), synthesis rates (FIG. 44E), and degradation rate (FIG. 44F) of target genes across perturbations compared with the control sgRNA. FIG. 44G through FIG. 44J: Scatter plots showing the extent and the significance of changes on the distributions of global synthesis (FIG. 44G), degradation (FIG. 44H), nascent exonic reads ratio (FIG. 44I), and mitochondrial transcriptome turnover (FIG. 44J) upon perturbations compared with the control sgRNA. The effect size was calculated using the fold changes in the median value of detected genes between each perturbation and the control sgRNAs. FIG. 44K: Boxplot showing the proportion of degradation-regulated differentially expressed genes (DEGs) in all DEGs showing significant changes in synthesis/degradation rates across perturbations. FIG. 44L: Scatter plot showing the number of synthesis/degradation-regulated DEGs of different perturbations. nDEGs: the number of DEGs. FIG. 44M: Top20 perturbations ordered by the number of degradation-regulated DEGs. Synthesis only: DEGs with significant changes in synthesis rates. Degradation only, DEGs with significant changes in degradation rates. Synthesis+degradation, DEGs with significant changes in both synthesis and degradation rates. FIG. 44N and FIG. 44O: The overlap of DEGs with significantly enhanced synthesis (FIG. 44N) or impaired degradation (FIG. 44O) between DROSHA and DICER1. FIG. 44P: Line plot showing the Ago2 binding patterns on the transcript regions of protein-coding genes in FIG. 44N and FIG. 44O. The transcript regions of genes were assembled by merging all exons, and were divided into 5′ UTR, coding sequence (CDS) and 3′UTR based on coordinates of the 5′ most start codon and the 3′ most stop codon. Single-base coverage of Ago2 eCLIP on each gene was calculated, binned, and scaled to 0-1. After merging scaled binned coverage of genes in the same group together, the lowest coverage value in the CDS was used to scale the merged coverage again to visualize the Ago2/RISC binding pattern. FIG. 44P and FIG. 44Q: Heatmaps showing the expression, synthesis and degradation rates of regulated genes upon DROSHA and DICER1 knockdown. Tiles of each row were colored by fold changes of values in perturbations relative to NTC. *: q-value<0.05 and fold change>1.5. #: 0.05<q-value<0.1. +: fold change>1.5 but 0.05<=q-value<0.1 or q-value<0.05 but fold change<1.5.

FIG. 45A through FIG. 45C. FIG. 45A: Heatmap showing the overall Pearson correlations of normalized sgRNA read counts between the plasmid library and bulk screen replicates at different sampling times. For each library, read counts of sgRNAs were firstly normalized by the sum of total counts to remove the batch effects brought by the sequencing depth, and the second normalization was performed by dividing the normalized counts of sgRNAs with the sum of normalized counts of sgNTC. FIG. 45B: Boxplot showing the reproducible trends of deletion upon CRISPRi between the present study and a prior report²⁷. Log 2FC was calculated by dividing normalized counts of samples collected at the end of the screen with the normalized counts of samples collected at the start point. FIG. 45C: Barplot showing the different extent of deletion of cells receiving sgRNAs targeting genes in different categories. The knockdown on genes with higher essentiality caused stronger cell growth arrest.

FIG. 46A through FIG. 46E. FIG. 46A: The distribution of sgRNA counts in sgRNA-based singlets and doublets. Top1-3, sgRNA with the highest/second/third highest abundance in single cells. Others, sgRNAs detected other than ones with top abundance. FIG. 46B through FIG. 46E: Dotplots showing the expression decreases of target genes upon CRISPRi compared to NTC at the sgRNA level. Target genes were reversely ordered by the mean expression reduction at the gene level. Fold change<0.6 was used for sgRNA filtering, and target genes with 3, 2, 1, 0 on-target sgRNA were shown in b-e, respectively. FC, fold change.

FIG. 47: A substantial defect in both global mRNA synthesis and degradation for some genes.

FIG. 48: The transcriptionally perturbed nuclear genes exhibited a strong enrichment of ATF4 and CEBPG motifs around their promoters.

FIG. 49: The knockdown of two critical regulators in this pathway (i.e., DROSHA and DICER1^{41, 142}) resulted in significantly overlapped DEGs.

### DETAILED DESCRIPTION

This is a technology for selectively synthesizing multi-indexed nucleic acid libraries from a plurality of cells or nuclei. In some embodiments, the multi-indexed library comprises a multi-indexed RNA library. In some embodiments, the multi-indexed library comprises a multi-indexed sgRNA library. In some embodiments, the multi-indexed library comprises a multi-indexed transposase accessible chromatin (ATAC) library.

In some embodiments, the multi-indexed library comprises a double-indexed library. In some embodiments, the multi-indexed library comprises a triple-indexed library.

In some embodiments, the present invention relates to methods for generating a sequencing library from single cells that can be used to determine cell-type specific temporal dynamics. In some embodiments, the methods of the invention include a combination of Ethynyl-2-deoxyuridine (EdU) labeling of newborn cells with single-cell combinatorial indexing to profile the single-cell transcriptome and chromatin landscape of cells in vivo. In some embodiments, the methods of the invention allow for both transcriptome and chromatin accessibility profiling. In some embodiments, the methods allow for tracking cell-type-specific proliferation and differentiation dynamics across conditions, and for identification of genetic and epigenetic signatures associated with the alteration of cellular dynamics.

In some embodiments, the invention provides a technology for integrating CRISPR-based pooled genetic screens, highly scalable single-cell RNA-seq by combinatorial indexing, and metabolic labeling to recover single-cell transcriptome dynamics across hundreds of genetic perturbations. The methods presented allow for quantitative characterization of the genome-wide mRNA kinetic rates (e.g., synthesis and degradation rates) across hundreds of genetic perturbations in a single experiment.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The terms “cells” and “population of cells” are used interchangeably and generally refer to a plurality of cells, i.e., more than one cell. The population may be a pure population comprising one cell type. Alternatively, the population may comprise more than one cell type. In the present invention, there is no limit on the number of cell types that a cell population may comprise.

“Isolated” means altered or removed from the natural state. For example, a nucleic acid or a peptide naturally present in a living organism is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a fixed nuclei.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient. The term “microarray” refers broadly to both “DNA microarrays” and “DNA chip(s),” and encompasses all art-recognized solid supports, and all art-recognized methods for affixing nucleic acid molecules thereto or for synthesis of nucleic acids thereon.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Barcoded Polynucleotides

In some embodiments, the invention provides methods of generating multi-barcoded polynucleotide molecules.

In some embodiments, the methods relate to contacting a sample containing RNA molecules with at least one set of barcoded reverse transcription primers, performing reverse transcription to generate singly barcoded DNA molecules, and contacting the singly barcoded DNA molecules with a set of barcoded PCR primers, and performing PCR amplification to generate a set of double barcoded polynucleotides. In some embodiments, the number of unique double barcoded polynucleotides corresponds to the number of unique combinations of barcodes that can be generated. Therefore, in various embodiments, a set of double barcoded polynucleotides comprises 5 to 10⁹ unique double barcoded polynucleotides.

In some embodiments, the methods relate to contacting a sample containing nucleic acid molecules with at least one set of barcoded transposases, performing tagmentation to generate singly barcoded DNA molecules, and contacting the singly barcoded DNA molecules with a set of barcoded PCR primers, and performing PCR amplification to generate a set of double barcoded polynucleotides. In some embodiments, the number of unique double barcoded polynucleotides corresponds to the number of unique combinations of barcodes that can be generated. Therefore, in various embodiments, a set of double barcoded polynucleotides comprises 5 to 10⁹ unique double barcoded polynucleotides.

In some embodiments, the methods relate to contacting a sample containing RNA molecules with at least one set of barcoded reverse transcription primers, performing reverse transcription to generate singly barcoded DNA molecules, contacting the singly barcoded DNA molecules with at least one set of barcoded ligation oligonucleotides, ligating the barcoded ligation oligonucleotides to the nucleic acid molecules to generate double barcoded DNA molecules, and contacting the double barcoded DNA molecules a set of barcoded PCR primers, and performing PCR amplification to generate a set of triple barcoded polynucleotides. In some embodiments, the number of unique triple barcoded polynucleotides corresponds to the number of unique combinations of barcodes that can be generated. Therefore, in various embodiments, a set of triple barcoded polynucleotides comprises 5 to 10⁹ unique triple barcoded polynucleotides.

Non-limiting examples of barcode primer sets for generating multi-barcoded polynucleotides of the present disclosure are provided in Tables 3-7 and 11, however the invention is not limited to these specific barcode sets as any number of alternative unique barcodes can be incorporated into the barcoded polynucleotides to generate a multi-indexed library of barcoded polynucleotides.

In one exemplary embodiment, for use in 96 well plate format, a set of barcoded polynucleotides comprises at least unique 96 barcodes. Exemplary sets of unique barcodes include, but are not limited to, those set forth in Table 3, Table 4, Table 5 or Table 6.

A barcode sequence is a unique sequence that can be used to distinguish a barcoded polynucleotide in a biological sample from other barcoded polynucleotides in the same biological sample. The concept of “barcodes” and appending barcodes to nucleic acids and other proteinaceous and non-proteinaceous materials is known to one of ordinary skill in the art (see, e.g., Liszczak G et al. Angew Chem Int Ed Engl. 2019 Mar. 22; 58 (13): 4144-4162). Thus, it should be understood that the term “unique” is with respect to the molecules of a single biological sample and means “only one” of a particular molecule or subset of molecules of the sample.

The length of a barcode sequence may vary. For example, a barcode sequence may have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a barcode sequence may have a length of 5, 10, 15, 20, 25, 30, 35, 40, 45, or 50 nucleotides.

In some embodiments, the methods comprise delivering to a biological tissue a first set of barcoded polynucleotides. A first set may include any number of barcoded polynucleotides. In some embodiments, a first set include 5 to 1000 barcoded polynucleotides. For example, a first set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a first set are contemplated herein.

In some embodiments, the methods comprise delivering to the biological sample a second set of barcoded polynucleotides. A second set may include any number of barcoded polynucleotides. In some embodiments, a second set include 5 to 1000 barcoded polynucleotides. For example, a second set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a second set are contemplated herein.

In some embodiments, the methods comprise delivering to the biological sample a third set of barcoded polynucleotides. A third set may include any number of barcoded polynucleotides. In some embodiments, a third set includes 5 to 1000 barcoded polynucleotides. For example, a third set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a third set are contemplated herein.

In one embodiment, the invention provides a method of performing reverse transcription (RT) comprising contacting an RNA sample with a set of RT primers and a reverse transcriptase.

In some embodiments, the methods comprise joining barcoded polynucleotides of the first set to barcoded polynucleotides of the second set. In some embodiments, the methods comprise exposing the biological sample to a ligation reaction, thereby producing double barcoded polynucleotides, wherein the double barcoded polynucleotides comprises a unique combination of barcoded polynucleotides from the first set and the second set.

In one embodiment, the method of the invention incorporates a step of combining two polynucleotide sequences into a single nucleic acid molecule using “tagmentation.” As used herein, the term “tagmentation” refers to the modification of DNA by a transposome complex comprising transposase enzyme complexed with adaptors comprising transposon end sequence. Tagmentation results in the simultaneous fragmentation of the target DNA molecule and ligation of a polynucleotide sequence (e.g. an adaptor or linker) to the 5′ ends of both strands of duplex fragments. Following a purification step to remove the transposase enzyme, additional sequences (e.g., barcodes) can be added to the ends of the adapted fragments, for example by PCR, ligation, or any other suitable methodology known to those of skill in the art.

The method of the invention can use any transposase that can accept a transposase end sequence and fragment a target nucleic acid, attaching a transferred end, but not a non-transferred end. A “transposome” is comprised of at least a transposase enzyme and a transposase recognition site. In some such systems, termed “transposomes”, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”. In some such insertion events, one strand of the transposase recognition site may be transferred into the target nucleic acid.

Some embodiments can include the use of a barcoded Tn5 transposase to incorporate a barcode into DNA molecules for preparation of a multi-indexed library.

In some embodiments, the methods comprise performing PCR amplification of using a set of PCR primers comprising a set of barcoded polynucleotides.

In some embodiments the multi-indexed library of the invention comprises a multitude of indexed nucleic acid products comprising two or more barcodes, wherein the combination of the two or more barcodes comprises a unique combination of barcoded polynucleotides. In some embodiments, the unique combination is a unique combination of a first and second barcode. In some embodiments, the unique combination is a unique combination of a first, a second, and a third barcode.

### Phosphorothioate Adaptor

Also provided herein is an adaptor sequence, which may be a polynucleotide comprising phosphorothioate bonds between the nucleotides which makes it resistant to tagmentation. The purpose of the adaptor is to serve as a bridge to join barcoded polynucleotides from two different sets (e.g., to aid in ligation of single barcoded polynucleotides to the polynucleotides comprising the second barcode). The length of the phosphorothioate adaptor may vary. For example, a phosphorothioate adaptor may have a length of 10 to 100 nucleotides (e.g., 10 to 90, 10 to 80, 10 to 70, 10 to 60, 10 to 50, 10 to 40, 10 to 30, 10 to 20, 20 to 100, 20 to 90, 20 to 80, 20 to 70, 20 to 60, 20 to 50, 20 to 40, or 20 to 30 nucleotides). In some embodiments, a phosphorothioate adaptor may have a length of 10, 15, 20, 25, 30, 35, 40, 45, 50, 55, 60, 65, 70, 75, 80, 85, 90, 95, or 100 nucleotides. Longer phosphorothioate adaptors are contemplated herein.

In some embodiments, the phosphorothioate adaptor is added to a singly barcoded polynucleotide sample concurrently with or following the delivery of a second set of barcoded polynucleotides, although, in some embodiments, the phosphorothioate adaptor may be annealed to the second set of barcoded polynucleotides prior to delivery.

In one embodiment, the phosphorothioate adaptor comprises a 3′ end modification. Exemplary 3′ end modifications include, but are not limited to, 3′ddC, 3′ddT, 3′ddU, 3′ Inverted dT, 3′ C3 spacer, 3′ amino, 3′ rU oxidized by periodate, 3′ phosphorylation, 3′ fluoro, 3′aldehyde, 3′carboxylate, 3′ thiol, 3′O-methyl, 3′azido, 3′alkyne, 3′alkene, 3′ (CH2)n-X (X═H, OCH3, CH3, SH, NH2, OH, etc.; n≥1), and 3′ (CH2CH2O)n (n≥1). In one embodiment, the phosphorothioate adaptor comprises at least one chemical group that blocks the 3′ hydroxyl group. In one embodiment, the phosphorothioate adaptor comprises at least one modification that removes the 3′ hydroxyl group.

In some embodiments, the phosphorothioate adaptor sequence for use in the ligation reaction comprises 5′-A*G*A*T*C*G*G*A*A*G*A*G*C*G*T*C*G*T*G*T*A*G*G*G*A*A*A*G*A*G*T*G*T*/3ddC/(SEQ ID NO: 2445), wherein ‘*’ represents phosphorothioate bonds between nucleotides, which prevents the tagmentation of the oligo, and wherein ‘/3ddC/’ represents a dideoxycytidine modification, which prevents the extension of the oligo on the 3′ end by DNA polymerases.

### Sequencing

In some embodiments, the methods include a sequencing step. For example, next generation sequencing (NGS) methods (or other sequencing methods) may be used to sequence the triple barcoded polynucleotide libraries. In some embodiments, the methods comprise preparing an NGS library in vitro. Thus, in some embodiments, the methods comprise sequencing the library of barcoded nucleic acid molecules to produce sequencing reads. Sequencing methods are known, and an example protocol is provided herein.

### Triple Indexed RNA Library

In some embodiments, the present invention relates to a method for generating a triple-indexed RNA sequencing library. In one embodiment, the method comprises the steps of:
Distributing nuclei or cells to wells of a multi-well plate;
Reverse Transcription (RT) of RNA molecules using a set of two indexed RT primers to generate a cDNA library having a first index;
Pooling of the cDNA library and Redistribution of the cDNA library into wells of a multi-well plate;
Ligation of a second index sequence onto the cDNA library using an adaptor sequence to aid in ligation;
Pooling of the cDNA library and Redistribution of the cDNA library into wells of a multi-well plate;
Second strand synthesis of the cDNA library;
Purification;
Tagmentation; and
PCR amplification of the dsDNA library with indexed primers to generate a triple indexed sequencing library.

In some embodiments, sets of indexed primers are provided in Tables 3-6 of Example 2 and in Table 11 of Example 4.

Table 3 of Example 2 provides indexed short dT primers for use in reverse transcription (RT) to index mRNA molecules having a polyA tail.

Table 4 of Example 2 provides random RT primers to index total RNA molecules.

Table 11 of Example 4 provides sgRNA capture primers for use in capturing sgRNA molecules.

Table 5 of Example 2 provides indexed ligation primers for use in adding a second index to cDNA molecules in a ligation step in combination with a ligation adaptor sequence.

In some embodiments, the adaptor sequence for use in the ligation reaction comprises 5′-A*G*A*T*C*G*G*A*A*G*A*G*C*G*T*C*G*T*G*T*A*G*G*G*A*A*A*G*A*G*T*G*T*/3ddC/(SEQ ID NO: 2445), wherein ‘*’ represents phosphorothioate bonds between nucleotides, which prevents the tagmentation of the oligo, and wherein ‘/3ddC/’ represents a dideoxycytidine modification, which prevents the extension of the oligo on the 3′ end by DNA polymerases.

Table 6 of Example 2 provides a set of indexed P7 primer sequences for use in adding a third index to the library during PCR.

### Using Triple-Barcoded RNA Molecules

Any method that would benefit from massive parallel sequencing can utilize the triple barcode methodology of the present invention. In various embodiments, triple barcoded nucleic acid molecule libraries prepared for use in an assay such as RT-PCR, qRT-PCR, RNA-structure mapping (such as SHAPE-seq or SHAPE-MaP, DMS-seq), transcriptome profiling, in-cell sequencing, next-generation RNA sequencing (RNA-seq), nanopore sequencing, PacBio sequencing, zero-mode waveguide sequencing, cDNA library synthesis, cDNA synthesis, and a combination thereof.

In some embodiments, the triple barcode method of the invention is incorporated into methods for determining transcriptome and chromatin landscape changes in cells. In some embodiments, the triple barcode method of the invention is incorporated into methods to dissect the critical regulators of gene-specific transcription, splicing, and degradation in a massive-parallel manner.

### Cell-Type-Specific Temporal Dynamics

In some embodiments, the present invention relates to methods for generating an RNA or ATAC sequencing library from single cells that can be used to determine cell-type specific temporal dynamics. In some embodiments, the methods of the invention include a combination of Ethynyl-2-deoxyuridine (EdU) labeling of newborn cells with single-cell combinatorial indexing to profile the single-cell transcriptome and chromatin landscape of cells in vivo. In some embodiments, the methods of the invention allow for both transcriptome and chromatin accessibility profiling. In some embodiments, the methods allow for tracking cell-type-specific proliferation and differentiation dynamics across conditions, and for identification of genetic and epigenetic signatures associated with the alteration of cellular dynamics.

In some embodiments, the method comprises the following steps: (i) label a cell, tissue or sample with 5-Ethynyl-2-deoxyuridine (EdU), a thymidine analog that can be incorporated into replicating DNA for labeling in vivo cellular proliferation, (ii) nuclei are extracted, fixed, and then subjected to click chemistry-based in situ ligation to an azide-containing fluorophore, followed by fluorescence-activated cell sorting (FACS) to enrich the EdU+ cells, (iii) indexed reverse transcription or transposition is used to introduce the first round of indexing, cells from all wells are pooled and then redistributed into multiple 96-well plates through FACS sorting to further purify the EdU+ cells, (iv) library preparation proceeds using protocols for multi-barcoding of polynucleotides such that most cells pass through a unique combination of wells, such that their contents are marked by a unique combination of barcodes that can be used to group reads derived from the same cell. In some embodiments, the two sorting steps are essential for excluding contaminating cells and enriching extremely rare proliferating cell populations.

### TrackerSci-RNA

In some embodiments, the method comprises EdU staining nuclei using Click-iT Plus EdU Alexa Fluor™ 647 Flow Cytometry assay Kit. Then, nuclei are spun down, washed once with 1× Click-iT saponin-based permeabilization and wash reagent, resuspended, stained with 4′,6-diamidino-2-phenylindole (DAPI, Invitrogen D1306) and FACS sorted. Next, Alexa647 and DAPI positive nuclei are sorted into multi-well plates with each well containing about 250˜500 nuclei. Reverse transcription is then performed on the RNA molecules with a barcoded oligo-dT primer (5′-(SEQ ID NO: 2447) ACGACGCTCTTCCGATCTNNNNNNNN [10 bp-index] TTTTTTTTTTTTTTTTTTTTTTTTTTTTTTVN-3′ (SEQ ID NO:2448). Nuclei are then pooled, stained with DAPI, and sorted at 25 nuclei per well into a second set of multi-well plates. Cells are gated based on DAPI and Alexa647 such that singlets are discriminated from doublets and EdU+ cells are purified. Second strand synthesis is then performed and tagmentation is performed. After tagmentation, each well is mixed with P5 primer (5′-(SEQ ID NO:2415) AATGATACGGCGACCACCGAGATCTACA [15] CCCTACACGACGCTCTTCCGAT CT-3′ (SEQ ID NO:2416), IDT), and P7 primer (5′-(SEQ ID NO: 2417) CAAGCAGAAGACGGCATACGAGAT [17] GTCTCGTGGGCTCGG-3′ (SEQ ID NO: 2418)), and PCR amplification is carried out. After PCR, samples are pooled and purified. Following purification, the samples can be sequenced.

### TrackerSci-ATAC

In some embodiments, the method comprises EdU staining nuclei using Click-iT Plus EdU Alexa Fluor™ 647 Flow Cytometry assay Kit (Thermo Fisher Scientific, 10634), nuclei are spun down, permeabilized Click-iT saponin-based permeabilization and wash reagent, and FACS sorted. Alexa647 and DAPI positive nuclei were sorted into multi-well plates with each well containing about 250˜500 nuclei. Barcoded Tn5 is added and Tagmentation is performed. All nuclei are then pooled, stained with DAPI, and sorted into multi-sell plates with the gating based on DAPI and Alexa647 such that singlets are discriminated from doublets and EdU+ cells are purified. After sorting, reverse crosslinking is performed. Then, indexed P5 primer (5′-(SEQ ID NO: 2415)

AATGATACGGCGACCACCGAGATCTACA [15] CCCTACACGACGC TCTTCCGATCT-3′ (SEQ ID NO:2449)), and indexed P7 primer (5′-(SEQ ID NO:2419) CAAGCAGAAGACGGCATACGAGAT [17] GTGACTGGAGTTCAGACGTGTGCTCT TCCGATCT-3′ (SEQ ID NO:2420)) are added into each well and PCR amplification is carried out. Final PCR products are pooled and purified. The TrackerSci ATAC-seq library can then be sequenced.

sgRNA Libraries

In some embodiments, the present invention relates to methods for generating an RNA sequencing library from single cells that can be used to dissect the critical regulators of gene-specific transcription, splicing, and degradation in a massive-parallel manner.

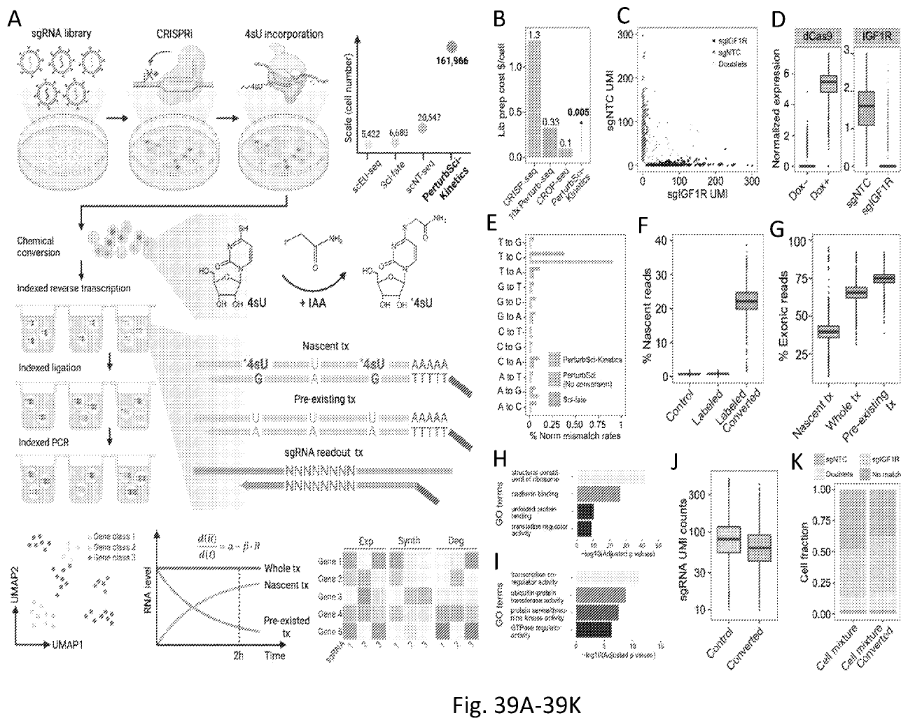

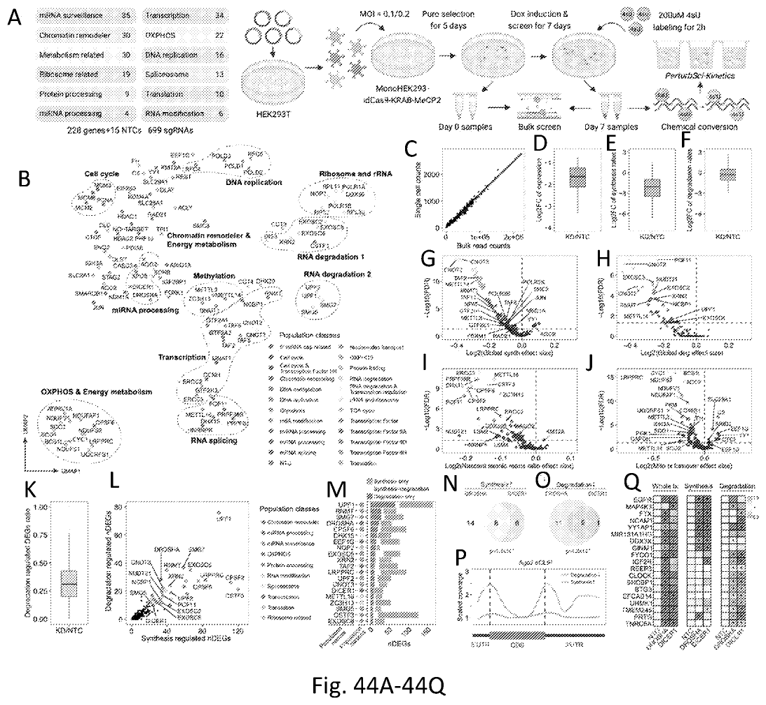

In one embodiment, the method comprises the steps as outlined in FIG. 39A and FIG. 44A. In one embodiment, the methods include the development of a novel combinatorial indexing strategy (referred to as ‘PerturbSci’) which was developed for targeted enrichment and amplification of the sgRNA region that carries the same cellular barcode with the whole transcriptome (FIG. 39A). PerturbSci yields a high capture rate of sgRNA (i.e., over 97%), comparable to previous approaches for single-cell profiling of pooled CRISPR screens. Furthermore, the method builds on a method of single-cell RNA-seq by three-level combinatorial indexing (i.e., EasySci-RNA, which is described in detail in Examples 1 and 2 herein). PerturbSci substantially reduces library preparation costs for single-cell RNA profiling of pooled CRISPR screens. In some embodiments, a multimeric fusion protein dCas9-KRAB-MeCP212 (idCas9), a highly potent transcriptional repressor that outperforms conventional dCas9 repressors is used for performing the library preparation assay(s) of the invention. In some embodiments, PerturbSci is integrated with a 4-thiouridine (4sU) labeling method. The integrated method (i.e., PerturbSci-Kinetics) exhibits an order of magnitude higher throughput than the previous single-cell metabolic profiling approaches. Following 4sU labeling and thiol (SH)-linked alkylation reaction (referred to as ‘chemical conversion’), the nascent transcriptome and the whole transcriptome from the same cell can be distinguished by T to C conversion in reads mapping to mRNAs. The kinetic rate of mRNA dynamics (e.g., synthesis and degradation) are then calculated as a multi-layer readout for each genetic perturbation.

In one embodiment, the method of the invention can be used to dissect key regulators of transcriptome kinetics. In such an embodiment, a PerturbSci-Kinetics screen can be performed on idCas9 cells transduced with a library of sgRNAs, containing guides targeting genes involved in a variety of biological processes including mRNA transcription, processing, degradation, and others. In one embodiment, the cloning and lentiviral packaging are performed in a pooled fashion. In one embodiment, the idCas9 cell line is transfected with the sgRNA virus library at a low multiplicity of infection to ensure most cells received only one sgRNA. After a 5-day puromycin selection to remove cells receiving no sgRNA, a fraction of cells for bulk library preparation. In one embodiment, the rest of the cells are treated with Doxycycline (Dox) to induce the dCas9-KRAB-MeCP2 expression. After at least seven days for efficient gene knockdown, 4sU labeling is performed on the cells (for about two hours) and samples of the cells are harvested for both bulk and single-cell PerturbSci-Kinetics library preparation. In some embodiments, chemical conversion of the 4sU label occurs before library preparation.

In some embodiments, the screening method of the invention can be used to uniquely capture multiple layers of information, including, but not limited to gene-specific synthesis and degradation rate in each perturbation, splicing information, the kinetics of genes targeted by CRISPRi, the impact of diverse genetic perturbations on the global dynamics (i.e., synthesis, splicing and degradation) of the transcriptome, and gene-specific synthesis and degradation regulation across all gene perturbations.

In one embodiment, the splicing dynamics of the transcriptome can be reflected by the ratio of nascent reads mapped to exonic regions.

In some embodiments, the methods of the invention involve the step of contacting a plurality of cells with an sgRNA library. In some embodiments, the sgRNA library comprises at least 50, 100, 150, 200, 250, 300, 350, 400, 450, 500, 550, 600, 650, 700, 750, 800, 850, 900, 950, 1000, or more than 1000 plasmids for expression of unique sgRNA species.

In some embodiments, the methods of the invention involve the step of contacting a plurality of cells with an sgRNA library. In some embodiments, the sgRNA library comprises at least 50, 100, 150, 200, 250, 300, 350, 400, 450, 500, 550, 600, 650, 700, 750, 800, 850, 900, 950, 1000, or more than 1000 plasmids for expression of unique sgRNA species.

In some embodiments, the plurality of cells are contacted with the sgRNA library at a concentration of at least about 1000× coverage/sgRNA. In some embodiments, the plurality of cells are contacted with the sgRNA library at a concentration of at least about 2000× coverage/sgRNA. In some embodiments, the cells are contacted with the sgRNA library such that each cell is transduced with a single sgRNA. In some embodiments, the plasmids of the sgRNA library express a selectable marker (e.g., an antibiotic resistance gene) and transduced cells are selected by contacting the plurality of cells with selection compound (e.g., an antibiotic) for at least one day.

In some embodiments, the methods of the invention involve the use of a catalytically dead Cas9 protein. In some embodiments, the catalytically dead Cas9 protein is inducible. In one embodiment, the inducible catalytically dead Cas9 protein is dCas9-KRAB-MeCP2 which is inducible in the presence of doxycycline. In some embodiments, expression of the catalytically dead Cas9 protein is induced for at least 1 day by the addition of an induction agent (e.g., doxycycline) to the cell culture media. In some embodiments, the sgRNA library transfected cells are cultured for at least 2, 3, 4, 5, 6, 7, or more than days in the presence of the induction agent for inducing expression of the catalytically dead Cas9 protein.

In some embodiments, the sgRNA library transfected cells are cultured in media to sensitize the cells to perturbation. For example, in some embodiments, the cells are cultured in L-glutamine+, sodium pyruvate−, high glucose DMEM to sensitize the cells to perturbations of energy metabolism genes. In some embodiments, the cells are cultured for at least 2, 3, 4, 5, 6, 7, or more than days in the presence of the media to sensitize the cells to perturbation.

In some embodiments, the sgRNA library transfected cells are cultured in media comprising a combination of an inducing agent to induce expression of catalytically dead Cas9 as well as one or more agent or condition to sensitize the cells to perturbation. In some embodiments, the cells are cultured for at least 2, 3, 4, 5, 6, 7, or more than days in the presence of the media to sensitize the cells to perturbation further comprising an inducing agent to induce expression of the catalytically dead Cas9. In some embodiments, the cells are cultured for at least 7 days in L-glutamine+, sodium pyruvate−, high glucose DMEM further comprising an induction agent to induce expression of the catalytically dead Cas9. In some embodiments, the cells are cultured for at least 7 days in L-glutamine+, sodium pyruvate−, high glucose DMEM further comprising doxycycline.

In some embodiments the method further comprises a step of labeling nascent transcripts to allow for separation of nascent transcripts from the pre-existing transcripts in the total transcriptome content in downstream sequencing data. Any method known in the art for labeling nascent transcripts can be used in the method of the invention to label nascent transcripts including, but not limited to, 5-Bromouridine (BrU) or 4-thiouridine (4sU) labeling. For example, in some embodiments the method further comprises adding 4sU to the cells to label nascent transcripts. In some embodiments, the sgRNA library transfected cells that have been cultured in the presence of an inducing agent to induce expression of catalytically dead Cas9 are contacted with 4sU for at least 30 min, 1 hour, 2 hours, 3 hours or for about four hours immediately prior to harvesting the cells for isolation of nucleic acid molecules (e.g., RNA, mRNA) for sequence library preparation.

In some embodiments, the incorporated RNA metabolic label(s) undergo chemical conversion prior to generation of a nucleic acid sequencing library. For example, in some embodiments, the 4sU is chemically converted to cytidine prior to library preparation. Methods for chemically converting RNA metabolic labels are known in the art and can be used for chemical conversion of the incorporated RNA metabolic label(s) in the method of the invention.

In some embodiments, a subset of cells is collected following selection of the sgRNA transfection for analysis as the “Day 0” or initial “bulk” sequencing library. In some embodiments, genomic DNA, transcriptomic RNA, or a combination there of is isolated and analyzed from this first bulk sequencing library. Tables 1 and 2 and Example 2 provides a set of primer sequences for use in generating a bulk analysis sequencing library.

In some embodiments, a subset of cells is collected following addition of the RNA metabolic label, but prior to chemical conversion of the label for analysis as a second “bulk” sequencing library. In some embodiments, genomic DNA, transcriptomic RNA, or a combination there of is isolated and analyzed from this second bulk sequencing library. Tables 11 and 12 and Example 5 provide exemplary primer sequences for use in generating a bulk analysis sequencing library.

### Samples

In some embodiments, a sample is a biological sample. Non-limiting examples of biological samples include tissues, cells, and bodily fluids (e.g., blood, urine, saliva, cerebrospinal fluid, and semen). The biological sample may be adult tissue, embryonic tissue, or fetal tissue, for example. In some embodiments, a biological sample is from a human or other animal. For example, a biological sample may be obtained from a murine (e.g., mouse or rat), feline (e.g., cat), canine (e.g., dog), equine (e.g., horse), bovine (e.g., cow), leporine (e.g., rabbit), porcine (e.g., pig), hircine (e.g., goat), ursine (e.g., bear), or piscine (e.g., fish). Other animals are contemplated herein.

In some embodiments, a biological sample is fixed, and thus is referred to as a fixed biological sample. Fixation (e.g., tissue fixation) refers to the process of chemically preserving the natural state of a biological sample, for example, for subsequent histological analysis. Various fixation agents are routinely used, including, for example, formalin (e.g., formalin fixed paraffin embedded (FFPE) tissue), formaldehyde, paraformaldehyde and glutaraldehyde, any of which may be used herein to fix a biological sample. Other fixation reagents (fixatives) are contemplated herein.

In some embodiments, the biological sample is a tissue. In some embodiments, the biological sample is a cell. A biological sample, such as a tissue or a cell, in some embodiments, is sectioned and mounted on a surface, such as a slide. In such embodiments, the sample may be fixed before or after it is sectioned. In some embodiments, the fixation process involves perfusion of the animal from which the sample is collected.

Nucleic acid molecules suitable as templates for use in generating a multi-indexed library of the invention include any nucleic acid molecule or population of nucleic acid molecules (e.g., DNA, RNA, mRNA, sgRNA), particularly those derived from a cell or tissue. In one aspect, a population of mRNA molecules (a number of different mRNA molecules, typically obtained from cells or tissue) are used to make a multi-indexed cDNA library, in accordance with the invention. Exemplary sources of nucleic acid templates include viruses, virally infected cells, bacterial cells, fungal cells, plant cells and animal cells.

### Reaction Solutions

Various reaction solutions can be used for performing the different reactions (RT, PCR, tagmentation, ligation, etc.) of the methods of the invention.

In some embodiments, one or more reaction solution comprises a buffering agent. The concentration of the buffering agent in the reaction solutions of the invention will vary with the particular buffering agent used. Typically, the working concentration (i.e., the concentration in the reaction mixture) of the buffering agent will be from about 5 mM to about 500 mM (e.g., about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, from about 5 mM to about 500 mM, from about 10 mM to about 500 mM, from about 20 mM to about 500 mM, from about 25 mM to about 500 mM, from about 30 mM to about 500 mM, from about 40 mM to about 500 mM, from about 50 mM to about 500 mM, from about 75 mM to about 500 mM, from about 100 mM to about 500 mM, from about 25 mM to about 50 mM, from about 25 mM to about 75 mM, from about 25 mM to about 100 mM, from about 25 mM to about 200 mM, from about 25 mM to about 300 mM, etc.). When Tris (e.g., Tris-HCl) is used, the Tris working concentration will typically be from about 5 mM to about 100 mM, from about 5 mM to about 75 mM, from about 10 mM to about 75 mM, from about 10 mM to about 60 mM, from about 10 mM to about 50 mM, from about 25 mM to about 50 mM, etc.

The final pH of solutions of the invention will generally be set and maintained by buffering agents present in reaction solutions of the invention. The pH of reaction solutions of the invention, and hence reaction mixtures of the invention, will vary with the particular use and the buffering agent present but will often be from about pH 5.5 to about pH 9.0 (e.g., about pH 6.0, about pH 6.5, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, from about pH 6.0 to about pH 8.5, from about pH 6.5 to about pH 8.5, from about pH 7.0 to about pH 8.5, from about pH 7.5 to about pH 8.5, from about pH 6.0 to about pH 8.0, from about pH 6.0 to about pH 7.7, from about pH 6.0 to about pH 7.5, from about pH 6.0 to about pH 7.0, from about pH 7.2 to about pH 7.7, from about pH 7.3 to about pH 7.7, from about pH 7.4 to about pH 7.6, from about pH 7.0 to about pH 7.4, from about pH 7.6 to about pH 8.0, from about pH 7.6 to about pH 8.5, from about pH 7.7 to about pH 8.5, from about pH 7.9 to about pH 8.5, from about pH 8.0 to about pH 8.5, from about pH 8.2 to about pH 8.5, from about pH 8.3 to about pH 8.5, from about pH 8.4 to about pH 8.5, from about pH 8.4 to about pH 9.0, from about pH 8.5 to about pH 9.0, etc.)

In some embodiments, one or more monovalent cationic salts (e.g., LiCl, NaCl, KCl, NH₄Cl, etc.) may be included in reaction solutions of the invention. In many instances, salts used in reaction solutions of the invention will dissociate in solution to generate at least one species which is monovalent (e.g., Li⁺, Na⁺, K⁺, NH₄⁺, etc.) When included in reaction solutions of the invention, salts will often be present either individually or in a combined concentration of from about 0.5 mM to about 500 mM (e.g., about 1 mM, about 2 mM, about 3 mM, about 5 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, about 55 mM, about 60 mM, about 64 mM, about 65 mM, about 70 mM, about 75 mM, about 80 mM, about 85 mM, about 90 mM, about 95 mM, about 100 mM, about 120 mM, about 140 mM, about 150 mM, about 175 mM, about 200 mM, about 225 mM, about 250 mM, about 275 mM, about 300 mM, about 325 mM, about 350 mM, about 375 mM, about 400 mM, from about 1 mM to about 500 mM, from about 5 mM to about 500 mM, from about 10 mM to about 500 mM, from about 20 mM to about 500 mM, from about 30 mM to about 500 mM, from about 40 mM to about 500 mM, from about 50 mM to about 500 mM, from about 60 mM to about 500 mM, from about 65 mM to about 500 mM, from about 75 mM to about 500 mM, from about 85 mM to about 500 mM, from about 90 mM to about 500 mM, from about 100 mM to about 500 mM, from about 125 mM to about 500 mM, from about 150 mM to about 500 mM, from about 200 mM to about 500 mM, from about 10 mM to about 100 mM, from about 10 mM to about 75 mM, from about 10 mM to about 50 mM, from about 20 mM to about 200 mM, from about 20 mM to about 150 mM, from about 20 mM to about 125 mM, from about 20 mM to about 100 mM, from about 20 mM to about 80 mM, from about 20 mM to about 75 mM, from about 20 mM to about 60 mM, from about 20 mM to about 50 mM, from about 30 mM to about 500 mM, from about 30 mM to about 100 mM, from about 30 mM to about 70 mM, from about 30 mM to about 50 mM, etc.).

In some embodiments, one or more reaction solution comprises a buffering agent, one or more divalent cationic salts (e.g., MnCl₂, MgCl₂, MgSO₄, CaCl₂), etc.) may be included in reaction solutions of the invention. In many instances, salts used in reaction solutions of the invention will dissociate in solution to generate at least one species which is divalent (e.g., Mg⁺⁺, Mn⁺⁺, Ca⁺⁺, etc.) When included in reaction solutions of the invention, salts will often be present either individually or in a combined concentration of from about 0.5 mM to about 500 mM (e.g., about 1 mM, about 2 mM, about 3 mM, about 4 mM, about 5 mM, about 6 mM, about 7 mM, about 8 mM, about 9 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, about 55 mM, about 60 mM, about 64 mM, about 65 mM, about 70 mM, about 75 mM, about 80 mM, about 85 mM, about 90 mM, about 95 mM, about 100 mM, about 120 mM, about 140 mM, about 150 mM, about 175 mM, about 200 mM, about 225 mM, about 250 mM, about 275 mM, about 300 mM, about 325 mM, about 350 mM, about 375 mM, about 400 mM, from about 1 mM to about 500 mM, from about 5 mM to about 500 mM, from about 10 mM to about 500 mM, from about 20 mM to about 500 mM, from about 30 mM to about 500 mM, from about 40 mM to about 500 mM, from about 50 mM to about 500 mM, from about 60 mM to about 500 mM, from about 65 mM to about 500 mM, from about 75 mM to about 500 mM, from about 85 mM to about 500 mM, from about 90 mM to about 500 mM, from about 100 mM to about 500 mM, from about 125 mM to about 500 mM, from about 150 mM to about 500 mM, from about 200 mM to about 500 mM, from about 10 mM to about 100 mM, from about 10 mM to about 75 mM, from about 10 mM to about 50 mM, from about 20 mM to about 200 mM, from about 20 mM to about 150 mM, from about 20 mM to about 125 mM, from about 20 mM to about 100 mM, from about 20 mM to about 80 mM, from about 20 mM to about 75 mM, from about 20 mM to about 60 mM, from about 20 mM to about 50 mM, from about 30 mM to about 500 mM, from about 30 mM to about 100 mM, from about 30 mM to about 70 mM, from about 30 mM to about 50 mM, etc.).

When included in reaction solutions of the invention, reducing agents (e.g., dithiothreitol, β-mercaptoethanol, etc.) will often be present either individually or in a combined concentration of from about 0.1 mM to about 50 mM (e.g., about 0.2 mM, about 0.3 mM, about 0.5 mM, about 0.7 mM, about 0.9 mM, about 1 mM, about 2 mM, about 3 mM, about 4 mM, about 5 mM, about 6 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, from about 0.1 mM to about 50 mM, from about 0.5 mM to about 50 mM, from about 1 mM to about 50 mM, from about 2 mM to about 50 mM, from about 3 mM to about 50 mM, from about 0.5 mM to about 20 mM, from about 0.5 mM to about 10 mM, from about 0.5 mM to about 5 mM, from about 0.5 mM to about 2.5 mM, from about 1 mM to about 20 mM, from about 1 mM to about 10 mM, from about 1 mM to about 5 mM, from about 1 mM to about 3.4 mM, from about 0.5 mM to about 3.0 mM, from about 1 mM to about 3.0 mM, from about 1.5 mM to about 3.0 mM, from about 2 mM to about 3.0 mM, from about 0.5 mM to about 2.5 mM, from about 1 mM to about 2.5 mM, from about 1.5 mM to about 2.5 mM, from about 2 mM to about 3.0 mM, from about 2.5 mM to about 3.0 mM, from about 0.5 mM to about 2 mM, from about 0.5 mM to about 1.5 mM, from about 0.5 mM to about 1.1 mM, from about 5.0 mM to about 10 mM, from about 5.0 mM to about 15 mM, from about 5.0 mM to about 20 mM, from about 10 mM to about 15 mM, from about 10 mM to about 20 mM, etc.).

Reaction solutions of the invention may also contain one or more ionic or non-ionic detergent (e.g., TRITON X-100™, NONIDET P40™, sodium dodecyl sulfate, etc.). When included in reaction solutions of the invention, detergents will often be present either individually or in a combined concentration of from about 0.01% to about 5.0% (e.g., about 0.01%, about 0.02%, about 0.03%, about 0.04%, about 0.05%, about 0.06%, about 0.07%, about 0.08%, about 0.09%, about 0.1%, about 0.15%, about 0.2%, about 0.3%, about 0.5%, about 0.7%, about 0.9%, about 1%, about 2%, about 3%, about 4%, about 5%, from about 0.01% to about 5.0%, from about 0.01% to about 4.0%, from about 0.01% to about 3.0%, from about 0.01% to about 2.0%, from about 0.01% to about 1.0%, from about 0.05% to about 5.0%, from about 0.05% to about 3.0%, from about 0.05% to about 2.0%, from about 0.05% to about 1.0%, from about 0.1% to about 5.0%, from about 0.1% to about 4.0%, from about 0.1% to about 3.0%, from about 0.1% to about 2.0%, from about 0.1% to about 1.0%, from about 0.1% to about 0.5%, etc.). For example, reaction solutions of the invention may contain TRITON X-100™ at a concentration of from about 0.01% to about 2.0%, from about 0.03% to about 1.0%, from about 0.04% to about 1.0%, from about 0.05% to about 0.5%, from about 0.04% to about 0.6%, from about 0.04% to about 0.3%, etc.

Reaction solutions of the invention may also contain one or more stabilizing agents (e.g., PEG8000, trehalose, betaine, BSA, glycerol). In some embodiments, when included in reaction solutions of the invention, stabilizing agents are present either individually or in a combined concentration from 0.01 M to about 50 M (e.g., about 0.05M, about 0.1 M, 0.2 M, about 0.3 M, about 0.5 M, about 0.6 M, about 0.7 M, about 0.9 M, about 1 M, about 2 M, about 3 M, about 4 M, about 5 M, about 6 M, about 10 M, about 12 M, about 15 M, about 17 M, about 20 M, about 22 M, about 23 M, about 24 M, about 25 M, about 27 M, about 30 M, about 35 M, about 40 M, about 45 M, about 50 M, from about 0.1 M to about 1 M, from about 0.5 M to about 5 M, from about 0.2 M to about 2 M, from about 0.3 M to about 3 M, from about 0.4 M to about 4 M, from about 0.5 M to about 5 M, from about 0.2 M to about 0.8 M, from about 0.5 M to about 1 M, from about 0.05 M to about 1 M, from about 0.05 M to about 10 M, from about 0.05 M to about 20M, etc.). In some embodiments, when included in reaction solutions of the invention, such stabilizing agents are present either individually or in a combined concentration of from about 0.01 mg/ml to about 100 mg/ml (e.g., about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.15 mg/ml, about 0.17 mg/ml, about 0.2 mg/ml, about 0.25 mg/ml, about 0.35 mg/ml, about 0.5 mg/ml, about 0.75 mg/ml, about 1.0 mg/ml, about 1.5 mg/ml, about 2.0 mg/ml, about 2.5 mg/ml, about 3.0 mg/ml, about 3.5 mg/ml, about 4.0 mg/ml, about 5.0 mg/ml, about 6.0 mg/ml, about 7.0 mg/ml, about 8.0 mg/ml, about 9.0 mg/ml, about 10.0 mg/ml, from about 0.05 mg/ml to about 3.0 mg/ml, from about 0.1 mg/ml to about 5.0 mg/ml, from about 0.2 mg/ml to about 2.0 mg/ml, etc.). In some embodiments, when included in reaction solutions of the invention, such stabilizing agents are be present either individually or in a combined concentration of from about 0.1% to about 50% (e.g., about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 5.0%, about 7.0%, about 9.0%, about 10%, about 11%, about 12%, about 13%, about 14%, about 15%, about 20%, about 22%, about 25%, about 27%, about 30%, about 35%, about 40%, about 45%, about 50%, from about 0.1% to about 50%, from about 0.1% to about 40%, from about 0.1% to about 30%, from about 0.0% to about 20%, from about 0.1% to about 10%, etc.

Reaction solutions the invention may also contain one or more additional additives that improve enzymatic activity, including agents that improve primer utilization efficiency and improve product yield.

In many instances, nucleotides (e.g., dNTPs, such as dGTP, dATP, dCTP, dTTP, etc.) will be present in reaction mixtures of the invention. Typically, individual nucleotides will be present in concentrations of from about 0.05 mM to about 50 mM (e.g., about 0.07 mM, about 0.1 mM, about 0.15 mM, about 0.18 mM, about 0.2 mM, about 0.3 mM, about 0.5 mM, about 0.7 mM, about 0.9 mM, about 1 mM, about 2 mM, about 3 mM, about 4 mM, about 5 mM, about 6 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, from about 0.1 mM to about 50 mM, from about 0.5 mM to about 50 mM, from about 1 mM to about 50 mM, from about 2 mM to about 50 mM, from about 3 mM to about 50 mM, from about 0.5 mM to about 20 mM, from about 0.5 mM to about 10 mM, from about 0.5 mM to about 5 mM, from about 0.5 mM to about 2.5 mM, from about 1 mM to about 20 mM, from about 1 mM to about 10 mM, from about 1 mM to about 5 mM, from about 1 mM to about 3.4 mM, from about 0.5 mM to about 3.0 mM, from about 1 mM to about 3.0 mM, from about 1.5 mM to about 3.0 mM, from about 2 mM to about 3.0 mM, from about 0.5 mM to about 2.5 mM, from about 1 mM to about 2.5 mM, from about 1.5 mM to about 2.5 mM, from about 2 mM to about 3.0 mM, from about 2.5 mM to about 3.0 mM, from about 0.5 mM to about 2 mM, from about 0.5 mM to about 1.5 mM, from about 0.5 mM to about 1.1 mM, from about 5.0 mM to about 10 mM, from about 5.0 mM to about 15 mM, from about 5.0 mM to about 20 mM, from about 10 mM to about 15 mM, from about 10 mM to about 20 mM, etc.). The combined nucleotide concentration, when more than one nucleotide is present, can be determined by adding the concentrations of the individual nucleotides together. When more than one nucleotide is present in reaction solutions of the invention, the individual nucleotides may not be present in equimolar amounts. Thus, a reaction solution may contain, for example, 1 mM dGTP, 1 mM dATP, 0.5 mM dCTP, and 1 mM dTTP.

Enzymes such as reverse transcriptases, ligases, polymerases, or transposases may also be present in reaction solutions. When present, enzymes will often be present in a concentration which results in about 0.01 to about 1,000 units of enzymatic activity/μl (e.g., about 0.01 unit/μl, about 0.05 unit/μl, about 0.1 unit/μl, about 0.2 unit/μl, about 0.3 unit/μl, about 0.4 unit/μl, about 0.5 unit/μl, about 0.7 unit/μl, about 1.0 unit/μl, about 1.5 unit/μl, about 2.0 unit/μl, about 2.5 unit/μl, about 5.0 unit/μl, about 7.5 unit/μl, about 10 unit/μl, about 20 unit/μl, about 25 unit/μl, about 50 unit/μl, about 100 unit/μl, about 150 unit/μl, about 200 unit/μl, about 250 unit/μl, about 350 unit/μl, about 500 unit/μl, about 750 unit/μl, about 1,000 unit/μl, from about 0.1 unit/μl to about 1,000 unit/μl, from about 0.2 unit/μl to about 1,000 unit/μl, from about 1.0 unit/μl to about 1,000 unit/μl, from about 5.0 unit/μl to about 1,000 unit/μl, from about 10 unit/μl to about 1,000 unit/μl, from about 20 unit/μl to about 1,000 unit/μl, from about 50 unit/μl to about 1,000 unit/μl, from about 100 unit/μl to about 1,000 unit/μl, from about 200 unit/μl to about 1,000 unit/μl, from about 400 unit/μl to about 1,000 unit/μl, from about 500 unit/μl to about 1,000 unit/μl, from about 0.1 unit/μl to about 300 unit/μl, from about 0.1 unit/μl to about 200 unit/μl, from about 0.1 unit/μl to about 100 unit/μl, from about 0.1 unit/μl to about 50 unit/μl, from about 0.1 unit/μl to about 10 unit/μl, from about 0.1 unit/μl to about 5.0 unit/μl, from about 0.1 unit/μl to about 1.0 unit/μl, from about 0.2 unit/μl to about 0.5 unit/μl, etc.

Reaction solutions of the invention may be prepared as concentrated solutions (e.g., 5× solutions) which are diluted to a working concentration for final use. With respect to a 5× reaction solution, a 5:1 dilution is required to bring such a 5× solution to a working concentration. Reaction solutions of the invention may be prepared, for examples, as a 2×, a 3×, a 4×, a 5×, a 6×, a 7×, a 8×, a 9×, a 10×, etc. solutions. One major limitation on the fold concentration of such solutions is that, when compounds reach particular concentrations in solution, precipitation occurs. Thus, concentrated reaction solutions will generally be prepared such that the concentrations of the various components are low enough so that precipitation of buffer components will not occur. As one skilled in the art would recognize, the upper limit of concentration which is feasible for each solution will vary with the particular solution and the components present.

In many instances, reaction solutions of the invention will be provided in sterile form. Sterilization may be performed on the individual components of reaction solutions prior to mixing or on reaction solutions after they are prepared. Sterilization of such solutions may be performed by any suitable means including autoclaving or ultrafiltration.

### Kits

The invention is also directed to kits for use in the library preparation methods of the invention. Such kits can be used for making multi-indexed sequencing libraries. Kits of the invention may comprise a carrier, such as a box or carton, having in close confinement therein one or more containers, such as vials, tubes, bottles and the like. In kits of the invention, a first container may contain one or more of the reverse transcriptase enzymes of the invention or one or more of the indexed reverse transcription primer sets and one or more additional container may contain one or more of the ligation enzymes of the invention or the indexed ligation primer set. Kits of the invention may also comprise, in the same or different containers, at least one component selected from one or more adaptor molecule, one or more indexed PCR primer, or other component for performing the library preparation method of the invention. In one embodiment, kits of the invention may also comprise, in the same or different containers, an optimized reaction buffer as described elsewhere herein, or components used to produce the optimized reaction buffer. Alternatively, the components of the kit may be divided into separate containers.

The invention is also directed to kits for use in methods of the invention. Such kits can be used for making, sequencing or amplifying nucleic acid molecules (single- or double-stranded), e.g., at the particular temperatures described herein. Kits of the invention may comprise a carrier, such as a box or carton, having in close confinement therein one or more (e.g., one, two, three, four, five, ten, twelve, fifteen, etc.) containers, such as vials, tubes, bottles and the like. In kits of the invention, a first container contains one or more of the indexed oligonucleotide sets of the present invention. Kits of the invention may also comprise, in the same or different containers, one or more reverse transcriptases, DNA ligases, DNA polymerases (e.g., thermostable DNA polymerases), transposases, one or more (e.g., one, two, three, four, five, ten, twelve, fifteen, etc.) suitable buffers for nucleic acid synthesis, one or more nucleotides and one or more (e.g., one, two, three, four, five, ten, twelve, fifteen, etc.) additional oligonucleotide primers. Kits of the invention also may comprise instructions or protocols for carrying out the methods of the invention.

In one embodiment, the kit includes instructional material that describes the use of the kit to generate a multi-indexed sequencing library, wherein the instructional material creates an increased functional relationship between the kit components and the individual using the kit. In one embodiment, the kit is utilized by one person or entity. In another embodiment, the kit is utilized by more than one person or entity. In one embodiment, the kit is used without any additional compositions or methods. In another embodiment, the kit is used with at least one additional composition or method.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: a Global View of Aging and Alzheimer's Pathogenesis-Associated Cell Population Dynamics in Mammalian Brain

In this example, a global view of aging and AD pathogenesis-associated cell population dynamics was obtained, by profiling ˜1.5 million single-cell transcriptomes at full gene body coverage and ˜380,000 single-cell chromatin accessibility profiles across the entire mammalian brains spanning various age and genotype groups. With the resulting datasets, over 300 cellular subtypes across the brain were identified, including extremely rare cell types (e.g., pinealocytes, tanycytes) that exist in less than 0.01% of the brain cell population. In addition, region-specific aging and AD effects were detected with high-resolution spatial transcriptomic analysis and the cell-type-specific manifestation of aging and AD-associated signatures were explored at both gene and isoform levels. With the EasySci method, a technical framework for individual laboratories to generate gene expression and chromatin accessibility profiles from millions of single cells cost-effectively is introduced. The EasySci pipeline, detailed experimental protocols, computation scripts, and datasets was made freely available to facilitate further exploration of the techniques and datasets.

As illustrated by the sub-cluster level analysis, the effects of aging and AD on the global brain cell population are highly cell-type-specific. While most brain cell types stay relatively stable the various conditions, many cell subtypes that are significantly changed (over two-fold change) in aged and AD model brains were identified, most of which were rare cell types and thus presumably missed in conventional “shallow” single-cell analysis. For example, the aged brain is characterized by the depletion of both rare neuronal progenitor cells and differentiating oligodendrocytes, associated with the enrichment of a C4b+ Serpina3n+ reactive oligodendrocyte subtype surrounding the subventricular zone (SVZ), suggesting a potential interplay between oligodendrocytes, local inflammatory signaling and the stem cell niche. Meanwhile, shared subtypes that were depleted (e.g., mt-Cytb+ mt-Rnr2− choroid plexus epithelial cell) or enriched (e.g., Col25a+ Ndrg1+ interbrain and midbrain neuron) in both early- and late-onset AD mutant brains were observed, validated by single-cell RNA-seq from both sexes as well as spatial transcriptomics analysis.

In summary, this example demonstrated the potential of novel ‘high-throughput’ single-cell genomics for quantifying the dynamics of rare cell types and novel subtypes associated with development, aging, and disease. Further development of high-throughput single-cell profiling strategies and computation approaches would make it possible to generate a comprehensive view of cell-type-specific dynamics across all mammalian organs through “saturate sequencing”, which may be especially critical for identifying rare cell types in human samples.

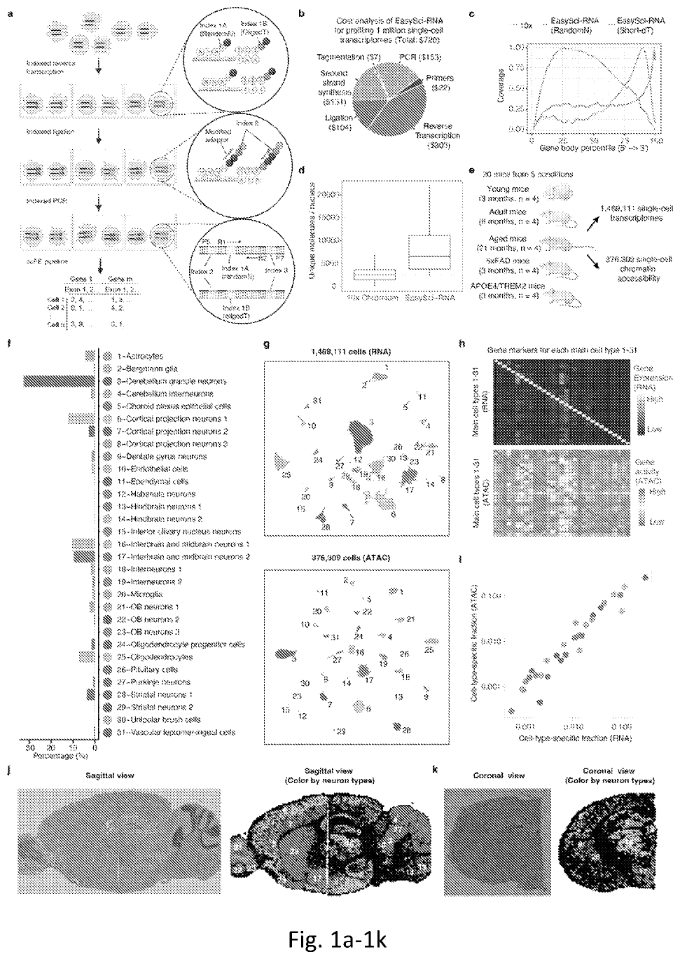

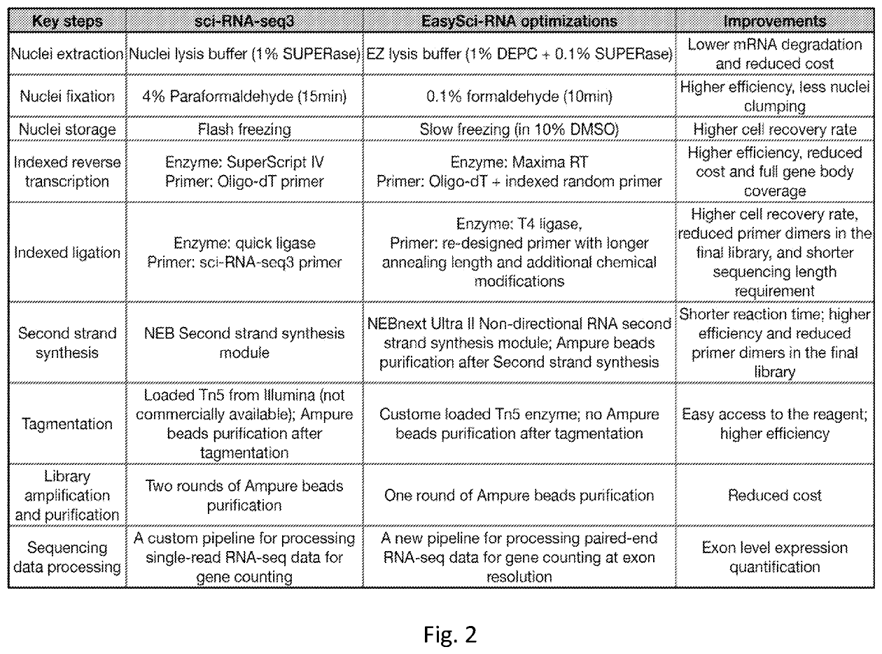

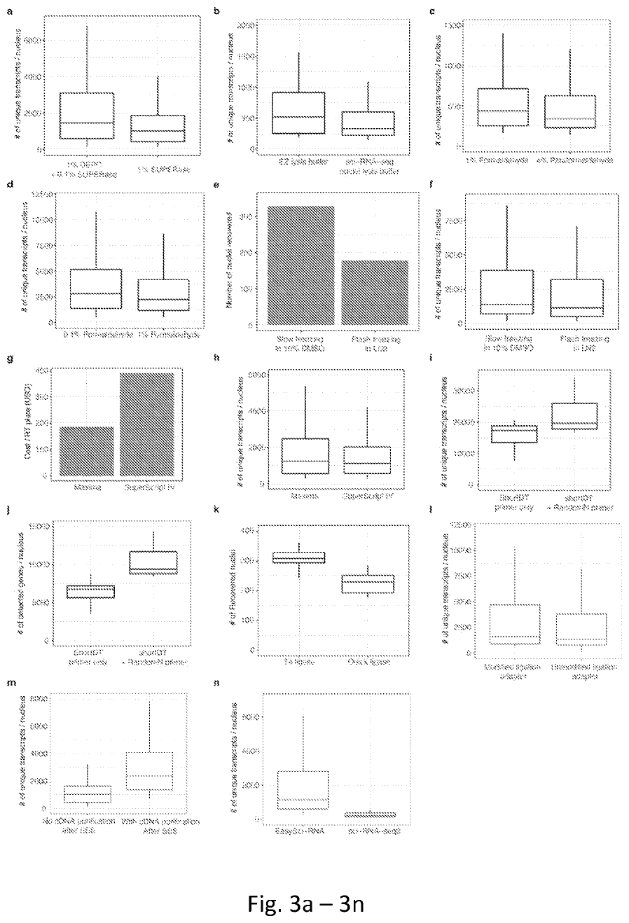

The major improvements of EasySci-RNA (FIG. 1a, FIG. 2, FIG. 3), include: (i) one million single-cell transcriptomes prepared at a library preparation cost of around $700, less than 1/300 the cost of the commercial platforms (Ding et al., Nat. Biotechnol. 38, 737-746 (2020)) (FIG. 1b). (ii) nuclei are deposited to different wells for reverse transcription with indexed oligo-dT and random hexamer primers (i.e., different molecular barcodes to separate reads primed by two types of primers and across different wells), thus recovering cell-type-specific gene expression at full gene body coverage (FIG. 1c). (iii) chemically modified oligos were included in the ligation reaction to prevent the formation of primer-dimers and increase the detection efficiency (FIG. 3); (iv) Cell recovery rate, as well as the number of transcripts detected per cell, were significantly improved through optimized nuclei storage and enzymatic reactions (FIG. 3). The optimized technique yields significantly higher signals per nucleus compared with the published sci-RNA-seq3 and the commercial platform (e.g., 10× Genomics) (FIG. 1d, FIG. 3n).

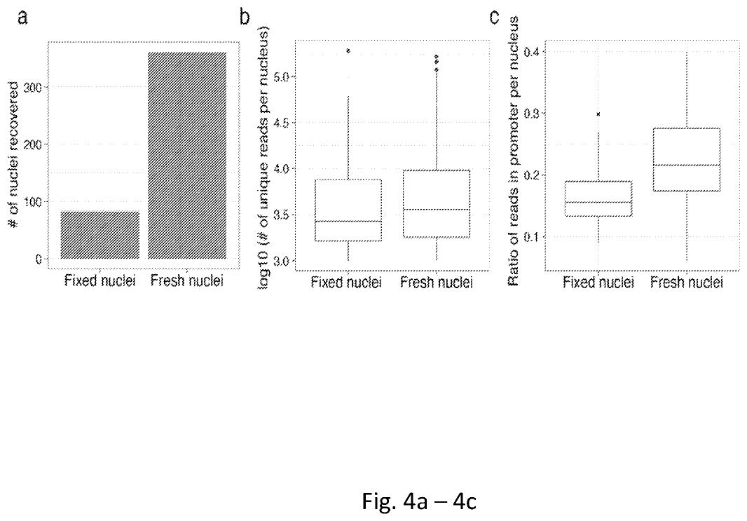

Leveraging the technical innovations from the development of EasySci-RNA, the recently published single-cell chromatin accessibility profiling method by combinatorial indexing was further optimized (sci-ATAC-seq3) (Domcke, S. et al., Science 370, (2020); Cusanovich, D. A. et al., Cell 174, 1309-1324.e18 (2018)). Critical additional improvements include: (i) tagmentation reaction with indexed Tn5 that are fully compatible with indexed ligation primers of EasySci-RNA; (ii) a modified nuclei extraction and cryostorage procedure to further increase the reaction efficiency and signal specificity (FIG. 4). The detailed protocols for the EasySci is provided as Example 2.

The Materials and Methods are now described.

### Animals

C57BL/6 wild-type mouse brains at three months (n=4), six months (n=4), and twenty-one months (n=4) were collected in this study. These age points correspond to approximately 20, 30, and 62 years in humans. Furthermore, to gain insight into the early cellular state changes underlying the pathophysiology of Alzheimer's disease, two AD models at 3-month-old from the same C57BL/6 background were added. These include an early-onset AD model (5×FAD) that overexpresses mutant human amyloid-beta precursor protein (APP) with the Swedish (K670N, M671L), Florida (I716V), and London (V717I) Familial Alzheimer's Disease (FAD) mutations and human presenilin 1 (PS1) harboring two FAD mutations, M146L and L286V. Brain-specific overexpression is achieved by neural-specific elements of the mouse Thy1 promoter (Oakley, H. et al., J. Neurosci. 26, 10129-10140 (2006)). The second, late-onset AD model (APOE*4/Trem2*R47H) in this study carries two of the highest risk factor mutations of LOAD (Karch, Biol. Psychiatry 77, 43-51 (2015)). including a humanized ApoE knock-in allele, where exons 2, 3, and most of exon 4 of the mouse gene were replaced by the human ortholog including exons 2, 3, 4 and some part of the 3′ UTR. Furthermore, a knock-in missense point mutation in the mouse Trem2 gene was also introduced, consisting of an R47H mutation, along with two other silent mutations (jax.org/strain/028709). Two male and two female mice are included in each condition.

By studying 3-month-old animals, the goal was to gain insight into the early changes underlying the pathophysiology of the AD models. Mature adult mice start at the age of 3 months, but multiple AD hallmarks, including amyloid beta plaques and gliosis, can be observed in the early-onset 5×FAD model (alzforum.org/research-models/5×fad-b6sjl). Therefore, this age might be the most appropriate to study early contributors of Alzheimer's disease pathogenesis.

### EasySci-RNA Library Preparation and Sequencing

Extracted mouse brains were snap-frozen in liquid nitrogen and stored at −80° C. Detailed step-by-step EasySci-RNA protocol is included as Example 2.

### Computational Procedures for Processing EasySci-RNA Libraries

A custom computational pipeline was developed to process the raw fastq files from the EasySci libraries. Similar to previous studies (Cao, J. et al., Science 370, (2020); Cao, J. et al., Nature 566, 496-502 (2019)), the barcodes of each read pair were extracted. Both adaptor and barcode sequences were trimmed from the reads. Second, an extra trimming step is implemented using Trim Galore (github.com/FelixKrueger/TrimGalore) with default settings to remove the poly (A) sequences and the low-quality base calls from the cDNA. Afterward, the paired-end sequences were aligned to the genome with the STAR aligner (Dobin et al., Bioinformatics 29, 15-21 (2013)), and the PCR duplicates removed based on the UMI sequence and the alignment location. Finally, the reads are split into SAM files per cell, and the gene expression is counted using a custom script. At this level, the reads from the same cell originating from the short dT and the random hexamer RT primers were counted as independent cells. During the gene counting step, reads were assigned to genes if the aligned coordinates overlapped with the gene locations on the genome. If a read was ambiguous between genes and derived from the short dT RT primer, the read was assigned to the gene with the closest 3′ end; otherwise, the reads were labeled as ambiguous and not counted. If no gene was found during this step, candidate genes 1000 bp upstream of the read or genes on the opposite strand were then searched for. Reads without any overlapped genes were discarded.

A similar strategy to generate an exon count matrix across cells was used. Specifically, the number of expressed exons based on the number of reads overlapping each exon was counted. If one read overlapped with multiple exons, this read was split between the exons. Read overlapped with multiple genes were discarded, except if the exact gene based on the other paired end read can be determined. For reads without overlapped genes, it was checked if there are any overlapped exons on the opposite strand. Reads without any overlapped exons were discarded.

### Cell Clustering and Cell Type Annotation of Single-Cell RNA-Seq Data

After gene counting, the cells with reads identified by both RT primers were kept. The reads from the same cells were then merged. Low-quality cells were removed based on one of the following criteria: (i) the percentage of unassigned reads>30%, (ii) the number of UMIs>20,000, and (iii) the detected number of genes<200. The Scrublet (Tong et al., Neurogenetics 11, 41-52 (2010)) computational pipeline was then used to identify and remove potential doublets, similar to a previous study (Cao, J. et al., Science 370, (2020)). At the end of these filtering steps, there were around 1.5 million brain cells in the dataset.

To identify distinct clusters of cells corresponding to different cell types, the 1,469,111 single-cell gene expression profiles were subjected to UMAP visualization and Louvain clustering, similar to a previous study (Cao, J. et al., Science 370, (2020)). the data was then co-embedded with the published datasets (Zeisel, A. et al., Front. Neuroinform. 12, 84 (2018); Yao et al., Nature 598, 103-110 (2021); Kozareva, V. et al., Nature 598, 214-219 (2021)) through Seurat (Stuart, T. et al., Cell 177, 1888-1902.e21 (2019)), and clusters were annotated based on overlapped cell types. The annotations were manually verified and refined based on marker genes. Differentially expressed genes across cell types were identified with the differentialGeneTest( ) function of Monocle 2 (Qiu, X. et al., Nat. Methods 14, 979-982 (2017)). To identify cell type-specific gene markers, genes that were differentially expressed across different cell types (FDR of 5%, likelihood) and also with a >2-fold expression difference between first and second-ranked cell types were selected.

### Isoform Expression Analysis

Isoform expression was quantified in EasySci data using an adapted version of the pipeline built by Booeshaghi et al. (Booeshaghi, A. S. et al., Nature 598, 195-199 (2021)). Short-dT and random hexamer reads for ˜1.5M single cells were merged into 617 pseudocells, grouping by individual mouse and cell types (31 cell types). The pseudocells were aligned to the mouse transcriptome with kallisto (Melsted, P. et al., Nat. Biotechnol. 1-6 (2021)), generating a raw isoform count matrix. To filter and preprocess the raw data, isoform counts were normalized by length, and genes and isoforms with a dispersion of less than 0.001 were removed. The gene count matrix was produced by aggregating counts of all isoforms of a given gene. Both isoform and gene count matrices were normalized by dividing the counts in each cell by the sum of the counts for that cell, then multiplying by 1,000,000 and transforming with numpy's log 1p( ) function. The filtered data contained 47,659 isoforms corresponding to 16,878 genes. Highly variable isoforms and genes were identified using scanpy, by binning into 20 bins and scaling the dispersion for each feature to zero mean and unit variance within each bin. The top 5,000 gene and isoforms in each matrix were retained based on normalized dispersion. Neighborhood components analysis was performed on the filtered and normalized isoform matrix after scaling the log(1+TPM) expression to zero mean and unit variance, training on cell type labels from each pseudocell with random state 42, and visualized using t-SNE with 5,000 iterations and random state 42. Differentially expressed isoforms were identified by looking for isoforms that were upregulated across a given cell type, while the genes containing those isoforms were not significantly expressed more among that cell type than its complement (the rest of the dataset). Isoforms expressed in less than 90% of pseudocells within a cell type were discarded. T-tests used a significance level of 0.01 with Bonferroni correction for multiple comparisons.

### Sub-Cluster Analysis of the Single-Cell RNA-Seq Data

To identify cell subtypes, each main cell type was selected and PCA, UMAP and Louvain clustering were applied similarly to the major cluster analysis, based on a combined matrix including the 30 principal components derived from the gene-level expression matrix and the first 10 principal components derived from the exon-level expression matrix. Sub-clusters that were not readily distinguishable in the UMAP space were then merged through an intra-dataset cross-validation procedure described before (Sziraki, A. et al., bioRxiv 2022.09.28.509825 (2022)). A total of 362 cell subtypes were identified, with a median of 1,030 cells in each group. All subtypes were contributed by at least two individuals (median of twenty). Differentially expressed genes and exons across cell types were identified with the differential Gene Test( ) function of Monocle 2 (Qiu, X. et al., Nat. Methods 14, 979-982 (2017)). To identify sub-cluster-specific differentially expressed genes associated with aging or AD models, a maximum of 5,000 cells per condition were sampled for downstream DE gene analysis using the differentialGeneTest function of the Monocle 2 package (Qiu, X. et al., Nat. Methods 14, 979-982 (2017)). The sex of the animals was included as a covariate to reduce gender-specific batch effects.

To detect cellular fraction changes at the subtype level across various conditions, a cell count matrix was first generated by computing the number of cells from every sub-cluster in each reverse transcription well profiled by EasySci-RNA. Each RT well was regarded as a replicate comprising cells from a specific mouse individual. the likelihood-ratio test was then applied to identify significantly changed sub-clusters between different conditions, with the differentialGeneTest( ) function of Monocle 2 (Qiu, X. et al., Nat. Methods 14, 979-982 (2017)). Sub-clusters were removed if they had less than 20 cells in either the male or female samples. In addition, subclusters were considered to change significantly only if there was at least a two-fold change between two groups and the q-value was less than 0.05.

### Gene Module Analysis

Gene module analysis was performed to identify the molecular programs underlying different cell types in the brain. First, the gene expression across all sub-clusters was aggregated. The aggregated gene count matrix was then normalized by the library size and then log-transformed (log 10(TPM/10+1)). Genes were removed if they exhibited low expression (less than 1 in all sub-clusters) or low variance of expression (i.e., the gene expression fold change between the maximum expressed sub-cluster and the median expression across sub-clusters are less than 5). The filtered matrix was used as input for UMAP/0.3.2 visualization (McInnes et al., Journal of Open Source Software vol. 3 861 (2018)) (metric=“cosine”, min_dist=0.01, n_neighbors=30). Genes were then clustered based on their 2D UMAP coordinates through densityClust package (rho=1, delta=1) (Rodriguez et al., Science 344, 1492-1496 (2014)).

### EasySci-ATAC Library Preparation and Sequencing

Mouse brain samples were snap-frozen in liquid nitrogen and stored at −80° C. For nuclei extraction, thawed brain samples were minced in PBS using a blade, re-frozen, stored at −80° C., and processed in multiple batches.

### Data Processing for EasySci-ATAC

Base calls were converted to fastq format and demultiplexed using Illumina's bcl2fastq/v2.19.0.316 tolerating one mismatched base in barcodes (edit distance (ED)<2). Downstream sequence processing were similar to sci-ATAC-seq (Cao, J. et al., Science 361, 1380-1385 (2018)). Indexed Tn5 barcodes and ligation barcodes were extracted, corrected to its nearest barcode (edit distance (ED)<2) and reads with uncorrected barcodes (ED>=2) were removed. Tn5 adaptors were removed from 5′-end and clipped from 3′-end using trim_galore/0.4.1 (github.com/FelixKrueger/TrimGalore). Trimmed reads were mapped to the mouse genome (mm39) using STAR/v2.5.2b (Dobin et al., Bioinformatics 29, 15-21 (2013)) with default settings. Aligned reads were filtered using samtools/v1.4.1 (Li et al., Bioinformatics 25, 2078-2079 (2009)) to retain reads mapped in proper pairs with quality score MAPQ>30 and to keep only the primary alignment. Duplicates were removed by picard MarkDuplicates/v2.25.2 (broadinstitute.github.io/picard/) per PCR sample. Deduplicated bam files were converted to bedpe format using bedtools/v2.30.0 (Quinlan et al., Bioinformatics 26, 841-842 (2010)), which were further converted to offset-adjusted (+4 bp for plus strand and −5 bp for minus) fragment files (.bed). Deduplicated reads were further split into constituent cellular indices by further demultiplexing reads using the Tn5 and ligation indexes. For each cell, sparse matrices counting reads falling into promoter regions (±1 kb around TSS) were also created for downstream analysis.

### Cell Filtering, Clustering and Annotation for EasySci-ATAC

SnapATAC273 (kzhang.org/SnapATAC2/index.html) was used to perform preprocessing steps for the EasySci-ATAC dataset. Cells with less than 1500 fragments and less than 2 TSS Enrichment were discarded. Potential doublet cells and doublet-derived subclusters were detected using an iterative clustering strategy (Cao, J. et al., Science 370, (2020)) modified to suit for scATAC-seq data. Briefly, cells were splitted by individual animals to overcome the large memory use when simulating doublets for the full dataset, and doublet scores were calculated using snap.pp.scrublet( ) (Wolock et al., Cell Syst 8, 281-291.e9 (2019)). Then, all cells were combined, followed by clustering and sub-clustering analysis with spectral embedding and graph-based clustering implemented in SnapATAC273 (kzhang.org/SnapATAC2/index.html). Cells labeled as doublets (defined by a doublet score cutoff of 0.2) or from doublet-derived sub-clusters (defined by a doublet ratio cutoff of 0.4) were filtered out. In addition, cells with high fragment numbers in each main cluster (defined as cells with fragments number higher than the 95th quantile within the main cluster) were also filtered out. A gene activity matrix was generated using snap.pp.make_gene_matrix( ) for the following integration analysis.

A deep-learning-based framework scJoint (Lin et al., Nat. Biotechnol. 40, 703-710 (2022)) was used to annotate main ATAC-seq cell types using the EasySci-ATAC dataset as a reference. First, 5,000 cells from each main cell type of the EasySci-RNA dataset were subsampled, and genes detected in more than 10 cells were selected. Then, the gene count matrix and cell type labels of EasySci-RNA, along with the gene activity matrix of EasySci-ATAC were input into the scJoint pipeline with default parameters. Jointed embedding layers calculated from scJoint were used for UMAP visualizations using python package umap/v0.5.3 (umap-learn.readthedocs.io/en/latest/). Cells were assigned to the prediction label with the highest abundance within each louvain cluster. Clusters with low purities (i.e., less than 80% cells were from the highest abundant cell type) were removed upon inspections. Finally, to validate the integration-based annotations, differentially expressed genes identified from the RNA-seq data were selected with the following criteria: fold change between the maximum and the second maximum expressed cell type>1.5, q-value<0.05, TPM (transcripts per million)>20 in the maximum RNA group and RPM (reads per million)>50 in the maximum ATAC group. Top 10 genes ranked by fold change between the maximum and the second maximum expressed group were selected using RNA-seq data for each cell type. If there were less than 10 genes passing the cutoff, the top genes ranked by the fold change between the maximum expressed cell type and the mean expression of other cell types were selected. The aggregated gene count and gene body accessibility (gene activity) for each cell type were calculated.

Subcluster level integrations for Microglia, OB neurons 1 and Oligodendrocytes were similar to the main cluster level integrations with mild modifications. For Microglia and OB neurons 1, all cells from the EasySci-RNA dataset were used as input for the integrations. For Oligodendrocytes, 2,000 cells from each subcluster were subsampled for integration analysis. Similarly, the subcluster level integrations were validated by inspecting the aggregated gene activity of subcluster-specific gene markers in the predicted ATAC subclusters. Subcluster marker genes were identified by differential expression analysis using scRNA-seq data and selected by the following criteria: fold change between the maximum expressed sub-cluster and the mean of all the other subclusters within the same main cell type>2, FDR<0.05, TPM (transcripts per million)>50 in the maximum expressed RNA group and RPM (reads per million)>50 in the maximum accessible ATAC group.

### Peak Calling, Peak-Based Dimension Reduction and Identifications of Differential Accessible Peaks

To define peaks of accessibility, MACS2/v2.1.176 was used. Nonduplicate ATAC-seq reads of cells from each main cell type were aggregated and peaks were called on each group separately with these parameters: --nomodel --extsize 200 --shift -100 -q 0.05. To correct for differences in read depth or the number of nuclei per cell type, MACS2 peak scores (−log 10(q-value)) were converted to ‘score per million’ (Corces, M. R. et al. Science 362, (2018)) and peaks were filtered by choosing a score-per-million cut-off of 1.3. Peak summits were extended by 250 bp on either side and then merged with bedtools/v2.30.0. Cells were determined to be accessible at a given peak if a read from a cell overlapped with the peak. The peak count matrix was generated by a custom python script with the HTseq package (Anders et al., Bioinformatics 31, 166-169 (2015)).

R package Signac/v1.7.0 (Stuart et al., Nat. Methods 18, 1333-1341 (2021)) was used to perform the dimension reduction analysis using the peak-count matrix. 5,000 cells from each main cell type were subsampled and TF-IDF normalization was performed using RunTFIDF( ), followed by singular value decomposition using RunSVD( ) and retained the 2nd to 30th dimensions for UMAP visualizations using RunUMAP( ).

Differentially accessible peaks across cell types were identified using monocle 2 (Qiu, X. et al., Nat. Methods 14, 979-982 (2017)) with the differentialGeneTest( ) function. 5,000 cells were subsampled from each cell type for this analysis. Peaks detected in less than 50 cells were filtered out. Peaks that were differentially accessible across cell types were selected by the following criteria: 5% FDR (likelihood ratio test), and with TPM>20 in the target cell type.

### Transcription Factor Motif Analysis

Chrom Var/v1.16.0 (Schep et al., Nat. Methods 14, 975-978 (2017)) was used to access the TF motif accessibility using a collection of the cisBP motif sets curated by chromVARmotifs/v0.2.0 (Schep et al., Nat. Methods 14, 975-978 (2017); github.com/GreenleafLab/chromVARmotifs). To investigate TF regulators at the main cluster level, 5,000 cells from each main cell type were subsampled, and the motif deviation score for each single cell was calculated using the Signac wrapper RunChromVAR( ). The motif deviation scores of each single cell were rescaled to (0, 10) using R function rescale( ) and then aggregated for each cell type. In addition, the gene expression of each TF in each cell type were also aggregated. The Pearson correlations between the aggregated motif matrix and aggregated TF expression matrix were then computed after scaling across all main cell types. TF analysis at the subcluster level was performed similarly with modifications. For each cell type of interest, peaks detected in more than 20 cells were selected and only cells with more than 500 reads in peaks were kept. Peaks were resized to 500 bp (±250 bp around the center) and motif occurrences were identified using matchMotifs( ) function from motifmatchr/v1.16.0 (github.com/GreenleafLab/motifmatchr). The Motif deviation matrix was calculated using the Chrom Var function computeDeviations( ). Then, the motif deviation scores were rescaled to (0, 10) and aggregated per subcluster. Pearson correlation was calculated between the aggregated motif activity and aggregated TF expression across subclusters after scaling. ATAC-seq subclusters with less than 20 cells were excluded from the correlation analysis

### Spatial Gene Expression Profiling of Mouse Brains

Spatial gene expression analysis experimental protocol was followed according to Visium Spatial Gene Expression User Guide (catalog no. CG000160), Visium Spatial Tissue Optimization User Guide (catalog no. CG000238 Rev A, 10× Genomics) and Visium Spatial Gene Expression User Guide (catalog no. CG000239 Rev A, 10× Genomics). Briefly, mice were sacrificed, and brains were extracted and frozen with liquid nitrogen. Frozen brain was embedded in OCT (Tissue TEK O.C.T compound) and cryosectioned at −15 C (Leica cryostat). Coronally placed brains were cut halfway, to place half coronally sectioned brains at 10 um on Visium tissue optimization, or gene expression analysis slides capture areas. User guide CG000160 from 10× Genomics was followed for methanol fixation and H&E stain. After fixation and staining, imaging was performed using Leica DMI8, and images were stitched using Leica Application Suite X and saved into tiff format. After tissue fixation and staining, Visium Spatial Tissue Optimization User Guide (catalog no. CG000238 Rev A, 10× Genomics) or Visium Spatial Gene Expression User Guide (catalog no. CG000239 Rev A, 10× Genomics) were followed for either protocol optimization, or gene expression analysis, respectively. Tissue optimization was performed according to CG000238, and according to optimization experiments, 18 min permeabilization provided the most optimal signal, and was followed for gene expression library preparation as well. Libraries were prepared according to Visium Spatial Gene Expression User Guide (CG000239, 10× Genomics)

### Library Preparation and Data Processing of Spatial Transcriptomics

Libraries were sequenced using a NextSeq1000 system. BCL files were converted to FASTQ, and raw FASTQ files and .tiff histology images were processed with spaceranger-1 2.2 software. Spaceranger-1.2.2 uses STAR for RNA reads genome alignment, and utilized the GRCm38 (mouse mm10) as the reference genome provided from 10× Genomics. The downstream visualization and clustering analysis of the spatial transcriptomic data following the tutorial of Seurat (satijalab.org/seurat/articles/spatial_vignette.html) was performed with default parameters.

### Spatial Transcriptomic Analysis to Locate the Spatial Distributions of Main Cell Types and Subtypes

To annotate the spatial locations of main cell types, the Easy Sci-RNA data was integrated with publicly available 10× Visium spatial transcriptomics dataset (satijalab.org/seurat/articles/spatial_vignette.html) through a non-negative least squares (NNLS) approach modified from a previous study (Cao, J. et al., Science 370, (2020)). Cell-type-specific UMI counts, normalized by the library size, multiplied by 100,000, and log-transformed after adding a pseudo-count were aggregated. A similar procedure was applied to calculate the normalized gene expression in each spatial spot captured in 10× Visium dataset. Non-negative least squares (NNLS) regression was applied to predict the gene expression of each spatial spot in 10× Visium data using the gene expression of all cell types recovered in Easy-RNA data:

$${T}_{a} = {β}_{0 a} + {β}_{1 a} {M}_{b}$$

where Tₐ and M_{b} represent filtered gene expression for target spatial spot from 10× Visium dataset A and all cell types from EasySci-RNA dataset B, respectively. To improve accuracy and specificity, cell type-specific genes were selected for each target cell type by: 1) ranking genes based on the expression fold-change between the target cell type vs. the median expression across all cell types, and then selecting the top 200 genes. 2) ranking genes based on the expression fold-change between the target cell type vs. the cell type with maximum expression among all other cell types, and then selecting the top 200 genes. 3) merging the gene lists from step (1) and (2). β₁ₐ is the correlation coefficient computed by NNLS regression.

Similarly, the order of datasets A and B were switched, and the gene expression of target cell type (T_{b}) in dataset B were predicted with the gene expression of all spatial spots (Mₐ) in dataset A:

$${T}_{b} = {β}_{0 b} + {β}_{1 b} {M}_{a}$$

Thus, each spatial spot a in 10× Visium dataset A and each cell type b in EasySci dataset B are linked by two correlation coefficients from the above analysis: β_{ab} for predicting the gene expression in each spatial spot a using b, and β_{ba} for predicting gene expression in each cell type b using a. The two values were combined by:

$$β = ( {β}_{ab} + 0.01 ) * ( {β}_{ba} + 0.01 )$$

The β is then capped to [1,3]. β reflects the cell-type-specific abundance across different spatial spots in 10× Visium datasets with high specificity. β was thus used as the alpha value (i.e., the opacity of a geom) to plot the spatial distribution of different cell types.

To characterize the expression of sub-cluster specific gene markers, the gene expression in each spatial spot of 10× Visium data was first normalized by the library size, multiplied by 100,000, and log-transformed after adding a pseudo-count. The expression of genes from sub-cluster specific gene markers was aggregated, scaled to z-score and capped to [3, 6]. Of note, the sub-cluster specific gene markers were selected by differentiation expression analysis described above and only DE genes (FDR of 5%, with a >2-fold expression difference between first and second ranked sub-clusters, expression TPM>50 in at least one sub-cluster) were selected as gene markers. In addition, the aggregated expression of the selected gene markers across all 362 sub-clusters were examined to further validate the specificity of gene markers for labeling target sub-clusters.

The Experimental Results are now described.

### a Comprehensive Cell Catalog of the Entire Mammalian Brain in Aging and AD

The EasySci method was applied to characterize cell-type-specific gene expression, and chromatin accessibility profile across the entire mouse brains sampling at different ages, sexes, and genotypes (FIG. 1c). C57BL/6 wild-type mouse brains were collected at three months (n=4), six months (n=4), and twenty-one months (n=4). To gain insight into the early molecular changes associated with the pathophysiology of AD, two AD models from the same C57BL/6 background at three months were included. These include an early-onset AD model (5×FAD) that overexpresses mutant human amyloid-beta precursor protein (APP) and human presenilin 1 (PS1) harboring multiple AD-associated mutations (Oakley, H. et al., J. Neurosci. 26, 10129-10140 (2006)); and a late-onset AD model (APOE*4/Trem2*R47H) that carries two of the highest risk factor mutations, including a humanized ApoE knock-in allele and missense mutations in the mouse Trem2 gene (Karch et al., Biol. Psychiatry 77, 43-51 (2015); jax.org/strain/028709).

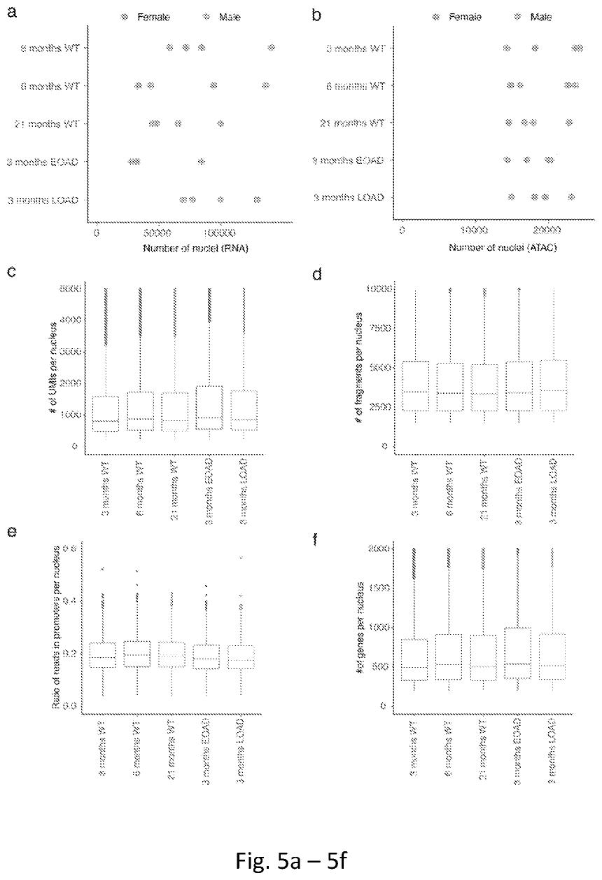

Nuclei were first extracted from the whole brain, then deposited to different wells for indexed reverse transcription or transposition, such that the first index identified the originating sample and assay type of any given well. The resulting EasySci libraries were sequenced in two Illumina NovaSeq run, yielding a total of 20 billion reads (around 10 billion for each library). After filtering out low-quality cells and potential doublets, gene expression profiles in 1,469,111 single cells (a median of 70,589 cells per brain sample, FIG. 5a) and chromatin accessibility profiles in 376,309 single cells (a median of 18,112 cells per brain sample, FIG. 5b) across conditions were recovered. Despite shallow sequencing depth (˜4500 and ˜10,000 raw reads per cell for RNA and ATAC, respectively), a median of 935 UMIs (RNA) and 3,918 unique fragments (ATAC) were recovered per nucleus (FIG. 5c-d), comparable to the recently published single-cell RNA-seq and ATAC-seq datasets (Cao, J. et al., Science 370, (2020); Cao, J. et al., Nature 566, 496-502 (2019); Domcke, S. et al., Science 370, (2020)). A median of 19% of ATAC-seq reads were near a TSS (±1 kb) (FIG. 5e), comparable to the published sci-ATAC-seq3 approach (Domcke et al., Cell 174, 1309-1324.e18 (2018)).

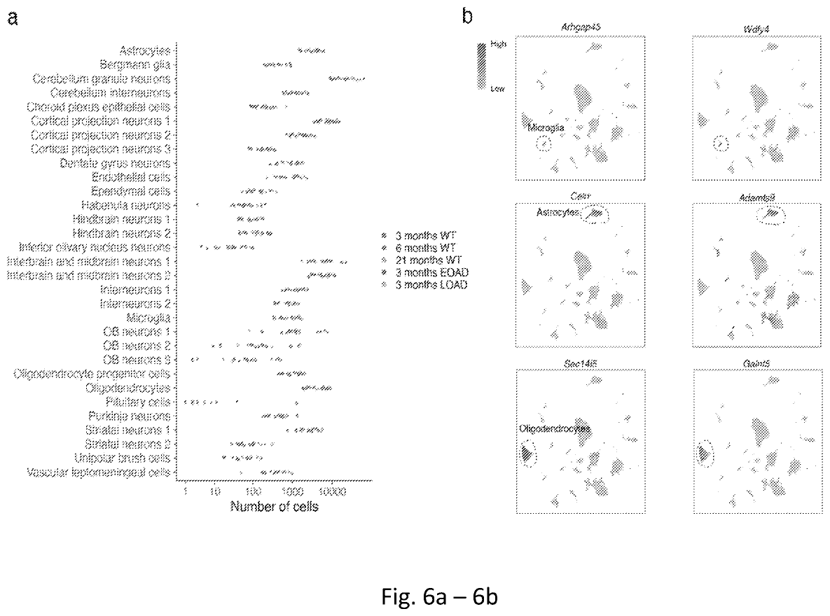

With UMAP visualization (McInnes et al., Journal of Open Source Software vol. 3 861 (2018)), Louvain clustering (Blondel et al., Journal of Statistical Mechanics: Theory and Experiment vol. 2008 P10008 (2008)), and annotation based on cell-type-specific gene markers (Zeisel et al., Cell 174, 999-1014.e22 (2018)), 31 main cell types were identified by gene expression clusters (a median of 16,370 cells per cell type; FIG. 1g). Each cell type was observed in almost every individual (except pituitary cells were missing in three out of twenty individuals) (FIG. 6a), ranging from 0.05% (Inferior olivary nucleus neurons) to 32.5% (Cerebellum granule neurons) of the brain cell population (FIG. 1f). An average of 74 marker genes were identified for each main cell type (defined as differentially expressed genes with at least a 2-fold difference between first and second-ranked cell types with respect to expression; FDR of 5%; and TPM>50 in the target cell type). In addition to the established marker genes, many novel markers that were not previously associated with the respective cell types were identified, such as markers for microglia (e.g., Arhgap45 and Wdfy4), astrocytes (e.g., Celrr and Adamts9) and oligodendrocytes (e.g., Sec14l5 and Galnt5) (FIG. 6b).

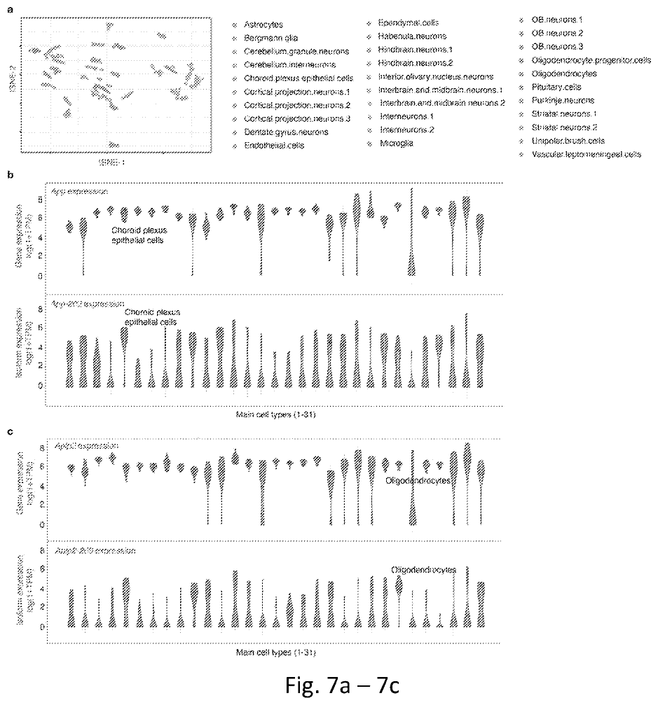

Isoform expression was then quantified through an adapted version of the published pipeline (Booeshaghi et al., Nature 598, 195-199 (2021)). Briefly, random hexamer reads from each cell type in every individual mouse brain were merged, yielding 613 pseudocells. The merged reads were then aligned to the mouse transcriptome, resulting in 33,361 isoforms corresponding to 12,636 genes. As expected, it was found that previously identified main clusters can be resolved through isoform expression (FIG. 7a). Certain isoforms were strongly expressed in a given cell type even though their corresponding genes were not cell-type-specific. For example, App-202, an isoform of the amyloid precursor protein gene, is preferentially expressed in choroid plexus epithelial cells, while its corresponding gene is not (FIG. 7b). Similarly, Aplp2-209, an isoform of the amyloid beta precursor-like protein 2 gene, is differentially expressed in oligodendrocytes. By contrast, the cell-type-specificity is not detected at the gene level (FIG. 7c)

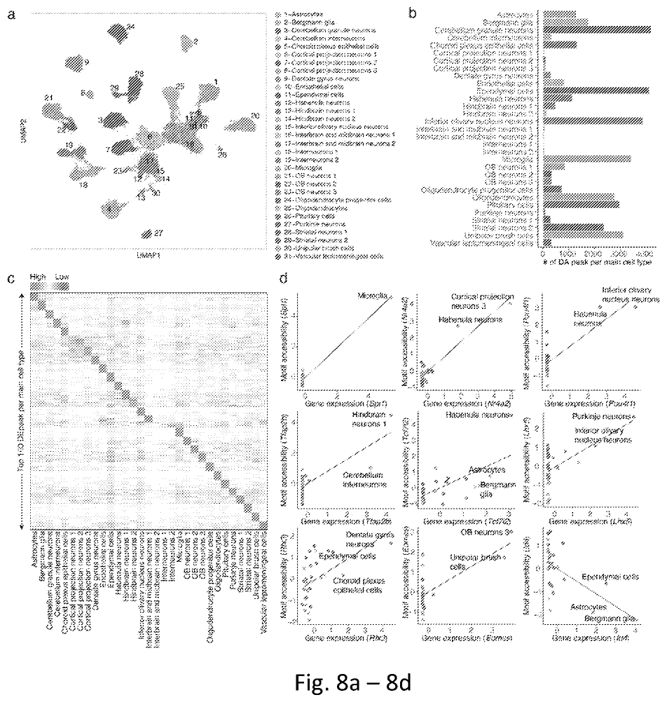

To reconstruct a brain cell atlas of both gene expression and chromatin accessibility, a deep learning-based strategy (Lin et al., Nat. Biotechnol. 40, 703-710 (2022)) was applied to integrate the chromatin accessibility profile of 376,309 single cells with gene expression data (FIG. 1g). As expected, the gene body accessibility and expression of marker genes across cell types were cross-validated (FIG. 1h). Furthermore, the fraction of each cell type was highly correlated between two molecular layers (FIG. 1i). To gain more insight into the epigenetic controls of the diverse cell types in the brain, peaks of accessibility within each cell type were next identified, yielding a master set of 339,951 peaks. There was a median of 34% of reads in peaks per nuclei. UMAP dimension reduction using the resulting peak count matrix readily separates main cell types, further validating the integration-based annotations (FIG. 8a). Through differential accessibility (DA) analysis, a median of 474 differential accessible peaks per cell type was identified (FDR of 5%, TPM>20 in the target cell type, FIG. 8b, c). Furthermore, key cell-type-specific TF regulators for diverse cell types were revealed by correlation analysis between motif accessibility and expression patterns (FIG. 8d), such as Spi1 in microglia (Yeh et al., Trends Mol. Med. 25, 96-111 (2019)), Nr4a2 in cortical projection neurons 3 (Watakabe et al., Cereb. Cortex 17, 1918-1933 (2007)), and Pou4f1 in inferior olivary nucleus neurons (McEvilly et al., Nature 384, 574-577 (1996))

Toward a spatially resolved brain atlas, the dataset was integrated with a 10× Visium spatial transcriptomics dataset (Ståhl et al., Science 353, 78-82 (2016)) through a modified non-negative least squares (NNLS) approach. Aggregated cell-type-specific gene expression data were used as input to decompose mRNA counts at individual spatial locations of both sagittal and coronal sections of the entire mouse brain, thereby estimating the cell-type-specific abundance across locations. As expected, specific brain cell types were mapped to distinct anatomical locations (FIG. 1j), especially for region-specific cell types such as cortical projection neurons (clusters 6,7,8), cerebellum granule neurons (cluster 3) and hippocampal dentate gyrus neurons (cluster 9). The integration analysis further confirmed the annotations and spatial locations of main cell types in the single-cell datasets.

### a Computational Framework Tailored to Characterize Cellular Subtypes in the Mammalian Brain

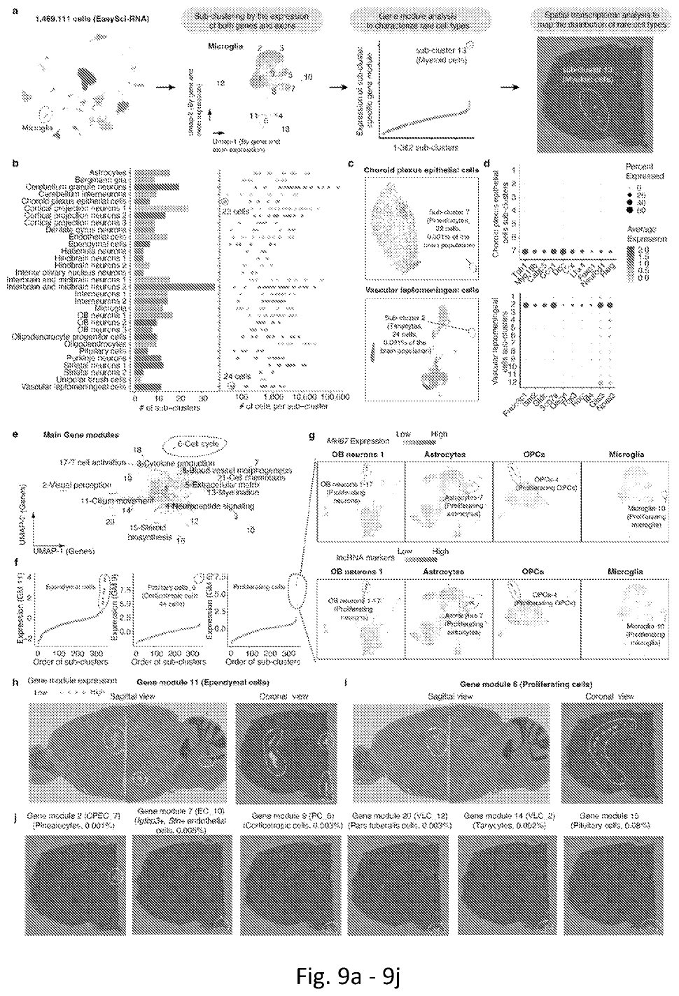

To investigate the molecular signatures and spatial distributions of diverse cellular subtypes in the brain, a novel computational framework tailored to sub-cluster level analysis was developed (FIG. 9a). Key steps include: (i) sub-clustering analysis by the expression of both genes and exons to increase the clustering resolution; (ii) gene module analysis to identify the signatures of main and rare cell types; (iii) spatial mapping rare cell subtypes through cell-type-specific gene module expression.

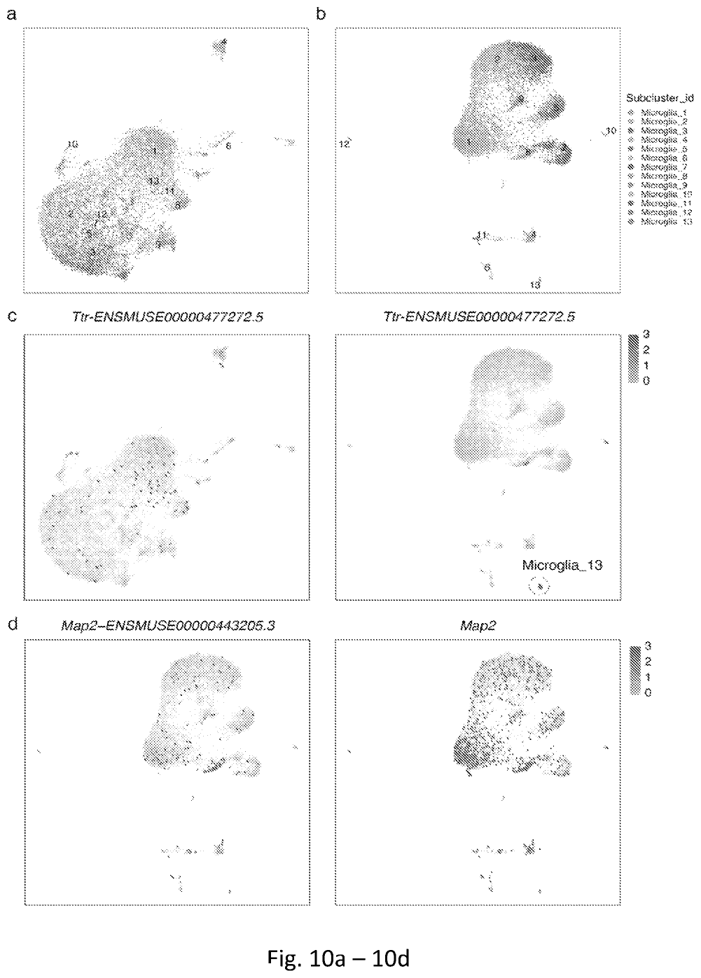

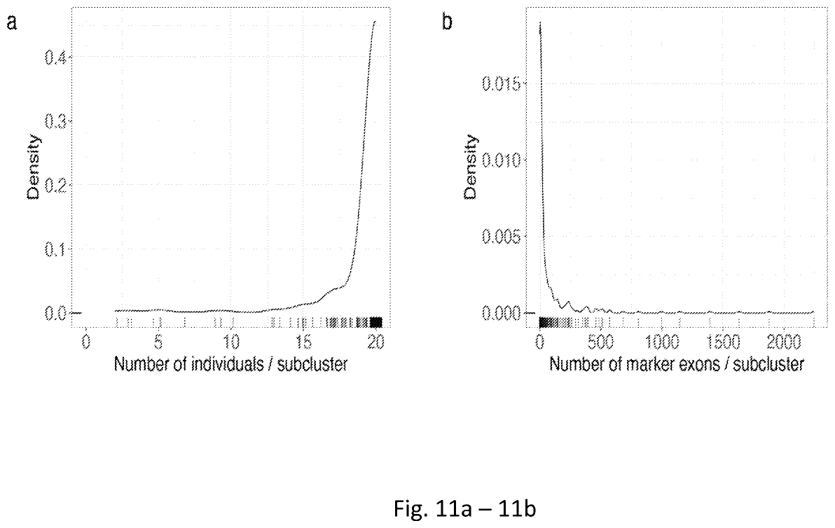

Rather than performing the sub-clustering analysis with the gene expression alone, the unique feature of EasySci-RNA (i.e., full gene body coverage) was exploited, by combining the top principal components of gene counts and exonic counts from each cell for unsupervised clustering. The added information enabled the recovery of sub-clusters with higher resolution. For example, several microglia subtypes that showed cell-type-specific exonic markers but were not easily separated by gene expression alone were identified (FIG. 10a-c). Leveraging this novel sub-clustering strategy, a total of 362 subclusters was identified, with a median of 1,030 cells in each group (FIG. 9b). All sub-clusters were contributed by at least two individuals (median of twenty), with a median of nine exonic markers enriched in each group (At least a 2-fold difference between first and second-ranked cell types with respect to expression; FDR of 5%; and TPM>50 in the target sub-cluster, FIG. 11). Some sub-cluster-specific exonic markers were not detected by conventional differential gene analysis (e.g., Map2-ENSMUSE00000443205.3, FIG. 10d). Notably, the sub-clustering strategy favors detecting extremely low-abundance cell types (FIG. 9c, d). For example, the smallest sub-cluster (choroid plexus epithelial cells-7) contained only 21 cells (0.001% of the brain population), representing rare pinealocytes in the brain based on gene markers such as Tph1 and Ddc. The second smallest sub-cluster (vascular leptomeningeal cells-2, 35 cells) represents the rare tanycytes, validated by multiple gene markers (e.g., Fndc3c1, Scn7a).

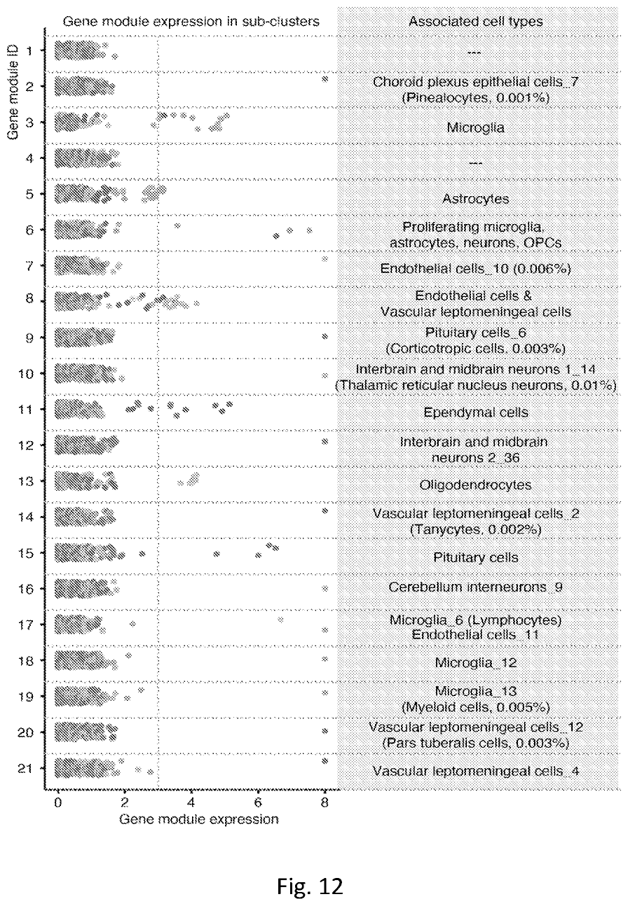

The key molecular programs underlying diverse cell subtypes was then examined by gene module analysis. Genes were clustered based on their expression variance across all 362 cell sub-clusters, revealing a total of 21 gene modules (GM) (FIG. 9e, FIG. 12). The largest gene module (GM1) corresponds to a group of housekeeping genes (e.g., ribosomal synthesis) universally expressed across all sub-clusters. Several gene modules were enriched in specific main cell types, such as an ependymal cell-specific gene module (GM11, enriched biological process: cilium movement, adjusted p-value=1.2e-26) (Kuleshov et al., Nucleic Acids Res. 44, W90-7 (2016)) (FIG. 9f). Meanwhile, gene modules that marked specific rare subtypes were detected. For example, GM9, including genes in neuropeptide signaling (e.g., Thx19, Pomc (Liu et al., Proc. Natl. Acad. Sci. U.S.A 98, 8674-8679 (2001)), was highly enriched in a subtype of pituitary cells (pituitary cells-6) corresponding to corticotropic cells (FIG. 9f). A similar analysis enabled characterization of other rare cell subtypes, including myeloid cells (Microglia sub-cluster 13, 67 cells, marked by GM19), pars tuberalis cells (Vascular leptomeningeal cells_12, 44 cells, marked by GM20), as well as aforementioned pinealocytes (choroid plexus epithelial cells sub-cluster 7, 21 cells, marked by GM2) (FIG. 12). Remarkably, rare proliferating cell types were identified through a cell-cycle-related gene module (GM6, enriched biological process: microtubule cytoskeleton organization involved in mitosis, adjusted p-value=1.2e-44) (Kuleshov et al., Nucleic Acids Res. 44, W90-7 (2016)), including proliferating cells of neurons (OB neurons 1-17, 511 cells), astrocyte (Astrocytes-7, 2,269 cells), OPCs (OPC-4, 641 cells) and microglia (Microglia-10, 82 cells) (FIG. 9f). These sub-clusters were marked by conventional proliferating markers such as Mki67, as well as a group of lncRNAs (e.g., Gm29260, Gm37065), most of which were not well-characterized in previous studies (FIG. 9g).

To spatially map the rare cell types, the expression patterns of cell-type-specific gene modules across spatial spots of the 10× Visium spatial transcriptomic datasets were next investigated (Liu et al., Proc. Natl. Acad. Sci. U.S.A 98, 8674-8679 (2001)). Strikingly, this approach enabled mapping of the anatomical locations of diverse cell types/subtypes with high accuracy. For example, ependymal cells, a critical cell type regulating cerebrospinal fluid (CSF) homeostasis, were mapped along brain ventricles as expected (FIG. 9h). Furthermore, rare proliferating cells were mapped to the subventricular zone area (FIG. 9i). A similar analysis enabled spatially mapping of other rare cell types with high resolution, including pinealocytes (CPEC_7, GM2), corticotropic cells (PC_6, GM9), pars tuberalis cells (VLC_12, GM20), tanycytes (VLC_2, GM14) and a less-characterized endothelial cell in the pituitary gland (Igfbp3− Sfn+ endothelial cells, EC_10, GM7) (FIG. 9j).

### a Global View of Mammalian Brain Cell Population Dynamics Across the Adult Lifespan at Subtype Resolution

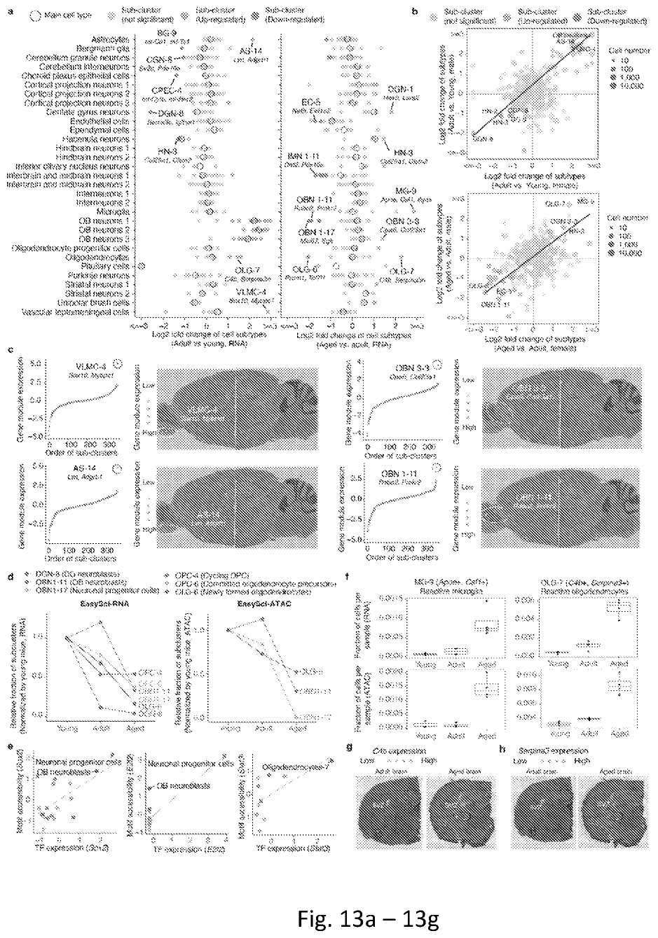

To obtain a global view of brain cell population dynamics at timepoints across the adult lifespan, the cell-type-specific fractions recovered from cell populations in each individual mouse were quantified. Differential abundance analysis was performed across all 362 sub-clusters, yielding 45 significantly changed sub-clusters during the early growth stage (between 3 and 6 months) and 29 significantly changed sub-clusters upon aging (between 6 and 21 months; FDR of 0.05, at least two-fold change of cellular fractions, FIG. 13a). Most significantly changed cell types were consistent between male and female mice (FIG. 13b).

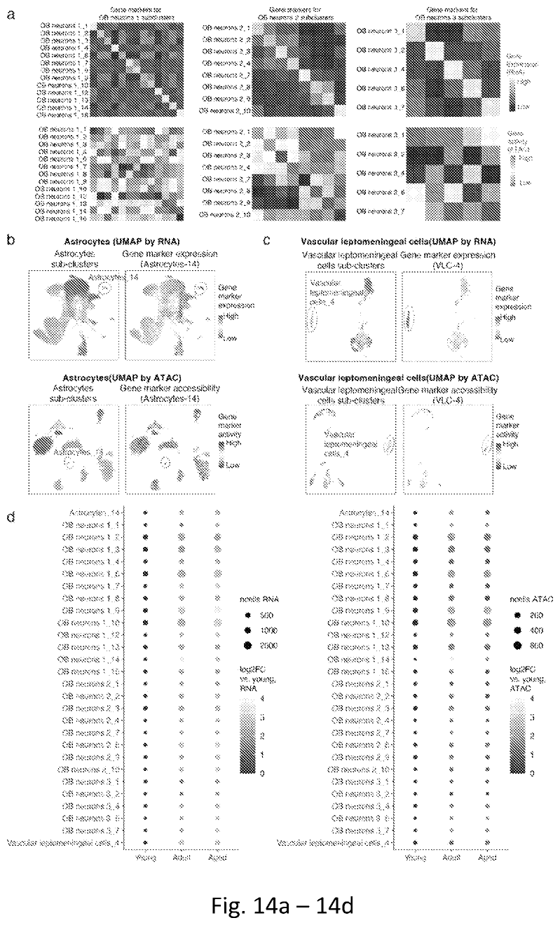

As expected, both main and subtypes of olfactory bulb (OB) neurons showed a significant population increase from young to adult mice (FIG. 13a, left), consistent with the expansion of the OB region in early growth (Tufo et al., Development 149, (2022)). Meanwhile, a rare astrocytes-14 subtype (Lyn+ Adgrb1+; 0.05% of the global population) and a vascular leptomeningeal cell subtype 4 (Sox10+ Mybpc1+; 0.06% of the global population) also showed substantial expansion in the same period. Strikingly, these two rare cell subtypes were spatially mapped to the same OB region based on the expression of cell-type-specific gene markers in 10× Visium spatial transcriptomic data (FIG. 13c, left), suggesting their potential roles in the OB expansion. The chromatin accessibility of these two rare cell types was further characterized, along with many OB neuron subtypes, by single-cell RNA-seq and ATAC-seq integration analysis through the deep-learning-based strategy (Lin et al., Nat. Biotechnol. 40, 703-710 (2022)) described above (FIG. 14a-c). The observed cell population dynamics can be further cross-validated by two molecular layers (i.e., RNA and ATAC) (FIG. 14d). In fact, the astrocytes-14 subtype shows a high expression of BAI1, which has been reported to be involved in the clean-up of apoptotic neuronal debris produced in the fast growth (Sokolowski et al., Brain Behav. Immun. 25, 915-921 (2011)). In addition, vascular leptomeningeal cell subtype 4 may correspond to olfactory ensheathing cells based on its high expression of Sox10 and Mybpc1 (Rosenberg et al., Science 360, 176-182 (2018); Tepe et al., Cell Rep. 25, 2689-2703.e3 (2018)).

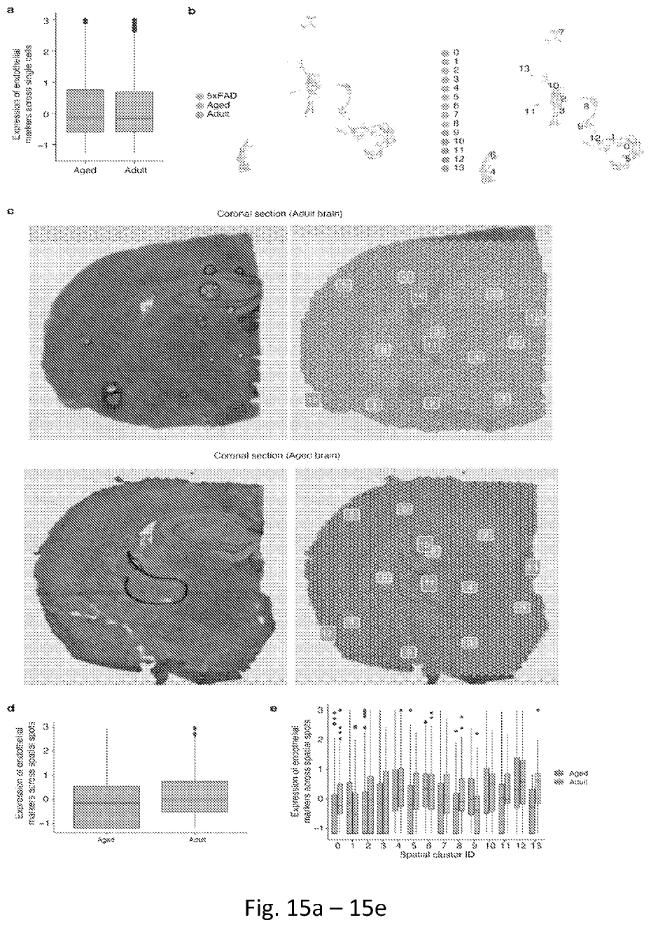

The aging-associated cell population changes (between 6 and 21 months) were remarkably distinct from cells present in the brains during the early growth stage. Different from the global expansion of OB neurons from young to adult, most cell types remained relatively stable at the main-cluster level (less than 2-fold change between 6 and 21 months) (FIG. 13a, right). Interestingly, an age-dependent reduction of the endothelial cell population in the scRNA-seq dataset was detected (FIG. 13a). A similar but milder trend was observed in the scATAC-seq dataset (i.e., endothelial cell fractions: 0.59% in adult brains vs. 0.56% in aged brains). To better understand the region-specific changes of endothelial cells in aging, a 10× Visium spatial transcriptome dataset profiling both adult and aged mouse brains was generated. A panel of endothelial-specific gene markers not associated with aging was selected and their expression was used to estimate the effect of aging on endothelial cell density across brain regions (FIG. 15a). Consistent with the single-cell data, a globally reduced expression of endothelial markers in the spatial transcriptomic analysis of the aged brain was detected, and the reduction varied in different brain regions (FIG. 15b-c). In addition to the vascular cells, the regional-specific effects of aging for certain neuron subtypes was detected. For example, the analysis revealed an aging-associated expansion of an OB neuron subtype (OBN3-3, marked by Cpa6 and Col23a1), while another OB neuron subtypes (OBN1-11, OB neuroblasts marked by Robo2 and Prokr2 (Zeisel et al., Cell 174, 999-1014.e22 (2018); Puverel et al., J. Comp. Neurol. 512, 232-242 (2009)) were substantially depleted in aged brains. Interestingly, these subtypes were spatially mapped to different areas of the olfactory bulb (FIG. 13d), indicating a region-specific change of OB neuron subtypes upon aging. Notably, the significantly altered cellular subtypes show consistent proportion changes in male and female mice (FIG. 13b).

A marked reduction in adult neurogenesis and oligodendrogenesis was detected across the lifespan of the mammalian brain (FIG. 13d, left). For example, the most depleted populations in the aged brain include OB neuroblasts (OB neurons 1-11, marked by Prokr2 and Robo2 (Zeisel et al., Cell 174, 999-1014.e22 (2018); Puverel et al., J. Comp. Neurol. 512, 232-242 (2009)), OB neuronal progenitor cells (OB neurons 1-17, marked by Mki67 and Egfr (Pastrana et al., Proc. Natl. Acad. Sci. U.S.A 106, 6387-6392 (2009)), and DG neuroblasts (DGN-8, marked by Sema3c and Igfbpl1 (Zeisel et al., Cell 174, 999-1014.e22 (2018); Puverel et al., J. Comp. Neurol. 512, 232-242 (2009); Kumar et al., IBRO Rep 9, 224-232 (2020)). Interestingly, DG neuroblasts present with a substantial deduction even before six months, suggesting an earlier decline of DG neurogenesis compared to OB neurogenesis. In contrast to the depleted progenitor pool involved in neurogenesis, there was no detection of significant changes in proliferating oligodendrocyte progenitor cells (Cycling OPCs, OPC-4, marked by Pdgfra and Mki67 (Pastrana et al., Proc. Natl. Acad. Sci. U.S.A 106, 6387-6392 (2009); Marques et al., Dev. Cell 46, 504-517.e7 (2018)) in aging. Instead, the newly formed oligodendrocytes (OLG-6, marked by Prom1 and Tef7l1 (Pastrana et al., Proc. Natl. Acad. Sci. U.S.A 106, 6387-6392 (2009); Marques et al., Dev. Cell 46, 504-517.e7 (2018)) and a committed oligodendrocyte precursor subtype (OPC-6, marked by Bmp4 and Bcas1 (Pastrana et al., Proc. Natl. Acad Sci. U.S.A 106, 6387-6392 (2009); Marques et al., Dev. Cell 46, 504-517.e7 (2018)) show significantly reduced proportion in the aged brain, suggesting a block of oligodendrocyte differentiation upon aging. Notably, the heterogenous age-dependent change in the cell-type-specific proliferation and differentiation were further validated in the companion study, where the newly proliferated cells were labeled and their differentiation dynamics in mammalian brains across the lifespan were tracked.

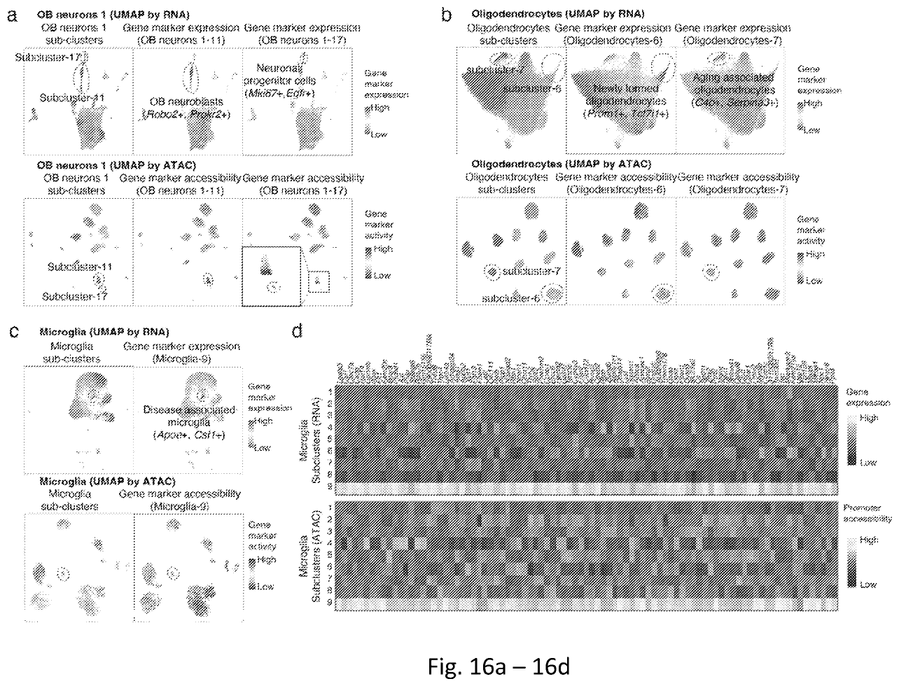

The atlas of chromatin accessibility was next leveraged to identify the epigenetic controls underlying the age-dependent decline in adult neurogenesis and oligodendrogenesis. While this aforementioned integrative approach successfully identified the chromatin landscape of all main cell types, there were several substantial challenges for the sub-clustering level analysis, including the relatively lower number of profiled cells and lower resolution of the single-cell chromatin accessibility dataset compared with the single-cell transcriptome analysis. However, several cell subtypes with either high abundance or unique epigenetic signatures were recovered. For example, OB neuroblasts (OB neurons 1-11), OB neuronal progenitors (OB neurons 1-17), and newly formed oligodendrocytes (OLG-6) were identified (FIG. 16a, b), all exhibiting sharply decreased dynamics in the aged brain similar to the single-cell transcriptome analysis (FIG. 13d, right). Moreover, potential TF regulators were identified and validated by both gene expression and TF motif accessibility enriched in specific cell types, such as known regulators of neurogenesis (e.g., Sox2 and E2f2 (Graham et al., Neuron 39, 749-765 (2003); Li et al., Cereb. Cortex 28, 3278-3294 (2018)) (FIG. 13f), which further validated this integration approach for characterizing key epigenetic signatures of aging-associated cell subtypes.

In contrast to the neural progenitor cells, several cellular sub-clusters exhibited a remarkable expansion in the aged brain. For example, the most up-regulated sub-cluster in aging is a microglia sub-cluster (sub-cluster 9, Apoe+, Csf1+), corresponding to a previously reported disease-associated microglia subtype (Keren-Shaul et al., Cell vol. 169 1276-1290.e17 (2017)). In addition, a reactive oligodendrocyte subtype (OLG-7, C4b+, Serpina3n+ (Zhou et al., Nat. Med. 26, 131-142 (2020); Kenigsbuch et al., Nat. Neurosci. 25, 876-886 (2022)) significantly enriched in the aged brain was identified. With the chromatin accessibility dataset, the expansion of this cell type was confirmed (FIG. 13e, FIG. 16b, c), and its associated transcription factors were identified, including the cell-state-specific expression and motif accessibility of Stat3 (FIG. 13f), a critical regulator involved in the control of inflammation and immunity in the brain (See et al., J. Neurooncol. 110, 359-368 (2012)). By spatial transcriptomics analysis, a striking enrichment of the reactive oligodendrocyte specific markers (e.g., C4b, Serpina3n) around the subventricular zone (SVZ) was detected, a region critical for the continual production of new neurons in adulthood (FIG. 13h-g), indicating an age-related activation of inflammation signaling around the adult neurogenesis niche.

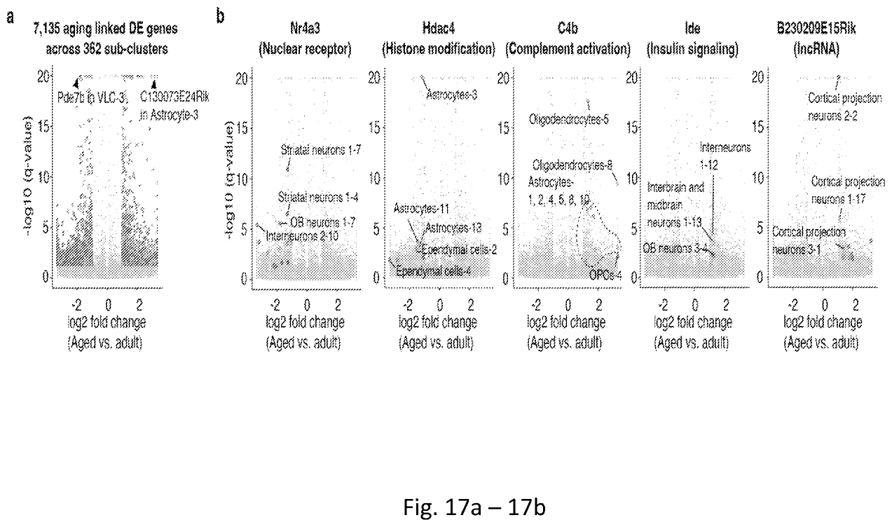

Next, the subtype-specific manifestation of key aging-related molecular signatures was explored. Differentially expressed gene analysis was performed and 7,135 aging-associated signatures across 363 sub-clusters was identified (FDR of 5%, with at least 2-fold change between aged and adult brains, FIG. 17a). 580 genes were changed across multiple (>=3) subtypes, of which 241 genes were regulated in the same direction (FIG. 17b). For example, Nr4a3, a component of DNA repair machinery and a potential anti-aging target (Paillasse et al., Med. Hypotheses 84, 135-140 (2015)), was significantly decreased only in aged neurons, including striatal neurons, OB neurons, and interneurons. Hdac4, encoding a histone deacetylase and a recognized regulator of cellular senescence (Di Giorgio et al., Genome Biol. 22, 129 (2021)), was significantly reduced only in aged astrocytes and ependymal cell subtypes. Meanwhile, the Insulin-degrading enzyme (IDE), a key factor involved in Amyloid-beta clearance (Zhang et al., Med. Sci. Monit. 24, 2446-2455 (2018)), was increased only in subtypes of neurons, including interneurons, OB neurons, interbrain, and midbrain neurons. While many of these genes have been previously reported to be associated with aging, this analysis represents the first global view of their alterations across over 300 subtypes. In addition, several non-coding RNAs that significantly changed in multiple aged subtypes were identified, most of which show high cell-type-specificity (e.g., B230209E15Rik in cortical projection neurons subtypes) but were not well-characterized before (FIG. 17b).

### A Global View of AD Pathogenesis-Associated Signatures and Subtypes

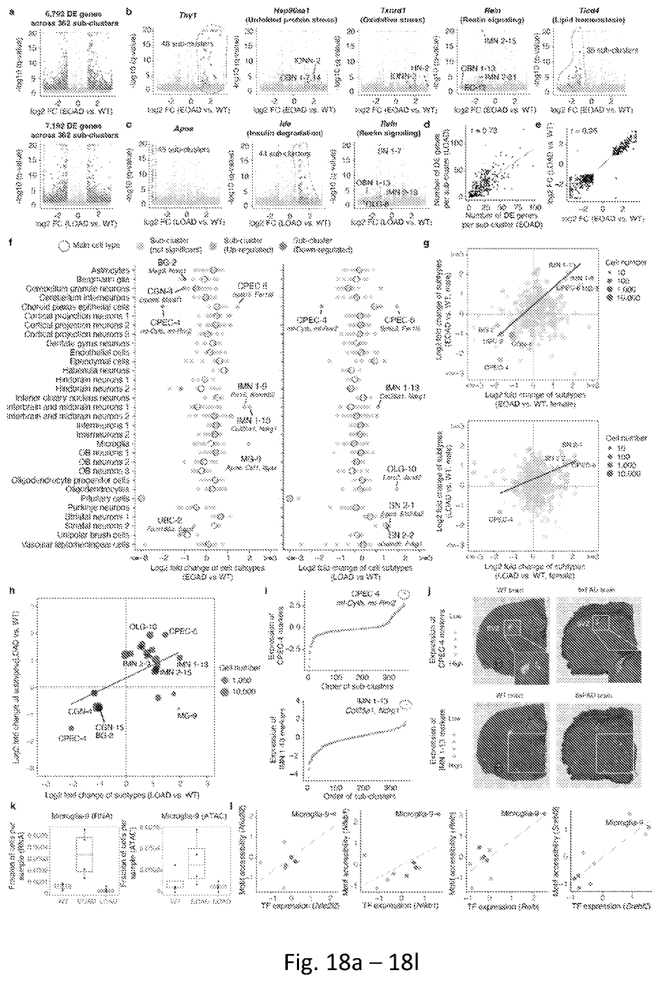

Hypothesized AD pathogenesis-associated signatures through differentially expressed gene analysis in AD mouse models were next explored. 6,792 and 7,192 sub-cluster-specific DE genes were detected in the 5×FAD (EOAD) model and the APOE*4/Trem2*R47H (LOAD) model, respectively (FIG. 18a). As expected, Apoe was significantly down-regulated across many sub-clusters in the APOE*4/Trem2*R47H mice (FIG. 18c). Meanwhile, a global change of Thy1 across many neuron types in the 5×FAD mice was detected, consistent with the fact that all transgenes introduced in the 5×FAD model were controlled under the Thy1 promoter (FIG. 18b).

Many AD-associated gene signatures exhibited remarkably concordant changes across cellular subtypes (FIG. 18b, c). For example, markers involved in unfolded protein stress (e.g., Hsp90aa1) and oxidative stress (e.g., Txnrd1) were significantly upregulated in an overlapped set of neuron subtypes in the early-onset 5×FAD mice (FIG. 18b), indicating increased stress levels and cellular damages in neurons across the brain. Meanwhile, Reln, which encodes a large secreted extracellular matrix protease involved in the ApoE biochemical pathway (Seripa et al., J. Alzheimers. Dis. 14, 335-344 (2008)), significantly decreased in multiple cell types (e.g., OB neurons, interbrain and midbrain neurons, vascular cells, oligodendrocytes) in both early- and late-onset models (FIG. 18b, c). This is consistent with previous reports that the depletion of Reln is detectable even before the onset of amyloid-β pathology in the human frontal cortex (Herring et al., J. Alzheimers. Dis. 30, 963-979 (2012)). Other interesting phenomena included the overall upregulation of Ide, a gene responsible for amyloid-β degradation, in the late-onset model similar to the aged brain (FIG. 18b, FIG. 17b), which could contribute to the delayed onset in APOE*4/Trem2*R47H mice. Less-characterized genes were identified as well. For example, Tlcd4, a gene potentially involved in lipid trafficking and metabolism (Attwood et al., Front Cell Dev Biol 9, 708754 (2021)), was significantly downregulated in thirty-five sub-clusters across broad cell types (e.g., OB neurons, Vascular cells, oligodendrocytes) in the early-onset 5×FAD mice (FIG. 18b), suggesting a potential interplay between lipid homeostasis and neurodegenerative phenotypes.

While the two AD mouse models are different in terms of genetic perturbations or disease onsets, their cell-type-specific molecular changes were surprisingly consistent. Illustrative of this, the number of DE genes per sub-cluster was highly correlated between the two models (Pearson correlation coefficient r=0.73, p-value<2.2e-16, FIG. 18d). Additionally, 559 sub-cluster-specific DE genes shared between two AD mutants was detected, such as genes involved in epilepsy (Adjusted p-value=0.02, e.g., Gria1, Med1, Plp1) (Kuleshov et al., Nucleic Acids Res. 44, W90-7 (2016)) and oxidative stress protection pathway (Adjusted p-value=0.05, e.g., Arnt, Nfe2l2) (Kuleshov et al., Nucleic Acids Res. 44, W90-7 (2016)). Intriguingly, 99% (555 of the 559) of the shared DE genes showed concordant changes in two AD mutants (Pearson correlation coefficient r=0.96, p-value<2.2e-16, FIG. 18e), indicating shared molecular programs between early- and late-onset AD models. Of note, this analysis further validates that the APOE*4/Trem2*R47H mice mutant, a mouse model recently developed, can serve as an informative model to study LOAD.

Toward a global view of AD-associated cell population dynamics, the relative fraction of sub-clusters in the two AD models was quantified for comparison with their age-matched wild-type controls (3-month-old). 16 and 14 significantly changed sub-clusters was detected (FDR of 5%, at least two-fold change) in the EOAD (5×FAD) model and LOAD (APOE*4/Trem2*R47H) model, respectively (FIG. 18f, Table 1 and Table 2). Most significantly altered subtypes showed consistent proportion changes in male and female mice (FIG. 18g). Interestingly, while these two AD mutants involved different genetic perturbations, the significantly altered cell subtypes were highly concordant (FIG. 18h). For example, a rare choroid plexus epithelial cell subtype (CPEC-4, 0.018% of the total brain cell population) was strongly depleted in both AD models. This cell type is marked by significant enrichment of mitochondrial genes, including mt-Rnr1, mt-Rnr2, mt-Col, mt-Cytb, mt-Nd1, mt-Nd2, mt-Nd5, and mt-Nd6. Some of these mitochondrial genes (e.g., mt-Rnr2) have been associated with synthesizing neuroprotective factors against neurodegeneration by suppressing apoptotic cell death (Hashimoto et al., Proc. Natl. Acad. Sci. U.S.A 98, 6336-6341 (2001)); others (e.g., mt-Rnr1 and mt-Nd5) were reported to be related to the phosphorylated Tau protein levels in cerebrospinal fluid (Cavalcante et al., Biomedicines 10, (2022)). While this cell type was only rarely identified in the single-cell ATAC data, it was possible to map the cell subtype to the subventricular zone by the expression of cell-type-specific markers in the spatial transcriptomics data (FIG. 18i-j). Consistent with the scRNA data, this cell type was strongly depleted in the spatial transcriptomic profiling of the EOAD (5×FAD) model (FIG. 18j), suggesting a potential interplay between cell-type-specific mitochondrial functions and neurodegenerative phenotypes. By contrast, another interbrain and midbrain neuron subtype (IMN 1-13, Col25a+ Ndrg1+) expanded considerably in both AD models (FIG. 18h). This subtype is marked by the expression of Col25a, a membrane-associated collagen that has been reported to promote intracellular amyloid plaque formation in mouse models (Tong et al., Neurogenetics 11, 41-52 (2010)). Indeed, an up-regulation of IMN 1-13 specific gene markers was identified in the thalamus region of the 5×FAD mouse brain (FIG. 18i-j), further validating the single-cell transcriptome analysis.

TABLE 1
Differentially abundant sub-clusters between wild type and LOAD model.

| | | Log2(Fold | Number | |
|---|---|---|---|---|
| Cell sub-cluster | Q-value | change) | of cells | Final change |
| Bergmann glia_2 | 0.001741648 | −1.001068724 | 881 | Downregulated |
| Cerebellum granule neurons_15 | 0.002539487 | −1.001599879 | 1421 | Downregulated |
| Cerebellum granule neurons_4 | 2.00E−26 | −1.067525696 | 34921 | Downregulated |
| Choroid plexus epithelial cells_4 | 6.91E−26 | −2.028294359 | 168 | Downregulated |
| Hindbrain neurons 2_4 | 7.64E−13 | −1.167696006 | 309 | Downregulated |
| Unipolar brush cells_2 | 0.002539487 | −1.204448696 | 146 | Downregulated |
| Choroid plexus epithelial cells_6 | 0.000634928 | 1.46049498 | 159 | Upregulated |
| Cortical projection neurons 1_17 | 7.70E−07 | 1.107595437 | 527 | Upregulated |
| Cortical projection neurons 1_23 | 5.76E−22 | 1.079606112 | 1506 | Upregulated |
| Cortical projection neurons 2_13 | 1.62E−06 | 1.105967385 | 442 | Upregulated |
| Interbrain and midbrain neurons 1_13 | 1.38E−15 | 1.990360624 | 296 | Upregulated |
| Interbrain and midbrain neurons 1_9 | 2.43E−05 | 1.770493437 | 136 | Upregulated |
| Interbrain and midbrain neurons 2_15 | 1.88E−07 | 1.17960744 | 208 | Upregulated |
| Interbrain and midbrain neurons 2_24 | 1.57E−05 | 1.188554014 | 396 | Upregulated |
| Interbrain and midbrain neurons 2_9 | 5.22E−21 | 1.104598658 | 1823 | Upregulated |
| Microglia_9 | 5.97E−09 | 1.951669875 | 75 | Upregulated |

TABLE 2
Differentially abundant sub-clusters between wild type and LOAD model.

| | | Log2(Fold | Number | |
|---|---|---|---|---|
| Cell sub-cluster | Q-value | change) | of cells | Final change |
| Choroid plexus epithelial cells_4 | 2.96E−26 | −1.525231318 | 204 | Downregulated |
| Cerebellum granule neurons_10 | 3.67E−115 | 1.206897519 | 8030 | Upregulated |
| Choroid plexus epithelial cells_1 | 1.38E−07 | 1.241757141 | 817 | Upregulated |
| Choroid plexus epithelial cells_5 | 0.019996558 | 1.130589882 | 84 | Upregulated |
| Choroid plexus epithelial cells_6 | 5.65E−11 | 1.948657495 | 346 | Upregulated |
| Ependymal cells_3 | 5.59E−14 | 1.382951706 | 423 | Upregulated |
| Interbrain and midbrain neurons 1_13 | 6.60E−07 | 1.079062043 | 321 | Upregulated |
| Interbrain and midbrain neurons 2_9 | 2.92E−20 | 1.019011372 | 2775 | Upregulated |
| Oligodendrocytes_10 | 5.18E−57 | 1.932849872 | 1919 | Upregulated |
| Striatal neurons 1_4 | 3.22E−33 | 1.267727954 | 2905 | Upregulated |
| Striatal neurons 2_1 | 2.60E−17 | 1.586281252 | 596 | Upregulated |
| Striatal neurons 2_2 | 3.16E−08 | 1.497962393 | 234 | Upregulated |
| Striatal neurons 2_4 | 4.39E−09 | 1.462076289 | 210 | Upregulated |
| Vascular leptomeningeal cells_10 | 0.001701393 | 1.143721078 | 228 | Upregulated |

Finally, a significant expansion of disease-associated ApoE+ Csf1+ microglia-9 subtype was detected in the early-onset 5-FAD mice, similar to the aged mice, consistent with previous reports (Keren-Shaul et al., Cell vol. 169 1276-1290.e17 (2017)). This cell type was not enriched in the late-onset APOE*4/Trem2*R47H model (3-month-old), indicating a correlation between the reactive microglia with disease onset (FIG. 18k). Consistent proportion changes were detected with the chromatin accessibility dataset (FIG. 18k). To further delineate the transcriptional control of microglia differentiation, 199 genes differentially expressed in the reactive microglia subtype were identified, many of which (44%) can be validated by the promoter accessibility (FIG. 15d). In addition, key transcription factors validated by both cell-type-specific gene expression and motif accessibility were identified (FIG. 18l), including TFs of the NF-kappa B signaling pathway (e.g., Nfkb1 and Relb (Oeckinghaus et al., Cold Spring Harb. Perspect. Biol. 1, a000034 (2009)) and TFs involved in oxidative stress protection (e.g., Nfe2l2 (Liu et al., Aging Cell 16, 934-942 (2017)), and cholesterol homeostasis (e.g., Srebf2 (Bommer et al., Cell Metab. 13, 241-247 (2011)), reflecting potential regulatory roles of these molecular pathways in microglia specification.

### Example 2: EasySci-RNA Protocol

Single-cell combinatorial indexing (‘sci-’) is a methodological framework that employs split-pool barcoding to uniquely label the nucleic acid contents of large numbers of single cells or nuclei. Although much progress has been made in making combinatorial indexing methods more efficient, easier to perform, and less costly, there are still major shortcomings in these high-throughput RNA-sequencing techniques. To address this, a new 3-level sci-RNA-seq method (EasySci-RNA) was employed which includes optimizations that drastically improve efficiency, lower cost per cell sequenced, and increased gene body coverage compared to the previous iteration of the method (sci-RNA-seq3).

### The Protocol Workflow is as Follows:

Buffer Preparation (Steps 1-12)
Ligation Primer Annealing (Steps 13-16)
Tn5 loading (Step 17)
Nuclei Extraction (˜2.5 hrs for 6 samples) (Steps 18-26)
Nuclei Wash (˜15-30 mins for 6-30 samples) (Steps 27-28)
Nuclei Counting (Step 29)
Reverse Transcription (˜1-2.5 hrs depending on the number of samples) (Steps 30-33)
Pool/Centrifuge/Resuspend/Redistribute (15 m) (Steps 34-35)
Ligation (˜2 hrs) (Steps 36-40)
Pool/Centrifuge/Resuspend/Redistribute/Quantify (30 m) (Steps 41-45)
Second-Strand Synthesis (˜1.25 hrs) (Steps 46-48)
0.8× Ampure Beads Purification (˜1 hr) (Steps 49-55)
Tagmentation (˜10 mins) (Steps 56-57)
SDS Treatment (˜1.5 hrs) (Steps 58-61)
PCR (45 m) (Step 62)
Library Purification (˜1 hr) (Steps 63-74)
   It is important to start with a species-mixing experiment for validating the experimental setup is working-normally mixture of human (HEK293T) and mouse (NIH/3T3) cells. A good run normally yields single-cell transcriptomes with over 5000 UMIs (with over 20,000 sequencing reads) per cell and >98% purity.

### Required Equipment:

Bioruptor Sonication Device
Hemocytometers (Neubauer Improved, Bulldog Bio VWR #102966-632) Centrifuge (Eppendorf 5702 RH)
DynaMag-96 Side Skirted Magnet (Invitrogen, 12027)/DynaMag-96 Side Magnet (Invitrogen, 12331D)
12-tube Magnetic Separation Rack (NEB, S1509S)
Eppendorf Mastercycler (4×)
Freezer (−20 C, −80 C) and Refrigerator (4 C)
Gel Box
Gel Imager
Ice Buckets
Microscope
Multi-channel Pipettes (2-20 μL, 20-200 μL) (Rainin Instruments)
NextSeq 500 Platform (Illumina)
Pipettors
96 well Pipetting System
Liquid nitrogen tank for sample storage
FreezeCell Cell Freezing Container (GeneSeeSci, catalog number: 27-802) Eppendorf ThermoMixer C (5382000023) OR Fisherbrand Nutating Mixer (88861043)

### Primer Sequences

All primer sequences including RT/Ligation/PCR primers are provided in Tables 3-6. All primers are ordered from IDT with standard desalting.

### List of Materials Used

Nuclease free water (Ambion, AM 9937)
10 cm cell culture dish (Genesee, 25-202)
6 cm cell culture dish (Genesee, 25-260)
OEMTOOLS 25181 Razor Blades, 100 Pack (VWR, 55411-0055)
Ward's 40 um Sterile Cell Strainer (VWR, 470236-276)
PluriStrainer Mini 40 um (PluriSelect 43-10040-70)
PluriStrainer Mini 20 um (PluriSelect 43-10020-70)
PluriStrainer Mini 5 um (PluriSelect 43-10005-70)
BD New STERILE, Sealed, 5 ML Syringes Only LUER Lock TIP, No Needle, Disposable (VWR, BD309646)
Pierce 16% Formaldehyde, Methanol Free (Thermofisher, 28906)
SUPERase In RNase Inhibitor 20 U/uL (Thermo Fisher Scientific, AM2696) BSA 20 mg/ml (NEB, B9000S)
1M Tris-HCl (pH 7.5) (Thermo Fisher Scientific, 15567027)
5M NaCl (Thermo Fisher Scientific, AM9759)
1M MgCl₂ (Thermo Fisher Scientific, AM9530G)
TE Buffer (IDTE, Nov. 5, 2001-05)
Dimethylformamide, 99.8% (Fisher Scientific, AC327175000)
Dimethyl Sulfoxide (VWR, 97063-136)
Nuclei Isolation Kit: Nuclei EZ Prep (Millipore Sigma, NUC101-1KT)
Diethyl Pyrocarbonate (DEPC) (VWR, 97062-652)
PBS, 1× (Genesee, 25-507)
Triton X-100 for molecular biology (Sigma Aldrich, 93443-100ML)
10 mM dNTP (Thermo Fisher Scientific, R0192)
192 indexed shortdT primers (100 μM, 5′-(SEQ ID NO: 2413)/5Phos/ACGACGCTCTTCCGATCTNNNNNNNN [10 bp barcode] TTTTTTTTTTTTTTTT-3′ (SEQ ID NO:2414), where “N” is any base; IDT)
192 indexed randomN primers (100 μM, 5′-/5Phos/ACGACGCTCTTCCGATCTNNNNNNNN (SEQ ID NO:2447) [10 bp barcode] NNNNNN-3′, where “N” is any base; IDT)
Maxima H Minus Reverse Transcriptase with Buffer (ThermoFisher, EP0753) T4 DNA Ligase (NEB, M0202L)
EDTA 0.5M Solution (VWR, 97062-656)
384 indexed ligation primers (100 μM, 5′-(SEQ ID NO: 2415) AATGATACGGCGACCACCGAGATCTACAC [10 bp barcode] ACACTCTTTCCCTAC-3′ (SEQ ID NO:2416))
Adapter Primer (100 μM, 5′-
A*G*A*T*C*G*G*A*A*G*A*G*C*G*T*C*G*T*G*T*A*G*G*G*A*A*A*G*A*G*T*G*T*/3ddC/) (SEQ ID NO: 2445) Elution buffer (Qiagen, 19086)
NEBNext® Ultra II Non-Directional RNA Second Strand Synthesis Module (NEB, E7550S) Nextera N7 adaptor loaded Tn5 (provided by Illumina) OR Custom Tn5
DNA binding buffer (Zymo Research, D4004-1-L)
AMPure XP beads (Beckman Coulter, A63882)
SDS, 20% Solution, RNase Free (ThermoFisher AM9820)
Tween 20 (Millipore Sigma, P9416-100ML)
Ethanol (Sigma Aldrich, 459844-4L)
10 μM Universal P5 primer ((SEQ ID NO: 2446) 5′-AATGATACGGCGACCACCGAGATCTACAC-3′, IDT) 10 μM P7 primer ((SEQ ID NO:2417) 5′-CAAGCAGAAGACGGCATACGAGAT [17] GTCTCGTGGGCTCGG-3′ (SEQ ID NO: 2418), IDT) NEBNext High-Fidelity 2× PCR Master Mix (NEB, M0541L)
Qubit dsDNA HS kit (Invitrogen, Q32854)
Qubit tubes (Invitrogen, Q32856)
E-Gel EX Agarose Gel, 2% (ThermoFisher, G402002)
E-Gel 50 bp DNA Ladder (ThermoFisher, 10488099)
Nextseq V2 75 cycle kit (Illumina, FC-404-2005)
Falcon Tubes, 15 ml (VWR Scientific, 21008-936)
Falcon Tubes, 50 ml (VWR Scientific, 21008-940)
Green pack LTS 200 ul filter tips (GP-L200F) (Rainin Instrument, 17002428)
Pipette Tips RT LTS 20 uL FL 960A/10 (Rainin, 30389226)
Pipette Tips RT LTS 200 μL F 960/10 (Rainin, 30389239)
Pipette Tips RT LTS 200 μL FLW 960A/10 (Rainin, 30389241)
4-Chip Disposable Hemocytometers, Neubauer Improved, Bulldog Bio (VWR, 102966-632)
DNA LoBind Tube 1.5 ml, PCR clean (Eppendorf North America, 22431021)
1.0 mL Self-Standing Cryovial (GeneSeeSci, catalog number: 24-200P)
LoBind clear, 96-well PCR Plate (Eppendorf North America, 30129512)
0.2 mL 8-Strip Tubes with Individual Caps (PCR Tubes) (Genesee, 27-125U)
Reagent reservoirs (Fisher Scientific, 07-200-127)
Falcon® 5 mL Round Bottom w/Cell Strainer (Fisher Scientific, 352235)
eXTReme FoilSeal Film (Genesee, 12-156)
eXTReme Clear Sealing Film (Genesee, 12-157)

### Buffer Preparation

500 mL Nuclei Buffer (Stored in 4 C)
10 mM Tris-HCl, pH 7.5; 10 mM NaCl; 3 mM MgCl2 in nuclease free water:

| | Stock | Final | Volume |
|---|---|---|---|
| Reagent | concentration | concentration | (ml) |
| Tris-HCl (pH 7.5) | 1M | 10 mM | 5 |
| NaCl | 5M | 10 mM | 1 |
| MgCl2 | 1M | 3 mM | 1.5 |
| Nuclease-free | | NA | 492.5 |
| water NA | | | |
| Final volume | | | 500 |

Filter the buffer through a 0.22 uM filter and store the buffer in 4 C for up to 1 year.
20 mL 10% (volume) Triton-X-100 in nuclease-free water (stored in 4 C)
Add 2 mL Triton X-100 to 18 mL nuclease-free water. Mix the solution by pipetting up and down 20 times. The mix can be stored in 4 C for up to 1 year.
EZ Lysis Buffer+0.1% RNase Inhibitor (Made fresh each time, stored on ice, 2 mL per tissue sample)
EZ lysis buffer with 0.1% (volume) SUPERase In RNase Inhibitor (20U/μL, Ambion). For each sample, combine 2 mL EZ lysis buffer and 2 μL SUPERase In RNase Inhibitor (20U/μL, Ambion).
EZ Lysis Buffer+1% DEPC (Made fresh each time, stored on ice, DEPC added just before lysis step, 1 mL per tissue sample)
EZ Lysis buffer with 1% (volume) DEPC. For each sample, combine 990 μL EZ lysis buffer and 10 μL DEPC
Nuclear Suspension Buffer (NSB) (Made fresh each time, stored on ice)
Nuclei Buffer with 1% SUPERase In RNase Inhibitor (20U/μL, Ambion) and 1% BSA (20 mg/mL, NEB): For every 1 mL NSB needed, combine 980 μL Nuclei Buffer, 10 μL SUPERase In RNase Inhibitor (20U/μL, Ambion), and 10 μL BSA (20 mg/mL, NEB).
Nuclear Suspension Buffer+10% DMSO (NSB+10% DMSO) (Made fresh each time, 100 μL needed per sample aliquot, stored on ice)
For every 1 mL needed, add 900 μL Nuclear Buffer and 100 μL DMSO.
Nuclear Suspension Buffer+0.1% Triton-X-100 (NSB+Triton) (Made fresh each time, 750 μL needed per sample, stored on ice)
For every 1 mL needed, add 990 μL Nuclei Buffer and 10 μL 10% Triton-X-100.
Nuclear Buffer+1% BSA+0.1% Triton-X-100 (NBB) (Made fresh each time, ˜8 mL needed, store on ice)
Add 7.84 mL Nuclei Buffer, 80 μL BSA (20 mg/mL, NEB), and 80 μL 10% Triton-X-100.
0.1% Formaldehyde in PBS (Made fresh each time, 1 mL needed per sample, store on ice)
For every 1 mL solution needed, add 1 mL PBS and 6.25 μL 16% Formaldehyde (Using 1 mL glass vial of 16% formaldehyde: open and use a fresh tube of formaldehyde each time)
2× Tagmentation Buffer (Stored in −20 C)
Prepare 200 mL of Tagmentation Buffer (filtered):
   1M Tris HCl (pH 7.5): 4 mL
   1M MgCl2: 2 mL.
   DMF: 40 mL
   H2O: add to 200 ml (˜154 mL)
Aliquot the solution into 15 mL or 1.5 mL tubes
1% SDS (Store at room temperature)
Mix 1 mL 10% SDS (brand, catalog #) and 9 mL H2O
10% Tween-20 (Store in 4 C)
Mix 1 mL Tween-20 and 9 mL H2O, let sit for 10 minutes before mixing again. Repeat until the solution is homogenous.

### Ligation Primer Loading (1 h)

Resuspend and dissolve the Ligation Adaptor Primer Oligo to 100 μM in TE Buffer
In each well of an empty 96-well plate, add 5 μL of 100 μM dissolved Ligation Adaptor Primer and 5 μL 100 μM Barcoded Ligation Primers-make sure to add the Barcoded Ligation Primers to their correct wells
Anneal the adaptor and ligation primers together by running the following thermocycler program:
   95 C for 2 minutes
   Cool to 20 C at a rate of −1 C per minute
   Hold at 4 C
The final annealed concentration will be 50 μM.
Dilute the primers to 3.125 μM by adding 150 μL of EB buffer. The resulting product is in stable, double-stranded form and can be stored at 4 C or frozen. In 4 C, the annealed primers should be stable for roughly three months and is suitable for short-term testing experiments.

### Tn5 Loading (1 h)

Protocol Derived from Hennig et al. 2018, Large-Scale Low-Cost NGS Library Preparation Using a Robust Tn5 Purification and Tagmentation Protocol-purified Tn5 protein is also from this publication.
The Tn5 loading protocol is derived from Hennig et al. 2018, Large-Scale Low-Cost NGS Library Preparation Using a Robust Tn5 Purification and Tagmentation Protocol. Their purified Tn5 protein was used. The procedure is listed below: 150 μL of 100 μM Tn5-ME-B oligo (5′-GTCTCGTGGGCTCGGAGATGTGTATAAGAGACAG-3′ (SEQ ID NO:2450), in TE buffer) was mixed with 150 μL of 100 μM Tn5MErev oligo (-/5′Phos/CTGTCTCTTATACACATCT-3′ (SEQ ID NO:2451), in TE buffer) reaching a final concentration of 50 μM. Then, the mixture was split into aliquots and the following thermocycler conditions was performed: 95 C for 5 minutes, slowly cooled to 65 C (0.1 C/sec or 2%), 65 C for 5 minutes, slowly cooled to 4 C (0.1 C/sec or 2%). The mixture was further diluted to 35 μM by mixing 10 μL of the oligo mixture with 4.28 μL of TE buffer. Then, 1 μL of the Tn5 enzyme at 4 mg/mL was combined with 19 μL of Tn5 Dilution Buffer (25 mM Tris pH 7.5, 800 mM NaCl, 0.1 mM EDTA, 1 mM DTT and 50% glycerol) and 2 μL of the 35 μM Tn5-ME-B/Tn5-MErev oligo mixture. This solution was placed on a thermomixer at 23 C for 30 minutes and diluted with 22 μL of glycerol and stored at −20 C for future usage.
Alternatively, use Nextera N7 loaded Tn5 from Illumina or Commercial Tn5 from Diagenode or another alternative

### Nuclei Extraction (˜2.5 hrs for 6 Samples)

Cool centrifuge to 4 C—make sure to use a bucket centrifuge for all centrifuging steps unless otherwise stated, as normal centrifuges may have difficulty making a neat pellet at the bottom of the tube, which is necessary to maximize nuclear recovery.
In a 6 cm dish on ice, cut each tissue section (0.1 g-0.5 g) into small pieces (<1 mm3) using a razor blade and 1 mL PBS with 10 μL DEPC added. Transfer the tissue and solution into a 1.5 mL tube and spin for 5 minutes at 200 g at 4 C.
*Make sure to add DEPC just before performing lysis, as DEPC has a short half-life in aqueous solutions*
*Perform this step in a fume hood, as chopping tissue in a DEPC solution may be toxic* *For larger tissue samples, may want to split into multiple 1.5 mL tubes to make pipetting the samples easier*
*Ideally, the tissue sections do not thaw until the sections are being cut in the DEPC-PBS solution. To prevent thawing, have a separate container filled with dry ice to place the sections that are currently not being minced with the razor blade*
*Generally, a maximum of six tissue sections is worked with at one time—it is theoretically possible to process more at the same time, but it may be difficult to manage*
Dump Supernatant
Add 1 mL ice-cold EZ lysis buffer+1% DEPC to the tissue for nuclei extraction. Pipet the tissue up and down with a 1 mL pipet tip 10 times (cut the top of 1 mL pipet tip if needed for easier pipetting). Incubate on ice for 5 minutes.
*Make sure to add DEPC just before performing lysis, as DEPC has a short half-life in aqueous solutions and will degrade if not added immediately before lysis*
*From this point on, use 1 mL pipet tips or wide bore tips when working with nuclei to avoid stress on nuclei*
Filter tissue with a 40 μm cell strainer into a 6 cm dish and grind tissue on the strainer using a 5 ml syringe plunger. Add 500 μL EZ Lysis Buffer+0.1% RNase Inhibitor and continue grinding tissue on the strainer. Move solution into a 1.5 mL microcentrifuge tube.
*It is not necessary to push the whole tissue through the filter! Make sure not to tear through the filter!*
Pellet the nuclei by centrifuging for 5 minutes, 500 g at 4 C. Dump supernatant. Resuspend each tube in 500 μL EZ Lysis Buffer+0.1% RNase Inhibitor by pipetting up and down three times.
Pellet the nuclei by centrifuging for 5 minutes, 500 g at 4 C. Dump supernatant.
Fixation: Take each tube and add 1 mL of ice-cold 0.1% Formaldehyde suspended in PBS. Start a 10-minute timer immediately after formaldehyde is added. Mix up and down to resuspend the pellet.
For multiple samples, add 1 mL directly to the top of tubes without changing tips and without touching the tubes; start timer once the first mL of formaldehyde is added and add to all tubes. Once done, go back and pipet up and down the solution in each sample to resuspend the pellet, making sure to switch tips for each sample.
*Perform this step in a fume hood as formaldehyde is toxic*.
Pellet the nuclei immediately afterward by centrifuging for 3 minutes, 500 g at 4 C. Dump supernatant in a chemical waste container. Resuspend each tube in 500 μL EZ Lysis Buffer+0.1% RNase Inhibitor by pipetting up and down three times.
Pellet the nuclei by centrifuging for 5 minutes, 500 g at 4 C. Dump supernatant. Resuspend each tube in 500 μL EZ Lysis Buffer+0.1% RNase Inhibitor by pipetting up and down three times.
PERFORM THIS STEP IF THERE IS A DESIRE TO STORE NUCLEI FOR LATER USE—OTHERWISE, SKIP TO THE SECOND PART OF THE NEXT STEP:
Pellet the nuclei by centrifuging for 5 minutes, 500 g at 4 C. Resuspend each tube in 100-500 μL NSB+10% DMSO and split into 100 μL aliquots. Slow freeze in a −80 C freezer and keep for storage. Optimally, use specialized slow-freezing chambers with 1.0 mL Self-Standing Cryovials (FreezeCell Cell Freezing Container, GeneSeeSci, catalog number: 27-802) (1.0 mL Self-Standing Cryovial, GeneSeeSci, catalog number: 24-200P) (STOP POINT).
   Nuclei Wash (˜15-30 minutes for 6-30 Samples)
1) PERFORM BELOW IF YOU ARE WORKING WITH PREVIOUSLY FROZEN, STORED NUCLEI:
Thaw cells for 30 seconds in a 37 C water bath. Add 400 μL NSB+Triton to each sample to resuspend pellet, and then sonicate for 12 seconds at low power. After, filter nuclei through a 20 um filter. Wash the filter with an additional 250 μL NSB+Triton and then pellet the nuclei for 5 minutes, 500 g at 4 C.
2) PERFORM BELOW IF DIRECTLY CONTINUING FROM NUCLEI EXTRACTION:
Add 500 μL NSB+Triton to each sample to resuspend pellet, and then sonicate for 12 seconds at low power. After, filter nuclei through a 20 um filter. Wash the filter with an additional 250 μL NSB+Triton and then pellet the nuclei for 5 minutes, 500 g at 4 C.
Resuspend the pellet in 100 μL of NSB.

### Nuclei Counting

Count the concentration for each sample.

A buffer with DAPI and a fluorescent microscope can be used to distinguish between actual nuclei and debris. To make the buffer, dissolve 10 mg DAPI in 2 ml of deionized water (dH2O) with a final concentration of 5 mg/ml Split the DAPI solution into multiple tubes (100 ul per tube). Take out one tube (100 μl, 5 mg/ml DAPI), add 1.9 ml deionized water (dH2O). Split the diluted DAPI solution into multiple tubes (100 ul per tube, 0.25 mg/ml DAPI). Store the DAPI solution in a common box in −20 C freezer.

Make the DAPI counting solution: in 500 μL of Nuclei Buffer, add 0.5 μL-1 μL of 0.25 mg/mL DAPI solution Take 1 μL of the sample and combine it with 9 μL of the counting solution. Mix the solution and take 6 μL to dispense into a hemocytometer.

### Reverse Transcription (˜1-2.5 hrs Depending on Number of Samples)

For each well of 2×96 well plates, add a maximum of 20,000 nuclei in 4 μL of NSB; also add 0.5 μL of 10 mM dNTP.
   a. *Nuclei generally distributed into PCR strips and then distributed into wells—make sure not to pipet up and down to avoid nuclei lysis*
   b. *To mix before distribution, use wide bore multichannel tips*
Add 1 μL 50 μM short-dT primer (Table 3) and 1 μL 50 μM randomN primer (Table 4). Incubate plates at 55 C for 5 minutes. Immediately place plates on ice afterward.
   a. *Again, try to avoid pipetting up and down*
Prepare the reverse transcription reaction mix by combining:
5× Maxima Buffer: 420 μL
Maxima Reverse Transcriptase: 105 μL
SUPERase In RNase Inhibitor: 105 μL.
Nuclease Free H2O: 105 μL
   a. Add 3.5 μL to each well for each of the plates, pipet up and down only once
Start the reverse transcription with the following thermocycler program:
   4 C for 2 minutes
   10 C for 2 minutes
   20 C for 2 minutes
   30 C for 2 minutes
   40 C for 2 minutes
   50 C for 2 minutes
   55 C for 15 minutes

TABLE 3
Short dT reverse transcription (RT) primer sequences

| | | SEQ ID | | SEQ ID |
|---|---|---|---|---|
| Name | Sequence | NO: | Barcode | NO: |
| shortDT_plate1_01 | /5Phos/ACGACGCTCTTCCGATCTNNNNNNNNTTCTCGCATGT | 1 | TTCTCGCATG | 193 |
| | TTTTTTTTTTTTTT | | | |
| shortDT_plate1_02 | /5Phos/ACGACGCTCTTCCGATCTNNNNNNNNTCCTACCAGTT | 2 | TCCTACCAGT | 194 |
| | TTTTTTTTTTTTTT | | | |
| shortDT_plate1_03 | /5Phos/ACGACGCTCTTCCGATCTNNNNNNNNGCGTTGGAGCT | 3 | GCGTTGGAGC | 195 |
| | TTTTTTTTTTTTTT | | | |
| shortDT_plate1_04 | /5Phos/ACGACGCTCTTCCGATCTNNNNNNNNGATCTTACGCT | 4 | GATCTTACGC | 196 |
| | TTTTTTTTTTTTTT | | | |
| shortDT_plate1_05 | /5Phos/ACGACGCTCTTCCGATCTNNNNNNNNCTGATGGTCAT | 5 | CTGATGGTCA | 197 |
| | TTTTTTTTTTTTTT | | | |
| shortDT_plate1_06 | /5Phos/ACGACGCTCTTCCGATCTNNNNNNNNCCGAGAATCCT | 6 | CCGAGAATCC | 198 |
| | TTTTTTTTTTTTTT | | | |
| shortDT_plate1_07 | /5Phos/ACGACGCTCTTCCGATCTNNNNNNNNGCCGCAACGAT | 7 | GCCGCAACGA | 199 |
| | TTTTTTTTTTTTTT | | | |
| shortDT_plate1_08 | /5Phos/ACGACGCTCTTCCGATCTNNNNNNNNTGAGTCTGGCT | 8 | TGAGTCTGGC | 200 |
| | TTTTTTTTTTTTTT | | | |
| shortDT_plate1_09 | /5Phos/ACGACGCTCTTCCGATCTNNNNNNNNTGCGGACCTAT | 9 | TGCGGACCTA | 201 |
| | TTTTTTTTTTTTTT | | | |
| shortDT_plate1_10 | /5Phos/ACGACGCTCTTCCGATCTNNNNNNNNACCTCGTTGAT | 10 | ACCTCGTTGA | 202 |
| | TTTTTTTTTTTTTT | | | |
| shortDT_plate1_11 | /5Phos/ACGACGCTCTTCCGATCTNNNNNNNNACGGAGGCGG | 11 | ACGGAGGCGG | 203 |
| | TTTTTTTTTTTTTTT | | | |
| shortDT_plate1_12 | /5Phos/ACGACGCTCTTCCGATCTNNNNNNNNTAGATCTACTT | 12 | TAGATCTACT | 204 |
| | TTTTTTTTTTTTTT | | | |
| shortDT_plate1_13 | /5Phos/ACGACGCTCTTCCGATCTNNNNNNNNAATTAAGACTT | 13 | AATTAAGACT | 205 |
| | TTTTTTTTTTTTTT | | | |
| shortDT_plate1_14 | /5Phos/ACGACGCTCTTCCGATCTNNNNNNNNCCATTGCGTTT | 14 | CCATTGCGTT | 206 |
| | TTTTTTTTTTTTTT | | | |
| shortDT_plate1_15 | /5Phos/ACGACGCTCTTCCGATCTNNNNNNNNTTATTCATTCTT | 15 | TTATTCATTC | 207 |
| | TTTTTTTTTTTTT | | | |
| shortDT_plate1_16 | /5Phos/ACGACGCTCTTCCGATCTNNNNNNNNATCTCCGAACT | 16 | ATCTCCGAAC | 208 |
| | TTTTTTTTTTTTTT | | | |
| shortDT_plate1_17 | /5Phos/ACGACGCTCTTCCGATCTNNNNNNNNTTGACTTCAGT | 17 | TTGACTTCAG | 209 |
| | TTTTTTTTTTTTTT | | | |
| shortDT_plate1_18 | /5Phos/ACGACGCTCTTCCGATCTNNNNNNNNGGCAGGTATTT | 18 | GGCAGGTATT | 210 |
| | TTTTTTTTTTTTTT | | | |
| shortDT_plate1_19 | /5Phos/ACGACGCTCTTCCGATCTNNNNNNNNAGAGCTATAAT | 19 | AGAGCTATAA | 211 |
| | TTTTTTTTTTTTTT | | | |
| shortDT_plate1_20 | /5Phos/ACGACGCTCTTCCGATCTNNNNNNNNCTAAGAGAAGT | 20 | CTAAGAGAAG | 212 |
| | TTTTTTTTTTTTTT | | | |
| shortDT_plate1_21 | /5Phos/ACGACGCTCTTCCGATCTNNNNNNNNACTCAATAGGT | 21 | ACTCAATAGG | 213 |
| | TTTTTTTTTTTTTT | | | |
| shortDT_plate1_22 | /5Phos/ACGACGCTCTTCCGATCTNNNNNNNNCTTGCGCCGCT | 22 | CTTGCGCCGC | 214 |
| | TTTTTTTTTTTTTT | | | |
| shortDT_plate1_23 | /5Phos/ACGACGCTCTTCCGATCTNNNNNNNNAATCGTAGCGT | 23 | AATCGTAGCG | 215 |
| | TTTTTTTTTTTTTT | | | |
| shortDT_plate1_24 | /5Phos/ACGACGCTCTTCCGATCTNNNNNNNNGGTACTGCCTT | 24 | GGTACTGCCT | 216 |
| | TTTTTTTTTTTTTT | | | |
| shortDT_plate1_25 | /5Phos/ACGACGCTCTTCCGATCTNNNNNNNNTAGAATTAACT | 25 | TAGAATTAAC | 217 |
| | TTTTTTTTTTTTTT | | | |
| shortDT_plate1_26 | /5Phos/ACGACGCTCTTCCGATCTNNNNNNNNGCCATTCTCCTT | 26 | GCCATTCTCC | 218 |
| | TTTTTTTTTTTTT | | | |
| shortDT_plate1_27 | /5Phos/ACGACGCTCTTCCGATCTNNNNNNNNTGCCGGCAGAT | 27 | TGCCGGCAGA | 219 |
| | TTTTTTTTTTTTTT | | | |
| shortDT_plate1_28 | /5Phos/ACGACGCTCTTCCGATCTNNNNNNNNTTACCGAGGCT | 28 | TTACCGAGGC | 220 |
| | TTTTTTTTTTTTTT | | | |
| shortDT_plate1_29 | /5Phos/ACGACGCTCTTCCGATCTNNNNNNNNATCATATTAGT | 29 | ATCATATTAG | 221 |
| | TTTTTTTTTTTTTT | | | |
| shortDT_plate1_30 | /5Phos/ACGACGCTCTTCCGATCTNNNNNNNNTGGTCAGCCAT | 30 | TGGTCAGCCA | 222 |
| | TTTTTTTTTTTTTT | | | |
| shortDT_plate1_31 | /5Phos/ACGACGCTCTTCCGATCTNNNNNNNNACTATGCAATT | 31 | ACTATGCAAT | 223 |
| | TTTTTTTTTTTTTT | | | |
| shortDT_plate1_32 | /5Phos/ACGACGCTCTTCCGATCTNNNNNNNNCGACGCGACTT | 32 | CGACGCGACT | 224 |
| | TTTTTTTTTTTTTT | | | |
| shortDT_plate1_33 | /5Phos/ACGACGCTCTTCCGATCTNNNNNNNNGATACGGAACT | 33 | GATACGGAAC | 225 |
| | TTTTTTTTTTTTTT | | | |
| shortDT_plate1_34 | /5Phos/ACGACGCTCTTCCGATCTNNNNNNNNTTATCCGGATT | 34 | TTATCCGGAT | 226 |
| | TTTTTTTTTTTTTT | | | |
| shortDT_plate1_35 | /5Phos/ACGACGCTCTTCCGATCTNNNNNNNNTAGAGTAATAT | 35 | TAGAGTAATA | 227 |
| | TTTTTTTTTTTTTT | | | |
| shortDT_plate1_36 | /5Phos/ACGACGCTCTTCCGATCTNNNNNNNNGCAGGTCCGTT | 36 | GCAGGTCCGT | 228 |
| | TTTTTTTTTTTTTT | | | |
| shortDT_plate1_37 | /5Phos/ACGACGCTCTTCCGATCTNNNNNNNNTCGGCCTTACT | 37 | TCGGCCTTAC | 229 |
| | TTTTTTTTTTTTTT | | | |
| shortDT_plate1_38 | /5Phos/ACGACGCTCTTCCGATCTNNNNNNNNAGAACGTCTCT | 38 | AGAACGTCTC | 230 |
| | TTTTTTTTTTTTTT | | | |
| shortDT_plate1_39 | /5Phos/ACGACGCTCTTCCGATCTNNNNNNNNCCAGTTCCAAT | 39 | CCAGTTCCAA | 231 |
| | TTTTTTTTTTTTTT | | | |
| shortDT_plate1_40 | /5Phos/ACGACGCTCTTCCGATCTNNNNNNNNGGCGTTAAGGT | 40 | GGCGTTAAGG | 232 |
| | TTTTTTTTTTTTTT | | | |
| shortDT_plate1_41 | /5Phos/ACGACGCTCTTCCGATCTNNNNNNNNACTTAACCTTTT | 41 | ACTTAACCTT | 233 |
| | TTTTTTTTTTTTT | | | |
| shortDT_plate1_42 | /5Phos/ACGACGCTCTTCCGATCTNNNNNNNNCAACCGCTAAT | 42 | CAACCGCTAA | 234 |
| | TTTTTTTTTTTTTT | | | |
| shortDT_plate1_43 | /5Phos/ACGACGCTCTTCCGATCTNNNNNNNNGACCTTGATAT | 43 | GACCTTGATA | 235 |
| | TTTTTTTTTTTTTT | | | |
| shortDT_plate1_44 | /5Phos/ACGACGCTCTTCCGATCTNNNNNNNNTCTGATACCAT | 44 | TCTGATACCA | 236 |
| | TTTTTTTTTTTTTT | | | |
| shortDT_plate1_45 | /5Phos/ACGACGCTCTTCCGATCTNNNNNNNNGAAGATCGAG | 45 | GAAGATCGAG | 237 |
| | TTTTTTTTTTTTTTT | | | |
| shortDT_plate1_46 | /5Phos/ACGACGCTCTTCCGATCTNNNNNNNNAGGAGCGGTA | 46 | AGGAGCGGTA | 238 |
| | TTTTTTTTTTTTTTT | | | |
| shortDT_plate1_47 | /5Phos/ACGACGCTCTTCCGATCTNNNNNNNNAAGAAGCTAGT | 47 | AAGAAGCTAG | 239 |
| | TTTTTTTTTTTTTT | | | |
| shortDT_plate1_48 | /5Phos/ACGACGCTCTTCCGATCTNNNNNNNNTCCGGCCTCGT | 48 | TCCGGCCTCG | 240 |
| | TTTTTTTTTTTTTT | | | |
| shortDT_plate1_49 | /5Phos/ACGACGCTCTTCCGATCTNNNNNNNNAGAGAAGGTTT | 49 | AGAGAAGGTT | 241 |
| | TTTTTTTTTTTTTT | | | |
| shortDT_plate1_50 | /5Phos/ACGACGCTCTTCCGATCTNNNNNNNNCATACTCCGAT | 50 | CATACTCCGA | 242 |
| | TTTTTTTTTTTTTT | | | |
| shortDT_plate1_51 | /5Phos/ACGACGCTCTTCCGATCTNNNNNNNNGCTAACTTGCT | 51 | GCTAACTTGC | 243 |
| | TTTTTTTTTTTTTT | | | |
| shortDT_plate1_52 | /5Phos/ACGACGCTCTTCCGATCTNNNNNNNNAATCCATCTTTT | 52 | AATCCATCTT | 244 |
| | TTTTTTTTTTTTT | | | |
| shortDT_plate1_53 | /5Phos/ACGACGCTCTTCCGATCTNNNNNNNNGGCTGAGCTCT | 53 | GGCTGAGCTC | 245 |
| | TTTTTTTTTTTTTT | | | |
| shortDT_plate1_54 | /5Phos/ACGACGCTCTTCCGATCTNNNNNNNNCCGATTCCTGT | 54 | CCGATTCCTG | 246 |
| | TTTTTTTTTTTTTT | | | |
| shortDT_plate1_55 | /5Phos/ACGACGCTCTTCCGATCTNNNNNNNNACCGCCAACCT | 55 | ACCGCCAACC | 247 |
| | TTTTTTTTTTTTTT | | | |
| shortDT_plate1_56 | /5Phos/ACGACGCTCTTCCGATCTNNNNNNNNTGGCCTGAAGT | 56 | TGGCCTGAAG | 248 |
| | TTTTTTTTTTTTTT | | | |
| shortDT_plate1_57 | /5Phos/ACGACGCTCTTCCGATCTNNNNNNNNAACCTCATTCTT | 57 | AACCTCATTC | 249 |
| | TTTTTTTTTTTTT | | | |
| shortDT_plate1_58 | /5Phos/ACGACGCTCTTCCGATCTNNNNNNNNATAAGGAGCAT | 58 | ATAAGGAGCA | 250 |
| | TTTTTTTTTTTTTT | | | |
| shortDT_plate1_59 | /5Phos/ACGACGCTCTTCCGATCTNNNNNNNNCGAACGCCGGT | 59 | CGAACGCCGG | 251 |
| | TTTTTTTTTTTTTT | | | |
| shortDT_plate1_60 | /5Phos/ACGACGCTCTTCCGATCTNNNNNNNNGGTATGCTTGT | 60 | GGTATGCTTG | 252 |
| | TTTTTTTTTTTTTT | | | |
| shortDT_plate1_61 | /5Phos/ACGACGCTCTTCCGATCTNNNNNNNNAACCTGCGTAT | 61 | AACCTGCGTA | 253 |
| | TTTTTTTTTTTTTT | | | |
| shortDT_plate1_62 | /5Phos/ACGACGCTCTTCCGATCTNNNNNNNNGGCAGACGCCT | 52 | GGCAGACGCC | 254 |
| | TTTTTTTTTTTTTT | | | |
| shortDT_plate1_63 | /5Phos/ACGACGCTCTTCCGATCTNNNNNNNNTAGCCGTCATT | 63 | TAGCCGTCAT | 255 |
| | TTTTTTTTTTTTTT | | | |
| shortDT_plate1_64 | /5Phos/ACGACGCTCTTCCGATCTNNNNNNNNCCTGGAAGAGT | 64 | CCTGGAAGAG | 256 |
| | TTTTTTTTTTTTTT | | | |
| shortDT_plate1_65 | /5Phos/ACGACGCTCTTCCGATCTNNNNNNNNGGAGGTTCTAT | 65 | GGAGGTTCTA | 257 |
| | TTTTTTTTTTTTTT | | | |
| shortDT_plate1_66 | /5Phos/ACGACGCTCTTCCGATCTNNNNNNNNCTAGTAGTCTT | 66 | CTAGTAGTCT | 258 |
| | TTTTTTTTTTTTTT | | | |
| shortDT_plate1_67 | /5Phos/ACGACGCTCTTCCGATCTNNNNNNNNATCATCAACGT | 67 | ATCATCAACG | 259 |
| | TTTTTTTTTTTTTT | | | |
| shortDT_plate1_68 | /5Phos/ACGACGCTCTTCCGATCTNNNNNNNNACGCGAGATTT | 68 | ACGCGAGATT | 260 |
| | TTTTTTTTTTTTTT | | | |
| shortDT_plate1_69 | /5Phos/ACGACGCTCTTCCGATCTNNNNNNNNGAAGAGGCAT | 69 | GAAGAGGCAT | 261 |
| | TTTTTTTTTTTTTTT | | | |
| shortDT_plate1_70 | /5Phos/ACGACGCTCTTCCGATCTNNNNNNNNGGTATCCGCCT | 70 | GGTATCCGCC | 262 |
| | TTTTTTTTTTTTTT | | | |
| shortDT_plate1_71 | /5Phos/ACGACGCTCTTCCGATCTNNNNNNNNAACTAGGCGCT | 71 | AACTAGGCGC | 263 |
| | TTTTTTTTTTTTTT | | | |
| shortDT_plate1_72 | /5Phos/ACGACGCTCTTCCGATCTNNNNNNNNTCGCTAAGCAT | 72 | TCGCTAAGCA | 264 |
| | TTTTTTTTTTTTTT | | | |
| shortDT_plate1_73 | /5Phos/ACGACGCTCTTCCGATCTNNNNNNNNTATATACTAAT | 73 | TATATACTAA | 265 |
| | TTTTTTTTTTTTTT | | | |
| shortDT_plate1_74 | /5Phos/ACGACGCTCTTCCGATCTNNNNNNNNACTTGCTAGAT | 74 | ACTTGCTAGA | 266 |
| | TTTTTTTTTTTTTT | | | |
| shortDT_plate1_75 | /5Phos/ACGACGCTCTTCCGATCTNNNNNNNNAACCATTGGAT | 75 | AACCATTGGA | 267 |
| | TTTTTTTTTTTTTT | | | |
| shortDT_plate1_76 | /5Phos/ACGACGCTCTTCCGATCTNNNNNNNNTCGCGGTTGGT | 76 | TCGCGGTTGG | 268 |
| | TTTTTTTTTTTTTT | | | |
| shortDT_plate1_77 | /5Phos/ACGACGCTCTTCCGATCTNNNNNNNNCGTAGTTACCT | 77 | CGTAGTTACC | 269 |
| | TTTTTTTTTTTTTT | | | |
| shortDT_plate1_78 | /5Phos/ACGACGCTCTTCCGATCTNNNNNNNNTCCAATCATCTT | 78 | TCCAATCATC | 270 |
| | TTTTTTTTTTTTT | | | |
| shortDT_plate1_79 | /5Phos/ACGACGCTCTTCCGATCTNNNNNNNNAATCGATAATT | 79 | AATCGATAAT | 271 |
| | TTTTTTTTTTTTTT | | | |
| shortDT_plate1_80 | /5Phos/ACGACGCTCTTCCGATCTNNNNNNNNCCATTATCTATT | 80 | CCATTATCTA | 272 |
| | TTTTTTTTTTTTT | | | |
| shortDT_plate1_81 | /5Phos/ACGACGCTCTTCCGATCTNNNNNNNNTCAACGTAAGT | 81 | TCAACGTAAG | 273 |
| | TTTTTTTTTTTTTT | | | |
| shortDT_plate1_82 | /5Phos/ACGACGCTCTTCCGATCTNNNNNNNNTCTAATAGTAT | 82 | TCTAATAGTA | 274 |
| | TTTTTTTTTTTTTT | | | |
| shortDT_plate1_83 | /5Phos/ACGACGCTCTTCCGATCTNNNNNNNNAACCGCTGGTT | 83 | AACCGCTGGT | 275 |
| | TTTTTTTTTTTTTT | | | |
| shortDT_plate1_84 | /5Phos/ACGACGCTCTTCCGATCTNNNNNNNNGATCGCTTCTT | 84 | GATCGCTTCT | 276 |
| | TTTTTTTTTTTTTT | | | |
| shortDT_plate1_85 | /5Phos/ACGACGCTCTTCCGATCTNNNNNNNNCTAACTAGATT | 85 | CTAACTAGAT | 277 |
| | TTTTTTTTTTTTTT | | | |
| shortDT_plate1_86 | /5Phos/ACGACGCTCTTCCGATCTNNNNNNNNGCTGGAACTTT | 86 | GCTGGAACTT | 278 |
| | TTTTTTTTTTTTTT | | | |
| shortDT_plate1_87 | /5Phos/ACGACGCTCTTCCGATCTNNNNNNNNAGGTTAGTTCT | 87 | AGGTTAGTTC | 279 |
| | TTTTTTTTTTTTTT | | | |
| shortDT_plate1_88 | /5Phos/ACGACGCTCTTCCGATCTNNNNNNNNCATTCGACGGT | 88 | CATTCGACGG | 280 |
| | TTTTTTTTTTTTTT | | | |
| shortDT_plate1_89 | /5Phos/ACGACGCTCTTCCGATCTNNNNNNNNCATTCAATCAT | 89 | CATTCAATCA | 281 |
| | TTTTTTTTTTTTTT | | | |
| shortDT_plate1_90 | /5Phos/ACGACGCTCTTCCGATCTNNNNNNNNCGGATTAGAAT | 90 | CGGATTAGAA | 282 |
| | TTTTTTTTTTTTTT | | | |
| shortDT_plate1_91 | /5Phos/ACGACGCTCTTCCGATCTNNNNNNNNATCGGCTATCT | 91 | ATCGGCTATC | 283 |
| | TTTTTTTTTTTTTT | | | |
| shortDT_plate1_92 | /5Phos/ACGACGCTCTTCCGATCTNNNNNNNNCCTTGATCGTT | 92 | CCTTGATCGT | 284 |
| | TTTTTTTTTTTTTT | | | |
| shortDT_plate1_93 | /5Phos/ACGACGCTCTTCCGATCTNNNNNNNNACGAAGTCAAT | 93 | ACGAAGTCAA | 285 |
| | TTTTTTTTTTTTTT | | | |
| shortDT_plate1_94 | /5Phos/ACGACGCTCTTCCGATCTNNNNNNNNTTACCTCGACT | 94 | TTACCTCGAC | 286 |
| | TTTTTTTTTTTTTT | | | |
| shortDT_plate1_95 | /5Phos/ACGACGCTCTTCCGATCTNNNNNNNNGGAGGATAGC | 95 | GGAGGATAGC | 287 |
| | TTTTTTTTTTTTTTT | | | |
| shortDT_plate1_96 | /5Phos/ACGACGCTCTTCCGATCTNNNNNNNNGGCTCTCTATT | 96 | GGCTCTCTAT | 288 |
| | TTTTTTTTTTTTTT | | | |
| shortDT_plate2_01 | /5Phos/ACGACGCTCTTCCGATCTNNNNNNNNGGTTGGCGACT | 97 | GGTTGGCGAC | 289 |
| | TTTTTTTTTTTTTT | | | |
| shortDT_plate2_02 | /5Phos/ACGACGCTCTTCCGATCTNNNNNNNNGTAGATCGTTT | 98 | GTAGATCGTT | 290 |
| | TTTTTTTTTTTTTT | | | |
| shortDT_plate2_03 | /5Phos/ACGACGCTCTTCCGATCTNNNNNNNNGAGGTCGGTTT | 99 | GAGGTCGGTT | 291 |
| | TTTTTTTTTTTTTT | | | |
| shortDT_plate2_04 | /5Phos/ACGACGCTCTTCCGATCTNNNNNNNNACGCGCTCCTT | 100 | ACGCGCTCCT | 292 |
| | TTTTTTTTTTTTTT | | | |
| shortDT_plate2_05 | /5Phos/ACGACGCTCTTCCGATCTNNNNNNNNAGCGTCGTATT | 101 | AGCGTCGTAT | 293 |
| | TTTTTTTTTTTTTT | | | |
| shortDT_plate2_06 | /5Phos/ACGACGCTCTTCCGATCTNNNNNNNNGACCAATGCGT | 102 | GACCAATGCG | 294 |
| | TTTTTTTTTTTTTT | | | |
| shortDT_plate2_07 | /5Phos/ACGACGCTCTTCCGATCTNNNNNNNNAGGTAGAGCTT | 103 | AGGTAGAGCT | 295 |
| | TTTTTTTTTTTTTT | | | |
| shortDT_plate2_08 | /5Phos/ACGACGCTCTTCCGATCTNNNNNNNNTTGCAGCATTT | 104 | TTGCAGCATT | 296 |
| | TTTTTTTTTTTTTT | | | |
| shortDT_plate2_09 | /5Phos/ACGACGCTCTTCCGATCTNNNNNNNNGTAGATGCGCT | 105 | GTAGATGCGC | 297 |
| | TTTTTTTTTTTTTT | | | |
| shortDT_plate2_10 | /5Phos/ACGACGCTCTTCCGATCTNNNNNNNNTCGGTAAGGCT | 106 | TCGGTAAGGC | 298 |
| | TTTTTTTTTTTTTT | | | |
| shortDT_plate2_11 | /5Phos/ACGACGCTCTTCCGATCTNNNNNNNNACGATAGACTT | 107 | ACGATAGACT | 299 |
| | TTTTTTTTTTTTTT | | | |
| shortDT_plate2_12 | /5Phos/ACGACGCTCTTCCGATCTNNNNNNNNGCGGCCAATCT | 108 | GCGGCCAATC | 300 |
| | TTTTTTTTTTTTTT | | | |
| shortDT_plate2_13 | /5Phos/ACGACGCTCTTCCGATCTNNNNNNNNACGCGTATCGT | 109 | ACGCGTATCG | 301 |
| | TTTTTTTTTTTTTT | | | |
| shortDT_plate2_14 | /5Phos/ACGACGCTCTTCCGATCTNNNNNNNNCATGACTCAAT | 110 | CATGACTCAA | 302 |
| | TTTTTTTTTTTTTT | | | |
| shortDT_plate2_15 | /5Phos/ACGACGCTCTTCCGATCTNNNNNNNNACTCCGCCAAT | 111 | ACTCCGCCAA | 303 |
| | TTTTTTTTTTTTTT | | | |
| shortDT_plate2_16 | /5Phos/ACGACGCTCTTCCGATCTNNNNNNNNACGTTGAATGT | 112 | ACGTTGAATG | 304 |
| | TTTTTTTTTTTTTT | | | |
| shortDT_plate2_17 | /5Phos/ACGACGCTCTTCCGATCTNNNNNNNNAGGACTGCGAT | 113 | AGGACTGCGA | 305 |
| | TTTTTTTTTTTTTT | | | |
| shortDT_plate2_18 | /5Phos/ACGACGCTCTTCCGATCTNNNNNNNNACTCGACGCCT | 114 | ACTCGACGCC | 306 |
| | TTTTTTTTTTTTTT | | | |
| shortDT_plate2_19 | /5Phos/ACGACGCTCTTCCGATCTNNNNNNNNCCTATCATAAT | 115 | CCTATCATAA | 307 |
| | TTTTTTTTTTTTTT | | | |
| shortDT_plate2_20 | /5Phos/ACGACGCTCTTCCGATCTNNNNNNNNAATCCGGTCAT | 116 | AATCCGGTCA | 308 |
| | TTTTTTTTTTTTTT | | | |
| shortDT_plate2_21 | /5Phos/ACGACGCTCTTCCGATCTNNNNNNNNCTATTAACCAT | 117 | CTATTAACCA | 309 |
| | TTTTTTTTTTTTTT | | | |
| shortDT_plate2_22 | /5Phos/ACGACGCTCTTCCGATCTNNNNNNNNGATCCAGCGTT | 118 | GATCCAGCGT | 310 |
| | TTTTTTTTTTTTTT | | | |
| shortDT_plate2_23 | /5Phos/ACGACGCTCTTCCGATCTNNNNNNNNTGAGACTCTAT | 119 | TGAGACTCTA | 311 |
| | TTTTTTTTTTTTTT | | | |
| shortDT_plate2_24 | /5Phos/ACGACGCTCTTCCGATCTNNNNNNNNGCGGAGTCGA | 120 | GCGGAGTCGA | 312 |
| | TTTTTTTTTTTTTTT | | | |
| shortDT_plate2_25 | /5Phos/ACGACGCTCTTCCGATCTNNNNNNNNGAGGCTTATTT | 121 | GAGGCTTATT | 313 |
| | TTTTTTTTTTTTTT | | | |
| shortDT_plate2_26 | /5Phos/ACGACGCTCTTCCGATCTNNNNNNNNCGCCAGGCATT | 122 | CGCCAGGCAT | 314 |
| | TTTTTTTTTTTTTT | | | |
| shortDT_plate2_27 | /5Phos/ACGACGCTCTTCCGATCTNNNNNNNNAATACCAGTTT | 123 | AATACCAGTT | 315 |
| | TTTTTTTTTTTTTT | | | |
| shortDT_plate2_28 | /5Phos/ACGACGCTCTTCCGATCTNNNNNNNNGCGGTTATTGT | 124 | GCGGTTATTG | 316 |
| | TTTTTTTTTTTTTT | | | |
| shortDT_plate2_29 | /5Phos/ACGACGCTCTTCCGATCTNNNNNNNNCATCCAGCCAT | 125 | CATCCAGCCA | 317 |
| | TTTTTTTTTTTTTT | | | |
| shortDT_plate2_30 | /5Phos/ACGACGCTCTTCCGATCTNNNNNNNNGGCTGCCTTAT | 126 | GGCTGCCTTA | 318 |
| | TTTTTTTTTTTTTT | | | |
| shortDT_plate2_31 | /5Phos/ACGACGCTCTTCCGATCTNNNNNNNNTTCTATAGAGT | 127 | TTCTATAGAG | 319 |
| | TTTTTTTTTTTTTT | | | |
| shortDT_plate2_32 | /5Phos/ACGACGCTCTTCCGATCTNNNNNNNNTCTAGTCAAGT | 128 | TCTAGTCAAG | 320 |
| | TTTTTTTTTTTTTT | | | |
| shortDT_plate2_33 | /5Phos/ACGACGCTCTTCCGATCTNNNNNNNNACGGAGAATAT | 129 | ACGGAGAATA | 321 |
| | TTTTTTTTTTTTTT | | | |
| shortDT_plate2_34 | /5Phos/ACGACGCTCTTCCGATCTNNNNNNNNATTAACTTAAT | 130 | ATTAACTTAA | 322 |
| | TTTTTTTTTTTTTT | | | |
| shortDT_plate2_35 | /5Phos/ACGACGCTCTTCCGATCTNNNNNNNNCGTATTGAGAT | 131 | CGTATTGAGA | 323 |
| | TTTTTTTTTTTTTT | | | |
| shortDT_plate2_36 | /5Phos/ACGACGCTCTTCCGATCTNNNNNNNNTAGCCAGCAAT | 132 | TAGCCAGCAA | 324 |
| | TTTTTTTTTTTTTT | | | |
| shortDT_plate2_37 | /5Phos/ACGACGCTCTTCCGATCTNNNNNNNNTCGGCGTCGTT | 133 | TCGGCGTCGT | 325 |
| | TTTTTTTTTTTTTT | | | |
| shortDT_plate2_38 | /5Phos/ACGACGCTCTTCCGATCTNNNNNNNNGCCTGATATAT | 134 | GCCTGATATA | 326 |
| | TTTTTTTTTTTTTT | | | |
| shortDT_plate2_39 | /5Phos/ACGACGCTCTTCCGATCTNNNNNNNNGCCTCAGCATT | 135 | GCCTCAGCAT | 327 |
| | TTTTTTTTTTTTTT | | | |
| shortDT_plate2_40 | /5Phos/ACGACGCTCTTCCGATCTNNNNNNNNATCTAGGTTCT | 136 | ATCTAGGTTC | 328 |
| | TTTTTTTTTTTTTT | | | |
| shortDT_plate2_41 | /5Phos/ACGACGCTCTTCCGATCTNNNNNNNNGACGAGGTTGT | 137 | GACGAGGTTG | 329 |
| | TTTTTTTTTTTTTT | | | |
| shortDT_plate2_42 | /5Phos/ACGACGCTCTTCCGATCTNNNNNNNNCTGGTTGGTTT | 138 | CTGGTTGGTT | 330 |
| | TTTTTTTTTTTTTT | | | |
| shortDT_plate2_43 | /5Phos/ACGACGCTCTTCCGATCTNNNNNNNNCCGCGCAGGTT | 139 | CCGCGCAGGT | 331 |
| | TTTTTTTTTTTTTT | | | |
| shortDT_plate2_44 | /5Phos/ACGACGCTCTTCCGATCTNNNNNNNNACTCTACTGGT | 140 | ACTCTACTGG | 332 |
| | TTTTTTTTTTTTTT | | | |
| shortDT_plate2_45 | /5Phos/ACGACGCTCTTCCGATCTNNNNNNNNCCTGAGAGCAT | 141 | CCTGAGAGCA | 333 |
| | TTTTTTTTTTTTTT | | | |
| shortDT_plate2_46 | /5Phos/ACGACGCTCTTCCGATCTNNNNNNNNACCAGTATAAT | 142 | ACCAGTATAA | 334 |
| | TTTTTTTTTTTTTT | | | |
| shortDT_plate2_47 | /5Phos/ACGACGCTCTTCCGATCTNNNNNNNNTCCGCCGGTCT | 143 | TCCGCCGGTC | 335 |
| | TTTTTTTTTTTTTT | | | |
| shortDT_plate2_48 | /5Phos/ACGACGCTCTTCCGATCTNNNNNNNNTGCCTAACTTTT | 144 | TGCCTAACTT | 336 |
| | TTTTTTTTTTTTT | | | |
| shortDT_plate2_49 | /5Phos/ACGACGCTCTTCCGATCTNNNNNNNNTTCTCTGAGAT | 145 | TTCTCTGAGA | 337 |
| | TTTTTTTTTTTTTT | | | |
| shortDT_plate2_50 | /5Phos/ACGACGCTCTTCCGATCTNNNNNNNNCCTGCATCAAT | 146 | CCTGCATCAA | 338 |
| | TTTTTTTTTTTTTT | | | |
| shortDT_plate2_51 | /5Phos/ACGACGCTCTTCCGATCTNNNNNNNNTCTGACGAGGT | 147 | TCTGACGAGG | 339 |
| | TTTTTTTTTTTTTT | | | |
| shortDT_plate2_52 | /5Phos/ACGACGCTCTTCCGATCTNNNNNNNNGATTCCGGAAT | 148 | GATTCCGGAA | 340 |
| | TTTTTTTTTTTTTT | | | |
| shortDT_plate2_53 | /5Phos/ACGACGCTCTTCCGATCTNNNNNNNNTGGCATAACGT | 149 | TGGCATAACG | 341 |
| | TTTTTTTTTTTTTT | | | |
| shortDT_plate2_54 | /5Phos/ACGACGCTCTTCCGATCTNNNNNNNNTCTCTCATCCTT | 150 | TCTCTCATCC | 342 |
| | TTTTTTTTTTTTT | | | |
| shortDT_plate2_55 | /5Phos/ACGACGCTCTTCCGATCTNNNNNNNNTTCGCTGCCTTT | 151 | TTCGCTGCCT | 343 |
| | TTTTTTTTTTTTT | | | |
| shortDT_plate2_56 | /5Phos/ACGACGCTCTTCCGATCTNNNNNNNNGGATTCTATCT | 152 | GGATTCTATC | 344 |
| | TTTTTTTTTTTTTT | | | |
| shortDT_plate2_57 | /5Phos/ACGACGCTCTTCCGATCTNNNNNNNNTAGAATAGCCT | 153 | TAGAATAGCC | 345 |
| | TTTTTTTTTTTTTT | | | |
| shortDT_plate2_58 | /5Phos/ACGACGCTCTTCCGATCTNNNNNNNNGCTCGAATCAT | 154 | GCTCGAATCA | 346 |
| | TTTTTTTTTTTTTT | | | |
| shortDT_plate2_59 | /5Phos/ACGACGCTCTTCCGATCTNNNNNNNNGGCTCGAGATT | 155 | GGCTCGAGAT | 347 |
| | TTTTTTTTTTTTTT | | | |
| shortDT_plate2_60 | /5Phos/ACGACGCTCTTCCGATCTNNNNNNNNTCCTCTCCGTTT | 156 | TCCTCTCCGT | 348 |
| | TTTTTTTTTTTTT | | | |
| shortDT_plate2_61 | /5Phos/ACGACGCTCTTCCGATCTNNNNNNNNATAACCGTTCT | 157 | ATAACCGTTC | 349 |
| | TTTTTTTTTTTTTT | | | |
| shortDT_plate2_62 | /5Phos/ACGACGCTCTTCCGATCTNNNNNNNNAGGTCTATGGT | 158 | AGGTCTATGG | 350 |
| | TTTTTTTTTTTTTT | | | |
| shortDT_plate2_63 | /5Phos/ACGACGCTCTTCCGATCTNNNNNNNNAGCAAGAACCT | 159 | AGCAAGAACC | 351 |
| | TTTTTTTTTTTTTT | | | |
| shortDT_plate2_64 | /5Phos/ACGACGCTCTTCCGATCTNNNNNNNNTTGATATGAAT | 160 | TTGATATGAA | 352 |
| | TTTTTTTTTTTTTT | | | |
| shortDT_plate2_65 | /5Phos/ACGACGCTCTTCCGATCTNNNNNNNNTGGCAGAAGTT | 161 | TGGCAGAAGT | 353 |
| | TTTTTTTTTTTTTT | | | |
| shortDT_plate2_66 | /5Phos/ACGACGCTCTTCCGATCTNNNNNNNNCTTCATTAGAT | 162 | CTTCATTAGA | 354 |
| | TTTTTTTTTTTTTT | | | |
| shortDT_plate2_67 | /5Phos/ACGACGCTCTTCCGATCTNNNNNNNNAATCGAACTCT | 163 | AATCGAACTC | 355 |
| | TTTTTTTTTTTTTT | | | |
| shortDT_plate2_68 | /5Phos/ACGACGCTCTTCCGATCTNNNNNNNNGGACGACGCA | 164 | GGACGACGCA | 356 |
| | TTTTTTTTTTTTTTT | | | |
| shortDT_plate2_69 | /5Phos/ACGACGCTCTTCCGATCTNNNNNNNNCGTCTATGAAT | 165 | CGTCTATGAA | 357 |
| | TTTTTTTTTTTTTT | | | |
| shortDT_plate2_70 | /5Phos/ACGACGCTCTTCCGATCTNNNNNNNNCGAATCTCCTT | 166 | CGAATCTCCT | 358 |
| | TTTTTTTTTTTTTT | | | |
| shortDT_plate2_71 | /5Phos/ACGACGCTCTTCCGATCTNNNNNNNNGGCTATTCGAT | 167 | GGCTATTCGA | 359 |
| | TTTTTTTTTTTTTT | | | |
| shortDT_plate2_72 | /5Phos/ACGACGCTCTTCCGATCTNNNNNNNNTCTATCGGTAT | 168 | TCTATCGGTA | 360 |
| | TTTTTTTTTTTTTT | | | |
| shortDT_plate2_73 | /5Phos/ACGACGCTCTTCCGATCTNNNNNNNNCGAAGGCATGT | 169 | CGAAGGCATG | 361 |
| | TTTTTTTTTTTTTT | | | |
| shortDT_plate2_74 | /5Phos/ACGACGCTCTTCCGATCTNNNNNNNNAATTGAGAGAT | 170 | AATTGAGAGA | 362 |
| | TTTTTTTTTTTTTT | | | |
| shortDT_plate2_75 | /5Phos/ACGACGCTCTTCCGATCTNNNNNNNNGTAGTTGGATT | 171 | GTAGTTGGAT | 363 |
| | TTTTTTTTTTTTTT | | | |
| shortDT_plate2_76 | /5Phos/ACGACGCTCTTCCGATCTNNNNNNNNCCTAAGCGGTT | 172 | CCTAAGCGGT | 364 |
| | TTTTTTTTTTTTTT | | | |
| shortDT_plate2_77 | /5Phos/ACGACGCTCTTCCGATCTNNNNNNNNCGTAAGGAGTT | 173 | CGTAAGGAGT | 365 |
| | TTTTTTTTTTTTTT | | | |
| shortDT_plate2_78 | /5Phos/ACGACGCTCTTCCGATCTNNNNNNNNAAGCATCCTAT | 174 | AAGCATCCTA | 366 |
| | TTTTTTTTTTTTTT | | | |
| shortDT_plate2_79 | /5Phos/ACGACGCTCTTCCGATCTNNNNNNNNCTGAAGAGACT | 175 | CTGAAGAGAC | 367 |
| | TTTTTTTTTTTTTT | | | |
| shortDT_plate2_80 | /5Phos/ACGACGCTCTTCCGATCTNNNNNNNNGGCTTCTGGAT | 176 | GGCTTCTGGA | 368 |
| | TTTTTTTTTTTTTT | | | |
| shortDT_plate2_81 | /5Phos/ACGACGCTCTTCCGATCTNNNNNNNNAGCGATCCGCT | 177 | AGCGATCCGC | 369 |
| | TTTTTTTTTTTTTT | | | |
| shortDT_plate2_82 | /5Phos/ACGACGCTCTTCCGATCTNNNNNNNNACGCTTCTCTTT | 178 | ACGCTTCTCT | 370 |
| | TTTTTTTTTTTTT | | | |
| shortDT_plate2_83 | /5Phos/ACGACGCTCTTCCGATCTNNNNNNNNATATGCCATCT | 179 | ATATGCCATC | 371 |
| | TTTTTTTTTTTTTT | | | |
| shortDT_plate2_84 | /5Phos/ACGACGCTCTTCCGATCTNNNNNNNNAAGTACGTTAT | 180 | AAGTACGTTA | 372 |
| | TTTTTTTTTTTTTT | | | |
| shortDT_plate2_85 | /5Phos/ACGACGCTCTTCCGATCTNNNNNNNNGAATGAGGAG | 181 | GAATGAGGAG | 373 |
| | TTTTTTTTTTTTTTT | | | |
| shortDT_plate2_86 | /5Phos/ACGACGCTCTTCCGATCTNNNNNNNNAGGCCGGTAAT | 182 | AGGCCGGTAA | 374 |
| | TTTTTTTTTTTTTT | | | |
| shortDT_plate2_87 | /5Phos/ACGACGCTCTTCCGATCTNNNNNNNNGCCATCAACTT | 183 | GCCATCAACT | 375 |
| | TTTTTTTTTTTTTT | | | |
| shortDT_plate2_88 | /5Phos/ACGACGCTCTTCCGATCTNNNNNNNNACTGGTAGATT | 184 | ACTGGTAGAT | 376 |
| | TTTTTTTTTTTTTT | | | |
| shortDT_plate2_89 | /5Phos/ACGACGCTCTTCCGATCTNNNNNNNNATGAGTTCTCT | 185 | ATGAGTTCTC | 377 |
| | TTTTTTTTTTTTTT | | | |
| shortDT_plate2_90 | /5Phos/ACGACGCTCTTCCGATCTNNNNNNNNCCATCGGACCT | 186 | CCATCGGACC | 378 |
| | TTTTTTTTTTTTTT | | | |
| shortDT_plate2_91 | /5Phos/ACGACGCTCTTCCGATCTNNNNNNNNGGCATCTACCT | 187 | GGCATCTACC | 379 |
| | TTTTTTTTTTTTTT | | | |
| shortDT_plate2_92 | /5Phos/ACGACGCTCTTCCGATCTNNNNNNNNTTCTCTACTATT | 188 | TTCTCTACTA | 380 |
| | TTTTTTTTTTTTT | | | |
| shortDT_plate2_93 | /5Phos/ACGACGCTCTTCCGATCTNNNNNNNNCCAGGCTCTTT | 189 | CCAGGCTCTT | 381 |
| | TTTTTTTTTTTTTT | | | |
| shortDT_plate2_94 | /5Phos/ACGACGCTCTTCCGATCTNNNNNNNNATCCATCAGGT | 190 | ATCCATCAGG | 382 |
| | TTTTTTTTTTTTTT | | | |
| shortDT_plate2_95 | /5Phos/ACGACGCTCTTCCGATCTNNNNNNNNCTCGGAGCAAT | 191 | CTCGGAGCAA | 383 |
| | TTTTTTTTTTTTTT | | | |
| shortDT_plate2_96 | /5Phos/ACGACGCTCTTCCGATCTNNNNNNNNGGCGGTTGACT | 192 | GGCGGTTGAC | 384 |
| | TTTTTTTTTTTTTT | | | |

TABLE 4
Random hexamer reverse transcription primer sequences

| | | SEQ ID | | SEQ ID |
|---|---|---|---|---|
| Name | Sequence | NO: | Barcode | NO: |
| randomN_plate1_01 | /5Phos/ACGACGCTCTTCCGATCTNNNNNNNNCGGTCAAGA | 385 | CGGTCAAGAA | 577 |
| | ANNNNNN | | | |
| randomN_plate1_02 | /5Phos/ACGACGCTCTTCCGATCTNNNNNNNNCGCTCCTAA | 386 | CGCTCCTAAC | 578 |
| | CNNNNNN | | | |
| randomN_plate1_03 | /5Phos/ACGACGCTCTTCCGATCTNNNNNNNNATCCATGAC | 387 | ATCCATGACT | 579 |
| | TNNNNNN | | | |
| randomN_plate1_04 | /5Phos/ACGACGCTCTTCCGATCTNNNNNNNNAACCTGGTC | 388 | AACCTGGTCT | 580 |
| | TNNNNNN | | | |
| randomN_plate1_05 | /5Phos/ACGACGCTCTTCCGATCTNNNNNNNNACCGAAGAC | 389 | ACCGAAGACC | 581 |
| | CNNNNNN | | | |
| randomN_plate1_06 | /5Phos/ACGACGCTCTTCCGATCTNNNNNNNNGGTACCGGC | 390 | GGTACCGGCA | 582 |
| | ANNNNNN | | | |
| randomN_plate1_07 | /5Phos/ACGACGCTCTTCCGATCTNNNNNNNNAAGCCAGTT | 391 | AAGCCAGTTA | 583 |
| | ANNNNNN | | | |
| randomN_plate1_08 | /5Phos/ACGACGCTCTTCCGATCTNNNNNNNNTCTTGCCGA | 392 | TCTTGCCGAC | 584 |
| | CNNNNNN | | | |
| randomN_plate1_09 | /5Phos/ACGACGCTCTTCCGATCTNNNNNNNNAAGACCGTT | 393 | AAGACCGTTG | 585 |
| | GNNNNNN | | | |
| randomN_plate1_10 | /5Phos/ACGACGCTCTTCCGATCTNNNNNNNNAGGTTAGCA | 394 | AGGTTAGCAT | 586 |
| | TNNNNNN | | | |
| randomN_plate1_11 | /5Phos/ACGACGCTCTTCCGATCTNNNNNNNNTTCGCCTCC | 395 | TTCGCCTCCA | 587 |
| | ANNNNNN | | | |
| randomN_plate1_12 | /5Phos/ACGACGCTCTTCCGATCTNNNNNNNNAGAGCCAA | 396 | AGAGCCAAGG | 588 |
| | GGNNNNNN | | | |
| randomN_plate1_13 | /5Phos/ACGACGCTCTTCCGATCTNNNNNNNNAATACCATC | 397 | AATACCATCC | 589 |
| | CNNNNNN | | | |
| randomN_plate1_14 | /5Phos/ACGACGCTCTTCCGATCTNNNNNNNNAGCTCTCCT | 398 | AGCTCTCCTC | 590 |
| | CNNNNNN | | | |
| randomN_plate1_15 | /5Phos/ACGACGCTCTTCCGATCTNNNNNNNNCTTGATTGC | 399 | CTTGATTGCC | 591 |
| | CNNNNNN | | | |
| randomN_plate1_16 | /5Phos/ACGACGCTCTTCCGATCTNNNNNNNNAGCTTATCC | 400 | AGCTTATCCG | 592 |
| | GNNNNNN | | | |
| randomN_plate1_17 | /5Phos/ACGACGCTCTTCCGATCTNNNNNNNNAAGAATCTG | 401 | AAGAATCTGA | 593 |
| | ANNNNNN | | | |
| randomN_plate1_18 | /5Phos/ACGACGCTCTTCCGATCTNNNNNNNNCATCTCTGC | 402 | CATCTCTGCA | 594 |
| | ANNNNNN | | | |
| randomN_plate1_19 | /5Phos/ACGACGCTCTTCCGATCTNNNNNNNNACCTGGCCA | 403 | ACCTGGCCAA | 595 |
| | ANNNNNN | | | |
| randomN_plate1_20 | /5Phos/ACGACGCTCTTCCGATCTNNNNNNNNTAACTGGTT | 404 | TAACTGGTTA | 596 |
| | ANNNNNN | | | |
| randomN_plate1_21 | /5Phos/ACGACGCTCTTCCGATCTNNNNNNNNTTGCTAACG | 405 | TTGCTAACGG | 597 |
| | GNNNNNN | | | |
| randomN_plate1_22 | /5Phos/ACGACGCTCTTCCGATCTNNNNNNNNACTAGAGA | 406 | ACTAGAGAGT | 598 |
| | GTNNNNNN | | | |
| randomN_plate1_23 | /5Phos/ACGACGCTCTTCCGATCTNNNNNNNNAATGCCGCT | 407 | AATGCCGCTT | 599 |
| | TNNNNNN | | | |
| randomN_plate1_24 | /5Phos/ACGACGCTCTTCCGATCTNNNNNNNNTATAGACGC | 408 | TATAGACGCA | 600 |
| | ANNNNNN | | | |
| randomN_plate1_25 | /5Phos/ACGACGCTCTTCCGATCTNNNNNNNNTCAATCGCA | 409 | TCAATCGCAT | 601 |
| | TNNNNNN | | | |
| randomN_plate1_26 | /5Phos/ACGACGCTCTTCCGATCTNNNNNNNNTTCTTAATA | 410 | TTCTTAATAA | 602 |
| | ANNNNNN | | | |
| randomN_plate1_27 | /5Phos/ACGACGCTCTTCCGATCTNNNNNNNNGTCCTAGAG | 411 | GTCCTAGAGG | 603 |
| | GNNNNNN | | | |
| randomN_plate1_28 | /5Phos/ACGACGCTCTTCCGATCTNNNNNNNNATATTGATA | 412 | ATATTGATAC | 604 |
| | CNNNNNN | | | |
| randomN_plate1_29 | /5Phos/ACGACGCTCTTCCGATCTNNNNNNNNCCGCTGCCA | 413 | CCGCTGCCAG | 605 |
| | GNNNNNN | | | |
| randomN_plate1_30 | /5Phos/ACGACGCTCTTCCGATCTNNNNNNNNCCTAGTACG | 414 | CCTAGTACGT | 606 |
| | TNNNNNN | | | |
| randomN_plate1_31 | /5Phos/ACGACGCTCTTCCGATCTNNNNNNNNCAATTACCG | 415 | CAATTACCGT | 607 |
| | TNNNNNN | | | |
| randomN_plate1_32 | /5Phos/ACGACGCTCTTCCGATCTNNNNNNNNGGCCGTAGT | 416 | GGCCGTAGTC | 608 |
| | CNNNNNN | | | |
| randomN_plate1_33 | /5Phos/ACGACGCTCTTCCGATCTNNNNNNNNCGATTACGG | 417 | CGATTACGGC | 609 |
| | CNNNNNN | | | |
| randomN_plate1_34 | /5Phos/ACGACGCTCTTCCGATCTNNNNNNNNTAATGAACG | 418 | TAATGAACGA | 610 |
| | ANNNNNN | | | |
| randomN_plate1_35 | /5Phos/ACGACGCTCTTCCGATCTNNNNNNNNCCGTTCCTT | 419 | CCGTTCCTTA | 611 |
| | ANNNNNN | | | |
| randomN_plate1_36 | /5Phos/ACGACGCTCTTCCGATCTNNNNNNNNGGTACCATA | 420 | GGTACCATAT | 612 |
| | TNNNNNN | | | |
| randomN_plate1_37 | /5Phos/ACGACGCTCTTCCGATCTNNNNNNNNCCGATTCGC | 421 | CCGATTCGCA | 613 |
| | ANNNNNN | | | |
| randomN_plate1_38 | /5Phos/ACGACGCTCTTCCGATCTNNNNNNNNATGGCTCTG | 422 | ATGGCTCTGC | 614 |
| | CNNNNNN | | | |
| randomN_plate1_39 | /5Phos/ACGACGCTCTTCCGATCTNNNNNNNNGTATAATAC | 423 | GTATAATACG | 615 |
| | GNNNNNN | | | |
| randomN_plate1_40 | /5Phos/ACGACGCTCTTCCGATCTNNNNNNNNATCAGCAAG | 424 | ATCAGCAAGT | 616 |
| | TNNNNNN | | | |
| randomN_plate1_41 | /5Phos/ACGACGCTCTTCCGATCTNNNNNNNNGGCGAACTC | 425 | GGCGAACTCG | 617 |
| | GNNNNNN | | | |
| randomN_plate1_42 | /5Phos/ACGACGCTCTTCCGATCTNNNNNNNNTTAATTGAA | 426 | TTAATTGAAT | 618 |
| | TNNNNNN | | | |
| randomN_plate1_43 | /5Phos/ACGACGCTCTTCCGATCTNNNNNNNNTTAGGACCG | 427 | TTAGGACCGG | 619 |
| | GNNNNNN | | | |
| randomN_plate1_44 | /5Phos/ACGACGCTCTTCCGATCTNNNNNNNNAAGTAAGA | 428 | AAGTAAGAGC | 620 |
| | GCNNNNNN | | | |
| randomN_plate1_45 | /5Phos/ACGACGCTCTTCCGATCTNNNNNNNNCCTTGGTCC | 429 | CCTTGGTCCA | 621 |
| | ANNNNNN | | | |
| randomN_plate1_46 | /5Phos/ACGACGCTCTTCCGATCTNNNNNNNNCATCAGAAT | 430 | CATCAGAATG | 622 |
| | GNNNNNN | | | |
| randomN_plate1_47 | /5Phos/ACGACGCTCTTCCGATCTNNNNNNNNTTATAGCAG | 431 | TTATAGCAGA | 623 |
| | ANNNNNN | | | |
| randomN_plate1_48 | /5Phos/ACGACGCTCTTCCGATCTNNNNNNNNTTACTTGGA | 432 | TTACTTGGAA | 624 |
| | ANNNNNN | | | |
| randomN_plate1_49 | /5Phos/ACGACGCTCTTCCGATCTNNNNNNNNGCTCAGCCG | 433 | GCTCAGCCGG | 625 |
| | GNNNNNN | | | |
| randomN_plate1_50 | /5Phos/ACGACGCTCTTCCGATCTNNNNNNNNACGTCCGCA | 434 | ACGTCCGCAG | 626 |
| | GNNNNNN | | | |
| randomN_plate1_51 | /5Phos/ACGACGCTCTTCCGATCTNNNNNNNNTTGACTGAC | 435 | TTGACTGACG | 627 |
| | GNNNNNN | | | |
| randomN_plate1_52 | /5Phos/ACGACGCTCTTCCGATCTNNNNNNNNTTGCGAGGC | 436 | TTGCGAGGCA | 628 |
| | ANNNNNN | | | |
| randomN_plate1_53 | /5Phos/ACGACGCTCTTCCGATCTNNNNNNNNTTCCAACCG | 437 | TTCCAACCGC | 629 |
| | CNNNNNN | | | |
| randomN_plate1_54 | /5Phos/ACGACGCTCTTCCGATCTNNNNNNNNTAACCTTCG | 438 | TAACCTTCGG | 630 |
| | GNNNNNN | | | |
| randomN_plate1_55 | /5Phos/ACGACGCTCTTCCGATCTNNNNNNNNTCAAGCCGA | 439 | TCAAGCCGAT | 631 |
| | TNNNNNN | | | |
| randomN_plate1_56 | /5Phos/ACGACGCTCTTCCGATCTNNNNNNNNCTTGCAACC | 440 | CTTGCAACCT | 632 |
| | TNNNNNN | | | |
| randomN_plate1_57 | /5Phos/ACGACGCTCTTCCGATCTNNNNNNNNCCATCGCGA | 441 | CCATCGCGAA | 633 |
| | ANNNNNN | | | |
| randomN_plate1_58 | /5Phos/ACGACGCTCTTCCGATCTNNNNNNNNTAGACTTCT | 442 | TAGACTTCTT | 634 |
| | TNNNNNN | | | |
| randomN_plate1_59 | /5Phos/ACGACGCTCTTCCGATCTNNNNNNNNGTCCTTAAG | 443 | GTCCTTAAGA | 635 |
| | ANNNNNN | | | |
| randomN_plate1_60 | /5Phos/ACGACGCTCTTCCGATCTNNNNNNNNAGTAACGGT | 444 | AGTAACGGTC | 636 |
| | CNNNNNN | | | |
| randomN_plate1_61 | /5Phos/ACGACGCTCTTCCGATCTNNNNNNNNGTTCGTCAG | 445 | GTTCGTCAGA | 637 |
| | ANNNNNN | | | |
| randomN_plate1_62 | /5Phos/ACGACGCTCTTCCGATCTNNNNNNNNCGCCTAATG | 446 | CGCCTAATGC | 638 |
| | CNNNNNN | | | |
| randomN_plate1_63 | /5Phos/ACGACGCTCTTCCGATCTNNNNNNNNACCGGAATT | 447 | ACCGGAATTA | 639 |
| | ANNNNNN | | | |
| randomN_plate1_64 | /5Phos/ACGACGCTCTTCCGATCTNNNNNNNNTAGGCCATA | 448 | TAGGCCATAG | 640 |
| | GNNNNNN | | | |
| randomN_plate1_65 | /5Phos/ACGACGCTCTTCCGATCTNNNNNNNNTAACTCTTA | 449 | TAACTCTTAG | 641 |
| | GNNNNNN | | | |
| randomN_plate1_66 | /5Phos/ACGACGCTCTTCCGATCTNNNNNNNNTATGAGTTA | 450 | TATGAGTTAA | 642 |
| | ANNNNNN | | | |
| randomN_plate1_67 | /5Phos/ACGACGCTCTTCCGATCTNNNNNNNNTATCATGAT | 451 | TATCATGATC | 643 |
| | CNNNNNN | | | |
| randomN_plate1_68 | /5Phos/ACGACGCTCTTCCGATCTNNNNNNNNGAGCATATG | 452 | GAGCATATGG | 644 |
| | GNNNNNN | | | |
| randomN_plate1_69 | /5Phos/ACGACGCTCTTCCGATCTNNNNNNNNTAACGATCC | 453 | TAACGATCCA | 645 |
| | ANNNNNN | | | |
| randomN_plate1_70 | /5Phos/ACGACGCTCTTCCGATCTNNNNNNNNCGGCGTAAC | 454 | CGGCGTAACT | 646 |
| | TNNNNNN | | | |
| randomN_plate1_71 | /5Phos/ACGACGCTCTTCCGATCTNNNNNNNNCGTCGCAGC | 455 | CGTCGCAGCC | 647 |
| | CNNNNNN | | | |
| randomN_plate1_72 | /5Phos/ACGACGCTCTTCCGATCTNNNNNNNNGTAGCTCCA | 456 | GTAGCTCCAT | 648 |
| | TNNNNNN | | | |
| randomN_plate1_73 | /5Phos/ACGACGCTCTTCCGATCTNNNNNNNNTTGCCTTGG | 457 | TTGCCTTGGC | 649 |
| | CNNNNNN | | | |
| randomN_plate1_74 | /5Phos/ACGACGCTCTTCCGATCTNNNNNNNNTGCTAATTC | 458 | TGCTAATTCT | 650 |
| | TNNNNNN | | | |
| randomN_plate1_75 | /5Phos/ACGACGCTCTTCCGATCTNNNNNNNNGTCCTACTT | 459 | GTCCTACTTG | 651 |
| | GNNNNNN | | | |
| randomN_plate1_76 | /5Phos/ACGACGCTCTTCCGATCTNNNNNNNNGGTAGGTTA | 460 | GGTAGGTTAG | 652 |
| | GNNNNNN | | | |
| randomN_plate1_77 | /5Phos/ACGACGCTCTTCCGATCTNNNNNNNNGAGCATCAT | 461 | GAGCATCATT | 653 |
| | TNNNNNN | | | |
| randomN_plate1_78 | /5Phos/ACGACGCTCTTCCGATCTNNNNNNNNCCGCTCCGG | 462 | CCGCTCCGGC | 654 |
| | CNNNNNN | | | |
| randomN_plate1_79 | /5Phos/ACGACGCTCTTCCGATCTNNNNNNNNTTCTTCCGG | 463 | TTCTTCCGGT | 655 |
| | TNNNNNN | | | |
| randomN_plate1_80 | /5Phos/ACGACGCTCTTCCGATCTNNNNNNNNAGGAGAGA | 464 | AGGAGAGAAC | 656 |
| | ACNNNNNN | | | |
| randomN_plate1_81 | /5Phos/ACGACGCTCTTCCGATCTNNNNNNNNTAACTCAAT | 465 | TAACTCAATT | 657 |
| | TNNNNNN | | | |
| randomN_plate1_82 | /5Phos/ACGACGCTCTTCCGATCTNNNNNNNNACTATAGGT | 466 | ACTATAGGTT | 658 |
| | TNNNNNN | | | |
| randomN_plate1_83 | /5Phos/ACGACGCTCTTCCGATCTNNNNNNNNCAAGATGCC | 467 | CAAGATGCCG | 659 |
| | GNNNNNN | | | |
| randomN_plate1_84 | /5Phos/ACGACGCTCTTCCGATCTNNNNNNNNAACGTCTAG | 468 | AACGTCTAGT | 660 |
| | TNNNNNN | | | |
| randomN_plate1_85 | /5Phos/ACGACGCTCTTCCGATCTNNNNNNNNAGGTATACT | 469 | AGGTATACTC | 661 |
| | CNNNNNN | | | |
| randomN_plate1_86 | /5Phos/ACGACGCTCTTCCGATCTNNNNNNNNTTCATAGGA | 470 | TTCATAGGAC | 662 |
| | CNNNNNN | | | |
| randomN_plate1_87 | /5Phos/ACGACGCTCTTCCGATCTNNNNNNNNGGAGGCCTC | 471 | GGAGGCCTCC | 663 |
| | CNNNNNN | | | |
| randomN_plate1_88 | /5Phos/ACGACGCTCTTCCGATCTNNNNNNNNTTCAATATA | 472 | TTCAATATAA | 664 |
| | ANNNNNN | | | |
| randomN_plate1_89 | /5Phos/ACGACGCTCTTCCGATCTNNNNNNNNACGTCATAT | 473 | ACGTCATATA | 665 |
| | ANNNNNN | | | |
| randomN_plate1_90 | /5Phos/ACGACGCTCTTCCGATCTNNNNNNNNTTGACCAGG | 474 | TTGACCAGGA | 666 |
| | ANNNNNN | | | |
| randomN_plate1_91 | /5Phos/ACGACGCTCTTCCGATCTNNNNNNNNCGGTTGCGC | 475 | CGGTTGCGCG | 667 |
| | GNNNNNN | | | |
| randomN_plate1_92 | /5Phos/ACGACGCTCTTCCGATCTNNNNNNNNCAAGGAGG | 476 | CAAGGAGGTC | 668 |
| | TCNNNNNN | | | |
| randomN_plate1_93 | /5Phos/ACGACGCTCTTCCGATCTNNNNNNNNTTACGATGA | 477 | TTACGATGAA | 669 |
| | ANNNNNN | | | |
| randomN_plate1_94 | /5Phos/ACGACGCTCTTCCGATCTNNNNNNNNTTGCTGGCA | 478 | TTGCTGGCAT | 670 |
| | TNNNNNN | | | |
| randomN_plate1_95 | /5Phos/ACGACGCTCTTCCGATCTNNNNNNNNGAGGCATCA | 479 | GAGGCATCAA | 671 |
| | ANNNNNN | | | |
| randomN_plate1_96 | /5Phos/ACGACGCTCTTCCGATCTNNNNNNNNATTCGACCA | 480 | ATTCGACCAA | 672 |
| | ANNNNNN | | | |
| randomN_plate2_01 | /5Phos/ACGACGCTCTTCCGATCTNNNNNNNNGCCGTATGC | 481 | GCCGTATGCT | 673 |
| | TNNNNNN | | | |
| randomN_plate2_02 | /5Phos/ACGACGCTCTTCCGATCTNNNNNNNNCTGAACTGG | 482 | CTGAACTGGT | 674 |
| | TNNNNNN | | | |
| randomN_plate2_03 | /5Phos/ACGACGCTCTTCCGATCTNNNNNNNNCATAACCAG | 483 | CATAACCAGC | 675 |
| | CNNNNNN | | | |
| randomN_plate2_04 | /5Phos/ACGACGCTCTTCCGATCTNNNNNNNNAAGTTGCCA | 484 | AAGTTGCCAT | 676 |
| | TNNNNNN | | | |
| randomN_plate2_05 | /5Phos/ACGACGCTCTTCCGATCTNNNNNNNNAGGCCGCTC | 485 | AGGCCGCTCG | 677 |
| | GNNNNNN | | | |
| randomN_plate2_06 | /5Phos/ACGACGCTCTTCCGATCTNNNNNNNNAGGTAATAG | 486 | AGGTAATAGG | 678 |
| | GNNNNNN | | | |
| randomN_plate2_07 | /5Phos/ACGACGCTCTTCCGATCTNNNNNNNNGTACTAGTA | 487 | GTACTAGTAA | 679 |
| | ANNNNNN | | | |
| randomN_plate2_08 | /5Phos/ACGACGCTCTTCCGATCTNNNNNNNNGCGCGGTA | 488 | GCGCGGTAGT | 680 |
| | GTNNNNNN | | | |
| randomN_plate2_09 | /5Phos/ACGACGCTCTTCCGATCTNNNNNNNNCTGGATTAG | 489 | CTGGATTAGT | 681 |
| | TNNNNNN | | | |
| randomN_plate2_10 | /5Phos/ACGACGCTCTTCCGATCTNNNNNNNNTTGGATCCT | 490 | TTGGATCCTT | 682 |
| | TNNNNNN | | | |
| randomN_plate2_11 | /5Phos/ACGACGCTCTTCCGATCTNNNNNNNNTTGGAATCT | 491 | TTGGAATCTC | 683 |
| | CNNNNNN | | | |
| randomN_plate2_12 | /5Phos/ACGACGCTCTTCCGATCTNNNNNNNNACCTGGACG | 492 | ACCTGGACGC | 684 |
| | CNNNNNN | | | |
| randomN_plate2_13 | /5Phos/ACGACGCTCTTCCGATCTNNNNNNNNCCTGACGTT | 493 | CCTGACGTTC | 685 |
| | CNNNNNN | | | |
| randomN_plate2_14 | /5Phos/ACGACGCTCTTCCGATCTNNNNNNNNGCGTTCAGC | 494 | GCGTTCAGCT | 686 |
| | TNNNNNN | | | |
| randomN_plate2_15 | /5Phos/ACGACGCTCTTCCGATCTNNNNNNNNTTAGCAATA | 495 | TTAGCAATAA | 687 |
| | ANNNNNN | | | |
| randomN_plate2_16 | /5Phos/ACGACGCTCTTCCGATCTNNNNNNNNTTGATGCTA | 496 | TTGATGCTAT | 688 |
| | TNNNNNN | | | |
| randomN_plate2_17 | /5Phos/ACGACGCTCTTCCGATCTNNNNNNNNCTCTGCGGC | 497 | CTCTGCGGCA | 689 |
| | ANNNNNN | | | |
| randomN_plate2_18 | /5Phos/ACGACGCTCTTCCGATCTNNNNNNNNAATAATACC | 498 | AATAATACCA | 690 |
| | ANNNNNN | | | |
| randomN_plate2_19 | /5Phos/ACGACGCTCTTCCGATCTNNNNNNNNACGCCGTTC | 499 | ACGCCGTTCA | 691 |
| | ANNNNNN | | | |
| randomN_plate2_20 | /5Phos/ACGACGCTCTTCCGATCTNNNNNNNNTTCGCTTAC | 500 | TTCGCTTACG | 692 |
| | GNNNNNN | | | |
| randomN_plate2_21 | /5Phos/ACGACGCTCTTCCGATCTNNNNNNNNTACGGCTAC | 501 | TACGGCTACG | 693 |
| | GNNNNNN | | | |
| randomN_plate2_22 | /5Phos/ACGACGCTCTTCCGATCTNNNNNNNNTTCTTATCG | 502 | TTCTTATCGA | 694 |
| | ANNNNNN | | | |
| randomN_plate2_23 | /5Phos/ACGACGCTCTTCCGATCTNNNNNNNNTTCCATGGC | 503 | TTCCATGGCA | 695 |
| | ANNNNNN | | | |
| randomN_plate2_24 | /5Phos/ACGACGCTCTTCCGATCTNNNNNNNNAAGTAGTCA | 504 | AAGTAGTCAG | 696 |
| | GNNNNNN | | | |
| randomN_plate2_25 | /5Phos/ACGACGCTCTTCCGATCTNNNNNNNNTCAGCTCTA | 505 | TCAGCTCTAA | 697 |
| | ANNNNNN | | | |
| randomN_plate2_26 | /5Phos/ACGACGCTCTTCCGATCTNNNNNNNNCGAATAGAT | 506 | CGAATAGATG | 698 |
| | GNNNNNN | | | |
| randomN_plate2_27 | /5Phos/ACGACGCTCTTCCGATCTNNNNNNNNCGGAGATCC | 507 | CGGAGATCCG | 699 |
| | GNNNNNN | | | |
| randomN_plate2_28 | /5Phos/ACGACGCTCTTCCGATCTNNNNNNNNACCGCAGAA | 508 | ACCGCAGAAT | 700 |
| | TNNNNNN | | | |
| randomN_plate2_29 | /5Phos/ACGACGCTCTTCCGATCTNNNNNNNNTCTCCTATA | 509 | TCTCCTATAA | 701 |
| | ANNNNNN | | | |
| randomN_plate2_30 | /5Phos/ACGACGCTCTTCCGATCTNNNNNNNNCAACCTATA | 510 | CAACCTATAT | 702 |
| | TNNNNNN | | | |
| randomN_plate2_31 | /5Phos/ACGACGCTCTTCCGATCTNNNNNNNNAGTCGAGA | 511 | AGTCGAGAAG | 703 |
| | AGNNNNNN | | | |
| randomN_plate2_32 | /5Phos/ACGACGCTCTTCCGATCTNNNNNNNNAAGACGGC | 512 | AAGACGGCCA | 704 |
| | CANNNNNN | | | |
| randomN_plate2_33 | /5Phos/ACGACGCTCTTCCGATCTNNNNNNNNGCCAACGCC | 513 | GCCAACGCCA | 705 |
| | ANNNNNN | | | |
| randomN_plate2_34 | /5Phos/ACGACGCTCTTCCGATCTNNNNNNNNTCTACCATT | 514 | TCTACCATTA | 706 |
| | ANNNNNN | | | |
| randomN_plate2_35 | /5Phos/ACGACGCTCTTCCGATCTNNNNNNNNCTTGCGGTC | 515 | CTTGCGGTCT | 707 |
| | TNNNNNN | | | |
| randomN_plate2_36 | /5Phos/ACGACGCTCTTCCGATCTNNNNNNNNTTACGTATA | 516 | TTACGTATAC | 708 |
| | CNNNNNN | | | |
| randomN_plate2_37 | /5Phos/ACGACGCTCTTCCGATCTNNNNNNNNCGATTGGTT | 517 | CGATTGGTTA | 709 |
| | ANNNNNN | | | |
| randomN_plate2_38 | /5Phos/ACGACGCTCTTCCGATCTNNNNNNNNACTTAACTA | 518 | ACTTAACTAG | 710 |
| | GNNNNNN | | | |
| randomN_plate2_39 | /5Phos/ACGACGCTCTTCCGATCTNNNNNNNNGCAGACCG | 519 | GCAGACCGGT | 711 |
| | GTNNNNNN | | | |
| randomN_plate2_40 | /5Phos/ACGACGCTCTTCCGATCTNNNNNNNNTGAGTCCAG | 520 | TGAGTCCAGA | 712 |
| | ANNNNNN | | | |
| randomN_plate2_41 | /5Phos/ACGACGCTCTTCCGATCTNNNNNNNNTGGAGAATT | 521 | TGGAGAATTC | 713 |
| | CNNNNNN | | | |
| randomN_plate2_42 | /5Phos/ACGACGCTCTTCCGATCTNNNNNNNNACCAGCCTT | 522 | ACCAGCCTTA | 714 |
| | ANNNNNN | | | |
| randomN_plate2_43 | /5Phos/ACGACGCTCTTCCGATCTNNNNNNNNGGCGAGCTT | 523 | GGCGAGCTTA | 715 |
| | ANNNNNN | | | |
| randomN_plate2_44 | /5Phos/ACGACGCTCTTCCGATCTNNNNNNNNTCGAGGAGT | 524 | TCGAGGAGTA | 716 |
| | ANNNNNN | | | |
| randomN_plate2_45 | /5Phos/ACGACGCTCTTCCGATCTNNNNNNNNCCTTACTCCT | 525 | CCTTACTCCT | 717 |
| | NNNNNN | | | |
| randomN_plate2_46 | /5Phos/ACGACGCTCTTCCGATCTNNNNNNNNTCAGACGAA | 526 | TCAGACGAAC | 718 |
| | CNNNNNN | | | |
| randomN_plate2_47 | /5Phos/ACGACGCTCTTCCGATCTNNNNNNNNCCGTCCAGT | 527 | CCGTCCAGTA | 719 |
| | ANNNNNN | | | |
| randomN_plate2_48 | /5Phos/ACGACGCTCTTCCGATCTNNNNNNNNGTTCCGCTA | 528 | GTTCCGCTAA | 720 |
| | ANNNNNN | | | |
| randomN_plate2_49 | /5Phos/ACGACGCTCTTCCGATCTNNNNNNNNCAGATTCGA | 529 | CAGATTCGAT | 721 |
| | TNNNNNN | | | |
| randomN_plate2_50 | /5Phos/ACGACGCTCTTCCGATCTNNNNNNNNTGCATATAA | 530 | TGCATATAAC | 722 |
| | CNNNNNN | | | |
| randomN_plate2_51 | /5Phos/ACGACGCTCTTCCGATCTNNNNNNNNTAGGCAGAT | 531 | TAGGCAGATA | 723 |
| | ANNNNNN | | | |
| randomN_plate2_52 | /5Phos/ACGACGCTCTTCCGATCTNNNNNNNNTATGCCGAG | 532 | TATGCCGAGT | 724 |
| | TNNNNNN | | | |
| randomN_plate2_53 | /5Phos/ACGACGCTCTTCCGATCTNNNNNNNNATAGTCGTA | 533 | ATAGTCGTAG | 725 |
| | GNNNNNN | | | |
| randomN_plate2_54 | /5Phos/ACGACGCTCTTCCGATCTNNNNNNNNGGATGCAG | 534 | GGATGCAGCA | 726 |
| | CANNNNNN | | | |
| randomN_plate2_55 | /5Phos/ACGACGCTCTTCCGATCTNNNNNNNNCCGCTATAT | 535 | CCGCTATATT | 727 |
| | TNNNNNN | | | |
| randomN_plate2_56 | /5Phos/ACGACGCTCTTCCGATCTNNNNNNNNATCGAGTCG | 536 | ATCGAGTCGC | 728 |
| | CNNNNNN | | | |
| randomN_plate2_57 | /5Phos/ACGACGCTCTTCCGATCTNNNNNNNNGCGACGCA | 537 | GCGACGCAGA | 729 |
| | GANNNNNN | | | |
| randomN_plate2_58 | /5Phos/ACGACGCTCTTCCGATCTNNNNNNNNAATGGTCGA | 538 | AATGGTCGAC | 730 |
| | CNNNNNN | | | |
| randomN_plate2_59 | /5Phos/ACGACGCTCTTCCGATCTNNNNNNNNTGGAACTAG | 539 | TGGAACTAGA | 731 |
| | ANNNNNN | | | |
| randomN_plate2_60 | /5Phos/ACGACGCTCTTCCGATCTNNNNNNNNGTCCAACTC | 540 | GTCCAACTCA | 732 |
| | ANNNNNN | | | |
| randomN_plate2_61 | /5Phos/ACGACGCTCTTCCGATCTNNNNNNNNGTTATGGAT | 541 | GTTATGGATC | 733 |
| | CNNNNNN | | | |
| randomN_plate2_62 | /5Phos/ACGACGCTCTTCCGATCTNNNNNNNNTTATAAGAA | 542 | TTATAAGAAC | 734 |
| | CNNNNNN | | | |
| randomN_plate2_63 | /5Phos/ACGACGCTCTTCCGATCTNNNNNNNNCAAGCTTCA | 543 | CAAGCTTCAT | 735 |
| | TNNNNNN | | | |
| randomN_plate2_64 | /5Phos/ACGACGCTCTTCCGATCTNNNNNNNNCTGATTAAG | 544 | CTGATTAAGA | 736 |
| | ANNNNNN | | | |
| randomN_plate2_65 | /5Phos/ACGACGCTCTTCCGATCTNNNNNNNNTACTTACTT | 545 | TACTTACTTA | 737 |
| | ANNNNNN | | | |
| randomN_plate2_66 | /5Phos/ACGACGCTCTTCCGATCTNNNNNNNNGGATCTGCA | 546 | GGATCTGCAG | 738 |
| | GNNNNNN | | | |
| randomN_plate2_67 | /5Phos/ACGACGCTCTTCCGATCTNNNNNNNNATGCAATAT | 547 | ATGCAATATG | 739 |
| | GNNNNNN | | | |
| randomN_plate2_68 | /5Phos/ACGACGCTCTTCCGATCTNNNNNNNNTTCCTAGAC | 548 | TTCCTAGACC | 740 |
| | CNNNNNN | | | |
| randomN_plate2_69 | /5Phos/ACGACGCTCTTCCGATCTNNNNNNNNACTGCCGAT | 549 | ACTGCCGATA | 741 |
| | ANNNNNN | | | |
| randomN_plate2_70 | /5Phos/ACGACGCTCTTCCGATCTNNNNNNNNTCCAGAAGG | 550 | TCCAGAAGGT | 742 |
| | TNNNNNN | | | |
| randomN_plate2_71 | /5Phos/ACGACGCTCTTCCGATCTNNNNNNNNTTCAAGACC | 551 | TTCAAGACCA | 743 |
| | ANNNNNN | | | |
| randomN_plate2_72 | /5Phos/ACGACGCTCTTCCGATCTNNNNNNNNTATTACTCA | 552 | TATTACTCAT | 744 |
| | TNNNNNN | | | |
| randomN_plate2_73 | /5Phos/ACGACGCTCTTCCGATCTNNNNNNNNAACTGATCT | 553 | AACTGATCTT | 745 |
| | TNNNNNN | | | |
| randomN_plate2_74 | /5Phos/ACGACGCTCTTCCGATCTNNNNNNNNCCGCGGACC | 554 | CCGCGGACCG | 746 |
| | GNNNNNN | | | |
| randomN_plate2_75 | /5Phos/ACGACGCTCTTCCGATCTNNNNNNNNAATACGCAG | 555 | AATACGCAGG | 747 |
| | GNNNNNN | | | |
| randomN_plate2_76 | /5Phos/ACGACGCTCTTCCGATCTNNNNNNNNGGTCGCGTC | 556 | GGTCGCGTCA | 748 |
| | ANNNNNN | | | |
| randomN_plate2_77 | /5Phos/ACGACGCTCTTCCGATCTNNNNNNNNAATTATCAG | 557 | AATTATCAGC | 749 |
| | CNNNNNN | | | |
| randomN_plate2_78 | /5Phos/ACGACGCTCTTCCGATCTNNNNNNNNCAGCTATCG | 558 | CAGCTATCGT | 750 |
| | TNNNNNN | | | |
| randomN_plate2_79 | /5Phos/ACGACGCTCTTCCGATCTNNNNNNNNATTGCGCTG | 559 | ATTGCGCTGA | 751 |
| | ANNNNNN | | | |
| randomN_plate2_80 | /5Phos/ACGACGCTCTTCCGATCTNNNNNNNNTTGGTAGGC | 560 | TTGGTAGGCG | 752 |
| | GNNNNNN | | | |
| randomN_plate2_81 | /5Phos/ACGACGCTCTTCCGATCTNNNNNNNNAGCTAAGGT | 561 | AGCTAAGGTA | 753 |
| | ANNNNNN | | | |
| randomN_plate2_82 | /5Phos/ACGACGCTCTTCCGATCTNNNNNNNNTCGTAGAGA | 562 | TCGTAGAGAA | 754 |
| | ANNNNNN | | | |
| randomN_plate2_83 | /5Phos/ACGACGCTCTTCCGATCTNNNNNNNNTGATGGCCT | 563 | TGATGGCCTT | 755 |
| | TNNNNNN | | | |
| randomN_plate2_84 | /5Phos/ACGACGCTCTTCCGATCTNNNNNNNNTGGAAGTAC | 564 | TGGAAGTACC | 756 |
| | CNNNNNN | | | |
| randomN_plate2_85 | /5Phos/ACGACGCTCTTCCGATCTNNNNNNNNCTCCAAGGA | 565 | CTCCAAGGAT | 757 |
| | TNNNNNN | | | |
| randomN_plate2_86 | /5Phos/ACGACGCTCTTCCGATCTNNNNNNNNAGATATATC | 566 | AGATATATCG | 758 |
| | GNNNNNN | | | |
| randomN_plate2_87 | /5Phos/ACGACGCTCTTCCGATCTNNNNNNNNCATGCTGGT | 567 | CATGCTGGTT | 759 |
| | TNNNNNN | | | |
| randomN_plate2_88 | /5Phos/ACGACGCTCTTCCGATCTNNNNNNNNTCCTCGAGT | 568 | TCCTCGAGTC | 760 |
| | CNNNNNN | | | |
| randomN_plate2_89 | /5Phos/ACGACGCTCTTCCGATCTNNNNNNNNGCAAGGAA | 569 | GCAAGGAATA | 761 |
| | TANNNNNN | | | |
| randomN_plate2_90 | /5Phos/ACGACGCTCTTCCGATCTNNNNNNNNGGCATAGCT | 570 | GGCATAGCTT | 762 |
| | TNNNNNN | | | |
| randomN_plate2_91 | /5Phos/ACGACGCTCTTCCGATCTNNNNNNNNCTACGGTAG | 571 | CTACGGTAGC | 763 |
| | CNNNNNN | | | |
| randomN_plate2_92 | /5Phos/ACGACGCTCTTCCGATCTNNNNNNNNAGTAAGCAT | 572 | AGTAAGCATA | 764 |
| | ANNNNNN | | | |
| randomN_plate2_93 | /5Phos/ACGACGCTCTTCCGATCTNNNNNNNNCGCCTCGAA | 573 | CGCCTCGAAC | 765 |
| | CNNNNNN | | | |
| randomN_plate2_94 | /5Phos/ACGACGCTCTTCCGATCTNNNNNNNNTTAGGATCT | 574 | TTAGGATCTA | 766 |
| | ANNNNNN | | | |
| randomN_plate2_95 | /5Phos/ACGACGCTCTTCCGATCTNNNNNNNNACTACTGAA | 575 | ACTACTGAAG | 767 |
| | GNNNNNN | | | |
| randomN_plate2_96 | /5Phos/ACGACGCTCTTCCGATCTNNNNNNNNAATCTGGAG | 576 | AATCTGGAGT | 768 |
| | TNNNNNN | | | |

### Pool/Centrifuge/Resuspend/Redistribute (15 m)

Add 10 μL NBB into each well, pool solution, and move solution into a 15 mL tube. Centrifuge the tube for 3 minutes, 1000 g at 4 C.
Use a pipet to aspirate supernatant. Resuspend nuclei in 1 mL NBB and then move into a 1.5 mL microcentrifuge tube. Centrifuge the tube for 3 minutes, 1000 g at 4 C to pellet the nuclei.

### Ligation (1 h)

Dump the supernatant. Resuspend the cells in 950 μL NBB. Distribute the nuclei into four PCR plates, with 2.5 μL of the solution going into each well.
To each well, add 1 μL of the appropriate DNA ligation primer (Table 5)/adaptor complex (3.125 μM).
Create a mixture of:
210 μL 10× T4 Ligation Buffer
21 μL SUPERase In RNase Inhibitor
210 μL T4 DNA Ligase
189 μL Nuclease Free Water
   Add 1.5 μL of the mixture to each of the PCR plate wells.
Incubate plates for 30 minutes at room temperature with gentle shaking (300 rpm with Thermomixer, 50 rpm on Fisherbrand Nutating Mixer).
From an aliquot of 0.5M EDTA, dilute to 18 mM EDTA. Add 1 μL EDTA (18 mM) into each well and pool all solution into a 15 mL tube.

TABLE 5
Ligation primer sequences (plate 1)

| | | SEQ ID | | SEQ ID |
|---|---|---|---|---|
| Name | Sequence | NO: | Barcode | NO: |
| EasySci- | AATGATACGGCGACCACCGAGATCTACACCCG | 769 | CCGCGGCTCA | 1153 |
| RNA_ligation1_01 | CGGCTCAACACTCTTTCCCTAC | | | |
| EasySci- | AATGATACGGCGACCACCGAGATCTACACGGC | 770 | GGCTCCTCGT | 1154 |
| RNA_ligation1_02 | TCCTCGTACACTCTTTCCCTAC | | | |
| EasySci- | AATGATACGGCGACCACCGAGATCTACACGTT | 771 | GTTACGCAAG | 1155 |
| RNA_ligation1_03 | ACGCAAGACACTCTTTCCCTAC | | | |
| EasySci- | AATGATACGGCGACCACCGAGATCTACACAGC | 772 | AGCCGGTACC | 1156 |
| RNA_ligation1_04 | CGGTACCACACTCTTTCCCTAC | | | |
| EasySci- | AATGATACGGCGACCACCGAGATCTACACACC | 773 | ACCTCTATCT | 1157 |
| RNA_ligation1_05 | TCTATCTACACTCTTTCCCTAC | | | |
| EasySci- | AATGATACGGCGACCACCGAGATCTACACGGA | 774 | GGACTACTAC | 1158 |
| RNA_ligation1_06 | CTACTACACACTCTTTCCCTAC | | | |
| EasySci- | AATGATACGGCGACCACCGAGATCTACACGTA | 775 | GTATCATCGA | 1159 |
| RNA_ligation1_07 | TCATCGAACACTCTTTCCCTAC | | | |
| EasySci- | AATGATACGGCGACCACCGAGATCTACACCCG | 776 | CCGCGATTAT | 1160 |
| RNA_ligation1_08 | CGATTATACACTCTTTCCCTAC | | | |
| EasySci- | AATGATACGGCGACCACCGAGATCTACACATT | 777 | ATTCAGGTAC | 1161 |
| RNA_ligation1_09 | CAGGTACACACTCTTTCCCTAC | | | |
| EasySci- | AATGATACGGCGACCACCGAGATCTACACATG | 778 | ATGGAATTGG | 1162 |
| RNA_ligation1_10 | GAATTGGACACTCTTTCCCTAC | | | |
| EasySci- | AATGATACGGCGACCACCGAGATCTACACGAC | 779 | GACGAAGCGT | 1163 |
| RNA_ligation1_11 | GAAGCGTACACTCTTTCCCTAC | | | |
| EasySci- | AATGATACGGCGACCACCGAGATCTACACCTT | 780 | CTTGCAGTAG | 1164 |
| RNA_ligation1_12 | GCAGTAGACACTCTTTCCCTAC | | | |
| EasySci- | AATGATACGGCGACCACCGAGATCTACACCTT | 781 | CTTGGTAATG | 1165 |
| RNA_ligation1_13 | GGTAATGACACTCTTTCCCTAC | | | |
| EasySci- | AATGATACGGCGACCACCGAGATCTACACCAA | 782 | CAAGTCGACC | 1166 |
| RNA_ligation1_14 | GTCGACCACACTCTTTCCCTAC | | | |
| EasySci- | AATGATACGGCGACCACCGAGATCTACACTAA | 783 | TAACGAATTG | 1167 |
| RNA_ligation1_15 | CGAATTGACACTCTTTCCCTAC | | | |
| EasySci- | AATGATACGGCGACCACCGAGATCTACACTGA | 784 | TGAGAACCAA | 1168 |
| RNA_ligation1_16 | GAACCAAACACTCTTTCCCTAC | | | |
| EasySci- | AATGATACGGCGACCACCGAGATCTACACTTA | 785 | TTATTCTGAG | 1169 |
| RNA_ligation1_17 | TTCTGAGACACTCTTTCCCTAC | | | |
| EasySci- | AATGATACGGCGACCACCGAGATCTACACTTA | 786 | TTATTATGGT | 1170 |
| RNA_ligation1_18 | TTATGGTACACTCTTTCCCTAC | | | |
| EasySci- | AATGATACGGCGACCACCGAGATCTACACATA | 787 | ATATGAGCCA | 1171 |
| RNA_ligation1_19 | TGAGCCAACACTCTTTCCCTAC | | | |
| EasySci- | AATGATACGGCGACCACCGAGATCTACACCAA | 788 | CAACCAGTAC | 1172 |
| RNA_ligation1_20 | CCAGTACACACTCTTTCCCTAC | | | |
| EasySci- | AATGATACGGCGACCACCGAGATCTACACCAT | 789 | CATCCGACTA | 1173 |
| RNA_ligation1_21 | CCGACTAACACTCTTTCCCTAC | | | |
| EasySci- | AATGATACGGCGACCACCGAGATCTACACATC | 790 | ATCATGGCTG | 1174 |
| RNA_ligation1_22 | ATGGCTGACACTCTTTCCCTAC | | | |
| EasySci- | AATGATACGGCGACCACCGAGATCTACACCCG | 791 | CCGCAAGTTC | 1175 |
| RNA_ligation1_23 | CAAGTTCACACTCTTTCCCTAC | | | |
| EasySci- | AATGATACGGCGACCACCGAGATCTACACCTT | 792 | CTTCTCATTG | 1176 |
| RNA_ligation1_24 | CTCATTGACACTCTTTCCCTAC | | | |
| EasySci- | AATGATACGGCGACCACCGAGATCTACACCAG | 793 | CAGGAGGAGA | 1177 |
| RNA_ligation1_25 | GAGGAGAACACTCTTTCCCTAC | | | |
| EasySci- | AATGATACGGCGACCACCGAGATCTACACGAT | 794 | GATATCGGCG | 1178 |
| RNA_ligation1_26 | ATCGGCGACACTCTTTCCCTAC | | | |
| EasySci- | AATGATACGGCGACCACCGAGATCTACACCCA | 795 | CCAGTCCTCT | 1179 |
| RNA_ligation1_27 | GTCCTCTACACTCTTTCCCTAC | | | |
| EasySci- | AATGATACGGCGACCACCGAGATCTACACCAT | 796 | CATAGTTCGG | 1180 |
| RNA_ligation1_28 | AGTTCGGACACTCTTTCCCTAC | | | |
| EasySci- | AATGATACGGCGACCACCGAGATCTACACCGT | 797 | CGTAATGCAG | 1181 |
| RNA_ligation1_29 | AATGCAGACACTCTTTCCCTAC | | | |
| EasySci- | AATGATACGGCGACCACCGAGATCTACACCCG | 798 | CCGTTCGGAT | 1182 |
| RNA_ligation1_30 | TTCGGATACACTCTTTCCCTAC | | | |
| EasySci- | AATGATACGGCGACCACCGAGATCTACACCCA | 799 | CCATAAGTCC | 1183 |
| RNA_ligation1_31 | TAAGTCCACACTCTTTCCCTAC | | | |
| EasySci- | AATGATACGGCGACCACCGAGATCTACACGGC | 800 | GGCAATGAGA | 1184 |
| RNA_ligation1_32 | AATGAGAACACTCTTTCCCTAC | | | |
| EasySci- | AATGATACGGCGACCACCGAGATCTACACCGG | 801 | CGGTTATGCC | 1185 |
| RNA_ligation1_33 | TTATGCCACACTCTTTCCCTAC | | | |
| EasySci- | AATGATACGGCGACCACCGAGATCTACACTGG | 802 | TGGCCGGCCT | 1186 |
| RNA_ligation1_34 | CCGGCCTACACTCTTTCCCTAC | | | |
| EasySci- | AATGATACGGCGACCACCGAGATCTACACAGC | 803 | AGCTGCAATA | 1187 |
| RNA_ligation1_35 | TGCAATAACACTCTTTCCCTAC | | | |
| EasySci- | AATGATACGGCGACCACCGAGATCTACACTGG | 804 | TGGCCATGCA | 1188 |
| RNA_ligation1_36 | CCATGCAACACTCTTTCCCTAC | | | |
| EasySci- | AATGATACGGCGACCACCGAGATCTACACTGA | 805 | TGACGCTCCG | 1189 |
| RNA_ligation1_37 | CGCTCCGACACTCTTTCCCTAC | | | |
| Easy_Sci- | AATGATACGGCGACCACCGAGATCTACACAAC | 806 | AACTGCTGCC | 1190 |
| RNA_ligation1_38 | TGCTGCCACACTCTTTCCCTAC | | | |
| EasySci- | AATGATACGGCGACCACCGAGATCTACACTGC | 807 | TGCGCGATGC | 1191 |
| RNA_ligation1_39 | GCGATGCACACTCTTTCCCTAC | | | |
| EasySci- | AATGATACGGCGACCACCGAGATCTACACATT | 808 | ATTGAGATTG | 1192 |
| RNA_ligation1_40 | GAGATTGACACTCTTTCCCTAC | | | |
| EasySci- | AATGATACGGCGACCACCGAGATCTACACTTG | 809 | TTGATATATT | 1193 |
| RNA_ligation1_41 | ATATATTACACTCTTTCCCTAC | | | |
| EasySci- | AATGATACGGCGACCACCGAGATCTACACCGG | 810 | CGGTAGGAAT | 1194 |
| RNA_ligation1_42 | TAGGAATACACTCTTTCCCTAC | | | |
| EasySci- | AATGATACGGCGACCACCGAGATCTACACACC | 811 | ACCAGCGCAG | 1195 |
| RNA_ligation1_43 | AGCGCAGACACTCTTTCCCTAC | | | |
| EasySci- | AATGATACGGCGACCACCGAGATCTACACCGA | 812 | CGAATGAGCT | 1196 |
| RNA_ligation1_44 | ATGAGCTACACTCTTTCCCTAC | | | |
| EasySci- | AATGATACGGCGACCACCGAGATCTACACAGT | 813 | AGTTCGAGTA | 1197 |
| RNA_ligation1_45 | TCGAGTAACACTCTTTCCCTAC | | | |
| EasySci- | AATGATACGGCGACCACCGAGATCTACACTTG | 814 | TTGGACGCTG | 1198 |
| RNA_ligation1_46 | GACGCTGACACTCTTTCCCTAC | | | |
| EasySci- | AATGATACGGCGACCACCGAGATCTACACATA | 815 | ATAGACTAGG | 1199 |
| RNA_ligation1_47 | GACTAGGACACTCTTTCCCTAC | | | |
| EasySci- | AATGATACGGCGACCACCGAGATCTACACTAT | 816 | TATAGTAAGC | 1200 |
| RNA_ligation1_48 | AGTAAGCACACTCTTTCCCTAC | | | |
| EasySci- | AATGATACGGCGACCACCGAGATCTACACCGG | 817 | CGGTCGTTAA | 1201 |
| RNA_ligation1_49 | TCGTTAAACACTCTTTCCCTAC | | | |
| EasySci- | AATGATACGGCGACCACCGAGATCTACACATG | 818 | ATGGCGGATC | 1202 |
| RNA_ligation1_50 | GCGGATCACACTCTTTCCCTAC | | | |
| EasySci- | AATGATACGGCGACCACCGAGATCTACACCTC | 819 | CTCTGATCAG | 1203 |
| RNA_ligation1_51 | TGATCAGACACTCTTTCCCTAC | | | |
| EasySci- | AATGATACGGCGACCACCGAGATCTACACGGC | 820 | GGCCAGTCCG | 1204 |
| RNA_ligation1_52 | CAGTCCGACACTCTTTCCCTAC | | | |
| EasySci- | AATGATACGGCGACCACCGAGATCTACACCGG | 821 | CGGAAGATAT | 1205 |
| RNA_ligation1_53 | AAGATATACACTCTTTCCCTAC | | | |
| EasySci- | AATGATACGGCGACCACCGAGATCTACACTGG | 822 | TGGCTGATGA | 1206 |
| RNA_ligation1_54 | CTGATGAACACTCTTTCCCTAC | | | |
| EasySci- | AATGATACGGCGACCACCGAGATCTACACGAA | 823 | GAAGGTTGCC | 1207 |
| RNA_ligation1_55 | GGTTGCCACACTCTTTCCCTAC | | | |
| EasySci- | AATGATACGGCGACCACCGAGATCTACACGTT | 824 | GTTGAAGGAT | 1208 |
| RNA_ligation1_56 | GAAGGATACACTCTTTCCCTAC | | | |
| EasySci- | AATGATACGGCGACCACCGAGATCTACACCCA | 825 | CCATTCGTAA | 1209 |
| RNA_ligation1_57 | TTCGTAAACACTCTTTCCCTAC | | | |
| EasySci- | AATGATACGGCGACCACCGAGATCTACACTGC | 826 | TGCGCCAGAA | 1210 |
| RNA_ligation1_58 | GCCAGAAACACTCTTTCCCTAC | | | |
| EasySci- | AATGATACGGCGACCACCGAGATCTACACCGA | 827 | CGAATAATTC | 1211 |
| RNA_ligation1_59 | ATAATTCACACTCTTTCCCTAC | | | |
| EasySci- | AATGATACGGCGACCACCGAGATCTACACGCG | 828 | GCGACGCCTT | 1212 |
| RNA_ligation1_60 | ACGCCTTACACTCTTTCCCTAC | | | |
| EasySci- | AATGATACGGCGACCACCGAGATCTACACATC | 829 | ATCAACGATT | 1213 |
| RNA_ligation1_61 | AACGATTACACTCTTTCCCTAC | | | |
| EasySci- | AATGATACGGCGACCACCGAGATCTACACGTT | 830 | GTTCTGAATT | 1214 |
| RNA_ligation1_62 | CTGAATTACACTCTTTCCCTAC | | | |
| EasySci- | AATGATACGGCGACCACCGAGATCTACACGCT | 831 | GCTAACCTCA | 1215 |
| RNA_ligation1_63 | AACCTCAACACTCTTTCCCTAC | | | |
| EasySci- | AATGATACGGCGACCACCGAGATCTACACCAA | 832 | CAAGCAACTG | 1216 |
| RNA_ligation1_64 | GCAACTGACACTCTTTCCCTAC | | | |
| EasySci- | AATGATACGGCGACCACCGAGATCTACACGGA | 833 | GGAGCGGCCG | 1217 |
| RNA_ligation1_65 | GCGGCCGACACTCTTTCCCTAC | | | |
| EasySci- | AATGATACGGCGACCACCGAGATCTACACCGC | 834 | CGCGTACGAC | 1218 |
| RNA_ligation1_66 | GTACGACACACTCTTTCCCTAC | | | |
| EasySci- | AATGATACGGCGACCACCGAGATCTACACCGA | 835 | CGATGGCGCC | 1219 |
| RNA_ligation1_67 | TGGCGCCACACTCTTTCCCTAC | | | |
| EasySci- | AATGATACGGCGACCACCGAGATCTACACTGG | 836 | TGGTATTCAT | 1220 |
| RNA_ligation1_68 | TATTCATACACTCTTTCCCTAC | | | |
| EasySci- | AATGATACGGCGACCACCGAGATCTACACGAT | 837 | GATAAGGCAA | 1221 |
| RNA_ligation1_69 | AAGGCAAACACTCTTTCCCTAC | | | |
| EasySci- | AATGATACGGCGACCACCGAGATCTACACGCC | 838 | GCCGGTCGAG | 1222 |
| RNA_ligation1_70 | GGTCGAGACACTCTTTCCCTAC | | | |
| EasySci- | AATGATACGGCGACCACCGAGATCTACACTGC | 839 | TGCGCCATCT | 1223 |
| RNA_ligation1_71 | GCCATCTACACTCTTTCCCTAC | | | |
| EasySci- | AATGATACGGCGACCACCGAGATCTACACAAG | 840 | AAGTCTTCCG | 1224 |
| RNA_ligation1_72 | TCTTCCGACACTCTTTCCCTAC | | | |
| Easy_Sci- | AATGATACGGCGACCACCGAGATCTACACAGA | 841 | AGACTCAAGC | 1225 |
| RNA_ligation1_73 | CTCAAGCACACTCTTTCCCTAC | | | |
| EasySci- | AATGATACGGCGACCACCGAGATCTACACGCA | 842 | GCAGGCGACG | 1226 |
| RNA_ligation1_74 | GGCGACGACACTCTTTCCCTAC | | | |
| EasySci- | AATGATACGGCGACCACCGAGATCTACACAAT | 843 | AATACTCTTC | 1227 |
| RNA_ligation1_75 | ACTCTTCACACTCTTTCCCTAC | | | |
| EasySci- | AATGATACGGCGACCACCGAGATCTACACCCA | 844 | CCAACTAACC | 1228 |
| RNA_ligation1_76 | ACTAACCACACTCTTTCCCTAC | | | |
| EasySci- | AATGATACGGCGACCACCGAGATCTACACTAT | 845 | TATCCTCAAT | 1229 |
| RNA_ligation1_77 | CCTCAATACACTCTTTCCCTAC | | | |
| EasySci- | AATGATACGGCGACCACCGAGATCTACACGCC | 846 | GCCGTCGCGT | 1230 |
| RNA_ligation1_78 | GTCGCGTACACTCTTTCCCTAC | | | |
| EasySci- | AATGATACGGCGACCACCGAGATCTACACCCG | 847 | CCGCTGCTTC | 1231 |
| RNA_ligation1_79 | CTGCTTCACACTCTTTCCCTAC | | | |
| EasySci- | AATGATACGGCGACCACCGAGATCTACACTGA | 848 | TGACCGAATC | 1232 |
| RNA_ligation1_80 | CCGAATCACACTCTTTCCCTAC | | | |
| EasySci- | AATGATACGGCGACCACCGAGATCTACACGTC | 849 | GTCTCCAGAG | 1233 |
| RNA_ligation1_81 | TCCAGAGACACTCTTTCCCTAC | | | |
| EasySci- | AATGATACGGCGACCACCGAGATCTACACAAT | 850 | AATGCTAGTC | 1234 |
| RNA_ligation1_82 | GCTAGTCACACTCTTTCCCTAC | | | |
| EasySci- | AATGATACGGCGACCACCGAGATCTACACGAC | 851 | GACGACCTGC | 1235 |
| RNA_ligation1_83 | GACCTGCACACTCTTTCCCTAC | | | |
| EasySci- | AATGATACGGCGACCACCGAGATCTACACAGA | 852 | AGAGCCAGCC | 1236 |
| RNA_ligation1_84 | GCCAGCCACACTCTTTCCCTAC | | | |
| EasySci- | AATGATACGGCGACCACCGAGATCTACACCCA | 853 | CCAGGCCGCA | 1237 |
| RNA_ligation1_85 | GGCCGCAACACTCTTTCCCTAC | | | |
| EasySci- | AATGATACGGCGACCACCGAGATCTACACCAG | 854 | CAGGTATGGA | 1238 |
| RNA_ligation1_86 | GTATGGAACACTCTTTCCCTAC | | | |
| EasySci- | AATGATACGGCGACCACCGAGATCTACACCCG | 855 | CCGGAGTTGC | 1239 |
| RNA_ligation1_87 | GAGTTGCACACTCTTTCCCTAC | | | |
| EasySci- | AATGATACGGCGACCACCGAGATCTACACTTA | 856 | TTAATTATTG | 1240 |
| RNA_ligation1_88 | ATTATTGACACTCTTTCCCTAC | | | |
| EasySci- | AATGATACGGCGACCACCGAGATCTACACAAT | 857 | AATCAGCTGC | 1241 |
| RNA_ligation1_89 | CAGCTGCACACTCTTTCCCTAC | | | |
| EasySci- | AATGATACGGCGACCACCGAGATCTACACCCG | 858 | CCGTTGACTT | 1242 |
| RNA_ligation1_90 | TTGACTTACACTCTTTCCCTAC | | | |
| EasySci- | AATGATACGGCGACCACCGAGATCTACACGCC | 859 | GCCAGGATCA | 1243 |
| RNA_ligation1_91 | AGGATCAACACTCTTTCCCTAC | | | |
| EasySci- | AATGATACGGCGACCACCGAGATCTACACCTT | 860 | CTTCGGCGCA | 1244 |
| RNA_ligation1_92 | CGGCGCAACACTCTTTCCCTAC | | | |
| EasySci- | AATGATACGGCGACCACCGAGATCTACACCAA | 861 | CAAGGCATTC | 1245 |
| RNA_ligation1_93 | GGCATTCACACTCTTTCCCTAC | | | |
| EasySci- | AATGATACGGCGACCACCGAGATCTACACAAG | 862 | AAGAATGGAA | 1246 |
| RNA_ligation1_94 | AATGGAAACACTCTTTCCCTAC | | | |
| EasySci- | AATGATACGGCGACCACCGAGATCTACACCGG | 863 | CGGATGAAGG | 1247 |
| RNA_ligation1_95 | ATGAAGGACACTCTTTCCCTAC | | | |
| EasySci- | AATGATACGGCGACCACCGAGATCTACACTAT | 864 | TATCGTCGGC | 1248 |
| RNA_ligation1_96 | CGTCGGCACACTCTTTCCCTAC | | | |
| EasySci- | AATGATACGGCGACCACCGAGATCTACACAGA | 865 | AGAGAACTTG | 1249 |
| RNA_ligation2_01 | GAACTTGACACTCTTTCCCTAC | | | |
| EasySci- | AATGATACGGCGACCACCGAGATCTACACAGT | 866 | AGTCGGCTCC | 1250 |
| RNA_ligation2_02 | CGGCTCCACACTCTTTCCCTAC | | | |
| EasySci- | AATGATACGGCGACCACCGAGATCTACACTAC | 867 | TACCAGAGTA | 1251 |
| RNA_ligation2_03 | CAGAGTAACACTCTTTCCCTAC | | | |
| EasySci- | AATGATACGGCGACCACCGAGATCTACACACC | 868 | ACCGACCTCA | 1252 |
| RNA_ligation2_04 | GACCTCAACACTCTTTCCCTAC | | | |
| EasySci- | AATGATACGGCGACCACCGAGATCTACACATC | 869 | ATCCTACCTC | 1253 |
| RNA_ligation2_05 | CTACCTCACACTCTTTCCCTAC | | | |
| EasySci- | AATGATACGGCGACCACCGAGATCTACACTGC | 870 | TGCAAGGCGT | 1254 |
| RNA_ligation2_06 | AAGGCGTACACTCTTTCCCTAC | | | |
| EasySci- | AATGATACGGCGACCACCGAGATCTACACTTG | 871 | TTGCTGCGCC | 1255 |
| RNA_ligation2_07 | CTGCGCCACACTCTTTCCCTAC | | | |
| EasySci- | AATGATACGGCGACCACCGAGATCTACACCCG | 872 | CCGCGCTATA | 1256 |
| RNA_ligation2_08 | CGCTATAACACTCTTTCCCTAC | | | |
| EasySci- | AATGATACGGCGACCACCGAGATCTACACGGA | 873 | GGACGGAGCC | 1257 |
| RNA_ligation2_09 | CGGAGCCACACTCTTTCCCTAC | | | |
| EasySci- | AATGATACGGCGACCACCGAGATCTACACAAT | 874 | AATACTTGCG | 1258 |
| RNA_ligation2_10 | ACTTGCGACACTCTTTCCCTAC | | | |
| EasySci- | AATGATACGGCGACCACCGAGATCTACACGGA | 875 | GGATTGACTC | 1259 |
| RNA_ligation2_11 | TTGACTCACACTCTTTCCCTAC | | | |
| EasySci- | AATGATACGGCGACCACCGAGATCTACACAGC | 876 | AGCTTACGAA | 1260 |
| RNA_ligation2_12 | TTACGAAACACTCTTTCCCTAC | | | |
| EasySci- | AATGATACGGCGACCACCGAGATCTACACTGA | 877 | TGATGCATCG | 1261 |
| RNA_ligation2_13 | TGCATCGACACTCTTTCCCTAC | | | |
| EasySci- | AATGATACGGCGACCACCGAGATCTACACATA | 878 | ATAATCTCGC | 1262 |
| RNA_ligation2_14 | ATCTCGCACACTCTTTCCCTAC | | | |
| EasySci- | AATGATACGGCGACCACCGAGATCTACACCAG | 879 | CAGCAGTATC | 1263 |
| RNA_ligation2_15 | CAGTATCACACTCTTTCCCTAC | | | |
| EasySci- | AATGATACGGCGACCACCGAGATCTACACACG | 880 | ACGACCAATA | 1264 |
| RNA_ligation2_16 | ACCAATAACACTCTTTCCCTAC | | | |
| EasySci- | AATGATACGGCGACCACCGAGATCTACACCGG | 881 | CGGATAGGTA | 1265 |
| RNA_ligation2_17 | ATAGGTAACACTCTTTCCCTAC | | | |
| EasySci- | AATGATACGGCGACCACCGAGATCTACACTCG | 882 | TCGAAGCGCG | 1266 |
| RNA_ligation2_18 | AAGCGCGACACTCTTTCCCTAC | | | |
| EasySci- | AATGATACGGCGACCACCGAGATCTACACGGT | 883 | GGTAAGCTCT | 1267 |
| RNA_ligation2_19 | AAGCTCTACACTCTTTCCCTAC | | | |
| EasySci- | AATGATACGGCGACCACCGAGATCTACACAGG | 884 | AGGTAATTCC | 1268 |
| RNA_ligation2_20 | TAATTCCACACTCTTTCCCTAC | | | |
| EasySci- | AATGATACGGCGACCACCGAGATCTACACAGA | 885 | AGACCATTCA | 1269 |
| RNA_ligation2_21 | CCATTCAACACTCTTTCCCTAC | | | |
| EasySci- | AATGATACGGCGACCACCGAGATCTACACCTG | 886 | CTGATCGACC | 1270 |
| RNA_ligation2_22 | ATCGACCACACTCTTTCCCTAC | | | |
| EasySci- | AATGATACGGCGACCACCGAGATCTACACGCA | 887 | GCAATTACTC | 1271 |
| RNA_ligation2_23 | ATTACTCACACTCTTTCCCTAC | | | |
| EasySci- | AATGATACGGCGACCACCGAGATCTACACGAG | 888 | GAGGAGTTCG | 1272 |
| RNA_ligation2_24 | GAGTTCGACACTCTTTCCCTAC | | | |
| EasySci- | AATGATACGGCGACCACCGAGATCTACACTAG | 889 | TAGTACTATC | 1273 |
| RNA_ligation2_25 | TACTATCACACTCTTTCCCTAC | | | |
| EasySci- | AATGATACGGCGACCACCGAGATCTACACCGA | 890 | CGACTTGGCG | 1274 |
| RNA_ligation2_26 | CTTGGCGACACTCTTTCCCTAC | | | |
| EasySci- | AATGATACGGCGACCACCGAGATCTACACCTA | 891 | CTATTCGGCC | 1275 |
| RNA_ligation2_27 | TTCGGCCACACTCTTTCCCTAC | | | |
| EasySci- | AATGATACGGCGACCACCGAGATCTACACCTT | 892 | CTTCCAAGAA | 1276 |
| RNA_ligation2_28 | CCAAGAAACACTCTTTCCCTAC | | | |
| EasySci- | AATGATACGGCGACCACCGAGATCTACACCGA | 893 | CGATCCTGGA | 1277 |
| RNA_ligation2_29 | TCCTGGAACACTCTTTCCCTAC | | | |
| EasySci- | AATGATACGGCGACCACCGAGATCTACACTAT | 894 | TATTCCGTTA | 1278 |
| RNA_ligation2_30 | TCCGTTAACACTCTTTCCCTAC | | | |
| EasySci- | AATGATACGGCGACCACCGAGATCTACACTTA | 895 | TTAGTACGCC | 1279 |
| RNA_ligation2_31 | GTACGCCACACTCTTTCCCTAC | | | |
| EasySci- | AATGATACGGCGACCACCGAGATCTACACTCG | 896 | TCGTAGCATC | 1280 |
| RNA_ligation2_32 | TAGCATCACACTCTTTCCCTAC | | | |
| EasySci- | AATGATACGGCGACCACCGAGATCTACACGTA | 897 | GTATTAAGTT | 1281 |
| RNA_ligation2_33 | TTAAGTTACACTCTTTCCCTAC | | | |
| EasySci- | AATGATACGGCGACCACCGAGATCTACACCAT | 898 | CATTCTAGAA | 1282 |
| RNA_ligation2_34 | TCTAGAAACACTCTTTCCCTAC | | | |
| EasySci- | AATGATACGGCGACCACCGAGATCTACACGGT | 899 | GGTAGATCAA | 1283 |
| RNA_ligation2_35 | AGATCAAACACTCTTTCCCTAC | | | |
| EasySci- | AATGATACGGCGACCACCGAGATCTACACATC | 900 | ATCTCCTACG | 1284 |
| RNA_ligation2_36 | TCCTACGACACTCTTTCCCTAC | | | |
| EasySci- | AATGATACGGCGACCACCGAGATCTACACACG | 901 | ACGAAGAAGC | 1285 |
| RNA_ligation2_37 | AAGAAGCACACTCTTTCCCTAC | | | |
| EasySci- | AATGATACGGCGACCACCGAGATCTACACCCG | 902 | CCGATCAGCC | 1286 |
| RNA_ligation2_38 | ATCAGCCACACTCTTTCCCTAC | | | |
| EasySci- | AATGATACGGCGACCACCGAGATCTACACCTG | 903 | CTGGCTTCCT | 1287 |
| RNA_ligation2_39 | GCTTCCTACACTCTTTCCCTAC | | | |
| EasySci- | AATGATACGGCGACCACCGAGATCTACACTTC | 904 | TTCATAATGG | 1288 |
| RNA_ligation2_40 | ATAATGGACACTCTTTCCCTAC | | | |
| EasySci- | AATGATACGGCGACCACCGAGATCTACACGTT | 905 | GTTGAACGCA | 1289 |
| RNA_ligation2_41 | GAACGCAACACTCTTTCCCTAC | | | |
| EasySci- | AATGATACGGCGACCACCGAGATCTACACTAA | 906 | TAACGGCTGA | 1290 |
| RNA_ligation2_42 | CGGCTGAACACTCTTTCCCTAC | | | |
| EasySci- | AATGATACGGCGACCACCGAGATCTACACGAA | 907 | GAAGTCCGTC | 1291 |
| RNA_ligation2_43 | GTCCGTCACACTCTTTCCCTAC | | | |
| EasySci- | AATGATACGGCGACCACCGAGATCTACACATA | 908 | ATACGCCGCC | 1292 |
| RNA_ligation2_44 | CGCCGCCACACTCTTTCCCTAC | | | |
| EasySci- | AATGATACGGCGACCACCGAGATCTACACACT | 909 | ACTGGATGCT | 1293 |
| RNA_ligation2_45 | GGATGCTACACTCTTTCCCTAC | | | |
| EasySci- | AATGATACGGCGACCACCGAGATCTACACGAG | 910 | GAGCGAATAT | 1294 |
| RNA_ligation2_46 | CGAATATACACTCTTTCCCTAC | | | |
| EasySci- | AATGATACGGCGACCACCGAGATCTACACTAT | 911 | TATATGAAGT | 1295 |
| RNA_ligation2_47 | ATGAAGTACACTCTTTCCCTAC | | | |
| EasySci- | AATGATACGGCGACCACCGAGATCTACACACG | 912 | ACGATACCGG | 1296 |
| RNA_ligation2_48 | ATACCGGACACTCTTTCCCTAC | | | |
| EasySci- | AATGATACGGCGACCACCGAGATCTACACTCA | 913 | TCATACCGCT | 1297 |
| RNA_ligation2_49 | TACCGCTACACTCTTTCCCTAC | | | |
| EasySci- | AATGATACGGCGACCACCGAGATCTACACCGC | 914 | CGCTAACCGT | 1298 |
| RNA_ligation2_50 | TAACCGTACACTCTTTCCCTAC | | | |
| EasySci- | AATGATACGGCGACCACCGAGATCTACACCGC | 915 | CGCATCCATC | 1299 |
| RNA_ligation2_51 | ATCCATCACACTCTTTCCCTAC | | | |
| EasySci- | AATGATACGGCGACCACCGAGATCTACACCGT | 916 | CGTCTTCCTT | 1300 |
| RNA_ligation2_52 | CTTCCTTACACTCTTTCCCTAC | | | |
| EasySci- | AATGATACGGCGACCACCGAGATCTACACAAC | 917 | AACGCTATTA | 1301 |
| RNA_ligation2_53 | GCTATTAACACTCTTTCCCTAC | | | |
| EasySci- | AATGATACGGCGACCACCGAGATCTACACTAA | 918 | TAAGATAGGT | 1302 |
| RNA_ligation2_54 | GATAGGTACACTCTTTCCCTAC | | | |
| EasySci- | AATGATACGGCGACCACCGAGATCTACACTGA | 919 | TGATAATAGC | 1303 |
| RNA_ligation2_55 | TAATAGCACACTCTTTCCCTAC | | | |
| EasySci- | AATGATACGGCGACCACCGAGATCTACACGGC | 920 | GGCCTCCATT | 1304 |
| RNA_ligation2_56 | CTCCATTACACTCTTTCCCTAC | | | |
| EasySci- | AATGATACGGCGACCACCGAGATCTACACTGC | 921 | TGCCGCCGAT | 1305 |
| RNA_ligation2_57 | CGCCGATACACTCTTTCCCTAC | | | |
| EasySci- | AATGATACGGCGACCACCGAGATCTACACTGC | 922 | TGCCTATTAT | 1306 |
| RNA_ligation2_58 | CTATTATACACTCTTTCCCTAC | | | |
| EasySci- | AATGATACGGCGACCACCGAGATCTACACCTG | 923 | CTGATACGTC | 1307 |
| RNA_ligation2_59 | ATACGTCACACTCTTTCCCTAC | | | |
| EasySci- | AATGATACGGCGACCACCGAGATCTACACGAC | 924 | GACCTGGAAT | 1308 |
| RNA_ligation2_60 | CTGGAATACACTCTTTCCCTAC | | | |
| EasySci- | AATGATACGGCGACCACCGAGATCTACACTCA | 925 | TCAGATCGGA | 1309 |
| RNA_ligation2_61 | GATCGGAACACTCTTTCCCTAC | | | |
| EasySci- | AATGATACGGCGACCACCGAGATCTACACGAG | 926 | GAGGCGGAAT | 1310 |
| RNA_ligation2_62 | GCGGAATACACTCTTTCCCTAC | | | |
| EasySci- | AATGATACGGCGACCACCGAGATCTACACCAG | 927 | CAGCGCATCC | 1311 |
| RNA_ligation2_63 | CGCATCCACACTCTTTCCCTAC | | | |
| EasySci- | AATGATACGGCGACCACCGAGATCTACACAAT | 928 | AATGCCAAGA | 1312 |
| RNA_ligation2_64 | GCCAAGAACACTCTTTCCCTAC | | | |
| EasySci- | AATGATACGGCGACCACCGAGATCTACACTGG | 929 | TGGTCTACGT | 1313 |
| RNA_ligation2_65 | TCTACGTACACTCTTTCCCTAC | | | |
| EasySci- | AATGATACGGCGACCACCGAGATCTACACGGT | 930 | GGTCGCCGCT | 1314 |
| RNA_ligation2_66 | CGCCGCTACACTCTTTCCCTAC | | | |
| EasySci- | AATGATACGGCGACCACCGAGATCTACACAGC | 931 | AGCAAGTAGT | 1315 |
| RNA_ligation2_67 | AAGTAGTACACTCTTTCCCTAC | | | |
| EasySci- | AATGATACGGCGACCACCGAGATCTACACAAG | 932 | AAGAAGTTCA | 1316 |
| RNA_ligation2_68 | AAGTTCAACACTCTTTCCCTAC | | | |
| EasySci- | AATGATACGGCGACCACCGAGATCTACACCGG | 933 | CGGCGCTGGC | 1317 |
| RNA_ligation2_69 | CGCTGGCACACTCTTTCCCTAC | | | |
| EasySci- | AATGATACGGCGACCACCGAGATCTACACTCG | 934 | TCGTCAACTT | 1318 |
| RNA_ligation2_70 | TCAACTTACACTCTTTCCCTAC | | | |
| EasySci- | AATGATACGGCGACCACCGAGATCTACACCAA | 935 | CAACTTGGAT | 1319 |
| RNA_ligation2_71 | CTTGGATACACTCTTTCCCTAC | | | |
| EasySci- | AATGATACGGCGACCACCGAGATCTACACTTG | 936 | TTGGAGCTCA | 1320 |
| RNA_ligation2_72 | GAGCTCAACACTCTTTCCCTAC | | | |
| EasySci- | AATGATACGGCGACCACCGAGATCTACACCTT | 937 | CTTAGTTCAA | 1321 |
| RNA_ligation2_73 | AGTTCAAACACTCTTTCCCTAC | | | |
| EasySci- | AATGATACGGCGACCACCGAGATCTACACTTG | 938 | TTGAATTATA | 1322 |
| RNA_ligation2_74 | AATTATAACACTCTTTCCCTAC | | | |
| EasySci- | AATGATACGGCGACCACCGAGATCTACACCTT | 939 | CTTCAGCTTC | 1323 |
| RNA_ligation2_75 | CAGCTTCACACTCTTTCCCTAC | | | |
| EasySci- | AATGATACGGCGACCACCGAGATCTACACGTA | 940 | GTATACCGAA | 1324 |
| RNA_ligation2_76 | TACCGAAACACTCTTTCCCTAC | | | |
| EasySci- | AATGATACGGCGACCACCGAGATCTACACGGA | 941 | GGATATAATA | 1325 |
| RNA_ligation2_77 | TATAATAACACTCTTTCCCTAC | | | |
| EasySci- | AATGATACGGCGACCACCGAGATCTACACGAA | 942 | GAATCGACGT | 1326 |
| RNA_ligation2_78 | TCGACGTACACTCTTTCCCTAC | | | |
| EasySci- | AATGATACGGCGACCACCGAGATCTACACTGA | 943 | TGAACGGTAA | 1327 |
| RNA_ligation2_79 | ACGGTAAACACTCTTTCCCTAC | | | |
| EasySci- | AATGATACGGCGACCACCGAGATCTACACAAG | 944 | AAGTCGCGCG | 1328 |
| RNA_ligation2_80 | TCGCGCGACACTCTTTCCCTAC | | | |
| EasySci- | AATGATACGGCGACCACCGAGATCTACACTCC | 945 | TCCGCCTACT | 1329 |
| RNA_ligation2_81 | GCCTACTACACTCTTTCCCTAC | | | |
| Easy_Sci- | AATGATACGGCGACCACCGAGATCTACACTTA | 946 | TTAATAGTTC | 1330 |
| RNA_ligation2_82 | ATAGTTCACACTCTTTCCCTAC | | | |
| EasySci- | AATGATACGGCGACCACCGAGATCTACACCAA | 947 | CAACCGGATC | 1331 |
| RNA_ligation2_83 | CCGGATCACACTCTTTCCCTAC | | | |
| EasySci- | AATGATACGGCGACCACCGAGATCTACACTTA | 948 | TTAGAGCAAC | 1332 |
| RNA_ligation2_84 | GAGCAACACACTCTTTCCCTAC | | | |
| EasySci- | AATGATACGGCGACCACCGAGATCTACACCGT | 949 | CGTCATTCCA | 1333 |
| RNA_ligation2_85 | CATTCCAACACTCTTTCCCTAC | | | |
| EasySci- | AATGATACGGCGACCACCGAGATCTACACTAG | 950 | TAGGAAGGCA | 1334 |
| RNA_ligation2_86 | GAAGGCAACACTCTTTCCCTAC | | | |
| EasySci- | AATGATACGGCGACCACCGAGATCTACACTTG | 951 | TTGGCCTATA | 1335 |
| RNA_ligation2_87 | GCCTATAACACTCTTTCCCTAC | | | |
| EasySci- | AATGATACGGCGACCACCGAGATCTACACGCG | 952 | GCGTCTATTC | 1336 |
| RNA_ligation2_88 | TCTATTCACACTCTTTCCCTAC | | | |
| EasySci- | AATGATACGGCGACCACCGAGATCTACACCAG | 953 | CAGAGTAGAC | 1337 |
| RNA_ligation2_89 | AGTAGACACACTCTTTCCCTAC | | | |
| EasySci- | AATGATACGGCGACCACCGAGATCTACACATG | 954 | ATGCCGGACG | 1338 |
| RNA_ligation2_90 | CCGGACGACACTCTTTCCCTAC | | | |
| EasySci- | AATGATACGGCGACCACCGAGATCTACACTAT | 955 | TATTCGATCT | 1339 |
| RNA_ligation2_91 | TCGATCTACACTCTTTCCCTAC | | | |
| EasySci- | AATGATACGGCGACCACCGAGATCTACACATG | 956 | ATGGATCCGA | 1340 |
| RNA_ligation2_92 | GATCCGAACACTCTTTCCCTAC | | | |
| EasySci- | AATGATACGGCGACCACCGAGATCTACACATA | 957 | ATAATGCATT | 1341 |
| RNA_ligation2_93 | ATGCATTACACTCTTTCCCTAC | | | |
| EasySci- | AATGATACGGCGACCACCGAGATCTACACAAG | 958 | AAGTAGACTA | 1342 |
| RNA_ligation2_94 | TAGACTAACACTCTTTCCCTAC | | | |
| EasySci- | AATGATACGGCGACCACCGAGATCTACACATG | 959 | ATGGAAGCAT | 1343 |
| RNA_ligation2_95 | GAAGCATACACTCTTTCCCTAC | | | |
| EasySci- | AATGATACGGCGACCACCGAGATCTACACTGG | 960 | TGGATCAGGC | 1344 |
| RNA_ligation2_96 | ATCAGGCACACTCTTTCCCTAC | | | |
| EasySci- | AATGATACGGCGACCACCGAGATCTACACGTT | 961 | GTTACTTAGC | 1345 |
| RNA_ligation3_01 | ACTTAGCACACTCTTTCCCTAC | | | |
| EasySci- | AATGATACGGCGACCACCGAGATCTACACACC | 962 | ACCGCCGCAA | 1346 |
| RNA_ligation3_02 | GCCGCAAACACTCTTTCCCTAC | | | |
| EasySci- | AATGATACGGCGACCACCGAGATCTACACCTC | 963 | CTCAAGTCCT | 1347 |
| RNA_ligation3_03 | AAGTCCTACACTCTTTCCCTAC | | | |
| EasySci- | AATGATACGGCGACCACCGAGATCTACACCGG | 964 | CGGTCGACTA | 1348 |
| RNA_ligation3_04 | TCGACTAACACTCTTTCCCTAC | | | |
| EasySci- | AATGATACGGCGACCACCGAGATCTACACTTC | 965 | TTCGCCGTAA | 1349 |
| RNA_ligation3_05 | GCCGTAAACACTCTTTCCCTAC | | | |
| EasySci- | AATGATACGGCGACCACCGAGATCTACACGCT | 966 | GCTCCGCTTG | 1350 |
| RNA_ligation3_06 | CCGCTTGACACTCTTTCCCTAC | | | |
| EasySci- | AATGATACGGCGACCACCGAGATCTACACACT | 967 | ACTTAAGATA | 1351 |
| RNA_ligation3_07 | TAAGATAACACTCTTTCCCTAC | | | |
| EasySci- | AATGATACGGCGACCACCGAGATCTACACGGC | 968 | GGCATGGCCA | 1352 |
| RNA_ligation3_08 | ATGGCCAACACTCTTTCCCTAC | | | |
| EasySci- | AATGATACGGCGACCACCGAGATCTACACCTT | 969 | CTTCGGTATA | 1353 |
| RNA_ligation3_09 | CGGTATAACACTCTTTCCCTAC | | | |
| EasySci- | AATGATACGGCGACCACCGAGATCTACACGAG | 970 | GAGATTCGCC | 1354 |
| RNA_ligation3_10 | ATTCGCCACACTCTTTCCCTAC | | | |
| EasySci- | AATGATACGGCGACCACCGAGATCTACACCTA | 971 | CTAGGCCGTT | 1355 |
| RNA_ligation3_11 | GGCCGTTACACTCTTTCCCTAC | | | |
| EasySci- | AATGATACGGCGACCACCGAGATCTACACGGC | 972 | GGCCAACGAT | 1356 |
| RNA_ligation3_12 | CAACGATACACTCTTTCCCTAC | | | |
| EasySci- | AATGATACGGCGACCACCGAGATCTACACACG | 973 | ACGGAACCTG | 1357 |
| RNA_ligation3_13 | GAACCTGACACTCTTTCCCTAC | | | |
| EasySci- | AATGATACGGCGACCACCGAGATCTACACTGA | 974 | TGATTCTCGT | 1358 |
| RNA_ligation3_14 | TTCTCGTACACTCTTTCCCTAC | | | |
| EasySci- | AATGATACGGCGACCACCGAGATCTACACTTG | 975 | TTGCGTCAAC | 1359 |
| RNA_ligation3_15 | CGTCAACACACTCTTTCCCTAC | | | |
| EasySci- | AATGATACGGCGACCACCGAGATCTACACGAA | 976 | GAATGCAACC | 1360 |
| RNA_ligation3_16 | TGCAACCACACTCTTTCCCTAC | | | |
| EasySci- | AATGATACGGCGACCACCGAGATCTACACTGC | 977 | TGCGGTTCAG | 1361 |
| RNA_ligation3_17 | GGTTCAGACACTCTTTCCCTAC | | | |
| EasySci- | AATGATACGGCGACCACCGAGATCTACACTTG | 978 | TTGGCCAACC | 1362 |
| RNA_ligation3_18 | GCCAACCACACTCTTTCCCTAC | | | |
| EasySci- | AATGATACGGCGACCACCGAGATCTACACTTG | 979 | TTGGTTAAGC | 1363 |
| RNA_ligation3_19 | GTTAAGCACACTCTTTCCCTAC | | | |
| EasySci- | AATGATACGGCGACCACCGAGATCTACACCTT | 980 | CTTAAGTTCG | 1364 |
| RNA_ligation3_20 | AAGTTCGACACTCTTTCCCTAC | | | |
| Easy_Sci- | AATGATACGGCGACCACCGAGATCTACACGTC | 981 | GTCCTCAGAA | 1365 |
| RNA_ligation3_21 | CTCAGAAACACTCTTTCCCTAC | | | |
| EasySci- | AATGATACGGCGACCACCGAGATCTACACGGA | 982 | GGAGTCGTCT | 1366 |
| RNA_ligation3_22 | GTCGTCTACACTCTTTCCCTAC | | | |
| EasySci- | AATGATACGGCGACCACCGAGATCTACACGGT | 983 | GGTACCTCTA | 1367 |
| RNA_ligation3_23 | ACCTCTAACACTCTTTCCCTAC | | | |
| EasySci- | AATGATACGGCGACCACCGAGATCTACACGAT | 984 | GATCGCTGAG | 1368 |
| RNA_ligation3_24 | CGCTGAGACACTCTTTCCCTAC | | | |
| EasySci- | AATGATACGGCGACCACCGAGATCTACACAGA | 985 | AGAGTACTCC | 1369 |
| RNA_ligation3_25 | GTACTCCACACTCTTTCCCTAC | | | |
| EasySci- | AATGATACGGCGACCACCGAGATCTACACTCA | 986 | TCATTCTATT | 1370 |
| RNA_ligation3_26 | TTCTATTACACTCTTTCCCTAC | | | |
| EasySci- | AATGATACGGCGACCACCGAGATCTACACGTT | 987 | GTTACTACCA | 1371 |
| RNA_ligation3_27 | ACTACCAACACTCTTTCCCTAC | | | |
| EasySci- | AATGATACGGCGACCACCGAGATCTACACCCA | 988 | CCAGCTCGCC | 1372 |
| RNA_ligation3_28 | GCTCGCCACACTCTTTCCCTAC | | | |
| EasySci- | AATGATACGGCGACCACCGAGATCTACACCGC | 989 | CGCCGGTATG | 1373 |
| RNA_ligation3_29 | CGGTATGACACTCTTTCCCTAC | | | |
| EasySci- | AATGATACGGCGACCACCGAGATCTACACTTA | 990 | TTAATTCGTA | 1374 |
| RNA_ligation3_30 | ATTCGTAACACTCTTTCCCTAC | | | |
| EasySci- | AATGATACGGCGACCACCGAGATCTACACGAA | 991 | GAAGGCTCCA | 1375 |
| RNA_ligation3_31 | GGCTCCAACACTCTTTCCCTAC | | | |
| EasySci- | AATGATACGGCGACCACCGAGATCTACACGAG | 992 | GAGACGTACG | 1376 |
| RNA_ligation3_32 | ACGTACGACACTCTTTCCCTAC | | | |
| EasySci- | AATGATACGGCGACCACCGAGATCTACACGAA | 993 | GAAGAGCCTC | 1377 |
| RNA_ligation3_33 | GAGCCTCACACTCTTTCCCTAC | | | |
| EasySci- | AATGATACGGCGACCACCGAGATCTACACCCG | 994 | CCGATGCATA | 1378 |
| RNA_ligation3_34 | ATGCATAACACTCTTTCCCTAC | | | |
| EasySci- | AATGATACGGCGACCACCGAGATCTACACGTA | 995 | GTAATGGTAT | 1379 |
| RNA_ligation3_35 | ATGGTATACACTCTTTCCCTAC | | | |
| EasySci- | AATGATACGGCGACCACCGAGATCTACACTTC | 996 | TTCTATCTCA | 1380 |
| RNA_ligation3_36 | TATCTCAACACTCTTTCCCTAC | | | |
| EasySci- | AATGATACGGCGACCACCGAGATCTACACGCA | 997 | GCAGCAGCTA | 1381 |
| RNA_ligation3_37 | GCAGCTAACACTCTTTCCCTAC | | | |
| EasySci- | AATGATACGGCGACCACCGAGATCTACACTTG | 998 | TTGCTCGATT | 1382 |
| RNA_ligation3_38 | CTCGATTACACTCTTTCCCTAC | | | |
| EasySci- | AATGATACGGCGACCACCGAGATCTACACCCT | 999 | CCTCATCGGC | 1383 |
| RNA_ligation3_39 | CATCGGCACACTCTTTCCCTAC | | | |
| EasySci- | AATGATACGGCGACCACCGAGATCTACACACT | 1000 | ACTTCAGCAA | 1384 |
| RNA_ligation3_40 | TCAGCAAACACTCTTTCCCTAC | | | |
| EasySci- | AATGATACGGCGACCACCGAGATCTACACAGG | 1001 | AGGTCATCCT | 1385 |
| RNA_ligation3_41 | TCATCCTACACTCTTTCCCTAC | | | |
| EasySci- | AATGATACGGCGACCACCGAGATCTACACAAC | 1002 | AACGCGTCAG | 1386 |
| RNA_ligation3_42 | GCGTCAGACACTCTTTCCCTAC | | | |
| EasySci- | AATGATACGGCGACCACCGAGATCTACACCTA | 1003 | CTATGCTTAC | 1387 |
| RNA_ligation3_43 | TGCTTACACACTCTTTCCCTAC | | | |
| EasySci- | AATGATACGGCGACCACCGAGATCTACACGTT | 1004 | GTTGCCGTTC | 1388 |
| RNA_ligation3_44 | GCCGTTCACACTCTTTCCCTAC | | | |
| EasySci- | AATGATACGGCGACCACCGAGATCTACACGCT | 1005 | GCTTACCGCC | 1389 |
| RNA_ligation3_45 | TACCGCCACACTCTTTCCCTAC | | | |
| EasySci- | AATGATACGGCGACCACCGAGATCTACACTGG | 1006 | TGGCAAGTCA | 1390 |
| RNA_ligation3_46 | CAAGTCAACACTCTTTCCCTAC | | | |
| EasySci- | AATGATACGGCGACCACCGAGATCTACACCAT | 1007 | CATCGAAGGA | 1391 |
| RNA_ligation3_47 | CGAAGGAACACTCTTTCCCTAC | | | |
| EasySci- | AATGATACGGCGACCACCGAGATCTACACAGA | 1008 | AGAATCCTCG | 1392 |
| RNA_ligation3_48 | ATCCTCGACACTCTTTCCCTAC | | | |
| EasySci- | AATGATACGGCGACCACCGAGATCTACACGCA | 1009 | GCAATCGGTT | 1393 |
| RNA_ligation3_49 | ATCGGTTACACTCTTTCCCTAC | | | |
| EasySci- | AATGATACGGCGACCACCGAGATCTACACCCT | 1010 | CCTAAGATTC | 1394 |
| RNA_ligation3_50 | AAGATTCACACTCTTTCCCTAC | | | |
| EasySci- | AATGATACGGCGACCACCGAGATCTACACCCT | 1011 | CCTGCGCGCG | 1395 |
| RNA_ligation3_51 | GCGCGCGACACTCTTTCCCTAC | | | |
| EasySci- | AATGATACGGCGACCACCGAGATCTACACATC | 1012 | ATCAGCGCGA | 1396 |
| RNA_ligation3_52 | AGCGCGAACACTCTTTCCCTAC | | | |
| EasySci- | AATGATACGGCGACCACCGAGATCTACACGTA | 1013 | GTACGATTCT | 1397 |
| RNA_ligation3_53 | CGATTCTACACTCTTTCCCTAC | | | |
| EasySci- | AATGATACGGCGACCACCGAGATCTACACTTA | 1014 | TTACCTTGCA | 1398 |
| RNA_ligation3_54 | CCTTGCAACACTCTTTCCCTAC | | | |
| EasySci- | AATGATACGGCGACCACCGAGATCTACACCCG | 1015 | CCGGCTCAGC | 1399 |
| RNA_ligation3_55 | GCTCAGCACACTCTTTCCCTAC | | | |
| Easy_Sci- | AATGATACGGCGACCACCGAGATCTACACTTC | 1016 | TTCTGCAAGA | 1400 |
| RNA_ligation3_56 | TGCAAGAACACTCTTTCCCTAC | | | |
| EasySci- | AATGATACGGCGACCACCGAGATCTACACATA | 1017 | ATATACGCTT | 1401 |
| RNA_ligation3_57 | TACGCTTACACTCTTTCCCTAC | | | |
| EasySci- | AATGATACGGCGACCACCGAGATCTACACCTC | 1018 | CTCAGCAACC | 1402 |
| RNA_ligation3_58 | AGCAACCACACTCTTTCCCTAC | | | |
| EasySci- | AATGATACGGCGACCACCGAGATCTACACCAA | 1019 | CAATTCTAGG | 1403 |
| RNA_ligation3_59 | TTCTAGGACACTCTTTCCCTAC | | | |
| EasySci- | AATGATACGGCGACCACCGAGATCTACACATC | 1020 | ATCAGTCTCG | 1404 |
| RNA_ligation3_60 | AGTCTCGACACTCTTTCCCTAC | | | |
| EasySci- | AATGATACGGCGACCACCGAGATCTACACAAT | 1021 | AATCCGCAAC | 1405 |
| RNA_ligation3_61 | CCGCAACACACTCTTTCCCTAC | | | |
| EasySci- | AATGATACGGCGACCACCGAGATCTACACCGG | 1022 | CGGTTACCTT | 1406 |
| RNA_ligation3_62 | TTACCTTACACTCTTTCCCTAC | | | |
| EasySci- | AATGATACGGCGACCACCGAGATCTACACACG | 1023 | ACGTTAAGAC | 1407 |
| RNA_ligation3_63 | TTAAGACACACTCTTTCCCTAC | | | |
| EasySci- | AATGATACGGCGACCACCGAGATCTACACCTA | 1024 | CTATCCAACC | 1408 |
| RNA_ligation3_64 | TCCAACCACACTCTTTCCCTAC | | | |
| EasySci- | AATGATACGGCGACCACCGAGATCTACACATA | 1025 | ATAAGCGAAT | 1409 |
| RNA_ligation3_65 | AGCGAATACACTCTTTCCCTAC | | | |
| EasySci- | AATGATACGGCGACCACCGAGATCTACACCTT | 1026 | CTTATATCGG | 1410 |
| RNA_ligation3_66 | ATATCGGACACTCTTTCCCTAC | | | |
| EasySci- | AATGATACGGCGACCACCGAGATCTACACATA | 1027 | ATATGACGAC | 1411 |
| RNA_ligation3_67 | TGACGACACACTCTTTCCCTAC | | | |
| EasySci- | AATGATACGGCGACCACCGAGATCTACACTTA | 1028 | TTACCGCATA | 1412 |
| RNA_ligation3_68 | CCGCATAACACTCTTTCCCTAC | | | |
| EasySci- | AATGATACGGCGACCACCGAGATCTACACATT | 1029 | ATTCATCGCC | 1413 |
| RNA_ligation3_69 | CATCGCCACACTCTTTCCCTAC | | | |
| EasySci- | AATGATACGGCGACCACCGAGATCTACACAGA | 1030 | AGAAGCAGAA | 1414 |
| RNA_ligation3_70 | AGCAGAAACACTCTTTCCCTAC | | | |
| EasySci- | AATGATACGGCGACCACCGAGATCTACACGTT | 1031 | GTTCGTCGTT | 1415 |
| RNA_ligation3_71 | CGTCGTTACACTCTTTCCCTAC | | | |
| EasySci- | AATGATACGGCGACCACCGAGATCTACACCAT | 1032 | CATGCTTCCA | 1416 |
| RNA_ligation3_72 | GCTTCCAACACTCTTTCCCTAC | | | |
| EasySci- | AATGATACGGCGACCACCGAGATCTACACTCG | 1033 | TCGGTACCAG | 1417 |
| RNA_ligation3_73 | GTACCAGACACTCTTTCCCTAC | | | |
| EasySci- | AATGATACGGCGACCACCGAGATCTACACTTG | 1034 | TTGAGCCAAT | 1418 |
| RNA_ligation3_74 | AGCCAATACACTCTTTCCCTAC | | | |
| EasySci- | AATGATACGGCGACCACCGAGATCTACACAGA | 1035 | AGATGACTGA | 1419 |
| RNA_ligation3_75 | TGACTGAACACTCTTTCCCTAC | | | |
| EasySci- | AATGATACGGCGACCACCGAGATCTACACACG | 1036 | ACGCTAGAAG | 1420 |
| RNA_ligation3_76 | CTAGAAGACACTCTTTCCCTAC | | | |
| EasySci- | AATGATACGGCGACCACCGAGATCTACACGTT | 1037 | GTTCAATTGC | 1421 |
| RNA_ligation3_77 | CAATTGCACACTCTTTCCCTAC | | | |
| EasySci- | AATGATACGGCGACCACCGAGATCTACACGGA | 1038 | GGACCGTCAA | 1422 |
| RNA_ligation3_78 | CCGTCAAACACTCTTTCCCTAC | | | |
| EasySci- | AATGATACGGCGACCACCGAGATCTACACCAT | 1039 | CATTAACGGA | 1423 |
| RNA_ligation3_79 | TAACGGAACACTCTTTCCCTAC | | | |
| EasySci- | AATGATACGGCGACCACCGAGATCTACACTAA | 1040 | TAAGCAGTCC | 1424 |
| RNA_ligation3_80 | GCAGTCCACACTCTTTCCCTAC | | | |
| EasySci- | AATGATACGGCGACCACCGAGATCTACACCCG | 1041 | CCGGTCAGTT | 1425 |
| RNA_ligation3_81 | GTCAGTTACACTCTTTCCCTAC | | | |
| EasySci- | AATGATACGGCGACCACCGAGATCTACACATA | 1042 | ATAACGGACT | 1426 |
| RNA_ligation3_82 | ACGGACTACACTCTTTCCCTAC | | | |
| EasySci- | AATGATACGGCGACCACCGAGATCTACACACG | 1043 | ACGAGAAGAT | 1427 |
| RNA_ligation3_83 | AGAAGATACACTCTTTCCCTAC | | | |
| EasySci- | AATGATACGGCGACCACCGAGATCTACACATC | 1044 | ATCCTCTTAA | 1428 |
| RNA_ligation3_84 | CTCTTAAACACTCTTTCCCTAC | | | |
| EasySci- | AATGATACGGCGACCACCGAGATCTACACAAT | 1045 | AATCCAATAA | 1429 |
| RNA_ligation3_85 | CCAATAAACACTCTTTCCCTAC | | | |
| EasySci- | AATGATACGGCGACCACCGAGATCTACACCTA | 1046 | CTAGCAGGAT | 1430 |
| RNA_ligation3_86 | GCAGGATACACTCTTTCCCTAC | | | |
| EasySci- | AATGATACGGCGACCACCGAGATCTACACTGG | 1047 | TGGTCTCGGA | 1431 |
| RNA_ligation3_87 | TCTCGGAACACTCTTTCCCTAC | | | |
| EasySci- | AATGATACGGCGACCACCGAGATCTACACCCG | 1048 | CCGAGTACTA | 1432 |
| RNA_ligation3_88 | AGTACTAACACTCTTTCCCTAC | | | |
| EasySci- | AATGATACGGCGACCACCGAGATCTACACGAT | 1049 | GATGACGAAG | 1433 |
| RNA_ligation3_89 | GACGAAGACACTCTTTCCCTAC | | | |
| EasySci- | AATGATACGGCGACCACCGAGATCTACACGGC | 1050 | GGCAGTCTTC | 1434 |
| RNA_ligation3_90 | AGTCTTCACACTCTTTCCCTAC | | | |
| Easy_Sci- | AATGATACGGCGACCACCGAGATCTACACAAT | 1051 | AATACGAATA | 1435 |
| RNA_ligation3_91 | ACGAATAACACTCTTTCCCTAC | | | |
| EasySci- | AATGATACGGCGACCACCGAGATCTACACACC | 1052 | ACCTAGGAGA | 1436 |
| RNA_ligation3_92 | TAGGAGAACACTCTTTCCCTAC | | | |
| EasySci- | AATGATACGGCGACCACCGAGATCTACACGAA | 1053 | GAAGCGCCAA | 1437 |
| RNA_ligation3_93 | GCGCCAAACACTCTTTCCCTAC | | | |
| EasySci- | AATGATACGGCGACCACCGAGATCTACACCGT | 1054 | CGTTACGTTG | 1438 |
| RNA_ligation3_94 | TACGTTGACACTCTTTCCCTAC | | | |
| EasySci- | AATGATACGGCGACCACCGAGATCTACACGTC | 1055 | GTCGCGAATA | 1439 |
| RNA_ligation3_95 | GCGAATAACACTCTTTCCCTAC | | | |
| EasySci- | AATGATACGGCGACCACCGAGATCTACACTTA | 1056 | TTAGAGCCTG | 1440 |
| RNA_ligation3_96 | GAGCCTGACACTCTTTCCCTAC | | | |
| EasySci- | AATGATACGGCGACCACCGAGATCTACACACG | 1057 | ACGGTCATCA | 1441 |
| RNA_ligation4_01 | GTCATCAACACTCTTTCCCTAC | | | |
| EasySci- | AATGATACGGCGACCACCGAGATCTACACACG | 1058 | ACGTAGCAGG | 1442 |
| RNA_ligation4_02 | TAGCAGGACACTCTTTCCCTAC | | | |
| EasySci- | AATGATACGGCGACCACCGAGATCTACACCGA | 1059 | CGACCGAGAG | 1443 |
| RNA_ligation4_03 | CCGAGAGACACTCTTTCCCTAC | | | |
| EasySci- | AATGATACGGCGACCACCGAGATCTACACAAG | 1060 | AAGCGGTTCT | 1444 |
| RNA_ligation4_04 | CGGTTCTACACTCTTTCCCTAC | | | |
| EasySci- | AATGATACGGCGACCACCGAGATCTACACTCG | 1061 | TCGGAATAAC | 1445 |
| RNA_ligation4_05 | GAATAACACACTCTTTCCCTAC | | | |
| EasySci- | AATGATACGGCGACCACCGAGATCTACACAAG | 1062 | AAGTTCGCTG | 1446 |
| RNA_ligation4_06 | TTCGCTGACACTCTTTCCCTAC | | | |
| EasySci- | AATGATACGGCGACCACCGAGATCTACACAAT | 1063 | AATAATCGGT | 1447 |
| RNA_ligation4_07 | AATCGGTACACTCTTTCCCTAC | | | |
| EasySci- | AATGATACGGCGACCACCGAGATCTACACAGG | 1064 | AGGCGAAGGC | 1448 |
| RNA_ligation4_08 | CGAAGGCACACTCTTTCCCTAC | | | |
| EasySci- | AATGATACGGCGACCACCGAGATCTACACAAG | 1065 | AAGCCGCCGC | 1449 |
| RNA_ligation4_09 | CCGCCGCACACTCTTTCCCTAC | | | |
| EasySci- | AATGATACGGCGACCACCGAGATCTACACTCG | 1066 | TCGGCCGATG | 1450 |
| RNA_ligation4_10 | GCCGATGACACTCTTTCCCTAC | | | |
| EasySci- | AATGATACGGCGACCACCGAGATCTACACAGC | 1067 | AGCGACTGCT | 1451 |
| RNA_ligation4_11 | GACTGCTACACTCTTTCCCTAC | | | |
| EasySci- | AATGATACGGCGACCACCGAGATCTACACCTT | 1068 | CTTAATGAGC | 1452 |
| RNA_ligation4_12 | AATGAGCACACTCTTTCCCTAC | | | |
| EasySci- | AATGATACGGCGACCACCGAGATCTACACAAT | 1069 | AATTCCTCTC | 1453 |
| RNA_ligation4_13 | TCCTCTCACACTCTTTCCCTAC | | | |
| EasySci- | AATGATACGGCGACCACCGAGATCTACACGCT | 1070 | GCTGGTCTCC | 1454 |
| RNA_ligation4_14 | GGTCTCCACACTCTTTCCCTAC | | | |
| EasySci- | AATGATACGGCGACCACCGAGATCTACACAGT | 1071 | AGTATTGCTA | 1455 |
| RNA_ligation4_15 | ATTGCTAACACTCTTTCCCTAC | | | |
| EasySci- | AATGATACGGCGACCACCGAGATCTACACTCT | 1072 | TCTAGGATAA | 1456 |
| RNA_ligation4_16 | AGGATAAACACTCTTTCCCTAC | | | |
| EasySci- | AATGATACGGCGACCACCGAGATCTACACGGT | 1073 | GGTCCTGCAA | 1457 |
| RNA_ligation4_17 | CCTGCAAACACTCTTTCCCTAC | | | |
| EasySci- | AATGATACGGCGACCACCGAGATCTACACCGC | 1074 | CGCTTCAATT | 1458 |
| RNA_ligation4_18 | TTCAATTACACTCTTTCCCTAC | | | |
| EasySci- | AATGATACGGCGACCACCGAGATCTACACGGA | 1075 | GGATTATTAT | 1459 |
| RNA_ligation4_19 | TTATTATACACTCTTTCCCTAC | | | |
| EasySci- | AATGATACGGCGACCACCGAGATCTACACTCC | 1076 | TCCGGCTGAT | 1460 |
| RNA_ligation4_20 | GGCTGATACACTCTTTCCCTAC | | | |
| EasySci- | AATGATACGGCGACCACCGAGATCTACACCCG | 1077 | CCGCCTCGTT | 1461 |
| RNA_ligation4_21 | CCTCGTTACACTCTTTCCCTAC | | | |
| EasySci- | AATGATACGGCGACCACCGAGATCTACACTTA | 1078 | TTATAATCAA | 1462 |
| RNA_ligation4_22 | TAATCAAACACTCTTTCCCTAC | | | |
| EasySci- | AATGATACGGCGACCACCGAGATCTACACCCA | 1079 | CCATTGAACG | 1463 |
| RNA_ligation4_23 | TTGAACGACACTCTTTCCCTAC | | | |
| EasySci- | AATGATACGGCGACCACCGAGATCTACACACT | 1080 | ACTCCAACGG | 1464 |
| RNA_ligation4_24 | CCAACGGACACTCTTTCCCTAC | | | |
| EasySci- | AATGATACGGCGACCACCGAGATCTACACACC | 1081 | ACCTCCTGAA | 1465 |
| RNA_ligation4_25 | TCCTGAAACACTCTTTCCCTAC | | | |
| EasySci- | AATGATACGGCGACCACCGAGATCTACACAGA | 1082 | AGAGGCCGGC | 1466 |
| RNA_ligation4_26 | GGCCGGCACACTCTTTCCCTAC | | | |
| EasySci- | AATGATACGGCGACCACCGAGATCTACACCTG | 1083 | CTGCCTCTTC | 1467 |
| RNA_ligation4_27 | CCTCTTCACACTCTTTCCCTAC | | | |
| EasySci- | AATGATACGGCGACCACCGAGATCTACACCAG | 1084 | CAGTATCCTT | 1468 |
| RNA_ligation4_28 | TATCCTTACACTCTTTCCCTAC | | | |
| EasySci- | AATGATACGGCGACCACCGAGATCTACACGTC | 1085 | GTCAACTAGC | 1469 |
| RNA_ligation4_29 | AACTAGCACACTCTTTCCCTAC | | | |
| EasySci- | AATGATACGGCGACCACCGAGATCTACACTGA | 1086 | TGACGCAGTC | 1470 |
| RNA_ligation4_30 | CGCAGTCACACTCTTTCCCTAC | | | |
| EasySci- | AATGATACGGCGACCACCGAGATCTACACGTC | 1087 | GTCAATACGA | 1471 |
| RNA_ligation4_31 | AATACGAACACTCTTTCCCTAC | | | |
| EasySci- | AATGATACGGCGACCACCGAGATCTACACTGA | 1088 | TGAACTTCGA | 1472 |
| RNA_ligation4_32 | ACTTCGAACACTCTTTCCCTAC | | | |
| EasySci- | AATGATACGGCGACCACCGAGATCTACACCGT | 1089 | CGTACCAACG | 1473 |
| RNA_ligation4_33 | ACCAACGACACTCTTTCCCTAC | | | |
| EasySci- | AATGATACGGCGACCACCGAGATCTACACAGA | 1090 | AGAGATGAAT | 1474 |
| RNA_ligation4_34 | GATGAATACACTCTTTCCCTAC | | | |
| EasySci- | AATGATACGGCGACCACCGAGATCTACACTAT | 1091 | TATTCCAATT | 1475 |
| RNA_ligation4_35 | TCCAATTACACTCTTTCCCTAC | | | |
| EasySci- | AATGATACGGCGACCACCGAGATCTACACGGA | 1092 | GGATGCGATT | 1476 |
| RNA_ligation4_36 | TGCGATTACACTCTTTCCCTAC | | | |
| EasySci- | AATGATACGGCGACCACCGAGATCTACACGTA | 1093 | GTAACCAGGT | 1477 |
| RNA_ligation4_37 | ACCAGGTACACTCTTTCCCTAC | | | |
| EasySci- | AATGATACGGCGACCACCGAGATCTACACCCT | 1094 | CCTCGTCATA | 1478 |
| RNA_ligation4_38 | CGTCATAACACTCTTTCCCTAC | | | |
| EasySci- | AATGATACGGCGACCACCGAGATCTACACAAT | 1095 | AATGGTCTTA | 1479 |
| RNA_ligation4_39 | GGTCTTAACACTCTTTCCCTAC | | | |
| EasySci- | AATGATACGGCGACCACCGAGATCTACACATG | 1096 | ATGAATGCCT | 1480 |
| RNA_ligation4_40 | AATGCCTACACTCTTTCCCTAC | | | |
| EasySci- | AATGATACGGCGACCACCGAGATCTACACGTC | 1097 | GTCCGTAGAT | 1481 |
| RNA_ligation4_41 | CGTAGATACACTCTTTCCCTAC | | | |
| EasySci- | AATGATACGGCGACCACCGAGATCTACACCCA | 1098 | CCATCCTAGT | 1482 |
| RNA_ligation4_42 | TCCTAGTACACTCTTTCCCTAC | | | |
| EasySci- | AATGATACGGCGACCACCGAGATCTACACTGG | 1099 | TGGTTCCTAC | 1483 |
| RNA_ligation4_43 | TTCCTACACACTCTTTCCCTAC | | | |
| EasySci- | AATGATACGGCGACCACCGAGATCTACACGCG | 1100 | GCGCCTTCCG | 1484 |
| RNA_ligation4_44 | CCTTCCGACACTCTTTCCCTAC | | | |
| EasySci- | AATGATACGGCGACCACCGAGATCTACACCGT | 1101 | CGTACTACGC | 1485 |
| RNA_ligation4_45 | ACTACGCACACTCTTTCCCTAC | | | |
| EasySci- | AATGATACGGCGACCACCGAGATCTACACGGC | 1102 | GGCCGCGGTT | 1486 |
| RNA_ligation4_46 | CGCGGTTACACTCTTTCCCTAC | | | |
| EasySci- | AATGATACGGCGACCACCGAGATCTACACTGG | 1103 | TGGATAGTTG | 1487 |
| RNA_ligation4_47 | ATAGTTGACACTCTTTCCCTAC | | | |
| EasySci- | AATGATACGGCGACCACCGAGATCTACACCGG | 1104 | CGGCGCCAGG | 1488 |
| RNA_ligation4_48 | CGCCAGGACACTCTTTCCCTAC | | | |
| EasySci- | AATGATACGGCGACCACCGAGATCTACACCAA | 1105 | CAAGCTCAGG | 1489 |
| RNA_ligation4_49 | GCTCAGGACACTCTTTCCCTAC | | | |
| EasySci- | AATGATACGGCGACCACCGAGATCTACACATC | 1106 | ATCATCCTTC | 1490 |
| RNA_ligation4_50 | ATCCTTCACACTCTTTCCCTAC | | | |
| EasySci- | AATGATACGGCGACCACCGAGATCTACACCCT | 1107 | CCTCCGGAGT | 1491 |
| RNA_ligation4_51 | CCGGAGTACACTCTTTCCCTAC | | | |
| EasySci- | AATGATACGGCGACCACCGAGATCTACACCCA | 1108 | CCATTGCTGG | 1492 |
| RNA_ligation4_52 | TTGCTGGACACTCTTTCCCTAC | | | |
| EasySci- | AATGATACGGCGACCACCGAGATCTACACTAT | 1109 | TATTCGCAGT | 1493 |
| RNA_ligation4_53 | TCGCAGTACACTCTTTCCCTAC | | | |
| EasySci- | AATGATACGGCGACCACCGAGATCTACACCCG | 1110 | CCGGTTAAGT | 1494 |
| RNA_ligation4_54 | GTTAAGTACACTCTTTCCCTAC | | | |
| EasySci- | AATGATACGGCGACCACCGAGATCTACACATA | 1111 | ATATTCTACC | 1495 |
| RNA_ligation4_55 | TTCTACCACACTCTTTCCCTAC | | | |
| EasySci- | AATGATACGGCGACCACCGAGATCTACACTAC | 1112 | TACGGATCGT | 1496 |
| RNA_ligation4_56 | GGATCGTACACTCTTTCCCTAC | | | |
| EasySci- | AATGATACGGCGACCACCGAGATCTACACTTC | 1113 | TTCTCTCCAG | 1497 |
| RNA_ligation4_57 | TCTCCAGACACTCTTTCCCTAC | | | |
| EasySci- | AATGATACGGCGACCACCGAGATCTACACCCA | 1114 | CCAAGAGCAA | 1498 |
| RNA_ligation4_58 | AGAGCAAACACTCTTTCCCTAC | | | |
| EasySci- | AATGATACGGCGACCACCGAGATCTACACTTG | 1115 | TTGGTTCGAG | 1499 |
| RNA_ligation4_59 | GTTCGAGACACTCTTTCCCTAC | | | |
| EasySci- | AATGATACGGCGACCACCGAGATCTACACAAC | 1116 | AACGGATTAC | 1500 |
| RNA_ligation4_60 | GGATTACACACTCTTTCCCTAC | | | |
| EasySci- | AATGATACGGCGACCACCGAGATCTACACCAT | 1117 | CATCTTCAGA | 1501 |
| RNA_ligation4_61 | CTTCAGAACACTCTTTCCCTAC | | | |
| EasySci- | AATGATACGGCGACCACCGAGATCTACACTTG | 1118 | TTGAACCTCC | 1502 |
| RNA_ligation4_62 | AACCTCCACACTCTTTCCCTAC | | | |
| EasySci- | AATGATACGGCGACCACCGAGATCTACACGGA | 1119 | GGAATTCCAA | 1503 |
| RNA_ligation4_63 | ATTCCAAACACTCTTTCCCTAC | | | |
| EasySci- | AATGATACGGCGACCACCGAGATCTACACATA | 1120 | ATAGGTCCAA | 1504 |
| RNA_ligation4_64 | GGTCCAAACACTCTTTCCCTAC | | | |
| EasySci- | AATGATACGGCGACCACCGAGATCTACACGCC | 1121 | GCCATGGTAC | 1505 |
| RNA_ligation4_65 | ATGGTACACACTCTTTCCCTAC | | | |
| EasySci- | AATGATACGGCGACCACCGAGATCTACACGAG | 1122 | GAGCTCTTCA | 1506 |
| RNA_ligation4_66 | CTCTTCAACACTCTTTCCCTAC | | | |
| EasySci- | AATGATACGGCGACCACCGAGATCTACACCCG | 1123 | CCGAGGCAAC | 1507 |
| RNA_ligation4_67 | AGGCAACACACTCTTTCCCTAC | | | |
| EasySci- | AATGATACGGCGACCACCGAGATCTACACGTC | 1124 | GTCTCTAGTT | 1508 |
| RNA_ligation4_68 | TCTAGTTACACTCTTTCCCTAC | | | |
| EasvSci- | AATGATACGGCGACCACCGAGATCTACACGCT | 1125 | GCTGGTTATA | 1509 |
| RNA_ligation4_69 | GGTTATAACACTCTTTCCCTAC | | | |
| EasySci- | AATGATACGGCGACCACCGAGATCTACACTCG | 1126 | TCGTAGGTCA | 1510 |
| RNA_ligation4_70 | TAGGTCAACACTCTTTCCCTAC | | | |
| EasySci- | AATGATACGGCGACCACCGAGATCTACACAAC | 1127 | AACTCAGACG | 1511 |
| RNA_ligation4_71 | TCAGACGACACTCTTTCCCTAC | | | |
| EasySci- | AATGATACGGCGACCACCGAGATCTACACTGC | 1128 | TGCTGCCGGA | 1512 |
| RNA_ligation4_72 | TGCCGGAACACTCTTTCCCTAC | | | |
| EasySci- | AATGATACGGCGACCACCGAGATCTACACTGG | 1129 | TGGAGGCAAG | 1513 |
| RNA_ligation4_73 | AGGCAAGACACTCTTTCCCTAC | | | |
| EasySci- | AATGATACGGCGACCACCGAGATCTACACACT | 1130 | ACTGATGCGA | 1514 |
| RNA_ligation4_74 | GATGCGAACACTCTTTCCCTAC | | | |
| EasySci- | AATGATACGGCGACCACCGAGATCTACACACG | 1131 | ACGACTCCTC | 1515 |
| RNA_ligation4_75 | ACTCCTCACACTCTTTCCCTAC | | | |
| EasySci- | AATGATACGGCGACCACCGAGATCTACACTGG | 1132 | TGGCAGCGAA | 1516 |
| RNA_ligation4_76 | CAGCGAAACACTCTTTCCCTAC | | | |
| EasySci- | AATGATACGGCGACCACCGAGATCTACACCCG | 1133 | CCGATACTCT | 1517 |
| RNA_ligation4_77 | ATACTCTACACTCTTTCCCTAC | | | |
| EasySci- | AATGATACGGCGACCACCGAGATCTACACCAA | 1134 | CAATATAGGC | 1518 |
| RNA_ligation4_78 | TATAGGCACACTCTTTCCCTAC | | | |
| EasySci- | AATGATACGGCGACCACCGAGATCTACACACC | 1135 | ACCGGCCGAC | 1519 |
| RNA_ligation4_79 | GGCCGACACACTCTTTCCCTAC | | | |
| EasySci- | AATGATACGGCGACCACCGAGATCTACACAAT | 1136 | AATAAGGCTC | 1520 |
| RNA_ligation4_80 | AAGGCTCACACTCTTTCCCTAC | | | |
| EasySci- | AATGATACGGCGACCACCGAGATCTACACCAT | 1137 | CATCATAGCA | 1521 |
| RNA_ligation4_81 | CATAGCAACACTCTTTCCCTAC | | | |
| EasySci- | AATGATACGGCGACCACCGAGATCTACACGAT | 1138 | GATGATCCAT | 1522 |
| RNA_ligation4_82 | GATCCATACACTCTTTCCCTAC | | | |
| EasySci- | AATGATACGGCGACCACCGAGATCTACACATG | 1139 | ATGGCAATAC | 1523 |
| RNA_ligation4_83 | GCAATACACACTCTTTCCCTAC | | | |
| EasySci- | AATGATACGGCGACCACCGAGATCTACACACC | 1140 | ACCAGAACCA | 1524 |
| RNA_ligation4_84 | AGAACCAACACTCTTTCCCTAC | | | |
| EasySci- | AATGATACGGCGACCACCGAGATCTACACGGT | 1141 | GGTTCGACCT | 1525 |
| RNA_ligation4_85 | TCGACCTACACTCTTTCCCTAC | | | |
| EasySci- | AATGATACGGCGACCACCGAGATCTACACCTT | 1142 | CTTGGACGGA | 1526 |
| RNA_ligation4_86 | GGACGGAACACTCTTTCCCTAC | | | |
| EasySci- | AATGATACGGCGACCACCGAGATCTACACCGG | 1143 | CGGTCTCATA | 1527 |
| RNA_ligation4_87 | TCTCATAACACTCTTTCCCTAC | | | |
| EasySci- | AATGATACGGCGACCACCGAGATCTACACAAT | 1144 | AATCAGAGCC | 1528 |
| RNA_ligation4_88 | CAGAGCCACACTCTTTCCCTAC | | | |
| EasySci- | AATGATACGGCGACCACCGAGATCTACACCCT | 1145 | CCTGAATACT | 1529 |
| RNA_ligation4_89 | GAATACTACACTCTTTCCCTAC | | | |
| EasySci- | AATGATACGGCGACCACCGAGATCTACACCTT | 1146 | CTTGGAGACT | 1530 |
| RNA_ligation4_90 | GGAGACTACACTCTTTCCCTAC | | | |
| EasySci- | AATGATACGGCGACCACCGAGATCTACACAAG | 1147 | AAGACCTTAC | 1531 |
| RNA_ligation4_91 | ACCTTACACACTCTTTCCCTAC | | | |
| EasySci- | AATGATACGGCGACCACCGAGATCTACACGCG | 1148 | GCGAGCGCTC | 1532 |
| RNA_ligation4_92 | AGCGCTCACACTCTTTCCCTAC | | | |
| EasySci- | AATGATACGGCGACCACCGAGATCTACACTCG | 1149 | TCGCAAGACG | 1533 |
| RNA_ligation4_93 | CAAGACGACACTCTTTCCCTAC | | | |
| EasySci- | AATGATACGGCGACCACCGAGATCTACACCAA | 1150 | CAATCTCGGA | 1534 |
| RNA_ligation4_94 | TCTCGGAACACTCTTTCCCTAC | | | |
| EasySci- | AATGATACGGCGACCACCGAGATCTACACTCG | 1151 | TCGACCTACC | 1535 |
| RNA_ligation4_95 | ACCTACCACACTCTTTCCCTAC | | | |
| EasySci- | AATGATACGGCGACCACCGAGATCTACACTTA | 1152 | TTATAGGCAT | 1536 |
| RNA_ligation4_96 | TAGGCATACACTCTTTCCCTAC | | | |

### Adaptor: Common Ligation Adaptor Sequence

(SEQ ID NO: 2445)
A*G*A*T*C*G*G*A*A*G*A*G*C*G*T*C*G*T*G*T*A*G*G*G*
A*A*A*G*A*G*T*G*T*/3ddC/

‘*’ represents phosphorothioate bonds between nucleotides, which prevents the tagmentation of the oligo. /3ddC/′ represents a dideoxycytidine modification, which prevents the extension of the oligo on the 3′ end by DNA polymerases.

### Pool/Centrifuge/Resuspend/Redistribute/Quantify (30 m)

Centrifuge the tube for 3 minutes, 1000 g at 4 C. Pipet out the supernatant.
Resuspend the nuclei in 1 mL NBB. Move into a microcentrifuge tube. Centrifuge the tube for 3 minutes, 1000 g at 4 C. Dump the supernatant.
Resuspend the nuclei in 500 μL NBB Filter the nuclei using a 40 μM filter and then wash the filter with an additional 250 μL NBB. Centrifuge the tube for 3 minutes, 1000 g at 4 C. Dump the supernatant.
Resuspend the nuclei in 500 μL NBB for nuclei counting—it is recommended to use a fluorescent microscope with a solution with DAPI to distinguish nuclei from debris.
Distribute the nuclei into a 96 well plate with 10,000 nuclei per well, suspended in 4 μL total volume (final concentration=2,500 nuclei/μL).
   *NOTE. Can directly freeze and store cells at this point, but it is recommended to proceed directly to second-strand synthesis as dsDNA should be more stable in storage compared to ssDNA*
   *If choosing to freeze, it is okay to place directly in −80 C freezer without flash-freezing* *it is possible to store nuclei directly into PCR strips if profiling a whole plate of cells is not needed*

### Second-Strand Synthesis (1 h 15 m)

Thaw Second-Strand Synthesis buffer in room temperature
Prepare Second-Strand Synthesis mix: for each well, add ⅔ μL Second-Strand Synthesis buffer+⅓ μL Second-Strand Synthesis Enzyme Mix.
Perform Second-Strand Synthesis: in Thermocycler, incubate samples at 16 C for one hour. (STOP POINT)

### 0.8× Ampure Beads Purification (˜1 hr for One Plate)

Take one plate of prepared cells after Second-Strand Synthesis and add SuL DNA binding buffer to each well, mix, and let the resulting solution sit for 5 minutes at room temperature.
   *Can also perform this protocol with PCR strips if there is no need to profile a whole plate*
Add 8 μL ampure beads to each well, mix well via pipetting, and let the resulting solution sit for 5 minutes at room temperature.
Place the solution on a magnetic rack and let the solution sit for 5 minutes.
Remove the resulting supernatant and add SOUL of 80% ethanol (do not mix up and down). Remove the ethanol.
Wash one more time with 50 μL of 80% ethanol (do not mix up and down). Remove the ethanol, centrifuge the pellet down, place the plate on the magnetic rack, and remove the remaining residual ethanol.
Take the plate off of the magnetic rack and elute the beads in 7.6 μL of elution buffer. Incubate the solution for three minutes at room temperature.
Place the plate back on the magnetic rack and let the plate sit for three minutes at room temperature Aspirate 6.6 μL of solution without touching the magnetic beads and transfer the solution into a new plate.

### Tagmentation (10 m)

Prepare a mixture of 1:100 Tagmentase: Tagmentation Buffer mix. Add 6.6 μL of the mix to each well and pipet up and down to mix.
Incubate plate in the thermocycler at 55 C for 5 minutes. Place on ice immediately following the reaction.

### SDS Treatment (45 m)

For each well, add a mixture of:
   0.4 μL 1% SDS
   0.4 μL BSA
   2 μL 10 μM Universal P5 Primer
Incubate the plate at 55 C for 15 minutes. Place the plate on ice immediately following the reaction.
Add 2 μL 10% Tween-20 to each well.
Add 2 μL Indexed p7 primer to each well (Table 6). Centrifuge the plate after this step.

TABLE 6
P7 PCR primer sequences

| | | SEQ ID | | SEQ ID |
|---|---|---|---|---|
| Name | Sequence | NO: | Barcode | NO: |
| EasySci-RNA_P7- | CAAGCAGAAGACGGCATACGAGATccgaatccga | 1537 | TCGGATTCGG | 1921 |
| 1_01 | GTCTCGTGGGCTCGG | | | |
| EasySci-RNA_P7- | CAAGCAGAAGACGGCATACGAGATataagccgga | 1538 | TCCGGCTTAT | 1922 |
| 1_02 | GTCTCGTGGGCTCGG | | | |
| EasySci-RNA_P7- | CAAGCAGAAGACGGCATACGAGATccggcggcg | 1539 | TCGCCGCCGG | 1923 |
| 1_03 | aGTCTCGTGGGCTCGG | | | |
| EasySci-RNA_P7- | CAAGCAGAAGACGGCATACGAGATggcttgccaa | 1540 | TTGGCAAGCC | 1924 |
| 1_04 | GTCTCGTGGGCTCGG | | | |
| EasySci-RNA_P7- | CAAGCAGAAGACGGCATACGAGATccgctagctg | 1541 | CAGCTAGCGG | 1925 |
| 1_05 | GTCTCGTGGGCTCGG | | | |
| EasySci-RNA_P7- | CAAGCAGAAGACGGCATACGAGATcttatcctacG | 1542 | GTAGGATAAG | 1926 |
| 1_06 | TCTCGTGGGCTCGG | | | |
| EasySci-RNA_P7- | CAAGCAGAAGACGGCATACGAGATtgagctacttG | 1543 | AAGTAGCTCA | 1927 |
| 1_07 | TCTCGTGGGCTCGG | | | |
| EasySci-RNA_P7- | CAAGCAGAAGACGGCATACGAGATtcaggactta | 1544 | TAAGTCCTGA | 1928 |
| 1_08 | GTCTCGTGGGCTCGG | | | |
| EasySci-RNA_P7- | CAAGCAGAAGACGGCATACGAGATccgcagccgc | 1545 | GCGGCTGCGG | 1929 |
| 1_09 | GTCTCGTGGGCTCGG | | | |
| EasySci-RNA_P7- | CAAGCAGAAGACGGCATACGAGATtgcgcctggt | 1546 | ACCAGGCGCA | 1930 |
| 1_10 | GTCTCGTGGGCTCGG | | | |
| EasySci-RNA_P7- | CAAGCAGAAGACGGCATACGAGATaatcatacgg | 1547 | CCGTATGATT | 1931 |
| 1_11 | GTCTCGTGGGCTCGG | | | |
| EasySci-RNA_P7- | CAAGCAGAAGACGGCATACGAGATcgccaatcaa | 1548 | TTGATTGGCG | 1932 |
| 1_12 | GTCTCGTGGGCTCGG | | | |
| EasySci-RNA_P7- | CAAGCAGAAGACGGCATACGAGATcaaggcttag | 1549 | CTAAGCCTTG | 1933 |
| 1_13 | GTCTCGTGGGCTCGG | | | |
| EasySci-RNA_P7- | CAAGCAGAAGACGGCATACGAGATgcgctcgacg | 1550 | CGTCGAGCGC | 1934 |
| 1_14 | GTCTCGTGGGCTCGG | | | |
| EasySci-RNA_P7- | CAAGCAGAAGACGGCATACGAGATtccagcaata | 1551 | TATTGCTGGA | 1935 |
| 1_15 | GTCTCGTGGGCTCGG | | | |
| EasySci-RNA_P7- | CAAGCAGAAGACGGCATACGAGATcatgagaact | 1552 | AGTTCTCATG | 1936 |
| 1_16 | GTCTCGTGGGCTCGG | | | |
| EasySci-RNA_P7- | CAAGCAGAAGACGGCATACGAGATaacgtaatct | 1553 | AGATTACGTT | 1937 |
| 1_17 | GTCTCGTGGGCTCGG | | | |
| EasySci-RNA_P7- | CAAGCAGAAGACGGCATACGAGATattctcctctG | 1554 | AGAGGAGAAT | 1938 |
| 1_18 | TCTCGTGGGCTCGG | | | |
| EasySci-RNA_P7- | CAAGCAGAAGACGGCATACGAGATtctgcgcgtt | 1555 | AACGCGCAGA | 1939 |
| 1_19 | GTCTCGTGGGCTCGG | | | |
| EasySci-RNA_P7- | CAAGCAGAAGACGGCATACGAGATgctcatatgc | 1556 | GCATATGAGC | 1940 |
| 1_20 | GTCTCGTGGGCTCGG | | | |
| EasySci-RNA_P7- | CAAGCAGAAGACGGCATACGAGATagcggtaacg | 1557 | CGTTACCGCT | 1941 |
| 1_21 | GTCTCGTGGGCTCGG | | | |
| EasySci-RNA_P7- | CAAGCAGAAGACGGCATACGAGATaatgaatagt | 1558 | ACTATTCATT | 1942 |
| 1_22 | GTCTCGTGGGCTCGG | | | |
| EasySci-RNA_P7- | CAAGCAGAAGACGGCATACGAGATccgtatctgg | 1559 | CCAGATACGG | 1943 |
| 1_23 | GTCTCGTGGGCTCGG | | | |
| EasySci-RNA_P7- | CAAGCAGAAGACGGCATACGAGATccttagtctgG | 1560 | CAGACTAAGG | 1944 |
| 1_24 | TCTCGTGGGCTCGG | | | |
| EasySci-RNA_P7- | CAAGCAGAAGACGGCATACGAGATacctagttag | 1561 | CTAACTAGGT | 1945 |
| 1_25 | GTCTCGTGGGCTCGG | | | |
| EasySci-RNA_P7- | CAAGCAGAAGACGGCATACGAGATataggagtac | 1562 | GTACTCCTAT | 1946 |
| 1_26 | GTCTCGTGGGCTCGG | | | |
| EasySci-RNA_P7- | CAAGCAGAAGACGGCATACGAGATctacgacgag | 1563 | CTCGTCGTAG | 1947 |
| 1_27 | GTCTCGTGGGCTCGG | | | |
| EasySci-RNA_P7- | CAAGCAGAAGACGGCATACGAGATagtcgagttc | 1564 | GAACTCGACT | 1948 |
| 1_28 | GTCTCGTGGGCTCGG | | | |
| EasySci-RNA_P7- | CAAGCAGAAGACGGCATACGAGATtggtccagtc | 1565 | GACTGGACCA | 1949 |
| 1_29 | GTCTCGTGGGCTCGG | | | |
| EasySci-RNA_P7- | CAAGCAGAAGACGGCATACGAGATatctaagcaa | 1566 | TTGCTTAGAT | 1950 |
| 1_30 | GTCTCGTGGGCTCGG | | | |
| EasySci-RNA_P7- | CAAGCAGAAGACGGCATACGAGATcgaattcgttG | 1567 | AACGAATTCG | 1951 |
| 1_31 | TCTCGTGGGCTCGG | | | |
| EasySci-RNA_P7- | CAAGCAGAAGACGGCATACGAGATcagcgataga | 1568 | TCTATCGCTG | 1952 |
| 1_32 | GTCTCGTGGGCTCGG | | | |
| EasySci-RNA_P7- | CAAGCAGAAGACGGCATACGAGATggtcgctatg | 1569 | CATAGCGACC | 1953 |
| 1_33 | GTCTCGTGGGCTCGG | | | |
| EasySci-RNA_P7- | CAAGCAGAAGACGGCATACGAGATatccgttagc | 1570 | GCTAACGGAT | 1954 |
| 1_34 | GTCTCGTGGGCTCGG | | | |
| EasySci-RNA_P7- | CAAGCAGAAGACGGCATACGAGATtcgcaattag | 1571 | CTAATTGCGA | 1955 |
| 1_35 | GTCTCGTGGGCTCGG | | | |
| EasySci-RNA_P7- | CAAGCAGAAGACGGCATACGAGATggctggctag | 1572 | CTAGCCAGCC | 1956 |
| 1_36 | GTCTCGTGGGCTCGG | | | |
| EasySci-RNA_P7- | CAAGCAGAAGACGGCATACGAGATacggtcttgc | 1573 | GCAAGACCGT | 1957 |
| 1_37 | GTCTCGTGGGCTCGG | | | |
| EasySci-RNA_P7- | CAAGCAGAAGACGGCATACGAGATgctccattcg | 1574 | CGAATGGAGC | 1958 |
| 1_38 | GTCTCGTGGGCTCGG | | | |
| EasySci-RNA_P7- | CAAGCAGAAGACGGCATACGAGATacgataagcg | 1575 | CGCTTATCGT | 1959 |
| 1_39 | GTCTCGTGGGCTCGG | | | |
| EasySci-RNA_P7- | CAAGCAGAAGACGGCATACGAGATaccatagcgc | 1576 | GCGCTATGGT | 1960 |
| 1_40 | GTCTCGTGGGCTCGG | | | |
| EasySci-RNA_P7- | CAAGCAGAAGACGGCATACGAGATctcttagcgg | 1577 | CCGCTAAGAG | 1961 |
| 1_41 | GTCTCGTGGGCTCGG | | | |
| EasySci-RNA_P7- | CAAGCAGAAGACGGCATACGAGATtgattcaactG | 1578 | AGTTGAATCA | 1962 |
| 1_42 | TCTCGTGGGCTCGG | | | |
| EasySci-RNA_P7- | CAAGCAGAAGACGGCATACGAGATtatggccgcg | 1579 | CGCGGCCATA | 1963 |
| 1_43 | GTCTCGTGGGCTCGG | | | |
| EasySci-RNA_P7- | CAAGCAGAAGACGGCATACGAGATagaggtcgca | 1580 | TGCGACCTCT | 1964 |
| 1_44 | GTCTCGTGGGCTCGG | | | |
| EasySci-RNA_P7- | CAAGCAGAAGACGGCATACGAGATaggagattga | 1581 | TCAATCTCCT | 1965 |
| 1_45 | GTCTCGTGGGCTCGG | | | |
| EasySci-RNA_P7- | CAAGCAGAAGACGGCATACGAGATggctatatag | 1582 | CTATATAGCC | 1966 |
| 1_46 | GTCTCGTGGGCTCGG | | | |
| EasySci-RNA_P7- | CAAGCAGAAGACGGCATACGAGATtcgcgtacttG | 1583 | AAGTACGCGA | 1967 |
| 1_47 | TCTCGTGGGCTCGG | | | |
| EasySci-RNA_P7- | CAAGCAGAAGACGGCATACGAGATaataataatg | 1584 | CATTATTATT | 1968 |
| 1_48 | GTCTCGTGGGCTCGG | | | |
| EasySci-RNA_P7- | CAAGCAGAAGACGGCATACGAGATttcgttccatG | 1585 | ATGGAACGAA | 1969 |
| 1_49 | TCTCGTGGGCTCGG | | | |
| EasySci-RNA_P7- | CAAGCAGAAGACGGCATACGAGATtacctaatca | 1586 | TGATTAGGTA | 1970 |
| 1_50 | GTCTCGTGGGCTCGG | | | |
| EasySci-RNA_P7- | CAAGCAGAAGACGGCATACGAGATaagtaatattG | 1587 | AATATTACTT | 1971 |
| 1_51 | TCTCGTGGGCTCGG | | | |
| EasySci-RNA_P7- | CAAGCAGAAGACGGCATACGAGATagctaagaat | 1588 | ATTCTTAGCT | 1972 |
| 1_52 | GTCTCGTGGGCTCGG | | | |
| EasySci-RNA_P7- | CAAGCAGAAGACGGCATACGAGATgtcgaggtat | 1589 | ATACCTCGAC | 1973 |
| 1_53 | GTCTCGTGGGCTCGG | | | |
| EasySci-RNA_P7- | CAAGCAGAAGACGGCATACGAGATttattagtagG | 1590 | CTACTAATAA | 1974 |
| 1_54 | TCTCGTGGGCTCGG | | | |
| EasySci-RNA_P7- | CAAGCAGAAGACGGCATACGAGATtgcgaagatc | 1591 | GATCTTCGCA | 1975 |
| 1_55 | GTCTCGTGGGCTCGG | | | |
| EasySci-RNA_P7- | CAAGCAGAAGACGGCATACGAGATaactacggct | 1592 | AGCCGTAGTT | 1976 |
| 1_56 | GTCTCGTGGGCTCGG | | | |
| EasySci-RNA_P7- | CAAGCAGAAGACGGCATACGAGATaacggaacgc | 1593 | GCGTTCCGTT | 1977 |
| 1_57 | GTCTCGTGGGCTCGG | | | |
| EasySci-RNA_P7- | CAAGCAGAAGACGGCATACGAGATgatgctacga | 1594 | TCGTAGCATC | 1978 |
| 1_58 | GTCTCGTGGGCTCGG | | | |
| EasySci-RNA_P7- | CAAGCAGAAGACGGCATACGAGATatctgccaat | 1595 | ATTGGCAGAT | 1979 |
| 1_59 | GTCTCGTGGGCTCGG | | | |
| EasySci-RNA_P7- | CAAGCAGAAGACGGCATACGAGATatcgtatcaa | 1596 | TTGATACGAT | 1980 |
| 1_60 | GTCTCGTGGGCTCGG | | | |
| EasySci-RNA_P7- | CAAGCAGAAGACGGCATACGAGATaacgcctcta | 1597 | TAGAGGCGTT | 1981 |
| 1_61 | GTCTCGTGGGCTCGG | | | |
| EasySci-RNA_P7- | CAAGCAGAAGACGGCATACGAGATacggcaacca | 1598 | TGGTTGCCGT | 1982 |
| 1_62 | GTCTCGTGGGCTCGG | | | |
| EasySci-RNA_P7- | CAAGCAGAAGACGGCATACGAGATcaggctaaga | 1599 | TCTTAGCCTG | 1983 |
| 1_63 | GTCTCGTGGGCTCGG | | | |
| EasySci-RNA_P7- | CAAGCAGAAGACGGCATACGAGATcgcaatatca | 1600 | TGATATTGCG | 1984 |
| 1_64 | GTCTCGTGGGCTCGG | | | |
| EasySci-RNA_P7- | CAAGCAGAAGACGGCATACGAGATttcgataacc | 1601 | GGTTATCGAA | 1985 |
| 1_65 | GTCTCGTGGGCTCGG | | | |
| EasySci-RNA_P7- | CAAGCAGAAGACGGCATACGAGATaacctcaaga | 1602 | TCTTGAGGTT | 1986 |
| 1_66 | GTCTCGTGGGCTCGG | | | |
| EasySci-RNA_P7- | CAAGCAGAAGACGGCATACGAGATcaggcgccat | 1603 | ATGGCGCCTG | 1987 |
| 1_67 | GTCTCGTGGGCTCGG | | | |
| EasySci-RNA_P7- | CAAGCAGAAGACGGCATACGAGATaactattataG | 1604 | TATAATAGTT | 1988 |
| 1_68 | TCTCGTGGGCTCGG | | | |
| EasySci-RNA_P7- | CAAGCAGAAGACGGCATACGAGATaagttaccta | 1605 | TAGGTAACTT | 1989 |
| 1_69 | GTCTCGTGGGCTCGG | | | |
| EasySci-RNA_P7- | CAAGCAGAAGACGGCATACGAGATcggcagagg | 1606 | TCCTCTGCCG | 1990 |
| 1_70 | aGTCTCGTGGGCTCGG | | | |
| EasySci-RNA_P7- | CAAGCAGAAGACGGCATACGAGATgcctcaataa | 1607 | TTATTGAGGC | 1991 |
| 1_71 | GTCTCGTGGGCTCGG | | | |
| EasySci-RNA_P7- | CAAGCAGAAGACGGCATACGAGATttaacgccgt | 1608 | ACGGCGTTAA | 1992 |
| 1_72 | GTCTCGTGGGCTCGG | | | |
| EasySci-RNA_P7- | CAAGCAGAAGACGGCATACGAGATcatacgatgc | 1609 | GCATCGTATG | 1993 |
| 1_73 | GTCTCGTGGGCTCGG | | | |
| EasySci-RNA_P7- | CAAGCAGAAGACGGCATACGAGATaagctgacct | 1610 | AGGTCAGCTT | 1994 |
| 1_74 | GTCTCGTGGGCTCGG | | | |
| EasySci-RNA_P7- | CAAGCAGAAGACGGCATACGAGATgagtccttatG | 1611 | ATAAGGACTC | 1995 |
| 1_75 | TCTCGTGGGCTCGG | | | |
| EasySci-RNA_P7- | CAAGCAGAAGACGGCATACGAGATcctacggcaa | 1612 | TTGCCGTAGG | 1996 |
| 1_76 | GTCTCGTGGGCTCGG | | | |
| EasySci-RNA_P7- | CAAGCAGAAGACGGCATACGAGATaatattcgaa | 1613 | TTCGAATATT | 1997 |
| 1_77 | GTCTCGTGGGCTCGG | | | |
| EasySci-RNA_P7- | CAAGCAGAAGACGGCATACGAGATttcaagaatc | 1614 | GATTCTTGAA | 1998 |
| 1_78 | GTCTCGTGGGCTCGG | | | |
| EasySci-RNA_P7- | CAAGCAGAAGACGGCATACGAGATatgctcgcaa | 1615 | TTGCGAGCAT | 1999 |
| 1_79 | GTCTCGTGGGCTCGG | | | |
| EasySci-RNA_P7- | CAAGCAGAAGACGGCATACGAGATggagtaagcc | 1616 | GGCTTACTCC | 2000 |
| 1_80 | GTCTCGTGGGCTCGG | | | |
| EasySci-RNA_P7- | CAAGCAGAAGACGGCATACGAGATttatcgtattG | 1617 | AATACGATAA | 2001 |
| 1_81 | TCTCGTGGGCTCGG | | | |
| EasySci-RNA_P7- | CAAGCAGAAGACGGCATACGAGATaagtctaata | 1618 | TATTAGACTT | 2002 |
| 1_82 | GTCTCGTGGGCTCGG | | | |
| EasySci-RNA_P7- | CAAGCAGAAGACGGCATACGAGATcggcttacta | 1619 | TAGTAAGCCG | 2003 |
| 1_83 | GTCTCGTGGGCTCGG | | | |
| EasySci-RNA_P7- | CAAGCAGAAGACGGCATACGAGATgatatggtct | 1620 | AGACCATATC | 2004 |
| 1_84 | GTCTCGTGGGCTCGG | | | |
| EasySci-RNA_P7- | CAAGCAGAAGACGGCATACGAGATtagtcgtcca | 1621 | TGGACGACTA | 2005 |
| 1_85 | GTCTCGTGGGCTCGG | | | |
| EasySci-RNA_P7- | CAAGCAGAAGACGGCATACGAGATtagctgctac | 1622 | GTAGCAGCTA | 2006 |
| 1_86 | GTCTCGTGGGCTCGG | | | |
| EasySci-RNA_P7- | CAAGCAGAAGACGGCATACGAGATctcttcaagc | 1623 | GCTTGAAGAG | 2007 |
| 1_87 | GTCTCGTGGGCTCGG | | | |
| EasySci-RNA_P7- | CAAGCAGAAGACGGCATACGAGATatgaacgcgc | 1624 | GCGCGTTCAT | 2008 |
| 1_88 | GTCTCGTGGGCTCGG | | | |
| EasySci-RNA_P7- | CAAGCAGAAGACGGCATACGAGATgtcgacggaa | 1625 | TTCCGTCGAC | 2009 |
| 1_89 | GTCTCGTGGGCTCGG | | | |
| EasySci-RNA_P7- | CAAGCAGAAGACGGCATACGAGATactaattgag | 1626 | CTCAATTAGT | 2010 |
| 1_90 | GTCTCGTGGGCTCGG | | | |
| EasySci-RNA_P7- | CAAGCAGAAGACGGCATACGAGATcttgcataatG | 1627 | ATTATGCAAG | 2011 |
| 1_91 | TCTCGTGGGCTCGG | | | |
| EasySci-RNA_P7- | CAAGCAGAAGACGGCATACGAGATtccttaccaa | 1628 | TTGGTAAGGA | 2012 |
| 1_92 | GTCTCGTGGGCTCGG | | | |
| EasySci-RNA_P7- | CAAGCAGAAGACGGCATACGAGATtgcagcctac | 1629 | GTAGGCTGCA | 2013 |
| 1_93 | GTCTCGTGGGCTCGG | | | |
| EasySci-RNA_P7- | CAAGCAGAAGACGGCATACGAGATggagctgagg | 1630 | CCTCAGCTCC | 2014 |
| 1_94 | GTCTCGTGGGCTCGG | | | |
| EasySci-RNA_P7- | CAAGCAGAAGACGGCATACGAGATgcagcggact | 1631 | AGTCCGCTGC | 2015 |
| 1_95 | GTCTCGTGGGCTCGG | | | |
| EasySci-RNA_P7- | CAAGCAGAAGACGGCATACGAGATcatcgcgctc | 1632 | GAGCGCGATG | 2016 |
| 1_96 | GTCTCGTGGGCTCGG | | | |
| EasySci-RNA_P7- | CAAGCAGAAGACGGCATACGAGATTTCTGGC | 1633 | TAGGCCAGAA | 2017 |
| 2_01 | CTAGTCTCGTGGGCTCGG | | | |
| EasySci-RNA_P7- | CAAGCAGAAGACGGCATACGAGATAATTGG | 1634 | ATCGCCAATT | 2018 |
| 2_02 | CGATGTCTCGTGGGCTCGG | | | |
| EasySci-RNA_P7- | CAAGCAGAAGACGGCATACGAGATAGGCAA | 1635 | GCGGTTGCCT | 2019 |
| 2_03 | CCGCGTCTCGTGGGCTCGG | | | |
| EasySci-RNA_P7- | CAAGCAGAAGACGGCATACGAGATACGCAA | 1636 | ATCATTGCGT | 2020 |
| 2_04 | TGATGTCTCGTGGGCTCGG | | | |
| EasySci-RNA_P7- | CAAGCAGAAGACGGCATACGAGATACTCGTT | 1637 | AGTAACGAGT | 2021 |
| 2_05 | ACTGTCTCGTGGGCTCGG | | | |
| EasySci-RNA_P7- | CAAGCAGAAGACGGCATACGAGATCAAGTC | 1638 | TGCTGACTTG | 2022 |
| 2_06 | AGCAGTCTCGTGGGCTCGG | | | |
| EasySci-RNA_P7- | CAAGCAGAAGACGGCATACGAGATGGTACG | 1639 | TATCCGTACC | 2023 |
| 2_07 | GATAGTCTCGTGGGCTCGG | | | |
| EasySci-RNA_P7- | CAAGCAGAAGACGGCATACGAGATCTCAAT | 1640 | GACCATTGAG | 2024 |
| 2_08 | GGTCGTCTCGTGGGCTCGG | | | |
| EasySci-RNA_P7- | CAAGCAGAAGACGGCATACGAGATACTCCG | 1641 | TCTTCGGAGT | 2025 |
| 2_09 | AAGAGTCTCGTGGGCTCGG | | | |
| EasySci-RNA_P7- | CAAGCAGAAGACGGCATACGAGATAATCCA | 1642 | GCCTTGGATT | 2026 |
| 2_10 | AGGCGTCTCGTGGGCTCGG | | | |
| EasySci-RNA_P7- | CAAGCAGAAGACGGCATACGAGATCGTCCA | 1643 | GCGATGGACG | 2027 |
| 2_11 | TCGCGTCTCGTGGGCTCGG | | | |
| EasySci-RNA_P7- | CAAGCAGAAGACGGCATACGAGATTTAGGT | 1644 | TCGTACCTAA | 2028 |
| 2_12 | ACGAGTCTCGTGGGCTCGG | | | |
| EasySci-RNA_P7- | CAAGCAGAAGACGGCATACGAGATCTTACC | 1645 | AGATGGTAAG | 2029 |
| 2_13 | ATCTGTCTCGTGGGCTCGG | | | |
| EasySci-RNA_P7- | CAAGCAGAAGACGGCATACGAGATCGAGAT | 1646 | TCTTATCTCG | 2030 |
| 2_14 | AAGAGTCTCGTGGGCTCGG | | | |
| EasySci-RNA_P7- | CAAGCAGAAGACGGCATACGAGATCGATCTT | 1647 | TAGAAGATCG | 2031 |
| 2_15 | CTAGTCTCGTGGGCTCGG | | | |
| EasySci-RNA_P7- | CAAGCAGAAGACGGCATACGAGATGTTCCG | 1648 | GGATCGGAAC | 2032 |
| 2_16 | ATCCGTCTCGTGGGCTCGG | | | |
| EasySci-RNA_P7- | CAAGCAGAAGACGGCATACGAGATGCCATA | 1649 | TTCTTATGGC | 2033 |
| 2_17 | AGAAGTCTCGTGGGCTCGG | | | |
| EasySci-RNA_P7- | CAAGCAGAAGACGGCATACGAGATAGATTC | 1650 | TTATGAATCT | 2034 |
| 2_18 | ATAAGTCTCGTGGGCTCGG | | | |
| EasySci-RNA_P7- | CAAGCAGAAGACGGCATACGAGATCAGAGC | 1651 | TGACGCTCTG | 2035 |
| 2_19 | GTCAGTCTCGTGGGCTCGG | | | |
| EasySci-RNA_P7- | CAAGCAGAAGACGGCATACGAGATGAGGAG | 1652 | CTGGCTCCTC | 2036 |
| 2_20 | CCAGGTCTCGTGGGCTCGG | | | |
| EasySci-RNA_P7- | CAAGCAGAAGACGGCATACGAGATATGAGG | 1653 | GGATCCTCAT | 2037 |
| 2_21 | ATCCGTCTCGTGGGCTCGG | | | |
| EasySci-RNA_P7- | CAAGCAGAAGACGGCATACGAGATCGGCGC | 1654 | TTGAGCGCCG | 2038 |
| 2_22 | TCAAGTCTCGTGGGCTCGG | | | |
| EasySci-RNA_P7- | CAAGCAGAAGACGGCATACGAGATCCTTAC | 1655 | TGACGTAAGG | 2039 |
| 2_23 | GTCAGTCTCGTGGGCTCGG | | | |
| EasySci-RNA_P7- | CAAGCAGAAGACGGCATACGAGATTTCTCAG | 1656 | TGACTGAGAA | 2040 |
| 2_24 | TCAGTCTCGTGGGCTCGG | | | |
| EasySci-RNA_P7- | CAAGCAGAAGACGGCATACGAGATATAGGA | 1657 | AAGCTCCTAT | 2041 |
| 2_25 | GCTTGTCTCGTGGGCTCGG | | | |
| EasySci-RNA_P7- | CAAGCAGAAGACGGCATACGAGATTAGCCG | 1658 | GAGTCGGCTA | 2042 |
| 2_26 | ACTCGTCTCGTGGGCTCGG | | | |
| EasySci-RNA_P7- | CAAGCAGAAGACGGCATACGAGATAAGAAC | 1659 | CGGAGTTCTT | 2043 |
| 2_27 | TCCGGTCTCGTGGGCTCGG | | | |
| EasySci-RNA_P7- | CAAGCAGAAGACGGCATACGAGATCTTGAG | 1660 | CGGTCTCAAG | 2044 |
| 2_28 | ACCGGTCTCGTGGGCTCGG | | | |
| EasySci-RNA_P7- | CAAGCAGAAGACGGCATACGAGATAACGCC | 1661 | GTATGGCGTT | 2045 |
| 2_29 | ATACGTCTCGTGGGCTCGG | | | |
| EasySci-RNA_P7- | CAAGCAGAAGACGGCATACGAGATCCTACTC | 1662 | GTTGAGTAGG | 2046 |
| 2_30 | AACGTCTCGTGGGCTCGG | | | |
| EasySci-RNA_P7- | CAAGCAGAAGACGGCATACGAGATTCTAGC | 1663 | GGAGGCTAGA | 2047 |
| 2_31 | CTCCGTCTCGTGGGCTCGG | | | |
| EasySci-RNA_P7- | CAAGCAGAAGACGGCATACGAGATCGCTTA | 1664 | GATCTAAGCG | 2048 |
| 2_32 | GATCGTCTCGTGGGCTCGG | | | |
| EasySci-RNA_P7- | CAAGCAGAAGACGGCATACGAGATGCTTAA | 1665 | ATGATTAAGC | 2049 |
| 2_33 | TCATGTCTCGTGGGCTCGG | | | |
| EasySci-RNA_P7- | CAAGCAGAAGACGGCATACGAGATAGCAGG | 1666 | TTGACCTGCT | 2050 |
| 2_34 | TCAAGTCTCGTGGGCTCGG | | | |
| EasySci-RNA_P7- | CAAGCAGAAGACGGCATACGAGATCCGCTT | 1667 | TTATAAGCGG | 2051 |
| 2_35 | ATAAGTCTCGTGGGCTCGG | | | |
| EasySci-RNA_P7- | CAAGCAGAAGACGGCATACGAGATCCTAAT | 1668 | TTCCATTAGG | 2052 |
| 2_36 | GGAAGTCTCGTGGGCTCGG | | | |
| EasySci-RNA_P7- | CAAGCAGAAGACGGCATACGAGATCATTGG | 1669 | GGAACCAATG | 2053 |
| 2_37 | TTCCGTCTCGTGGGCTCGG | | | |
| EasySci-RNA_P7- | CAAGCAGAAGACGGCATACGAGATACCAGT | 1670 | AATAACTGGT | 2054 |
| 2_38 | TATTGTCTCGTGGGCTCGG | | | |
| EasySci-RNA_P7- | CAAGCAGAAGACGGCATACGAGATGACTCG | 1671 | TAAGCGAGTC | 2055 |
| 2_39 | CTTAGTCTCGTGGGCTCGG | | | |
| EasySci-RNA_P7- | CAAGCAGAAGACGGCATACGAGATAATTGC | 1672 | CGGAGCAATT | 2056 |
| 2_40 | TCCGGTCTCGTGGGCTCGG | | | |
| EasySci-RNA_P7- | CAAGCAGAAGACGGCATACGAGATTGCGAA | 1673 | GGCATTCGCA | 2057 |
| 2_41 | TGCCGTCTCGTGGGCTCGG | | | |
| EasySci-RNA_P7- | CAAGCAGAAGACGGCATACGAGATTGCCTCC | 1674 | GCTGGAGGCA | 2058 |
| 2_42 | AGCGTCTCGTGGGCTCGG | | | |
| EasySci-RNA_P7- | CAAGCAGAAGACGGCATACGAGATTTAGGC | 1675 | TCAAGCCTAA | 2059 |
| 2_43 | TTGAGTCTCGTGGGCTCGG | | | |
| EasySci-RNA_P7- | CAAGCAGAAGACGGCATACGAGATACTCTG | 1676 | AGCTCAGAGT | 2060 |
| 2_44 | AGCTGTCTCGTGGGCTCGG | | | |
| EasySci-RNA_P7- | CAAGCAGAAGACGGCATACGAGATTTCGAA | 1677 | GTCGTTCGAA | 2061 |
| 2_45 | CGACGTCTCGTGGGCTCGG | | | |
| EasySci-RNA_P7- | CAAGCAGAAGACGGCATACGAGATTACTGCT | 1678 | CCGAGCAGTA | 2062 |
| 2_46 | CGGGTCTCGTGGGCTCGG | | | |
| EasySci-RNA_P7- | CAAGCAGAAGACGGCATACGAGATTCTAGA | 1679 | GTCTTCTAGA | 2063 |
| 2_47 | AGACGTCTCGTGGGCTCGG | | | |
| EasySci-RNA_P7- | CAAGCAGAAGACGGCATACGAGATTATTGG | 1680 | TATTCCAATA | 2064 |
| 2_48 | AATAGTCTCGTGGGCTCGG | | | |
| EasySci-RNA_P7- | CAAGCAGAAGACGGCATACGAGATAAGATA | 1681 | TTGATATCTT | 2065 |
| 2_49 | TCAAGTCTCGTGGGCTCGG | | | |
| EasySci-RNA_P7- | CAAGCAGAAGACGGCATACGAGATAGTCCA | 1682 | ACCATGGACT | 2066 |
| 2_50 | TGGTGTCTCGTGGGCTCGG | | | |
| EasySci-RNA_P7- | CAAGCAGAAGACGGCATACGAGATGGTTGA | 1683 | GTATTCAACC | 2067 |
| 2_51 | ATACGTCTCGTGGGCTCGG | | | |
| EasySci-RNA_P7- | CAAGCAGAAGACGGCATACGAGATCTAGCG | 1684 | CCATCGCTAG | 2068 |
| 2_52 | ATGGGTCTCGTGGGCTCGG | | | |
| EasySci-RNA_P7- | CAAGCAGAAGACGGCATACGAGATCCAATA | 1685 | GGCGTATTGG | 2069 |
| 2_53 | CGCCGTCTCGTGGGCTCGG | | | |
| EasySci-RNA_P7- | CAAGCAGAAGACGGCATACGAGATGAGATA | 1686 | GAGGTATCTC | 2070 |
| 2_54 | CCTCGTCTCGTGGGCTCGG | | | |
| EasySci-RNA_P7- | CAAGCAGAAGACGGCATACGAGATGCTCAG | 1687 | GCTCCTGAGC | 2071 |
| 2_55 | GAGCGTCTCGTGGGCTCGG | | | |
| EasySci-RNA_P7- | CAAGCAGAAGACGGCATACGAGATACTAGT | 1688 | TGCAACTAGT | 2072 |
| 2_56 | TGCAGTCTCGTGGGCTCGG | | | |
| EasySci-RNA_P7- | CAAGCAGAAGACGGCATACGAGATCCGCTA | 1689 | TGGATAGCGG | 2073 |
| 2_57 | TCCAGTCTCGTGGGCTCGG | | | |
| EasySci-RNA_P7- | CAAGCAGAAGACGGCATACGAGATATGCAA | 1690 | TAAGTTGCAT | 2074 |
| 2_58 | CTTAGTCTCGTGGGCTCGG | | | |
| EasySci-RNA_P7- | CAAGCAGAAGACGGCATACGAGATAGGACC | 1691 | ACTTGGTCCT | 2075 |
| 2_59 | AAGTGTCTCGTGGGCTCGG | | | |
| EasySci-RNA_P7- | CAAGCAGAAGACGGCATACGAGATGGCGTC | 1692 | TGAGGACGCC | 2076 |
| 2_60 | CTCAGTCTCGTGGGCTCGG | | | |
| EasySci-RNA_P7- | CAAGCAGAAGACGGCATACGAGATGGAAGC | 1693 | GCGAGCTTCC | 2077 |
| 2_61 | TCGCGTCTCGTGGGCTCGG | | | |
| EasySci-RNA_P7- | CAAGCAGAAGACGGCATACGAGATAATCGT | 1694 | TATAACGATT | 2078 |
| 2_62 | TATAGTCTCGTGGGCTCGG | | | |
| EasySci-RNA_P7- | CAAGCAGAAGACGGCATACGAGATCCGAGA | 1695 | CCTCTCTCGG | 2079 |
| 2_63 | GAGGGTCTCGTGGGCTCGG | | | |
| EasySci-RNA_P7- | CAAGCAGAAGACGGCATACGAGATGAGGAA | 1696 | TGAGTTCCTC | 2080 |
| 2_64 | CTCAGTCTCGTGGGCTCGG | | | |
| EasySci-RNA_P7- | CAAGCAGAAGACGGCATACGAGATGGTCTG | 1697 | TGCTCAGACC | 2081 |
| 2_65 | AGCAGTCTCGTGGGCTCGG | | | |
| EasySci-RNA_P7- | CAAGCAGAAGACGGCATACGAGATACCATT | 1698 | GCTTAATGGT | 2082 |
| 2_66 | AAGCGTCTCGTGGGCTCGG | | | |
| EasySci-RNA_P7- | CAAGCAGAAGACGGCATACGAGATAACTCT | 1699 | TGGTAGAGTT | 2083 |
| 2_67 | ACCAGTCTCGTGGGCTCGG | | | |
| EasySci-RNA_P7- | CAAGCAGAAGACGGCATACGAGATTGCTCA | 1700 | GAGTTGAGCA | 2084 |
| 2_68 | ACTCGTCTCGTGGGCTCGG | | | |
| EasySci-RNA_P7- | CAAGCAGAAGACGGCATACGAGATCCGGCA | 1701 | AGGCTGCCGG | 2085 |
| 2_69 | GCCTGTCTCGTGGGCTCGG | | | |
| EasySci-RNA_P7- | CAAGCAGAAGACGGCATACGAGATCTCGCG | 1702 | TGGTCGCGAG | 2086 |
| 2_70 | ACCAGTCTCGTGGGCTCGG | | | |
| EasySci-RNA_P7- | CAAGCAGAAGACGGCATACGAGATTGGACC | 1703 | CTAAGGTCCA | 2087 |
| 2_71 | TTAGGTCTCGTGGGCTCGG | | | |
| EasySci-RNA_P7- | CAAGCAGAAGACGGCATACGAGATGGAATG | 1704 | CGGTCATTCC | 2088 |
| 2_72 | ACCGGTCTCGTGGGCTCGG | | | |
| EasySci-RNA_P7- | CAAGCAGAAGACGGCATACGAGATCAGTAT | 1705 | CATCATACTG | 2089 |
| 2_73 | GATGGTCTCGTGGGCTCGG | | | |
| EasySci-RNA_P7- | CAAGCAGAAGACGGCATACGAGATCTCATG | 1706 | TATTCATGAG | 2090 |
| 2_74 | AATAGTCTCGTGGGCTCGG | | | |
| EasySci-RNA_P7- | CAAGCAGAAGACGGCATACGAGATCTGCGT | 1707 | TACTACGCAG | 2091 |
| 2_75 | AGTAGTCTCGTGGGCTCGG | | | |
| EasySci-RNA_P7- | CAAGCAGAAGACGGCATACGAGATGGAGCA | 1708 | AGGTTGCTCC | 2092 |
| 2_76 | ACCTGTCTCGTGGGCTCGG | | | |
| EasySci-RNA_P7- | CAAGCAGAAGACGGCATACGAGATTCAGTA | 1709 | GTATTACTGA | 2093 |
| 2_77 | ATACGTCTCGTGGGCTCGG | | | |
| EasySci-RNA_P7- | CAAGCAGAAGACGGCATACGAGATGACGCT | 1710 | ATGCAGCGTC | 2094 |
| 2_78 | GCATGTCTCGTGGGCTCGG | | | |
| EasySci-RNA_P7- | CAAGCAGAAGACGGCATACGAGATATCTCC | 1711 | AGCTGGAGAT | 2095 |
| 2_79 | AGCTGTCTCGTGGGCTCGG | | | |
| EasySci-RNA_P7- | CAAGCAGAAGACGGCATACGAGATTTAGAA | 1712 | GCAGTTCTAA | 2096 |
| 2_80 | CTGCGTCTCGTGGGCTCGG | | | |
| EasySci-RNA_P7- | CAAGCAGAAGACGGCATACGAGATCCGTAA | 1713 | GGCGTTACGG | 2097 |
| 2_81 | CGCCGTCTCGTGGGCTCGG | | | |
| EasySci-RNA_P7- | CAAGCAGAAGACGGCATACGAGATGACTAT | 1714 | AGGTATAGTC | 2098 |
| 2_82 | ACCTGTCTCGTGGGCTCGG | | | |
| EasySci-RNA_P7- | CAAGCAGAAGACGGCATACGAGATTGCGAG | 1715 | ACCTCTCGCA | 2099 |
| 2_83 | AGGTGTCTCGTGGGCTCGG | | | |
| EasySci-RNA_P7- | CAAGCAGAAGACGGCATACGAGATATAGGC | 1716 | TGAGGCCTAT | 2100 |
| 2_84 | CTCAGTCTCGTGGGCTCGG | | | |
| EasySci-RNA_P7- | CAAGCAGAAGACGGCATACGAGATTCAATTC | 1717 | GTTGAATTGA | 2101 |
| 2_85 | AACGTCTCGTGGGCTCGG | | | |
| EasySci-RNA_P7- | CAAGCAGAAGACGGCATACGAGATGGCTAC | 1718 | CCAGGTAGCC | 2102 |
| 2_86 | CTGGGTCTCGTGGGCTCGG | | | |
| EasySci-RNA_P7- | CAAGCAGAAGACGGCATACGAGATCTTCTTG | 1719 | GTTCAAGAAG | 2103 |
| 2_87 | AACGTCTCGTGGGCTCGG | | | |
| EasySci-RNA_P7- | CAAGCAGAAGACGGCATACGAGATTTACGC | 1720 | TGCTGCGTAA | 2104 |
| 2_88 | AGCAGTCTCGTGGGCTCGG | | | |
| EasySci-RNA_P7- | CAAGCAGAAGACGGCATACGAGATATAACC | 1721 | TGGCGGTTAT | 2105 |
| 2_89 | GCCAGTCTCGTGGGCTCGG | | | |
| EasySci-RNA_P7- | CAAGCAGAAGACGGCATACGAGATCCAGTC | 1722 | ATTCGACTGG | 2106 |
| 2_90 | GAATGTCTCGTGGGCTCGG | | | |
| EasySci-RNA_P7- | CAAGCAGAAGACGGCATACGAGATTAACCA | 1723 | TAGTTGGTTA | 2107 |
| 2_91 | ACTAGTCTCGTGGGCTCGG | | | |
| EasySci-RNA_P7- | CAAGCAGAAGACGGCATACGAGATTAGCTG | 1724 | TCGCCAGCTA | 2108 |
| 2_92 | GCGAGTCTCGTGGGCTCGG | | | |
| EasySci-RNA_P7- | CAAGCAGAAGACGGCATACGAGATCAATGA | 1725 | CAAGTCATTG | 2109 |
| 2_93 | CTTGGTCTCGTGGGCTCGG | | | |
| EasySci-RNA_P7- | CAAGCAGAAGACGGCATACGAGATCTCTGC | 1726 | GATCGCAGAG | 2110 |
| 2_94 | GATCGTCTCGTGGGCTCGG | | | |
| EasySci-RNA_P7- | CAAGCAGAAGACGGCATACGAGATGCATAC | 1727 | ATTGGTATGC | 2111 |
| 2_95 | CAATGTCTCGTGGGCTCGG | | | |
| EasySci-RNA_P7- | CAAGCAGAAGACGGCATACGAGATACCTGA | 1728 | CCTATCAGGT | 2112 |
| 2_96 | TAGGGTCTCGTGGGCTCGG | | | |
| EasySci-RNA_P7- | CAAGCAGAAGACGGCATACGAGATATGGTT | 1729 | CGGTAACCAT | 2113 |
| 3_01 | ACCGGTCTCGTGGGCTCGG | | | |
| EasySci-RNA_P7- | CAAGCAGAAGACGGCATACGAGATCTTAGG | 1730 | ATAACCTAAG | 2114 |
| 3_02 | TTATGTCTCGTGGGCTCGG | | | |
| EasySci-RNA_P7- | CAAGCAGAAGACGGCATACGAGATCGCCTT | 1731 | GGCTAAGGCG | 2115 |
| 3_03 | AGCCGTCTCGTGGGCTCGG | | | |
| EasySci-RNA_P7- | CAAGCAGAAGACGGCATACGAGATACGTAA | 1732 | TACGTTACGT | 2116 |
| 3_04 | CGTAGTCTCGTGGGCTCGG | | | |
| EasySci-RNA_P7- | CAAGCAGAAGACGGCATACGAGATTTATTCT | 1733 | TAGAGAATAA | 2117 |
| 3_05 | CTAGTCTCGTGGGCTCGG | | | |
| EasySci-RNA_P7- | CAAGCAGAAGACGGCATACGAGATGCATAG | 1734 | TATACTATGC | 2118 |
| 3_06 | TATAGTCTCGTGGGCTCGG | | | |
| EasySci-RNA_P7- | CAAGCAGAAGACGGCATACGAGATCATGCG | 1735 | TATGCGCATG | 2119 |
| 3_07 | CATAGTCTCGTGGGCTCGG | | | |
| EasySci-RNA_P7- | CAAGCAGAAGACGGCATACGAGATGTACCA | 1736 | GAATTGGTAC | 2120 |
| 3_08 | ATTCGTCTCGTGGGCTCGG | | | |
| EasySci-RNA_P7- | CAAGCAGAAGACGGCATACGAGATAACGAG | 1737 | TGATCTCGTT | 2121 |
| 3_09 | ATCAGTCTCGTGGGCTCGG | | | |
| EasySci-RNA_P7- | CAAGCAGAAGACGGCATACGAGATTCTCGAT | 1738 | TTAATCGAGA | 2122 |
| 3_10 | TAAGTCTCGTGGGCTCGG | | | |
| EasySci-RNA_P7- | CAAGCAGAAGACGGCATACGAGATCGTCGA | 1739 | CCGGTCGACG | 2123 |
| 3_11 | CCGGGTCTCGTGGGCTCGG | | | |
| EasySci-RNA_P7- | CAAGCAGAAGACGGCATACGAGATGTTGAT | 1740 | AGCTATCAAC | 2124 |
| 3_12 | AGCTGTCTCGTGGGCTCGG | | | |
| EasySci-RNA_P7- | CAAGCAGAAGACGGCATACGAGATCAGCGT | 1741 | ACCAACGCTG | 2125 |
| 3_13 | TGGTGTCTCGTGGGCTCGG | | | |
| EasySci-RNA_P7- | CAAGCAGAAGACGGCATACGAGATATCGGC | 1742 | CAATGCCGAT | 2126 |
| 3_14 | ATTGGTCTCGTGGGCTCGG | | | |
| EasySci-RNA_P7- | CAAGCAGAAGACGGCATACGAGATGGTAGT | 1743 | TAGGACTACC | 2127 |
| 3_15 | CCTAGTCTCGTGGGCTCGG | | | |
| EasySci-RNA_P7- | CAAGCAGAAGACGGCATACGAGATTAGCAT | 1744 | CGCGATGCTA | 2128 |
| 3_16 | CGCGGTCTCGTGGGCTCGG | | | |
| EasySci-RNA_P7- | CAAGCAGAAGACGGCATACGAGATACTGGT | 1745 | GGTTACCAGT | 2129 |
| 3_17 | AACCGTCTCGTGGGCTCGG | | | |
| EasySci-RNA_P7- | CAAGCAGAAGACGGCATACGAGATTCTACTG | 1746 | GGTCAGTAGA | 2130 |
| 3_18 | ACCGTCTCGTGGGCTCGG | | | |
| EasySci-RNA_P7- | CAAGCAGAAGACGGCATACGAGATCAAGAG | 1747 | ATAACTCTTG | 2131 |
| 3_19 | TTATGTCTCGTGGGCTCGG | | | |
| EasySci-RNA_P7- | CAAGCAGAAGACGGCATACGAGATCAATTA | 1748 | TTCCTAATTG | 2132 |
| 3_20 | GGAAGTCTCGTGGGCTCGG | | | |
| EasySci-RNA_P7- | CAAGCAGAAGACGGCATACGAGATTTGCGG | 1749 | GGTTCCGCAA | 2133 |
| 3_21 | AACCGTCTCGTGGGCTCGG | | | |
| EasySci-RNA_P7- | CAAGCAGAAGACGGCATACGAGATAGCCGA | 1750 | TACTTCGGCT | 2134 |
| 3_22 | AGTAGTCTCGTGGGCTCGG | | | |
| EasySci-RNA_P7- | CAAGCAGAAGACGGCATACGAGATGGTCCG | 1751 | CGTACGGACC | 2135 |
| 3_23 | TACGGTCTCGTGGGCTCGG | | | |
| EasySci-RNA_P7- | CAAGCAGAAGACGGCATACGAGATAGCATG | 1752 | CTTGCATGCT | 2136 |
| 3_24 | CAAGGTCTCGTGGGCTCGG | | | |
| EasySci-RNA_P7- | CAAGCAGAAGACGGCATACGAGATTCTTCGT | 1753 | GCAACGAAGA | 2137 |
| 3_25 | TGCGTCTCGTGGGCTCGG | | | |
| EasySci-RNA_P7- | CAAGCAGAAGACGGCATACGAGATGTTGGA | 1754 | TTCTTCCAAC | 2138 |
| 3_26 | AGAAGTCTCGTGGGCTCGG | | | |
| EasySci-RNA_P7- | CAAGCAGAAGACGGCATACGAGATGGAATA | 1755 | TCCGTATTCC | 2139 |
| 3_27 | CGGAGTCTCGTGGGCTCGG | | | |
| EasySci-RNA_P7- | CAAGCAGAAGACGGCATACGAGATACCGAC | 1756 | TACCGTCGGT | 2140 |
| 3_28 | GGTAGTCTCGTGGGCTCGG | | | |
| EasySci-RNA_P7- | CAAGCAGAAGACGGCATACGAGATATACTT | 1757 | AATCAAGTAT | 2141 |
| 3_29 | GATTGTCTCGTGGGCTCGG | | | |
| EasySci-RNA_P7- | CAAGCAGAAGACGGCATACGAGATGTCGTTC | 1758 | GGCGAACGAC | 2142 |
| 3_30 | GCCGTCTCGTGGGCTCGG | | | |
| EasySci-RNA_P7- | CAAGCAGAAGACGGCATACGAGATAATGAT | 1759 | TGCAATCATT | 2143 |
| 3_31 | TGCAGTCTCGTGGGCTCGG | | | |
| EasySci-RNA_P7- | CAAGCAGAAGACGGCATACGAGATCCATTA | 1760 | CTGCTAATGG | 2144 |
| 3_32 | GCAGGTCTCGTGGGCTCGG | | | |
| EasySci-RNA_P7- | CAAGCAGAAGACGGCATACGAGATCTGACT | 1761 | CATTAGTCAG | 2145 |
| 3_33 | AATGGTCTCGTGGGCTCGG | | | |
| EasySci-RNA_P7- | CAAGCAGAAGACGGCATACGAGATCCTTAC | 1762 | GTCCGTAAGG | 2146 |
| 3_34 | GGACGTCTCGTGGGCTCGG | | | |
| EasySci-RNA_P7- | CAAGCAGAAGACGGCATACGAGATCGCATA | 1763 | TAGTTATGCG | 2147 |
| 3_35 | ACTAGTCTCGTGGGCTCGG | | | |
| EasySci-RNA_P7- | CAAGCAGAAGACGGCATACGAGATAACCGG | 1764 | TCCTCCGGTT | 2148 |
| 3_36 | AGGAGTCTCGTGGGCTCGG | | | |
| EasySci-RNA_P7- | CAAGCAGAAGACGGCATACGAGATAATGCA | 1765 | CCGCTGCATT | 2149 |
| 3_37 | GCGGGTCTCGTGGGCTCGG | | | |
| EasySci-RNA_P7- | CAAGCAGAAGACGGCATACGAGATCAGTCG | 1766 | TTGCCGACTG | 2150 |
| 3_38 | GCAAGTCTCGTGGGCTCGG | | | |
| EasySci-RNA_P7- | CAAGCAGAAGACGGCATACGAGATATAGAA | 1767 | TGGATTCTAT | 2151 |
| 3_39 | TCCAGTCTCGTGGGCTCGG | | | |
| EasySci-RNA_P7- | CAAGCAGAAGACGGCATACGAGATTCTCAG | 1768 | ATATCTGAGA | 2152 |
| 3_40 | ATATGTCTCGTGGGCTCGG | | | |
| EasySci-RNA_P7- | CAAGCAGAAGACGGCATACGAGATACGAAG | 1769 | AATACTICGT | 2153 |
| 3_41 | TATTGTCTCGTGGGCTCGG | | | |
| EasySci-RNA_P7- | CAAGCAGAAGACGGCATACGAGATAGACTT | 1770 | TGATAAGTCT | 2154 |
| 3_42 | ATCAGTCTCGTGGGCTCGG | | | |
| EasySci-RNA_P7- | CAAGCAGAAGACGGCATACGAGATTCGCGC | 1771 | TACGGCGCGA | 2155 |
| 3_43 | CGTAGTCTCGTGGGCTCGG | | | |
| EasySci-RNA_P7- | CAAGCAGAAGACGGCATACGAGATAGCTTG | 1772 | TCTTCAAGCT | 2156 |
| 3_44 | AAGAGTCTCGTGGGCTCGG | | | |
| EasySci-RNA_P7- | CAAGCAGAAGACGGCATACGAGATCGGTAG | 1773 | GTAGCTACCG | 2157 |
| 3_45 | CTACGTCTCGTGGGCTCGG | | | |
| EasySci-RNA_P7- | CAAGCAGAAGACGGCATACGAGATGCGGCA | 1774 | CGCATGCCGC | 2158 |
| 3_46 | TGCGGTCTCGTGGGCTCGG | | | |
| EasySci-RNA_P7- | CAAGCAGAAGACGGCATACGAGATAAGACT | 1775 | AGCCAGTCTT | 2159 |
| 3_47 | GGCTGTCTCGTGGGCTCGG | | | |
| EasySci-RNA_P7- | CAAGCAGAAGACGGCATACGAGATCGCATT | 1776 | TAAGAATGCG | 2160 |
| 3_48 | CTTAGTCTCGTGGGCTCGG | | | |
| EasySci-RNA_P7- | CAAGCAGAAGACGGCATACGAGATTACCGT | 1777 | GGAGACGGTA | 2161 |
| 3_49 | CTCCGTCTCGTGGGCTCGG | | | |
| EasySci-RNA_P7- | CAAGCAGAAGACGGCATACGAGATAATATA | 1778 | ATACTATATT | 2162 |
| 3_50 | GTATGTCTCGTGGGCTCGG | | | |
| EasySci-RNA_P7- | CAAGCAGAAGACGGCATACGAGATATCATA | 1779 | ATCTTATGAT | 2163 |
| 3_51 | AGATGTCTCGTGGGCTCGG | | | |
| EasySci-RNA_P7- | CAAGCAGAAGACGGCATACGAGATATCAAT | 1780 | GGATATTGAT | 2164 |
| 3_52 | ATCCGTCTCGTGGGCTCGG | | | |
| EasySci-RNA_P7- | CAAGCAGAAGACGGCATACGAGATTATTACC | 1781 | GTTGGTAATA | 2165 |
| 3_53 | AACGTCTCGTGGGCTCGG | | | |
| EasySci-RNA_P7- | CAAGCAGAAGACGGCATACGAGATGAGAAG | 1782 | TGGTCTTCTC | 2166 |
| 3_54 | ACCAGTCTCGTGGGCTCGG | | | |
| EasySci-RNA_P7- | CAAGCAGAAGACGGCATACGAGATTGGCGC | 1783 | GAGAGCGCCA | 2167 |
| 3_55 | TCTCGTCTCGTGGGCTCGG | | | |
| EasySci-RNA_P7- | CAAGCAGAAGACGGCATACGAGATGCGACG | 1784 | TTATCGTCGC | 2168 |
| 3_56 | ATAAGTCTCGTGGGCTCGG | | | |
| EasySci-RNA_P7- | CAAGCAGAAGACGGCATACGAGATAACGTC | 1785 | TCGCGACGTT | 2169 |
| 3_57 | GCGAGTCTCGTGGGCTCGG | | | |
| EasySci-RNA_P7- | CAAGCAGAAGACGGCATACGAGATATGAAG | 1786 | GAAGCTTCAT | 2170 |
| 3_58 | CTTCGTCTCGTGGGCTCGG | | | |
| EasySci-RNA_P7- | CAAGCAGAAGACGGCATACGAGATGCCATA | 1787 | ACTCTATGGC | 2171 |
| 3_59 | GAGTGTCTCGTGGGCTCGG | | | |
| EasySci-RNA_P7- | CAAGCAGAAGACGGCATACGAGATTGACTG | 1788 | TTCTCAGTCA | 2172 |
| 3_60 | AGAAGTCTCGTGGGCTCGG | | | |
| EasySci-RNA_P7- | CAAGCAGAAGACGGCATACGAGATCGGCTC | 1789 | TTCGGAGCCG | 2173 |
| 3_61 | CGAAGTCTCGTGGGCTCGG | | | |
| EasySci-RNA_P7- | CAAGCAGAAGACGGCATACGAGATCAGCCA | 1790 | CAATTGGCTG | 2174 |
| 3_62 | ATTGGTCTCGTGGGCTCGG | | | |
| EasySci-RNA_P7- | CAAGCAGAAGACGGCATACGAGATGCGACC | 1791 | AACTGGTCGC | 2175 |
| 3_63 | AGTTGTCTCGTGGGCTCGG | | | |
| EasySci-RNA_P7- | CAAGCAGAAGACGGCATACGAGATCCTAAC | 1792 | CGTCGTTAGG | 2176 |
| 3_64 | GACGGTCTCGTGGGCTCGG | | | |
| EasySci-RNA_P7- | CAAGCAGAAGACGGCATACGAGATCTTCGC | 1793 | GATTGCGAAG | 2177 |
| 3_65 | AATCGTCTCGTGGGCTCGG | | | |
| EasySci-RNA_P7- | CAAGCAGAAGACGGCATACGAGATTGACTC | 1794 | AACGGAGTCA | 2178 |
| 3_66 | CGTTGTCTCGTGGGCTCGG | | | |
| EasySci-RNA_P7- | CAAGCAGAAGACGGCATACGAGATGATAGT | 1795 | AGCGACTATC | 2179 |
| 3_67 | CGCTGTCTCGTGGGCTCGG | | | |
| EasySci-RNA_P7- | CAAGCAGAAGACGGCATACGAGATAAGGTA | 1796 | TTAGTACCTT | 2180 |
| 3_68 | CTAAGTCTCGTGGGCTCGG | | | |
| EasySci-RNA_P7- | CAAGCAGAAGACGGCATACGAGATTTGCAT | 1797 | CCTCATGCAA | 2181 |
| 3_69 | GAGGGTCTCGTGGGCTCGG | | | |
| EasySci-RNA_P7- | CAAGCAGAAGACGGCATACGAGATGTATTAT | 1798 | TATATAATAC | 2182 |
| 3_70 | ATAGTCTCGTGGGCTCGG | | | |
| EasySci-RNA_P7- | CAAGCAGAAGACGGCATACGAGATATTCTTG | 1799 | AGCCAAGAAT | 2183 |
| 3_71 | GCTGTCTCGTGGGCTCGG | | | |
| EasySci-RNA_P7- | CAAGCAGAAGACGGCATACGAGATTGCATCT | 1800 | CCAAGATGCA | 2184 |
| 3_72 | TGGGTCTCGTGGGCTCGG | | | |
| EasySci-RNA_P7- | CAAGCAGAAGACGGCATACGAGATGTTGGC | 1801 | TTGAGCCAAC | 2185 |
| 3_73 | TCAAGTCTCGTGGGCTCGG | | | |
| EasySci-RNA_P7- | CAAGCAGAAGACGGCATACGAGATAATATC | 1802 | TAATGATATT | 2186 |
| 3_74 | ATTAGTCTCGTGGGCTCGG | | | |
| EasySci-RNA_P7- | CAAGCAGAAGACGGCATACGAGATTAACTA | 1803 | GACTTAGTTA | 2187 |
| 3_75 | AGTCGTCTCGTGGGCTCGG | | | |
| EasySci-RNA_P7- | CAAGCAGAAGACGGCATACGAGATCAATAA | 1804 | TTGGTTATTG | 2188 |
| 3_76 | CCAAGTCTCGTGGGCTCGG | | | |
| EasySci-RNA_P7- | CAAGCAGAAGACGGCATACGAGATTTATACT | 1805 | TGCAGTATAA | 2189 |
| 3_77 | GCAGTCTCGTGGGCTCGG | | | |
| EasySci-RNA_P7- | CAAGCAGAAGACGGCATACGAGATTGAGCA | 1806 | GCTCTGCTCA | 2190 |
| 3_78 | GAGCGTCTCGTGGGCTCGG | | | |
| EasySci-RNA_P7- | CAAGCAGAAGACGGCATACGAGATTGCAAG | 1807 | TTGGCTTGCA | 2191 |
| 3_79 | CCAAGTCTCGTGGGCTCGG | | | |
| EasySci-RNA_P7- | CAAGCAGAAGACGGCATACGAGATTGGAGA | 1808 | TCGTTCTCCA | 2192 |
| 3_80 | ACGAGTCTCGTGGGCTCGG | | | |
| EasySci-RNA_P7- | CAAGCAGAAGACGGCATACGAGATATCGGA | 1809 | TGAATCCGAT | 2193 |
| 3_81 | TTCAGTCTCGTGGGCTCGG | | | |
| EasySci-RNA_P7- | CAAGCAGAAGACGGCATACGAGATACTAGA | 1810 | TCGGTCTAGT | 2194 |
| 3_82 | CCGAGTCTCGTGGGCTCGG | | | |
| EasySci-RNA_P7- | CAAGCAGAAGACGGCATACGAGATCGAGAT | 1811 | AAGCATCTCG | 2195 |
| 3_83 | GCTTGTCTCGTGGGCTCGG | | | |
| EasySci-RNA_P7- | CAAGCAGAAGACGGCATACGAGATTTCTATT | 1812 | ATTAATAGAA | 2196 |
| 3_84 | AATGTCTCGTGGGCTCGG | | | |
| EasySci-RNA_P7- | CAAGCAGAAGACGGCATACGAGATAATTAG | 1813 | TGGACTAATT | 2197 |
| 3_85 | TCCAGTCTCGTGGGCTCGG | | | |
| EasySci-RNA_P7- | CAAGCAGAAGACGGCATACGAGATGCTCCA | 1814 | GGCTTGGAGC | 2198 |
| 3_86 | AGCCGTCTCGTGGGCTCGG | | | |
| EasySci-RNA_P7- | CAAGCAGAAGACGGCATACGAGATCTCTTCC | 1815 | TTAGGAAGAG | 2199 |
| 3_87 | TAAGTCTCGTGGGCTCGG | | | |
| EasySci-RNA_P7- | CAAGCAGAAGACGGCATACGAGATCCGCGT | 1816 | GTTAACGCGG | 2200 |
| 3_88 | TAACGTCTCGTGGGCTCGG | | | |
| EasySci-RNA_P7- | CAAGCAGAAGACGGCATACGAGATGACGGA | 1817 | CTATTCCGTC | 2201 |
| 3_89 | ATAGGTCTCGTGGGCTCGG | | | |
| EasySci-RNA_P7- | CAAGCAGAAGACGGCATACGAGATCTCAGA | 1818 | CAACTCTGAG | 2202 |
| 3_90 | GTTGGTCTCGTGGGCTCGG | | | |
| EasySci-RNA_P7- | CAAGCAGAAGACGGCATACGAGATGGACGT | 1819 | TCATACGTCC | 2203 |
| 3_91 | ATGAGTCTCGTGGGCTCGG | | | |
| EasySci-RNA_P7- | CAAGCAGAAGACGGCATACGAGATAAGATG | 1820 | GACTCATCTT | 2204 |
| 3_92 | AGTCGTCTCGTGGGCTCGG | | | |
| EasySci-RNA_P7- | CAAGCAGAAGACGGCATACGAGATATGCGC | 1821 | GGTAGCGCAT | 2205 |
| 3_93 | TACCGTCTCGTGGGCTCGG | | | |
| EasySci-RNA_P7- | CAAGCAGAAGACGGCATACGAGATGGACGC | 1822 | TTAGGCGTCC | 2206 |
| 3_94 | CTAAGTCTCGTGGGCTCGG | | | |
| EasySci-RNA_P7- | CAAGCAGAAGACGGCATACGAGATCAGTTA | 1823 | GGTCTAACTG | 2207 |
| 3_95 | GACCGTCTCGTGGGCTCGG | | | |
| EasySci-RNA_P7- | CAAGCAGAAGACGGCATACGAGATCCGTCTC | 1824 | ATTGAGACGG | 2208 |
| 3_96 | AATGTCTCGTGGGCTCGG | | | |
| EasySci-RNA_P7- | CAAGCAGAAGACGGCATACGAGATATGGTA | 1825 | AACGTACCAT | 2209 |
| 4_01 | CGTTGTCTCGTGGGCTCGG | | | |
| EasySci-RNA_P7- | CAAGCAGAAGACGGCATACGAGATGTAACT | 1826 | GTTCAGTTAC | 2210 |
| 4_02 | GAACGTCTCGTGGGCTCGG | | | |
| EasySci-RNA_P7- | CAAGCAGAAGACGGCATACGAGATTAAGGT | 1827 | GTTAACCTTA | 2211 |
| 4_03 | TAACGTCTCGTGGGCTCGG | | | |
| EasySci-RNA_P7- | CAAGCAGAAGACGGCATACGAGATCTACTA | 1828 | GGAGTAGTAG | 2212 |
| 4_04 | CTCCGTCTCGTGGGCTCGG | | | |
| EasySci-RNA_P7- | CAAGCAGAAGACGGCATACGAGATTCTCAA | 1829 | CAGGTTGAGA | 2213 |
| 4_05 | CCTGGTCTCGTGGGCTCGG | | | |
| EasvSci-RNA_P7- | CAAGCAGAAGACGGCATACGAGATGTTATTG | 1830 | AACCAATAAC | 2214 |
| 4_06 | GTTGTCTCGTGGGCTCGG | | | |
| EasySci-RNA_P7- | CAAGCAGAAGACGGCATACGAGATAATAGG | 1831 | GGTACCTATT | 2215 |
| 4_07 | TACCGTCTCGTGGGCTCGG | | | |
| EasySci-RNA_P7- | CAAGCAGAAGACGGCATACGAGATTGAGGC | 1832 | AGCTGCCTCA | 2216 |
| 4_08 | AGCTGTCTCGTGGGCTCGG | | | |
| EasySci-RNA_P7- | CAAGCAGAAGACGGCATACGAGATTACCAA | 1833 | TTGGTTGGTA | 2217 |
| 4_09 | CCAAGTCTCGTGGGCTCGG | | | |
| EasySci-RNA_P7- | CAAGCAGAAGACGGCATACGAGATCCGATA | 1834 | CTGATATCGG | 2218 |
| 4_10 | TCAGGTCTCGTGGGCTCGG | | | |
| EasySci-RNA_P7- | CAAGCAGAAGACGGCATACGAGATGTTCCAT | 1835 | TTGATGGAAC | 2219 |
| 4_11 | CAAGTCTCGTGGGCTCGG | | | |
| EasySci-RNA_P7- | CAAGCAGAAGACGGCATACGAGATCTTCTGG | 1836 | GGACCAGAAG | 2220 |
| 4_12 | TCCGTCTCGTGGGCTCGG | | | |
| EasySci-RNA_P7- | CAAGCAGAAGACGGCATACGAGATGACCTC | 1837 | ACCTGAGGTC | 2221 |
| 4_13 | AGGTGTCTCGTGGGCTCGG | | | |
| EasySci-RNA_P7- | CAAGCAGAAGACGGCATACGAGATCTCATTG | 1838 | TTGCAATGAG | 2222 |
| 4_14 | CAAGTCTCGTGGGCTCGG | | | |
| EasySci-RNA_P7- | CAAGCAGAAGACGGCATACGAGATGTCAGT | 1839 | ACTAACTGAC | 2223 |
| 4_15 | TAGTGTCTCGTGGGCTCGG | | | |
| EasySci-RNA_P7- | CAAGCAGAAGACGGCATACGAGATACCTCTT | 1840 | GGTAAGAGGT | 2224 |
| 4_16 | ACCGTCTCGTGGGCTCGG | | | |
| EasySci-RNA_P7- | CAAGCAGAAGACGGCATACGAGATTTGCGA | 1841 | GTAATCGCAA | 2225 |
| 4_17 | TTACGTCTCGTGGGCTCGG | | | |
| EasySci-RNA_P7- | CAAGCAGAAGACGGCATACGAGATTTCATCA | 1842 | ATATGATGAA | 2226 |
| 4_18 | TATGTCTCGTGGGCTCGG | | | |
| EasySci-RNA_P7- | CAAGCAGAAGACGGCATACGAGATCTTCCGT | 1843 | CCTACGGAAG | 2227 |
| 4_19 | AGGGTCTCGTGGGCTCGG | | | |
| EasySci-RNA_P7- | CAAGCAGAAGACGGCATACGAGATTCGGAG | 1844 | GACTCTCCGA | 2228 |
| 4_20 | AGTCGTCTCGTGGGCTCGG | | | |
| EasySci-RNA_P7- | CAAGCAGAAGACGGCATACGAGATACGTAT | 1845 | ATAGATACGT | 2229 |
| 4_21 | CTATGTCTCGTGGGCTCGG | | | |
| EasySci-RNA_P7- | CAAGCAGAAGACGGCATACGAGATTTGCTTC | 1846 | TATGAAGCAA | 2230 |
| 4_22 | ATAGTCTCGTGGGCTCGG | | | |
| EasySci-RNA_P7- | CAAGCAGAAGACGGCATACGAGATTCGTCTC | 1847 | GTAGAGACGA | 2231 |
| 4_23 | TACGTCTCGTGGGCTCGG | | | |
| EasySci-RNA_P7- | CAAGCAGAAGACGGCATACGAGATGTTATG | 1848 | TTCGCATAAC | 2232 |
| 4_24 | CGAAGTCTCGTGGGCTCGG | | | |
| EasySci-RNA_P7- | CAAGCAGAAGACGGCATACGAGATGGCGAA | 1849 | TAGATTCGCC | 2233 |
| 4_25 | TCTAGTCTCGTGGGCTCGG | | | |
| EasySci-RNA_P7- | CAAGCAGAAGACGGCATACGAGATCCGCGA | 1850 | TTCTTCGCGG | 2234 |
| 4_26 | AGAAGTCTCGTGGGCTCGG | | | |
| EasySci-RNA_P7- | CAAGCAGAAGACGGCATACGAGATAGACCA | 1851 | TTCTTGGTCT | 2235 |
| 4_27 | AGAAGTCTCGTGGGCTCGG | | | |
| EasySci-RNA_P7- | CAAGCAGAAGACGGCATACGAGATTAATCT | 1852 | GTATAGATTA | 2236 |
| 4_28 | ATACGTCTCGTGGGCTCGG | | | |
| EasySci-RNA_P7- | CAAGCAGAAGACGGCATACGAGATAGTCAT | 1853 | GACTATGACT | 2237 |
| 4_29 | AGTCGTCTCGTGGGCTCGG | | | |
| EasySci-RNA_P7- | CAAGCAGAAGACGGCATACGAGATTTCGCG | 1854 | GCTCCGCGAA | 2238 |
| 4_30 | GAGCGTCTCGTGGGCTCGG | | | |
| EasySci-RNA_P7- | CAAGCAGAAGACGGCATACGAGATGAATCG | 1855 | GGAACGATTC | 2239 |
| 4_31 | TTCCGTCTCGTGGGCTCGG | | | |
| EasySci-RNA_P7- | CAAGCAGAAGACGGCATACGAGATACGAAG | 1856 | GTACCTTCGT | 2240 |
| 4_32 | GTACGTCTCGTGGGCTCGG | | | |
| EasySci-RNA_P7- | CAAGCAGAAGACGGCATACGAGATAGTCGC | 1857 | TTATGCGACT | 2241 |
| 4_33 | ATAAGTCTCGTGGGCTCGG | | | |
| EasySci-RNA_P7- | CAAGCAGAAGACGGCATACGAGATACCAAC | 1858 | AACGGTTGGT | 2242 |
| 4_34 | CGTTGTCTCGTGGGCTCGG | | | |
| EasySci-RNA_P7- | CAAGCAGAAGACGGCATACGAGATTTCCTTC | 1859 | CTAGAAGGAA | 2243 |
| 4_35 | TAGGTCTCGTGGGCTCGG | | | |
| EasySci-RNA_P7- | CAAGCAGAAGACGGCATACGAGATTCCTCCA | 1860 | GTATGGAGGA | 2244 |
| 4_36 | TACGTCTCGTGGGCTCGG | | | |
| EasySci-RNA_P7- | CAAGCAGAAGACGGCATACGAGATGCTACTT | 1861 | CGTAAGTAGC | 2245 |
| 4_37 | ACGGTCTCGTGGGCTCGG | | | |
| EasySci-RNA_P7- | CAAGCAGAAGACGGCATACGAGATTTGACG | 1862 | TAGTCGTCAA | 2246 |
| 4_38 | ACTAGTCTCGTGGGCTCGG | | | |
| EasySci-RNA_P7- | CAAGCAGAAGACGGCATACGAGATTCCATA | 1863 | GTAGTATGGA | 2247 |
| 4_39 | CTACGTCTCGTGGGCTCGG | | | |
| EasySci-RNA_P7- | CAAGCAGAAGACGGCATACGAGATTACGTC | 1864 | AATGGACGTA | 2248 |
| 4_40 | CATTGTCTCGTGGGCTCGG | | | |
| EasySci-RNA_P7- | CAAGCAGAAGACGGCATACGAGATCAGCGA | 1865 | CCGTTCGCTG | 2249 |
| 4_41 | ACGGGTCTCGTGGGCTCGG | | | |
| EasySci-RNA_P7- | CAAGCAGAAGACGGCATACGAGATATATTG | 1866 | CAGTCAATAT | 2250 |
| 4_42 | ACTGGTCTCGTGGGCTCGG | | | |
| EasySci-RNA_P7- | CAAGCAGAAGACGGCATACGAGATTCAGTC | 1867 | GTCGGACTGA | 2251 |
| 4_43 | CGACGTCTCGTGGGCTCGG | | | |
| EasySci-RNA_P7- | CAAGCAGAAGACGGCATACGAGATGCGCAT | 1868 | TTCCATGCGC | 2252 |
| 4_44 | GGAAGTCTCGTGGGCTCGG | | | |
| EasySci-RNA_P7- | CAAGCAGAAGACGGCATACGAGATCATGCC | 1869 | GGACGGCATG | 2253 |
| 4_45 | GTCCGTCTCGTGGGCTCGG | | | |
| EasySci-RNA_P7- | CAAGCAGAAGACGGCATACGAGATACGTTG | 1870 | GGAGCAACGT | 2254 |
| 4_46 | CTCCGTCTCGTGGGCTCGG | | | |
| EasySci-RNA_P7- | CAAGCAGAAGACGGCATACGAGATAGCTAG | 1871 | CGTCCTAGCT | 2255 |
| 4_47 | GACGGTCTCGTGGGCTCGG | | | |
| EasySci-RNA_P7- | CAAGCAGAAGACGGCATACGAGATCTACTA | 1872 | ATATTAGTAG | 2256 |
| 4_48 | ATATGTCTCGTGGGCTCGG | | | |
| EasySci-RNA_P7- | CAAGCAGAAGACGGCATACGAGATAGAAGG | 1873 | AGTTCCTTCT | 2257 |
| 4_49 | AACTGTCTCGTGGGCTCGG | | | |
| EasySci-RNA_P7- | CAAGCAGAAGACGGCATACGAGATCCTTGA | 1874 | GCCTTCAAGG | 2258 |
| 4_50 | AGGCGTCTCGTGGGCTCGG | | | |
| EasySci-RNA_P7- | CAAGCAGAAGACGGCATACGAGATTGAGGC | 1875 | TAACGCCTCA | 2259 |
| 4_51 | GTTAGTCTCGTGGGCTCGG | | | |
| EasySci-RNA_P7- | CAAGCAGAAGACGGCATACGAGATAGACGT | 1876 | GAATACGTCT | 2260 |
| 4_52 | ATTCGTCTCGTGGGCTCGG | | | |
| EasySci-RNA_P7- | CAAGCAGAAGACGGCATACGAGATAAGGCT | 1877 | GATGAGCCTT | 2261 |
| 4_53 | CATCGTCTCGTGGGCTCGG | | | |
| EasySci-RNA_P7- | CAAGCAGAAGACGGCATACGAGATAGTCTC | 1878 | TACGGAGACT | 2262 |
| 4_54 | CGTAGTCTCGTGGGCTCGG | | | |
| EasySci-RNA_P7- | CAAGCAGAAGACGGCATACGAGATAATGAC | 1879 | AGAGGTCATT | 2263 |
| 4_55 | CTCTGTCTCGTGGGCTCGG | | | |
| EasySci-RNA_P7- | CAAGCAGAAGACGGCATACGAGATTAACTG | 1880 | CGGCCAGTTA | 2264 |
| 4_56 | GCCGGTCTCGTGGGCTCGG | | | |
| EasySci-RNA_P7- | CAAGCAGAAGACGGCATACGAGATTTAAGC | 1881 | TAGCGCTTAA | 2265 |
| 4_57 | GCTAGTCTCGTGGGCTCGG | | | |
| EasySci-RNA_P7- | CAAGCAGAAGACGGCATACGAGATCATAAG | 1882 | CAACCTTATG | 2266 |
| 4_58 | GTTGGTCTCGTGGGCTCGG | | | |
| EasySci-RNA_P7- | CAAGCAGAAGACGGCATACGAGATTTCGTCG | 1883 | CTTCGACGAA | 2267 |
| 4_59 | AAGGTCTCGTGGGCTCGG | | | |
| EasySci-RNA_P7- | CAAGCAGAAGACGGCATACGAGATCTCGGA | 1884 | AGAGTCCGAG | 2268 |
| 4_60 | CTCTGTCTCGTGGGCTCGG | | | |
| EasySci-RNA_P7- | CAAGCAGAAGACGGCATACGAGATCGAACC | 1885 | CTATGGTTCG | 2269 |
| 4_61 | ATAGGTCTCGTGGGCTCGG | | | |
| EasySci-RNA_P7- | CAAGCAGAAGACGGCATACGAGATGCCAAT | 1886 | AACTATTGGC | 2270 |
| 4_62 | AGTTGTCTCGTGGGCTCGG | | | |
| EasySci-RNA_P7- | CAAGCAGAAGACGGCATACGAGATACCTCG | 1887 | CTTGCGAGGT | 2271 |
| 4_63 | CAAGGTCTCGTGGGCTCGG | | | |
| EasySci-RNA_P7- | CAAGCAGAAGACGGCATACGAGATACGAGC | 1888 | TTCGGCTCGT | 2272 |
| 4_64 | CGAAGTCTCGTGGGCTCGG | | | |
| EasySci-RNA_P7- | CAAGCAGAAGACGGCATACGAGATCAGACT | 1889 | CTCAAGTCTG | 2273 |
| 4_65 | TGAGGTCTCGTGGGCTCGG | | | |
| EasySci-RNA_P7- | CAAGCAGAAGACGGCATACGAGATCAGGCC | 1890 | ATTAGGCCTG | 2274 |
| 4_66 | TAATGTCTCGTGGGCTCGG | | | |
| EasySci-RNA_P7- | CAAGCAGAAGACGGCATACGAGATACGTTA | 1891 | TGGCTAACGT | 2275 |
| 4_67 | GCCAGTCTCGTGGGCTCGG | | | |
| EasySci-RNA_P7- | CAAGCAGAAGACGGCATACGAGATAATATT | 1892 | AGCTAATATT | 2276 |
| 4_68 | AGCTGTCTCGTGGGCTCGG | | | |
| EasySci-RNA_P7- | CAAGCAGAAGACGGCATACGAGATGAAGAT | 1893 | AGGAATCTTC | 2277 |
| 4_69 | TCCTGTCTCGTGGGCTCGG | | | |
| EasySci-RNA_P7- | CAAGCAGAAGACGGCATACGAGATGTCTGG | 1894 | AAGACCAGAC | 2278 |
| 4_70 | TCTTGTCTCGTGGGCTCGG | | | |
| EasySci-RNA_P7- | CAAGCAGAAGACGGCATACGAGATCGCTTAT | 1895 | ACCATAAGCG | 2279 |
| 4_71 | GGTGTCTCGTGGGCTCGG | | | |
| EasySci-RNA_P7- | CAAGCAGAAGACGGCATACGAGATGTAATA | 1896 | TGCTTATTAC | 2280 |
| 4_72 | AGCAGTCTCGTGGGCTCGG | | | |
| EasySci-RNA_P7- | CAAGCAGAAGACGGCATACGAGATAATGCT | 1897 | ATAGAGCATT | 2281 |
| 4_73 | CTATGTCTCGTGGGCTCGG | | | |
| EasySci-RNA_P7- | CAAGCAGAAGACGGCATACGAGATCAAGAT | 1898 | AATTATCTTG | 2282 |
| 4_74 | AATTGTCTCGTGGGCTCGG | | | |
| EasySci-RNA_P7- | CAAGCAGAAGACGGCATACGAGATCGCGCG | 1899 | GTTCCGCGCG | 2283 |
| 4_75 | GAACGTCTCGTGGGCTCGG | | | |
| EasySci-RNA_P7- | CAAGCAGAAGACGGCATACGAGATCGGACT | 1900 | CCGAAGTCCG | 2284 |
| 4_76 | TCGGGTCTCGTGGGCTCGG | | | |
| EasySci-RNA_P7- | CAAGCAGAAGACGGCATACGAGATATCATG | 1901 | TTACCATGAT | 2285 |
| 4_77 | GTAAGTCTCGTGGGCTCGG | | | |
| EasySci-RNA_P7- | CAAGCAGAAGACGGCATACGAGATGCCATC | 1902 | TATAGATGGC | 2286 |
| 4_78 | TATAGTCTCGTGGGCTCGG | | | |
| EasySci-RNA_P7- | CAAGCAGAAGACGGCATACGAGATCCAGAT | 1903 | GCATATCTGG | 2287 |
| 4_79 | ATGCGTCTCGTGGGCTCGG | | | |
| EasySci-RNA_P7- | CAAGCAGAAGACGGCATACGAGATCTTCTAG | 1904 | ACTCTAGAAG | 2288 |
| 4_80 | AGTGTCTCGTGGGCTCGG | | | |
| EasySci-RNA_P7- | CAAGCAGAAGACGGCATACGAGATCATATTC | 1905 | CAAGAATATG | 2289 |
| 4_81 | TTGGTCTCGTGGGCTCGG | | | |
| EasySci-RNA_P7- | CAAGCAGAAGACGGCATACGAGATCGCGCA | 1906 | CTGCTGCGCG | 2290 |
| 4_82 | GCAGGTCTCGTGGGCTCGG | | | |
| EasySci-RNA_P7- | CAAGCAGAAGACGGCATACGAGATCCGTAA | 1907 | CATATTACGG | 2291 |
| 4_83 | TATGGTCTCGTGGGCTCGG | | | |
| EasySci-RNA_P7- | CAAGCAGAAGACGGCATACGAGATCATTCC | 1908 | CGGCGGAATG | 2292 |
| 4_84 | GCCGGTCTCGTGGGCTCGG | | | |
| EasySci-RNA_P7- | CAAGCAGAAGACGGCATACGAGATTTCAGA | 1909 | TGCTTCTGAA | 2293 |
| 4_85 | AGCAGTCTCGTGGGCTCGG | | | |
| EasySci-RNA_P7- | CAAGCAGAAGACGGCATACGAGATAGAAGA | 1910 | GTATTCTTCT | 2294 |
| 4_86 | ATACGTCTCGTGGGCTCGG | | | |
| EasySci-RNA_P7- | CAAGCAGAAGACGGCATACGAGATATTACT | 1911 | CAGTAGTAAT | 2295 |
| 4_87 | ACTGGTCTCGTGGGCTCGG | | | |
| EasySci-RNA_P7- | CAAGCAGAAGACGGCATACGAGATGTCTCC | 1912 | CCGCGGAGAC | 2296 |
| 4_88 | GCGGGTCTCGTGGGCTCGG | | | |
| EasySci-RNA_P7- | CAAGCAGAAGACGGCATACGAGATGCCGAC | 1913 | GCTCGTCGGC | 2297 |
| 4_89 | GAGCGTCTCGTGGGCTCGG | | | |
| EasySci-RNA_P7- | CAAGCAGAAGACGGCATACGAGATTGCCTCT | 1914 | CTTAGAGGCA | 2298 |
| 4_90 | AAGGTCTCGTGGGCTCGG | | | |
| EasySci-RNA_P7- | CAAGCAGAAGACGGCATACGAGATCGTCTTG | 1915 | GACCAAGACG | 2299 |
| 4_91 | GTCGTCTCGTGGGCTCGG | | | |
| EasySci-RNA_P7- | CAAGCAGAAGACGGCATACGAGATACCATC | 1916 | AGCAGATGGT | 2300 |
| 4_92 | TGCTGTCTCGTGGGCTCGG | | | |
| EasySci-RNA_P7- | CAAGCAGAAGACGGCATACGAGATATGGTT | 1917 | AATTAACCAT | 2301 |
| 4_93 | AATTGTCTCGTGGGCTCGG | | | |
| EasySci-RNA_P7- | CAAGCAGAAGACGGCATACGAGATAGCGAG | 1918 | CAGACTCGCT | 2302 |
| 4_94 | TCTGGTCTCGTGGGCTCGG | | | |
| EasySci-RNA_P7- | CAAGCAGAAGACGGCATACGAGATAACGGC | 1919 | CTTCGCCGTT | 2303 |
| 4_95 | GAAGGTCTCGTGGGCTCGG | | | |
| EasySci-RNA_P7- | CAAGCAGAAGACGGCATACGAGATGGTTGG | 1920 | CCTGCCAACC | 2304 |
| 4_96 | CAGGGTCTCGTGGGCTCGG | | | |

### PCR (45 m))

Add 20 μL NEBNext Master Mix into each well and pipet up and down. Place samples into a thermocycler and run the following reaction:
   72 C for 5 minutes
   98 C for 30 seconds
   12-15 cycles of 98 C for 10 seconds, 66 C for 30 seconds, 72 C for 30 seconds
   72 C for 5 minutes
   *may be helpful to run a qPCR to determine the optimal number of cycles for amplification*.
Can store the resulting PCR products in −20 C (STOP POINT).

### Library Purification (1 h)

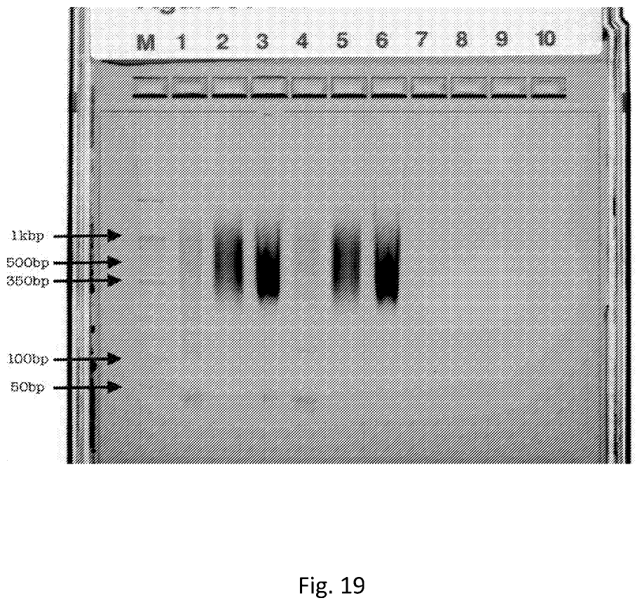

Pool all the wells together and take 200 μL of the PCR product and perform a 0.8× ampure beads purification: start with adding 160 μL beads to the 200 μL of solution. Mix the solution via vortexing and let the resulting solution sit at room temperature for 5 minutes.
Place the solution on a magnetic rack and let the solution sit for 5 minutes until the beads are removed from the solution.
Aspirate and remove the solution, making sure not to touch the beads. Add 1 mL of 80% ethanol to rinse beads and then remove the ethanol.
Add 1 mL of 80% ethanol for a second wash and then remove the ethanol.
Elute the bead using 105 μL of elution buffer and mix by vortexing. Let the resulting solution sit at room temperature for 3 minutes.
Place the solution on the magnetic rack, and let the solution incubate for 3 minutes.
Transfer 100 μL of the solution into a new tube and add 90 μL ampure beads for a second, 0.9× ampure beads purification. Vortex to mix and let the solution sit at room temperature for 5 minutes.
Place the solution on a magnetic rack and let the solution sit for 5 minutes. Afterwards, aspirate the supernatant.
Wash twice with 1 mL 80% ethanol and then add 20 μL EB buffer to the tube and vortex. Let the solution sit for 3 minutes at room temperature.
Place the solution on the magnetic rack and let the solution sit for 3 minutes. Take out 18 μL of the remaining solution and transfer it to a new tube.
Quantify the library concentration and visualize the library via electrophoresis (performed using a Qubit and a 2% Agarose E-Gel). An example library is shown in FIG. 19.
Sequence the library on the Novaseq Platform.

### Example 3: Tracking Cell-Type-Specific Proliferation and Differentiation Dynamics in Mammalian Brains Across the Lifespan

Herein is described a novel method, TrackerSci, to track the proliferation and differentiation dynamics of newborn cells at the scale of the entire mammalian brain. TrackerSci integrated protocols for labeling newly synthesized DNA with a thymidine analog 5-Ethynyl-2-deoxyuridine (EdU) (Salic et al., Proc. Natl. Acad. Sci. U.S.A 105, 2415-2420 (2008)) and single-cell combinatorial indexing sequencing for both transcriptome (Cao et al., Nature 566, 496-502 (2019)) and chromatin accessibility profiling (Domcke et al., Science 370, (2020)). As a demonstration, TrackerSci was applied to profile the single-cell transcriptome or chromatin accessibility dynamics for a total of 14,689 newborn cells from entire mouse brains spanning three age stages and two genotypes. With the resulting datasets, rare progenitor cell populations often missed in conventional single-cell analysis were recovered and their cell-type-specific proliferation and differentiation dynamics were tracked across conditions. Furthermore, the genetic and epigenetic signatures associated with the alteration of cellular dynamics (e.g., adult neurogenesis, oligodendrogenesis) upon ageing were identified. The experimental and computational methods described here could be broadly applied to track the regenerative capacity and differentiation potential of cells across main mammalian organs and other biological systems.

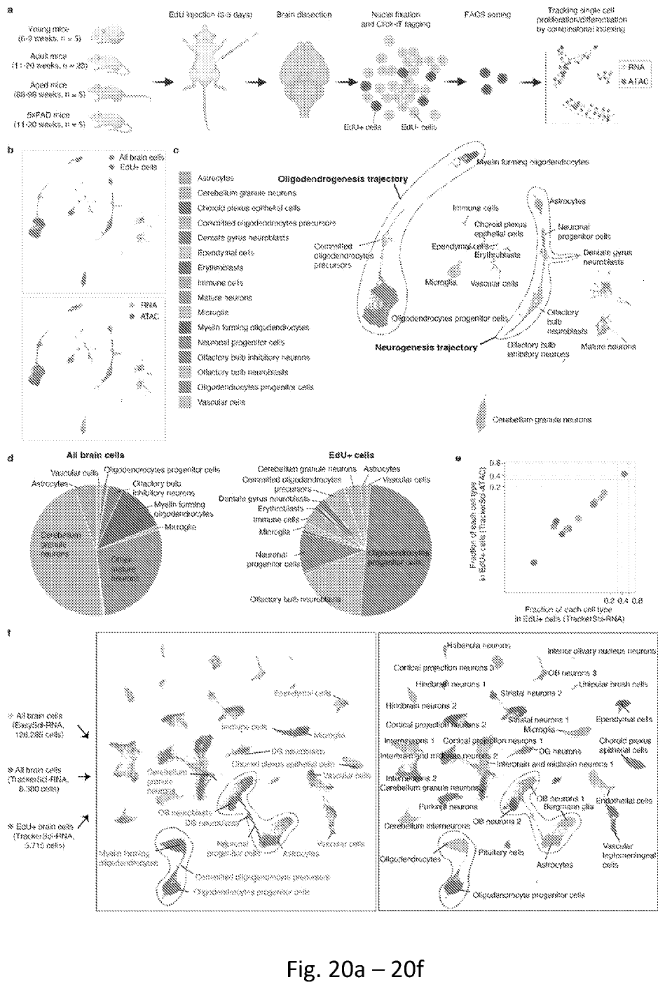

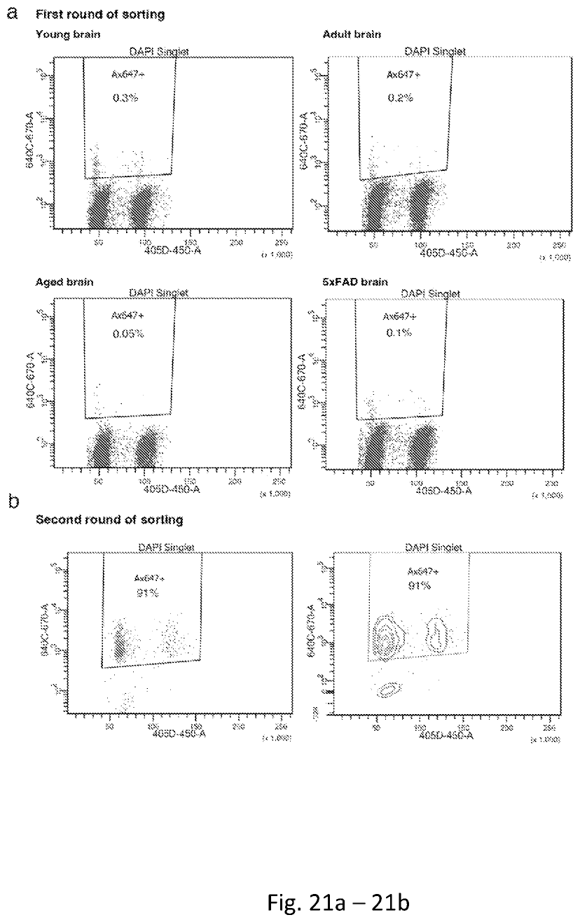

TrackerSci relies on the following steps (FIG. 20a): (i) Mice are labeled with 5-Ethynyl-2-deoxyuridine (EdU), a thymidine analog that can be incorporated into replicating DNA for labeling in vivo cellular proliferation (Salic et al., Proc. Natl. Acad. Sci. U.S.A 105, 2415-2420 (2008); Lin et al., Cytotherapy 11, 864-873 (2009)). (ii) Brain are dissected, and nuclei are extracted, fixed, and then subjected to click chemistry-based in situ ligation (Clarke et al., Curr. Protoc. Cytom. 82, 7.49.1-7.49.30 (2017)) to an azide-containing fluorophore, followed by fluorescence-activated cell sorting (FACS) to enrich the EdU+ cells (FIG. 21a). (iii) Indexed reverse transcription or transposition is used to introduce the first round of indexing. Cells from all wells are pooled and then redistributed into multiple 96-well plates through FACS sorting to further purify the EdU+ cells (FIG. 21b). (iv) Library preparation protocols were followed similar to sci-RNA-seq (Cao et al., Nature 566, 496-502 (2019)) for transcriptome profiling or sci-ATAC-seq (Domcke et al., Science 370, (2020)) for chromatin accessibility analysis. Most cells pass through a unique combination of wells, such that their contents are marked by a unique combination of barcodes that can be used to group reads derived from the same cell. Notably, the two sorting steps implemented in TrackerSci are essential for excluding contaminating cells and enriching extremely rare proliferating cell populations, especially in the aged brain (less than 0.1% of the total cell population are EdU+ cells).

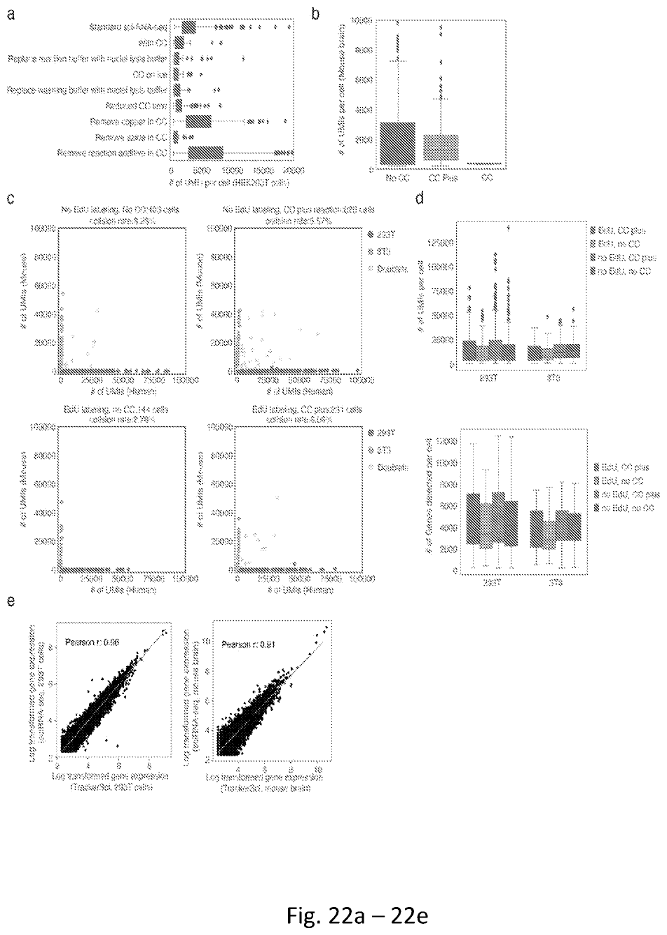

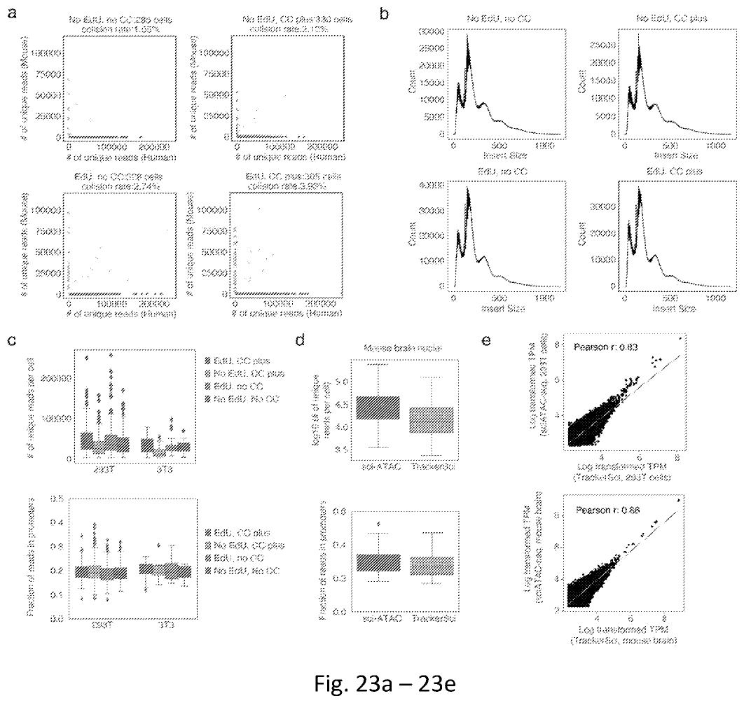

The reaction conditions were extensively optimized (e.g., fixation, permeabilization, and click-chemistry reaction) to ensure the approach is fully compatible with FACS sorting and single-cell transcriptome and chromatin accessibility profiling (FIG. 22-FIG. 23). For instance, the active Cu(I) catalyst and additive included in the conventional click-chemistry reaction (Habib et al., Science 353, 925-928 (2016)) significantly reduced the nuclei quality for single-cell gene expression analysis (FIG. 22a). To solve this problem, a click-chemistry method was tested using picolyl azide dye and copper protectant, which resulted in a minimal defect on library complexity (FIG. 22b) or cell purity for single-cell RNA-seq analysis, as shown in an experiment profiling a mixture of human HEK293T and mouse NIH/3T3 cells (FIG. 22c,d). As a quality control, the TrackerSci chromatin accessibility profile was compared with the conventional sci-ATAC-seq profile in a mixture of human HEK293T and mouse NIH/3T3 cells. Both methods showed similar cellular purity (FIG. 23a), fragment length distributions (FIG. 23b), a comparable number of unique fragments per cell, and a similar ratio of reads overlapping with promoters in both cell lines and mouse brain nuclei (FIG. 23c, d).

Additionally, the aggregated transcriptome and chromatin accessibility profiles derived from TrackerSci (both cultured cell lines and tissues) were highly correlated with conventional single-cell combinatorial indexing profiling (FIG. 22e, FIG. 23e), suggesting that the labeling and conjugating reactions (e.g., EdU labeling and click-chemistry) in TrackerSci do not substantially interfere with downstream single-cell transcriptome and chromatin accessibility profiling by combinatorial indexing.

The analysis illustrates the unique advantage of TrackerSci over solely profiling global brain populations. For example, TrackerSci enabled reconstruction of continuous cellular differentiation trajectories in adult or even aged organs by detecting intermediate progenitor cell states that are often missed in traditional single-cell analysis. Moreover, it was possible to calculate the proliferation and differentiation potential of rare progenitor cells, facilitating the quantitative investigation of the impact of ageing on adult neurogenesis and oligodendrogenesis. In addition, age-dependent changes in cell-type-specific proliferation and differentiation dynamics were investigated and novel insights into underlying transcriptional and epigenetic mechanisms are provided.

The field of single-cell biology is progressing at an astonishing rate to catalog and characterize every single cell type across diverse biological systems. Although the adult or aged brains have been intensively profiled with single-cell methods (Saunders et al., Cell 174, 1015-1030.e16 (2018); Zeisel et al., Cell 174, 999-1014.e22 (2018); Li et al., Nature 598, 129-136 (2021)), capturing progenitor cells and revealing their proliferation and differentiation dynamics has been challenging. The TrackerSci method is the first technique to track both transcriptional and epigenetic dynamics of proliferating cells based on combinatorial indexing. Like other sci-seq techniques (Cao et al., Science 370, (2020); Domcke et al., Science 370, (2020)), TrackerSci is compatible with fresh or fixed nuclei, and can process multiple samples concurrently per experiment to reduce the batch effect. In this study, TrackerSci was applied to profile the single-cell transcriptome or chromatin accessibility dynamics for a total of 14,689 newborn cells from entire mouse brains spanning three age stages and two genotypes. Considering the rarity of the progenitor cells in the adult and aged brains, it required deep sequencing of up to 15 million brain cells to recover the same amount of progenitor cells.

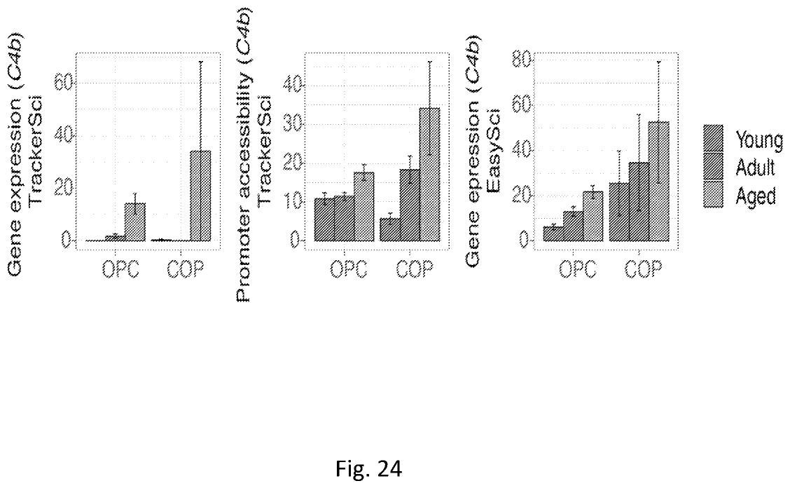

There is a consensus that the self-renewal and regeneration capacity of progenitor cells reduces during aging. By a comprehensive and quantitative view of the cell-type-specific proliferation and differentiation dynamics, however, a heterogeneous cell response to ageing was observed across newborn cell types. While ageing impairs neurogenesis mainly through a depleted pool of neuronal progenitors as expected, newborn oligodendrocyte progenitors were found to be mildly affected. Instead, the intermediate differentiation precursors are remarkably lower in frequency, suggesting that ageing affects oligodendrocytes mainly by blocking their differentiation process. Intriguingly, an age-dependent increase of Smpd4 (sphingomyelin synthase) and a decrease of Sgms1 (sphingomyelin phosphodiesterase) in the oligodendrocytes progenitor cells was detected, indicating a high cellular ceramide level in the aged OPCs. The data suggest a critical role of sphingomyelin metabolism in ageing-induced block of oligodendrocyte differentiation. In addition, dysregulated immune responses during ageing, such as the accelerated proliferation of an Apoe+Csf1+ microglia subtype and an increased C4b expression in OPCs from both the EdU+ population and the global pool was detected (FIG. 24). Further investigation could be helpful in deciphering the links between increased inflammation burden and the failure of oligodendrocyte differentiation in the aged brain.

In summary, the study represents a crucial step toward understanding the impact of ageing on the proliferation and differentiation of newborn cells across the entire brain. The continued development of methods and integration of other sci-seq techniques for concurrent profiling gene expression and chromatin accessibility state in concert with spatial, proteomics, and lineage history will facilitate a comprehensive view of the global molecular programs regulating cell-type-specific proliferation and differential dynamics during ageing, thereby informing potential pathways to restore tissue homeostasis for patients with ageing-related diseases.

The Materials and Methods used for the experiments are now described.

### Data Reporting

No statistical methods were used to predetermine sample size. Animals used in experiments were randomized before sample preparation. Investigators were blinded to group allocation during data collection and analysis.

### Animal

The C57BL/6 mice were obtained from The Jackson Laboratory.

### EdU Labeling of Mammalian Cell Culture

HEK293T and NIH/3T3 cells (gift from B. Martin, University of Washington) were cultured in 10 cm dishes at 37° C. with 5% CO₂ in high glucose DMEM (Gibco, 11965-118) supplemented with 10% Fetal Bovine Serum (Sigma-Aldrich, F4135) and 1× penicillin-streptomycin (Gibco, 15140-122).

EdU (5-ethynyl-2′-deoxyuridine) (Thermo Fisher Scientific, A10044) was added to culture media at 10 μM final concentration for 1 hour. After labeling, cells were harvested with 0.25% trypsin-EDTA. HEK293T and NIH/3T3 cells were combined at a 1:1 ratio, washed with ice-cold PBS, and lysed in 1 mL ice-cold EZ lysis buffer (Millipore Sigma, NUC101). The nuclei were then fixed on ice with 1% formaldehyde (Thermo Fisher Scientific, 28906) for 10 minutes and washed with EZ lysis buffer, filtered with 40 μm cell strainers (Ward's Science, 470236-276), and resuspended in Nuclei Suspension Buffer (NSB) (10 mM Tris-HCl pH 7.5 (VWR, 97062-936), 10 mM NaCl (VWR, 97062-858), 3 mM MgCl₂ (VWR, 97062-848) supplemented with 0.1% SUPERase⋅In™ RNase Inhibitor (Thermo Fisher Scientific, AM2696) and 1% BSA for TrackerSci-RNA or supplemented with 0.1% Tween-20 (Sigma, P9416-100ML), 1× cOmplete™, EDTA-free Protease Inhibitor Cocktail (Sigma, 11873580001) and 0.1% IGEPAL® CA-630 (VWR, IC0219859650) for TrackerSci-ATAC experiment.

### EdU Labeling of Mouse Tissues

C57BL/6J mice of different age groups and 5×FAD transgenic mice (MMRRC Strain #034840-JAX) were obtained from The Jackson Laboratory. Mice were injected intraperitoneally with 50 mg/kg of EdU in PBS at 24-hour intervals for five days, and mouse brains were harvested 24 hours after the final injection.

C57BL/6J mice obtained from The Jackson Laboratory were labeled and harvested for pulse-chase labeling at various time points. Specifically, four mice (two male and two female) were injected intraperitoneally with 50 mg/kg of EdU in PBS for 3 days at 24-hour intervals, and brains were harvested 24 hours after the final injection. 12 mice were injected intraperitoneally with 50 mg/kg of EdU in PBS for five days at 24-hour intervals. In addition, for five-day injections, four mice (two male and two female) were harvested 1 day, 3 days, and 5 days after the final injection.

### Tissue Collection and Nuclei Isolation

Whole brains were extracted from mice, immediately snap-frozen in liquid nitrogen, and stored at −80° C. upon further usage. For nuclei isolations, thawed brains were cut into small pieces with fine scissors (Fine Science Tools, 14060-09) in 1 mL ice-cold PBS with 1% SUPERase⋅In™ RNase Inhibitor and 1% BSA, pelleted, resuspended in 1.5 mL Nuclei Isolation Buffer (EZ Lysis Buffer supplemented with 1% SUPERase⋅In™ RNase Inhibitor, 1% BSA and 1× cOmplete™ EDTA-free Protease Inhibitor Cocktail) for 5 minutes on ice, and homogenized through 40 μm cell strainers (VWR, 470236-276) with the rubber tips of syringes. Then, extracted nuclei were pelleted, fixed in 1% formaldehyde on ice for 10 minutes, washed twice with NSB, and divided into two aliquots for both sci-RNA-seq and sci-ATAC-seq profiling. Nuclei subjected to sci-RNA-seq were briefly sonicated (Diagenode, low power mode for 12 seconds) to reduce clumping. Finally, nuclei were filtered through pluriStrainer Mini 20 um filters (Pluriselect, 43-10020-70), resuspended in 100 μL NSB, snap frozen in liquid nitrogen, and stored at −80° C. until further usage.

### TrackerSci-RNA

EdU staining was performed on thawed nuclei using Click-iT Plus EdU Alexa Fluor™ 647 Flow Cytometry assay Kit (Thermo Fisher Scientific, 10634). A 500 μL reaction buffer (prepared following the manufacturer's protocol) supplemented with 1% SUPERase⋅In™ RNase Inhibitor was added directly to the nuclei suspension, mixed well and left in RT for 30 minutes. Then, nuclei were spun down for 5 minutes at 500 g (4° C.), washed once with 500 μL of 1× Click-iT saponin-based permeabilization and wash reagent, resuspended in 1 mL NSB with 1:20 dilution of 0.25 mg/ml 4′,6-diamidino-2-phenylindole (DAPI, Invitrogen D1306) and FACS sorted. Alexa647 and DAPI positive nuclei were sorted into 96-well plates with each well (250˜500 nuclei/well) containing 4 μL of NSB. Sorted plates were briefly centrifuged, mixed with 1 μL of 50 μM oligo-dT primer (5′-(SEQ ID NO: 2447) ACGACGCTCTTCCGATCTNNNNNNNN [10 bp-index] TTTTTTTTTTTTTTTTTTTTTTTTTTTTTTVN-3′ (SEQ ID NO:2448), where “N” is any base and “V” is either “A”, “C” or “G”, IDT) and 0.5 μL 10 mM dNTP mix (Thermo Fisher Scientific, R0194) and denatured at 55° C. for 5 minutes and immediately placed on ice. 3.5 μL of first-strand reaction mix, containing 2 μL 5× SuperScript™ IV Reverse Transcriptase Buffer (Invitrogen, 18090200), 0.5 μL 100 mM DTT (Invitrogen, P2325), 0.5 μL SuperScript™ IV Reverse Transcriptase (Invitrogen, 18090200), 0.5 μL RNaseOUT™ Recombinant Ribonuclease Inhibitor (Invitrogen, 10777019) was then added to each well. Reverse transcription was carried out by incubating plates at the following temperature gradient: 4° C. 2 minutes, 10° C. 2 minutes, 20° C. 2 minutes, 30° C. 2 minutes, 40° C. 2 minutes, 50° C. 2 minutes and 55° C. 10 minutes, and was stopped by adding 1 μL of 18 mM EDTA (VWR, 97062-656) to each well. All nuclei were then pooled, stained with DAPI at a final concentration of 3 μM, and sorted at 25 nuclei per well into 5 μL EB buffer. Cells were gated based on DAPI and Alexa647 such that singlets were discriminated from doublets and EdU+ cells were purified. 0.66 μL mRNA Second Strand Synthesis buffer and 0.34 μL mRNA Second Strand Synthesis enzyme (NEB, E6111L) were then added to each well. Second strand synthesis was carried out at 16° C. for 1 hour. 6 μL tagmentation reaction mix (made by mixing 0.5 μL self-loaded Tn5 with 200 μL Tagmentation buffer containing 20 mM Tris-HCl PH 7.5, 20 mM MgCl₂, 20% Dimethylformamide (Fisher, AC327175000)) was added to each well and tagmentation was performed at 55° C. for 5 minutes. After tagmentation, each well was mixed with 0.4 μL 1% SDS, 0.4 μL BSA (NEB, B90000S), and 2 μL of 10 μM P5 primer (5′-(SEQ ID NO:2415) AATGATACGGCGACCACCGAGATCTACA [15] CCCTACACGACGCTCTTCCGAT CT-3′ (SEQ ID NO:2416), IDT), and incubated at 55° C. for 15 minutes. Then, 2 μL 10% Tween-20, 1.2 μL nuclease-free water and 2 μL of 10 μM indexed P7 primer (5′-(SEQ ID NO: 2417) CAAGCAGAAGACGGCATACGAGAT [17] GTCTCGTGGGCTCGG-3′ (SEQ ID NO: 2418), IDT), and 20 μL NEBNext High-Fidelity 2× PCR Master Mix (NEB, M0541L) were added to each well. Amplification was carried out using the following program: 72° C. for 5 minutes, 98° C. for 30 seconds, 18-22 cycles of (98° C. for 10 seconds, 66° C. for 30 seconds, 72° C. for 1 minute), and a final 72° C. for 5 minutes. After PCR, samples were pooled and purified using 0.8 volumes of AMPure XP beads (Beckman Coulter, A63882) twice. Library concentrations were determined by Qubit (Invitrogen, Q33231), and the libraries were visualized by electrophoresis on a 2% E-Gel™ EX Agarose Gels (Invitrogen, G402022). All RNA-seq libraries were sequenced on the NextSeq 1000 platform (Illumina) using a 100 cycle kit (Read 1:58 cycles, Read 2:60 cycles, Index 1:10 cycles, Index 2:10 cycles). The TrackerSci RNA-seq library was sequenced to ˜20,000 reads per cell.

### TrackerSci-ATAC

EdU staining was performed on thawed nuclei using Click-iT Plus EdU Alexa Fluor™ 647 Flow Cytometry assay Kit (Thermo Fisher Scientific, 10634). A 500 μL reaction buffer (prepared following the manufacturer's protocol) supplemented with 1× cOmplete™ EDTA-free Protease Inhibitor Cocktail was added directly to the nuclei suspension, mixed well, and left in RT for 30 minutes. Then, nuclei were spun down for 5 minutes at 500 g (4° C.), washed once with 500 μL of 1× Click-iT saponin-based permeabilization and wash reagent, resuspended in 1 mL NSB with 1:20 dilution of 0.25 mg/ml 4′,6-diamidino-2-phenylindole (DAPI) and FACS sorted. Alexa647 and DAPI positive nuclei were sorted into 96-well plates with each well (250˜500 nuclei/well) containing 4 μL of NSB. Sorted plates were briefly centrifuged, mixed with 5 μL 2× TD buffer (20 mM Tris-HCl pH 7.5, 20 mM MgCl₂, 20% Dimethylformamide) and 1 μL barcoded Tn5. Tagmentation reaction was performed at 55° C. for 30 minutes and stopped by adding 11 μL 2× Stop buffer (40 mM EDTA, 1 mM Spermidine (Sigma, S0266)) to each well. All nuclei were then pooled, stained with DAPI at a final concentration of 3 μM, and sorted at 25 nuclei per well into 5 μL EB buffer. Cells were gated based on DAPI and Alexa647 such that singlets were discriminated from doublets and EdU+ cells were purified. After sorting, each well was mixed with 0.25 μL 18.9 mg/mL proteinase K (Sigma, 3115828001), 0.25 μL 1% SDS and 0.5 μL nuclease-free water, and reverse crosslinking was performed at 65° C. for 16 hours. Then, 2 μL 10% Tween-20 was added to each well to quench the SDS. Following on, 1 μL of 10 μM indexed P5 primer (5′-(SEQ ID NO:2415)

AATGATACGGCGACCACCGAGATCTACA [15] CCCTACACGACGC TCTTCCGATCT-3′ (SEQ ID NO:2449), IDT), 1 μL of 10 μM indexed P7 primer (5′-′-(SEQ ID NO:2419) CAAGCAGAAGACGGCATACGAGAT [17] GTGACTGGAGTTCAGACGTGTGCTCT TCCGATCT-3′ (SEQ ID NO:2420), IDT) and 10 μL NEBNext High-Fidelity 2× PCR Master Mix were added into each well. Amplification was carried out using the following program: 72° C. for 5 minutes, 98° C. for 30 seconds, 15-16 cycles of (98° C. for 10 seconds, 66° C. for 30 seconds, 72° C. for 1 minute), and a final 72° C. for 5 minutes. Final PCR products were pooled and purified by a Zymoclean DNA clean and concentration kit (Zymoresearch, D4014). Library concentrations were determined by Qubit, and the libraries were visualized by electrophoresis on a 2% E-Gel™ EX Agarose Gels. All ATAC-seq libraries were sequenced on the NextSeq 1000 platform (Illumina) using a 100 cycle kit (Read 1:58 cycles, Read 2:60 cycles, Index 1:10 cycles, Index 2:10 cycles). The TrackerSci ATAC-seq library was sequenced to ˜50,000 reads per cell.

### TrackerSci-RNA Data Processing

Read alignment and gene count matrix generation for the scRNA-seq were performed using the pipeline that was previously developed (Cao, J. et al. Science 357, 661-667 (2017)). Briefly, base calls were converted to fastq format and demultiplexed using Illumina's bcl2fastq/v2.19.0.316 tolerating one mismatched base in barcodes (edit distance (ED)<2). The RT barcode for each read was corrected to its nearest barcode (edit distance (ED)<2), and reads with uncorrected barcodes (ED>=2) were removed. Demultiplexed reads were then adaptor clipped using trim_galore/v0.4.1 (https://github.com/FelixKrueger/TrimGalore) with default settings. Trimmed reads were mapped to a chimeric reference genome of human and mouse (hg19/mm10) for the species-mixing experiment and to the mouse only (mm39) for mouse brain experiments, using STAR/v2.5.2b (Dobin et al., Bioinformatics 29, 15-21 (2013)) with default settings. Uniquely mapping reads were extracted, and duplicates were removed using the unique molecular identifier (UMI) sequence, reverse transcription (RT) index, and read 2 end-coordinate (i.e. reads with identical UMI, RT index, and tagmentation site were considered duplicates). Finally, mapped reads were split into constituent cellular indices by further demultiplexing reads using the RT index.

To generate digital expression matrices, the number of strand-specific UMIs for each cell mapping to the exonic and intronic regions of each gene was calculated with python/v2.7.18 HTseq package (Anders et al., Bioinformatics 31, 166-169 (2015)). For multi-mapped reads, reads were assigned to the closest gene, except in cases where another intersected gene fell within 100 bp to the end of the closest gene, in which case the read was discarded. For most analyses, both expected-strand intronic and exonic UMIs in per-gene single-cell expression matrices were included. Exonic and intronic gene count matrices were used in RNA velocity analysis.

For the species-mixing experiment, RNA barcodes with more than 200 UMIs and 100 unique genes were identified as real cells, and those with fewer than that were discarded. The percentage of uniquely mapping reads for genomes of each species was calculated. Cells with over 90% of UMIs assigned to one species were regarded as species-specific cells, with the remaining cells classified as mixed cells or “collisions”. The collision rate was calculated as the ratio of mixed cells.

### TrackerSci-ATAC Data Processing

Single-cell ATAC-seq data was performed using a published pipeline (Cusanovich et al., Science 348, 910-914 (2015); Cao et al., Science 361, 1380-1385 (2018)) with mild modifications. Base calls were converted to fastq format and demultiplexed using Illumina's bcl2fastq/v2.19.0.316 tolerating one mismatched base in barcodes (edit distance (ED)<2). The indexed Tn5 barcode for each read was corrected to its nearest barcode (edit distance (ED)<2), and reads with uncorrected barcodes (ED>=2) were removed. Demultiplexed reads were then adaptor-clipped using trim_galore/0.4.1 with default settings. Trimmed reads were mapped to a chimeric reference genome of human and mouse (hg19/mm10) for the species-mixing experiment and to the mouse only (mm39) for mouse brain experiments, using STAR/v2.5.2b (Dobin et al., Bioinformatics 29, 15-21 (2013)) with default settings. Duplicates were removed by picard MarkDuplicates/v2.25.2 (broadinstitute.github.io/picard/) per PCR sample. Deduplicated reads were split into constituent cellular indices by further demultiplexing reads using the Tn5 index.

A snap-format (Single-Nucleus Accessibility Profiles) file was generated from deduplicated bam files using SnapTools/v1.4.8 with default settings (github.com/r3fang/SnapTools) (Fang et al., Nat. Commun. 12, 1337 (2021)). A cell-by-bin count matrix with 5 kb bin size was created from the resulting snapfile. The promoter ratio for each cell was calculated as the number of fragments mapping to genomic bins overlapping with promoter regions (defined as 2 kb upstream of the gene body).

For the species-mixing experiment, ATAC barcodes with more than 1000 fragments and more than 0.2 promoter ratio were identified as real cells, and those with fewer than that were discarded. The percentage of uniquely mapping reads for genomes of each species was calculated. Cells with over 90% of reads assigned to one species were considered species-specific cells, with the remaining cells classified as mixed cells or “collisions”. The collision rate was calculated as the ratio of mixed cells.

### Cell Filtering, Clustering, and Annotation for TrackerSci RNA

A digital gene expression matrix was constructed from the raw sequencing data as described above. EdU+ cells and global cells were combined and analyzed together. Cells with less than 200 UMIs and 100 unique genes were discarded. Potential doublet cells and doublet-derived subclusters were detected using an iterative clustering strategy similar to before (Cao et al., Science 370, (2020)). Cells labeled as doublets (by scrublet/v0.2.3) (Wolock et al., Cell Syst 8, 281-291.e9 (2019)). or from doublet-derived sub-clusters were filtered out. The downstream dimension reduction and clustering analysis were done by Seurat/v4.0.2 (Hao et al., Cell 184, 3573-3587.e29 (2021)). Briefly, the dimensionality of the data was reduced by PCA (30 components) first and then with UMAP, followed by Louvain clustering. Clusters were assigned to known cell types based on cell type-specific markers (Table 7).

TABLE 7
Main cell types annotated in TrackerSci-
RNA and TrackerSci-ATAC

| | Gene markers supporting |
|---|---|
| Main cell type annotation | annotation |
| Astrocytes | Aqp4, Aldh1l1 |
| Cerebellum granule neurons | Gabra6, Fat2 |
| Choroid plexus epithelial cells | Ttr, Tmem72 |
| Committed oligodendrocytes precursors | Bmp4, Bcas1 |
| Dentate gyrus neuroblasts | Sema3c, Igfbpl1 |
| Ependymal cells | Foxj1, Ccdc153 |
| Erythroblasts | Hbb-bt, Hba-a1, Gypa |
| Immune cells | Ptprc |
| Mature neurons | Syt1 |
| Microglia | C1qb, P2ry12, Tmem119 |
| Myelin forming oligodendrocytes | Mog, Mag |
| Neuronal progenitor cells | Egfr, Mki67, Ascl1 |
| Olfactory bulb inhibitory neurons | Dlx6, Gng4 |
| Olfactory bulb neuroblasts | Dlx6, Prokr2, Robo2 |
| Oligodendrocytes progenitor cells | Pdgfra, Lhfpl3 |
| Vascular cells | Fn1, Vtn |

Differentially expressed genes across different cell types were identified using monocle2 (Qui et al., Nat. Methods 14, 979-982 (2017)) with the differentialGeneTest( ) function. Genes detected in less than 10 cells were filtered out before the analysis. To identify cell type-specific gene markers, genes were selected that were differentially expressed across different cell types (5% FDR, likelihood ratio test), with FC>2 between the target cell type and the second highest expressed cell type, and with maximum transcripts per million (TPM)>10 in the target cell types.

### Cell Filtering, Clustering, and Annotation for TrackerSci ATAC

Single-cell ATAC-seq profiles were generated as described above. EdU+ cells and global cells are combined and analyzed together. Cells with less than 1000 fragments and less than 0.2 promoter ratio were discarded. Dimensionality reduction for ATAC-seq data was performed using the snapATAC/v1.0.0 (Fang et al., Nat. Commun. 12, 1337 (2021)). A cell-by-bin matrix at 5-kb resolution was used. There was focus on bins on chromosomes 1-19, X and Y. High-coverage bins (top 5% bins that overlap with invariant features) or low-coverage bins (bottom 5% bins that represent general inaccessible regions) were filtered out before the analysis. Diffusion maps dimensionality reduction was performed on the filtered cell-by-bin matrix after binarization. UMAP analyses were performed on the top 20 eigenvectors, followed by unsupervised clustering via the densityPeak algorithm implemented in R package densityClust/v0.3 (Rodriguez et al., Science 344, 1492-1496 (2014)).

Integration analysis was performed between the TrackerSci-RNA dataset and TrackerSci-ATAC dataset to annotate the ATAC dataset. The gene activity score for ATAC cells was computed using the snapATAC function createGmatFromMat( ) by summing up the counts of bins overlapping with the gene body. A Seurat object was generated using the gene activity matrix and previously calculated diffusion map embeddings for single cell ATAC-seq. Then, variable genes were identified from TrackerSci-RNA data and used for identifying anchors between these two modalities. Next, the RNA-seq and ATAC-seq profiles were co-embedded in the same low-dimensional space to visualize all the cells together. Overlapped RNA clusters were used to annotate ATAC cells in the integrated UMAP space. ATAC cells without overlapped RNA cells were removed with careful inspection since they usually represent potential doublets or low-quality cells. Finally, single-cell ATAC dimension reduction, clustering, and integration analysis were rerun on the remaining dataset following the same procedure.

### Peak Calling and Identifications of Cell-Type-Specific Peaks

To define peaks of accessibility across all sites, MACS2/v2.1.1 (Zhang et al., Genome Biol. 9, R137 (2008)) was used. Nonduplicate ATAC-seq reads of cells from each main cell type were aggregated, and peaks were called on each group separately with these parameters: --nomodel --extsize 200 --shift -100 -q 0.1. Peak summits were extended by 250 bp on either side and then merged with bedtools/v2.30.0 (Zhang et al., Genome Biol. 9, R137 (2008); Quinlan et al., Bioinformatics 26, 841-842 (2010)), together with gene promoter regions (annotated transcription start site (TSS) in GENCODE VM27 minus/plus 1000 base pairs in a strand-specific manner). Each read alignment was extended by 100 bp upstream and downstream from the insertion site of tagmentation. Cells were determined to be accessible at a given peak if a read from a cell overlapped with the peak. The peak count matrix was generated by a custom python script with the HTseq package (Anders et al., Bioinformatics 31, 166-169 (2015); Zhang et al., Genome Biol. 9, R137 (2008); Quinlan et al., Bioinformatics 26, 841-842 (2010)). Differentially accessible peaks across cell types were identified using monocle 2 (Qiu, X. et al., Nat. Methods 14, 979-982 (2017)) with the differentialGeneTest( ) function. Peaks detected in less than 10 cells were filtered out before the analysis. To determine cell-type-specific peak markers, peaks that were selected were ones that were differentially accessible across different cell types (5% FDR, likelihood ratio test), with FC>2 between the target cell type and the second highest expressed cell type, and with TPM>10 in the target cell types.

### Analysis for Linking Cis-Regulatory Elements (CRE) to Regulated Genes

Links between chromatin accessible sites and regulated genes based on their covariance are identified. Only EdU+ cells were kept in this analysis. Pseudo-cells were first constructed by aggregating the RNA-seq and ATAC-seq profile of highly similar cells through k-means clustering the integrative UMAP coordinates. The k was selected so that the average cell number per subcluster is 150. Subclusters overrepresented by one molecular layer (the percentage of cells from either RNA-seq or ATAC-seq profile greater than ninety percent) were merged with a nearby subcluster. After aggregating cells within each sub-cluster, a total of 88 pseudo-cells were obtained, with a median of 54 cells from RNA-seq profile and 93 cells from ATAC-seq profile. Aggregated count matrices for RNA-seq and ATAC-seq were normalized to transcripts per million (TPM) and log 1p transformed. Genes and peaks with TPM value greater than 10 in the maximum expressed pseudo-cells were retained. Then, for each gene, the Pearson Correlation Coefficient (PCC) between its gene expression and the chromatin accessibility of its nearby accessible sites (minus/plus 500 kb from the TSS) across pseudo-cells was calculated. Sites overlapping with minus/plus 1 kb from the TSS were considered promoters, while the rest were considered distal regions. To define a threshold at PCC score, a set of background pairs were generated by permuting the pseudo cell id of the ATAC-seq matrix and with an empirically defined significance threshold of FDR<0.05, to select significant positively correlated cCRE-gene pairs. The linkage was further filtered by requiring that either the maximum expressed cell types in the RNA profile and the ATAC profile were the same or the top two or top three highest expressed cell types were in the same cell trajectory (Oligodendrogenesis trajectory: OPC, COP, OLG; Astrocytes trajectory: ASC, NPC; DG neurogenesis trajectory: NPC, DGNB; OB neurogenesis trajectory: NPC, OBNB, OBIN). Finally, only the one top linked gene with the highest PCC for each peak was kept.

### Transcription Factor Analysis

To identify key TF regulators of each main cell type, there was a search for TF that can be validated in two molecular layers by correlating gene expression and motif accessibility. First, using the TrackerSci-ATAC dataset, the top 300 sites per main cell type were selected (from the differential peak analysis described above, filtered by q-value<0.05, maximum expressed TPM>10 and ranked by FC between the highest and the second expressed cell type) to a combined peak set. The peaks were then resized to a fixed length of 500 bp (±250 bp around the center) and a binarized peak-by-motif matrix was generated using the R package motifmatchr/v1.16.0 (github.com/GreenleafLab/motifmatchr) with the matchMotifs( ) function to identify the occurrences of motifs in each peak from a filtered collection of the cisBP motif database curated by chromVARmotifs (Weirauch et al., Cell 158, 1431-1443 (2014); Schep et al., Nat. Methods 14, 975-978 (2017)). A matrix of motif-by-cell counts was obtained by multiplying the peak-by-cell matrix with the peak-by-motif matrix, and was aggregated into pseudo-cells based on the k-means clustering described before. The PCC between the scaled TF motif accessibility and the scaled TF gene expression across pseudo-cells was then computed. To select significantly positive and negative correlations of TF gene expression and motif accessibility pairs, the pseudo cell id of the motif-by-cell matrix was permuted to compute a background PCC distribution and selected the TF pairs with an empirically defined significance threshold of FDR<0.05. In addition, only TF with TPM>10 in the maximum expressed cell type was kept.

### Trajectory Analysis

Cells corresponding to the neurogenesis trajectory (ASC, NPC, DGNB, OBNB and OBIN) or the oligodendrogenesis trajectory (OPC, COP and OLG) from both RNA-seq data and ATAC-seq data were selected for detailed investigation. UMAP dimension reduction at the trajectory level was performed using the integration function from Seurat (Hao et al., Cell 184, 3573-3587.e29 (2021)), using the top 3,000 highly variable genes and top 50 PCs. Each cell was assigned a pseudotime value based on its position along the trajectory using monocle 2 function order_cells( ). RNA velocity analyses were performed using scVelo/v0.2.3 (Bergen et al., Nat. Biotechnol. 38, 1408-1414 (2020)) using the exonic and intronic gene count matrix generated from sciRNA pipeline to validate the cell differentiation direction and estimate the position of the progenitor cell state. For the two neurogenesis trajectories (DG neurogenesis and OB neurogenesis), pseudotime assignment was calculated separately and scaled so that the cells shared between two trajectories received the same pseudotime value. Specifically, the pseudotime value calculated from the OB trajectory was used for common progenitor cells in both DG and OB trajectories. A linear regression line was fitted using R function lm( ) to predict the OB-pseudotime based on the DG-pseudotime. Then, for cells unique to the DG neurogenesis, their pseudotime was adjusted using the predict( ) function using DG-pseudotime as input. Gene expression and peak accessibility dynamics along pseudotime were identified using monocle 2 (Qiu, X. et al., Nat. Methods 14, 979-982 (2017)) with the differentialGeneTest( ) function with pseudotime values and their main cluster identity as variables. Genes or peaks that passed a significant test (FDR of 5%) were considered as dynamically regulated genes or sites. Furthermore, differential accessible sites along pseudotime were used to infer TF motif accessibility dynamics. A motif deviation score for each single cell was computed using chromVar/v1.4.1 (Schep et al., Nat. Methods 14, 975-978 (2017)) with the dynamic peak set (resized to 500 bp) as input. Then, the motif deviation scores of each single cell were rescaled to (0, 10) using R function rescale( ) and differential accessible motifs were identified using monocle 2 with the differentialGeneTest( ) function. TF motifs that passed a significant test (FDR of 5%) were considered as dynamically regulated motifs. For gene enrichment analysis the enrichR (Chen et al., BMC Bioinformatics 14, 128 (2013)) was used and the following pathways collections were considered: Panther_2016, Reactome_2016, KEGG_2019_Mouse, GO_Biological_Process_2018, GO_Molecular_Function_2018. For visualizing the dynamics of gene expression, peak accessibility and motif accessibility, R package ComplexHeatmap/v2.10.0 (Gu et al., Bioinformatics 32, 2847-2849 (2016)) was used.

### Cell Proportion Analysis

To quantify the cell-type-specific changes in the proliferation dynamics across conditions, the fraction of each cell type within EdU+ population from each condition for RNA-seq data and ATAC-seq data separately was calculated, which was further multiplied by the median of EdU+ ratio for each group obtained from FACS sorting. For Adult WT mice, only those that were harvested 24 h after five-day labeling were included to avoid artifacts introduced by the labeling time.

To quantify the effects of ageing on cell differentiation dynamics along neurogenesis and oligodendrogenesis trajectories, miloR/v1.3.1 (Dann et al., Nat. Biotechnol. (2021), doi:10.1038/s41587-021-01033-z) was applied, a single-cell differential abundance testing framework using k-nearest neighbor (KNN) graphs. The KNN graph was first constructed on the UMAP space for each trajectory using the buildGraph( ) function with k=120 for the neurogenesis trajectory and k=250 for the oligodendrogenesis trajectory. Cell neighborhoods were then defined using the makeNhoods( ) function and the number of cells from each experiment sample were counted for each neighborhood using the countCells( ) function. Testing for differential abundance in neighborhoods was performed using the testNhoods( ) function and significance levels for Spatial FDR of 0.05 were used. Visualization of differential abundance neighborhoods was done using the plotNhoodGraphDA( ) function.

### Differential Analysis of NPC and OPC Across Aged Groups

Differential gene expression analysis across young, adult, and aged groups of NPC and OPC was performed using monocle 2 (Qiu, X. et al., Nat. Methods 14, 979-982 (2017)) function differentialGeneTest( ) with the number of genes detected per cell included as a covariant. For Adult WT mice, only cells from the animals harvested at 24 h after 5-day labeling were included to avoid artifacts introduced by the labeling time. In addition, only differentially expressed genes (>expressed in more than 10 cells) along the neurogenesis or the oligodendrogenesis trajectory were included in the differential gene test. Differentially expressed genes were selected by a q-value cutoff of 0.1, a TPM cutoff of 50 in the maximum expressed group, and with at least 1.5 FC between the maximum expressed group and the minimum expressed group. Next, differentially expressed genes were grouped to aged-depleted genes and aged-enriched genes by the following criteria: for ageing-depleted genes, the genes with minimum expression in aged mice were first selected, and only those with either maximum expression in young mice or within less than 2 FC between the young group and the adult group were kept. For ageing-enriched genes, the genes with maximum expression in aged mice were first selected, and only those with either minimum expression in young mice or with less than 2 FC between the young group and the adult group were kept. The DE genes were further filtered based on the consistency on their promoters or linked sites. For ageing-depleted genes, there was a requirement that the mean of promoter accessibility or linked site accessibility was at the minimum level in the aged group compared to young and adults. For ageing-enriched genes, there was a requirement that the mean of promoter accessibility or the linked site accessibility was at the maximum level in the aged group compared to young and adults. Genes that were lowly detected in both promoter accessibility and linked sites (represented by the mean of TPM<10 in all conditions) were also discarded.

### Integration Analysis Between TrackerSci-RNA and EasySci-RNA

Integration analysis of scRNA-seq dataset profiled using TrackerSci and Easy Sci was performed using Seurat/v4.0.2 (Hao et al., Cell 184, 3573-3587.e29 (2021)). 14,095 TrackerSci-RNA cells (including 5,715 EdU+ cells and 8,380 all brain cells without EdU enrichment) were integrated with 126,285 EasySci-RNA cells (up to 5,000 cells randomly sampled from each of 31 cell types) in the companion study (Cao et al., Science 370, 924-925 (2020)). Shared variable genes, selected by SelectIntegrationFeatures( ) function, were used for identifying anchors using FindIntegrationAnchors( ). The two datasets were then integrated together with IntegrateData( ) function. To visualize all the cells together, all the cells were co-embedded in the same low-dimensional space. The same integrative analysis strategy was further applied to cells matching the same cellular state from both datasets. Specifically, for the neurogenesis trajectory, 1,214 EdU+ cells from TrackerSci-RNA (NPC, OBNB, and OBIN) were integrated with 37,258 OB neurons-1 cells from EasySci-RNA. For the oligodendrogenesis trajectory, 3,044 EdU+ cells from TrackerSci-RNA (OPC and COP) were integrated to 22,718 Oligodendrocyte progenitor cells from EasySci-RNA. For the microglia, 600 EdU+ microglia from TrackerSci-RNA were integrated to 15,754 Microglia from EasySci-RNA. Microglia subclusters corresponding to peripheral immune cells were excluded before the analysis.

### Quantifications of the Self-Renewal Potential and the Differentiation Potential

The self-renewal potential was defined as the ratio of newly generated progenitor cells within 5 days of EdU labeling divided by the ratio of total progenitor cells detected from the global population. To account for potential variations due to slight differences of animal ages between TrackerSci and the brain cell atlas, a linear model between the ages and the ratio of progenitor cells was first fitted using the EasySci data for the following cell type: neuronal progenitor cells, oligodendrocyte progenitor cells, and microglia. That was used to predict the ratio of progenitor cells for each individual mice profiled by TrackerSci. The ratio of newly generated progenitor cells from each 5-day labeled mice was then divided by the predicted cellular fraction of the global progenitor pool for the same cell type. A line plot was generated using the median values of proliferation potential for each aged group normalized to the young mice. RNA and ATAC cells were both included, and samples with less than 50 cells were excluded from the calculation.

The differentiation potential was quantified by the ratio of differentiated cells divided by all EdU+ cells in the same trajectory. Such a ratio was calculated only for oligodendrogenesis trajectory since it's a unidirectional route. For this analysis, the ratio of committed oligodendrocytes and myelin-forming oligodendrocytes was divided to the ratio of oligodendrocytes progenitor cells for each sample and median values of each age group were used to generate the line plot. RNA and ATAC cells were included, and samples with less than 50 cells were excluded from the calculation.

The Experimental Results are now described.

### a Global View of Rare Newborn Cells Across the Mammalian Brain

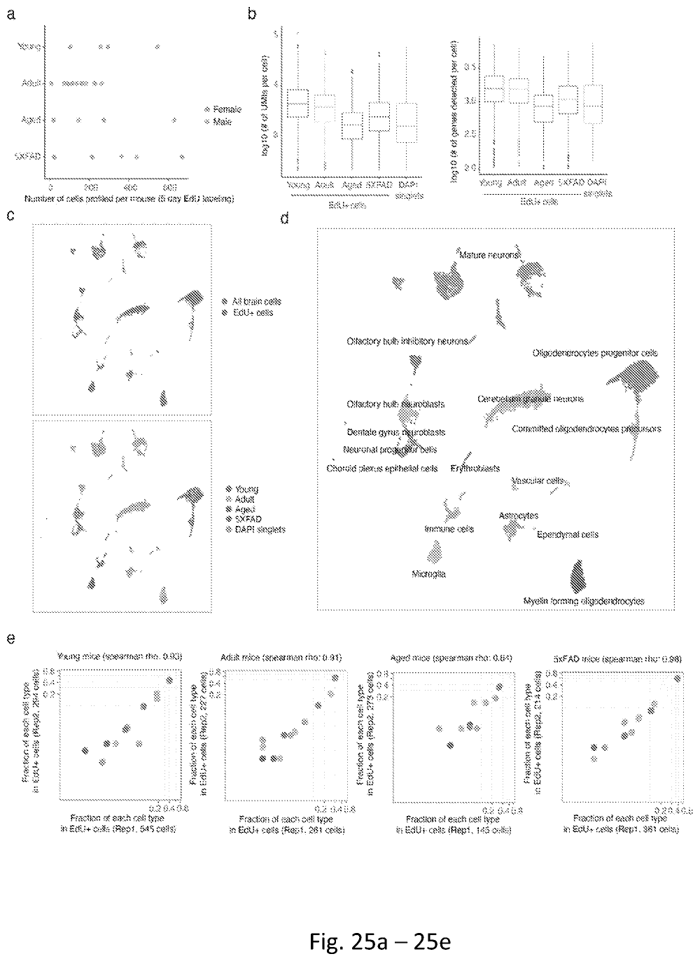

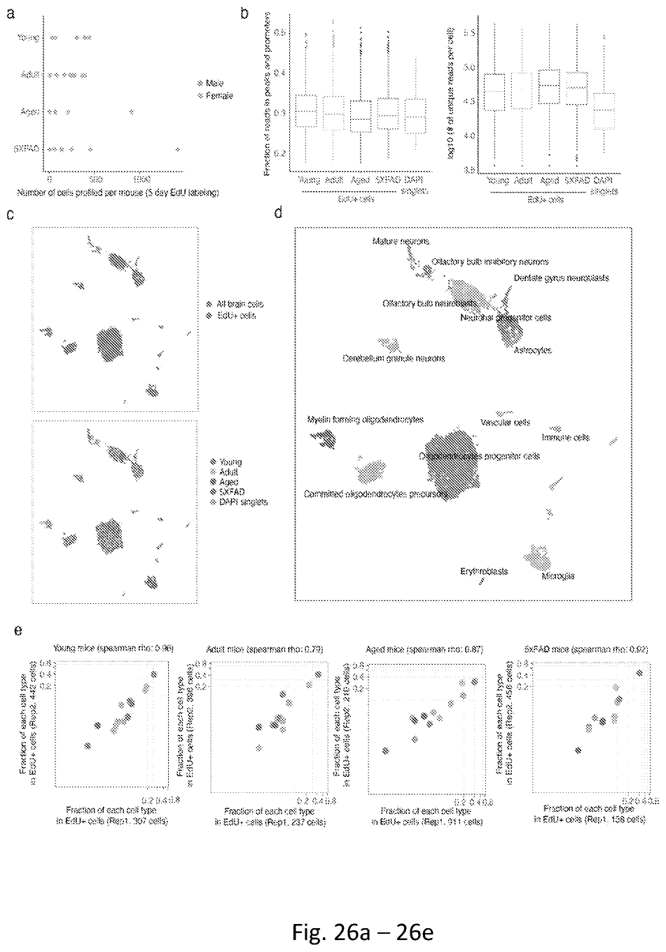

TrackerSci was applied to capture rare newborn cells from entire mouse brains spanning three age stages and two genotypes. Briefly, following three to five days of continuous EdU labeling, nuclei of the whole brain from thirty-eight sex-balanced C57BL/6 mice were isolated (FIG. 20a), including thirty-three wild-type mice across multiple development stages (Young: 6-9 weeks, Adult: 11-20 weeks, and Aged: 88-98 weeks) as well as five 5×FAD mutant mice (11-20 weeks) harboring multiple Alzheimer's Disease mutations¹³. Following TrackerSci protocol, transcriptomic profiles for 5,715 newborn cells (median 2,909 UMIs) (FIG. 25a,b) and chromatin accessibility profiles for 8,974 newborn cells (median 50,225 unique reads) (FIG. 26a,b) were obtained. In addition, to characterize the global brain cell population as a background control, DAPI singlets representing ‘all’ brain cells were included (i.e., without enrichment of the EdU+ cells) and transcriptomic profiles for 8,380 nuclei (median 1,553 UMIs) and chromatin accessibility profiles for 342 nuclei (median 24,521 unique reads) were obtained. The EdU+ nuclei and DAPI singlets were collected from the same set of samples and processed in parallel to minimize any batch effect.

The 14, 129 TrackerSci transcriptome profiles, including both EdU+ nuclei and DAPI singlets, were subjected to Louvain clustering (Blondel et al., Journal of Statistical Mechanics: Theory and Experiment vol. 2008 P10008 (2008)) and UMAP visualization (McInnes et al., Journal of Open Source Software vol. 3 861 (2018)) (FIG. 25c). Sixteen cell clusters were identified and annotated based on established markers (FIG. 25d), ranging in size from 25 cells (Choroid plexus epithelial cells) to 3,134 cells (Mature neurons). A semi-supervised clustering analysis of 9,316 TrackerSci chromatin accessibility profiles was performed (8,974 EdU+ nuclei and 342 DAPI singlets), and fourteen clusters (FIG. 26c,d) was identified, which mapped 1:1 to the main cell types identified in the transcriptome analysis. As expected, the corresponding cell types defined by the two layers overlapped well in the integration analysis (FIG. 20b). Two rare cell types (i.e., ependymal cells and choroid plexus epithelial cells) were only detected in the RNA dataset, potentially due to the low abundance of these cell types.

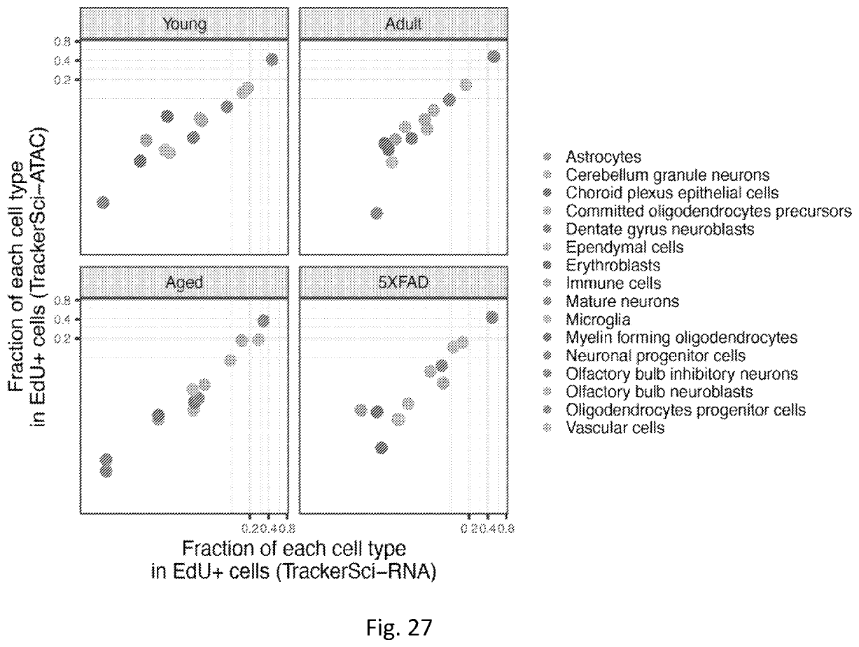

While EdU+ nuclei from replicate mouse brain groups were similarly distributed (FIG. 25e, FIG. 26e), a notably altered distribution of cell-type-specific fractions between ‘all’ brain cells and the EdU+ cells was observed (FIG. 20d). For example, in contrast to the ‘all’ brain cells that are dominated by mature neurons (e.g., cerebellum granule neurons: 32.7% in DAPI singlets vs. 2.85% in EdU+ cells) and differentiated glial cells (e.g., myelin-forming oligodendrocytes: 11.9% in DAPI singlets vs. 0.75% in EdU+ cells), the EdU+ population showed prominent enrichment of progenitor cells such as immature neurons (e.g., Olfactory bulb neuroblasts: 0.14% in DAPI singlets vs. 13.4% in EdU+ cells) and glia progenitors (e.g., oligodendrocyte progenitor cells: 1.11% in DAPI singlets vs. 45.4% in EdU+ cells). Intriguingly, newly-generated erythroblasts (Hbb-bt+, Hbb-bs+) and immune cells (Ptprc+) were detected, which may correspond to newborn blood cells circulating in the brain, as they exclusively exist in the EdU+ nuclei. Of note, the cell-type-specific distribution of newborn cells was highly correlated between TrackerSci transcriptome and chromatin accessibility datasets (mean Spearman's correlation r=0.92; FIG. 20e) and across conditions (FIG. 27).

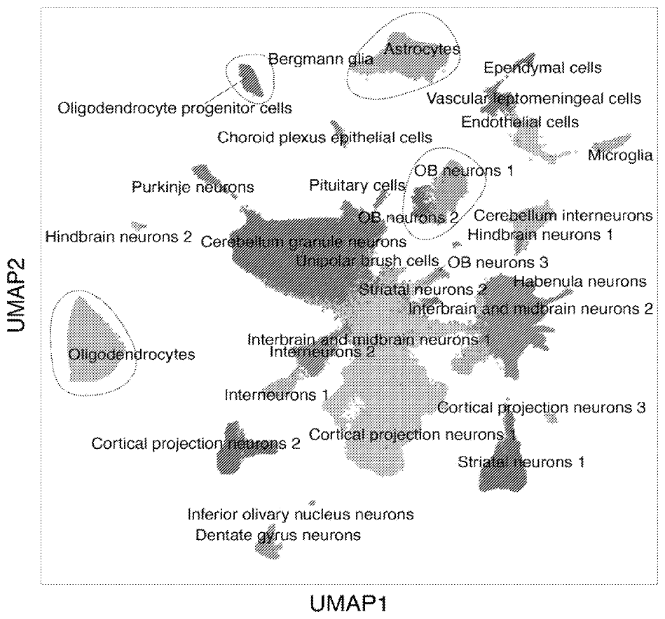

TrackerSci datasets were integrated with a global brain cell atlas from a companion study (Cao et al., Science 370, 924-925 (2020)), for which 1.5 million cells from entire mouse brains spanning three age groups and two mutants associated with Alzheimer's disease were profiled. Briefly, EdU+ brain cells (5,715 single-cell transcriptomes from TrackerSci), ‘All’ brain cells (8,380 DAPI singlets from TrackerSci), and “All” brain cells from the global brain cell atlas (sampling 5000 cells for each main cell type) were integrated into the same UMAP space. As expected, ‘All’ brain cells from the TrackerSci highly overlapped with ‘All’ brain cells from the global brain cell atlas in the integrated UMAP space (FIG. 20f). Remarkably, with the assistance of EdU+ cells profiled from TrackerSci, continuous cellular differentiation trajectories bridging several terminally differentiated cell types were formed, including the oligodendrogenesis trajectory from the oligodendrocyte progenitor cells to differentiated oligodendrocytes, and the neurogenesis trajectory connecting astrocytes and OB neurons (FIG. 20f). While the 1.5 million global brain cell atlas is one of the most extensive single-cell analysis of adult mouse brains, these “bridge” cells were still missing in the trajectory analysis (FIG. 28), highlighting the importance of the TrackerSci method in the characterization of extremely rare proliferating/differentiating cells to reconstruct continuous differentiation trajectory of cells.

### Transcriptional and Epigenetic Signatures of Newborn Cells

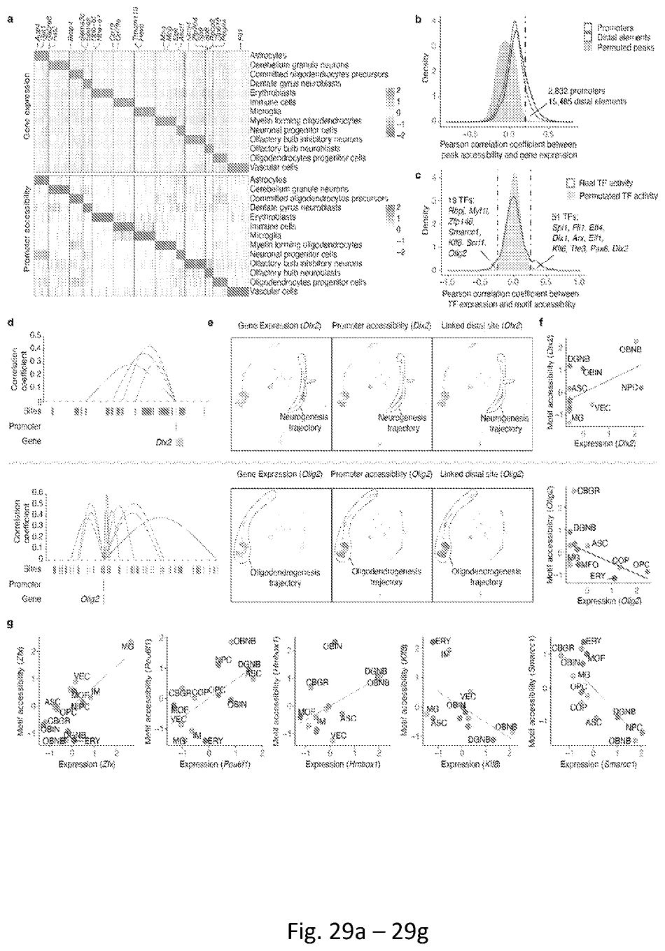

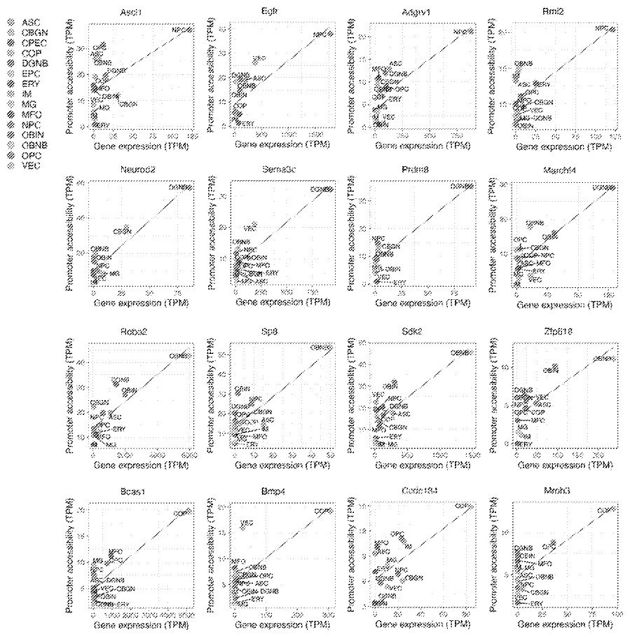

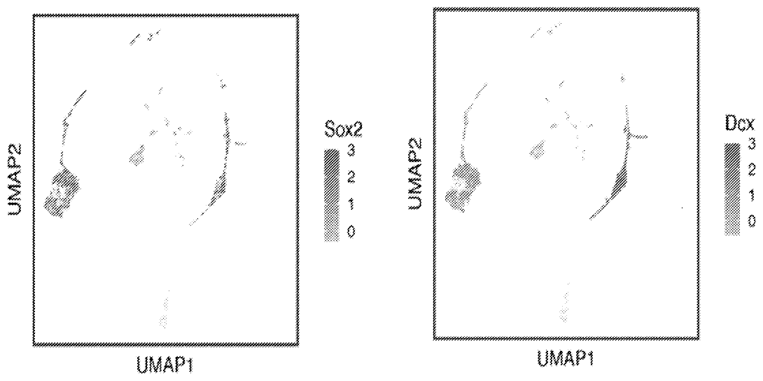

Toward a better understanding of the molecular signatures of newborn cells, differential expression (DE) and differential accessibility (DA) analysis was performed, yielding 5,610 DE genes (FDR of 5%, FIG. 29a) and 68,556 DA sites (FDR of 5) with significant changes across cell types. 1,744 (34.8%) of DE genes have DA promoters enriched in the same cell type (median Pearson rho=0.81, FIG. 29a). While canonical gene markers were observed and used for the annotation analysis (FIG. 30), many novel markers that are highly cell-type-specific but have not been reported in prior research were detected (FIG. 30), including markers for neuronal progenitor cells (e.g., Adgrv1 and Rmi2), DG neuroblasts (e.g., Prdm8 and Marchf4), OB neuroblasts (e.g., Zfp618 and Sdk2) and committed oligodendrocyte precursors (e.g., Ccdc134 and Mroh3). These markers were cross-validated by cell-type-specific gene expression and promoter accessibility. Of note, some of the widely used neurogenesis markers, such as Sox2 and Dcx (Hodge et al., Dev. Neurobiol. 71, 680-689 (2011)), were expressed across multiple cell types (e.g., oligodendrocyte progenitor cells; FIG. 31), which may lead to the limited accuracy in capturing cells undergoing neurogenesis.

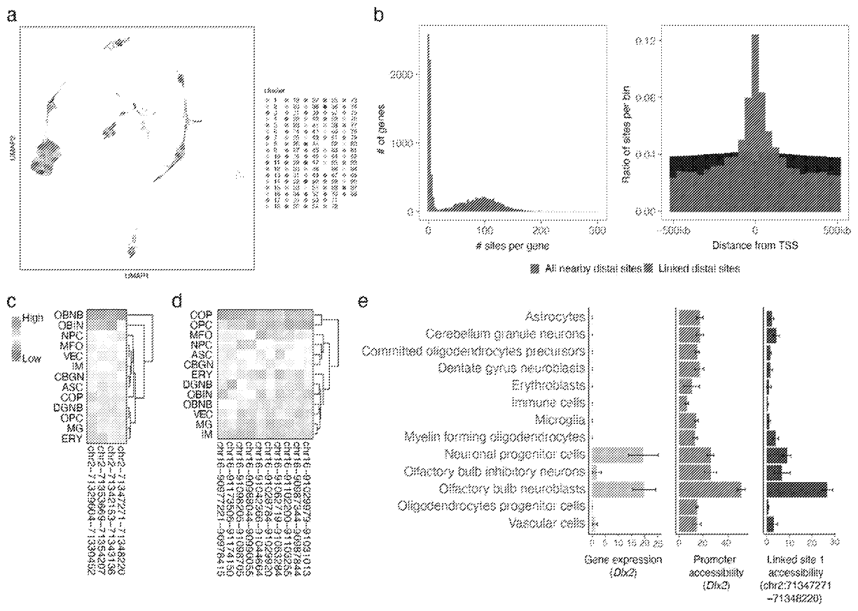

To investigate the epigenetic landscape that shapes the gene expression of newborn cells, the cis-regulatory elements were linked to the expression of putative target genes based on their covariance across different cell states. the correlation between the expression of each gene and the accessibility of its nearby DA sites across 88 ‘pseudo-cells’ was computed (a subset of cells with adjacent integrative UMAP coordinates grouped by k-means clustering, FIG. 32a). To control for artifacts of the analysis, the sample IDs of the chromatin accessibility matrix was permuted and the same analysis was performed. Altogether, 15,485 positive links between genes and distal sites (plus 2,832 associations between genes and promoters) were identified at an empirically defined significance threshold of FDR=0.05 and based on their cell-type-specificity (FIG. 29b).

The identified distal site-gene linkages were significantly closer than all possible pairs tested (median 159 kb for identified links vs. 251 kb for all pairs tested; P-value<5×10⁻⁵, unpaired permutation test based on 20,000 simulations, FIG. 32b). Most genes were associated with a few links (median two distal sites per gene, out of a median of 94 distal sites within 500 kb of the TSS tested, FIG. 32b). For example, Dlx2, a canonical neurogenesis marker, was significantly linked to four distal peaks, all exhibiting remarkable cell-type-specificity similar to its gene expression and promoter accessibility (FIG. 29d, FIG. 32c). By contrast, a small subset of genes (3.5%) were linked with a large number of peaks (>=10 peaks). One such example is Olig2, an oligodendrogenesis marker, which was linked with 10 distal peaks (FIG. 29d), all highly enriched in the oligodendrocytes progenitor cells (OPC) and committed oligodendrocytes precursors (COP) (FIG. 29e, FIG. 32d). For some genes (e.g., Dlx2), the linked distal sites showed stronger cell-type-specificity compared to their promoters (FIG. 32e), suggesting the long-range transcriptional control could play a critical role in the cell-type-specificity of newborn brain cells.

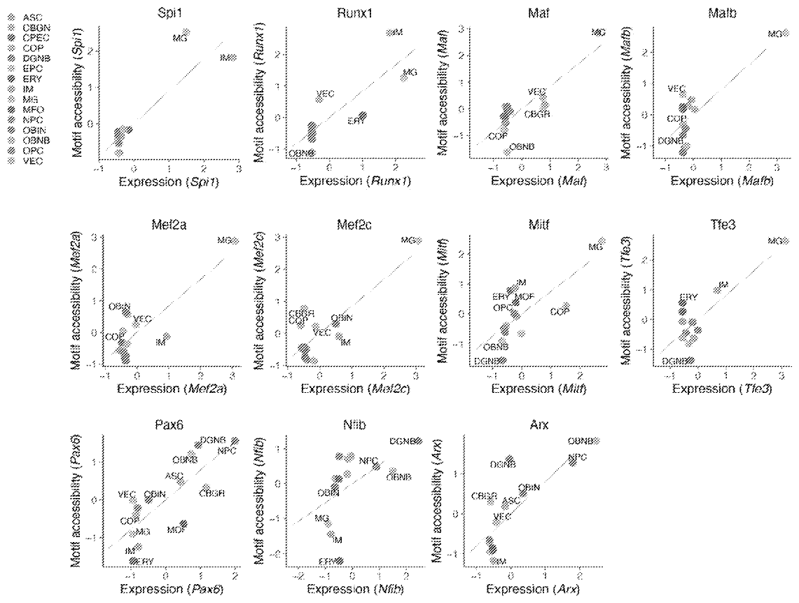

Transcription factors (TFs) determining the cell type specificity of newborn cells were systematically characterized. The occurrence of each TF motif within cell-type-specific accessible sites was first quantified and the Pearson correlation coefficient between TF expression and motif accessibility across all afore-described “pseudo-cells” was computed. Meanwhile, the same analysis was performed using the permuted data as a background control. With this approach, 51 potential TF activators with positively correlated gene expression and motif accessibility were identified (e.g., Dlx2, FIG. 29f), and 19 TF repressors showed negative correlations between gene expression and motif accessibility (e.g., Oligo2, FIG. 29f). In fact, Oligo2 has been reported to encode a transcriptional repressor during motor neuron differentiation and myelinogenesis (Zhang et al., Nat. Commun. 13, 1423 (2022)). In addition, most top enriched cell-type-specific TFs can be validated by previous studies, such as Spi1 and Runx1 in microglia and other immune cells (Yeh et al., Trends Mol. Med. 25, 96-111 (2019); Iwasaki et al., Immunity 26, 726-740 (2007)); Maf, Mef2a, and Tfe3 in microglia only (Yeh et al., Trends Mol. Med. 25, 96-111 (2019); Solé-Domènech et al., Ageing Res. Rev. 32, 89-103 (2016)); and Pax6, Nfib, and Arx in neuronal progenitor cells and neuroblasts (Osumi et al., Stem Cells 26, 1663-1672 (2008); Ninkovic et al., Cell Stem Cell 13, 403-418 (2013); Colombo et al., Journal of Neuroscience vol. 27 4786-4798 (2007)). Notably, several less-characterized TF regulators showing strong enrichment in certain cell types were identified, such as Zfx in microglia, Pou6f1, Hmbox1, Klf8, and Smarcc1 in immature neurons (FIG. 29g, FIG. 33), validated by both gene expression and motif accessibility.

### a Highly Heterogeneous Cell Response to Ageing Across Newborn Brain Cells

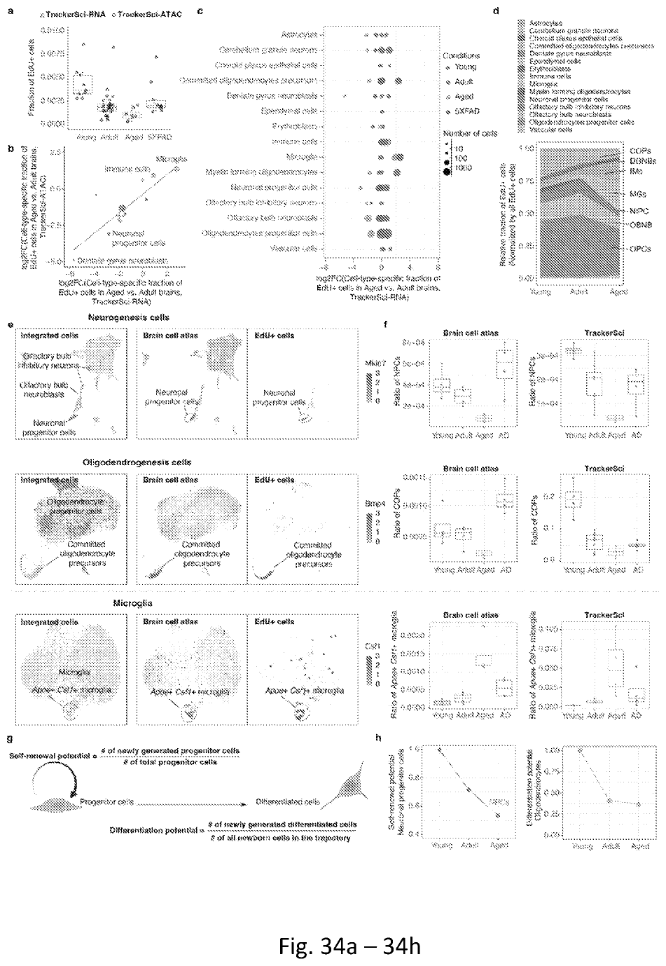

Through comparing the fraction of EdU+ cells across young, adult, and aged brains, as expected, a significant reduction of newborn brain cells was observed over time, indicating a globally reduced proliferation behavior upon ageing (FIG. 34a). To further investigate the cell-type-specific response in ageing, the relative fraction of each newborn cell type was quantified by their fractions in the EdU labeled cell population, multiplied by the ratio of all EdU+ cells in the global cell population. Interestingly, a highly heterogeneous cell response to ageing was detected across various newborn cell types. For example, while most cell types exhibited reduced proliferation upon ageing, microglia and other immune cells showed a remarkable boost in the fraction of newborn cells (FIG. 34b-d). This is consistent with the elevated inflammatory responses in the aged brain (Corlier et al., Neuroimage 172, 118-129 (2018)). In addition, even those cell types with decreased proliferation still present to varying degrees. For example, one of the most altered cell types in ageing, dentate gyrus neuroblasts, showed an 18-fold reduction in the aged brain (vs. adult brain), while the proliferation rate of vascular cells was only mildly affected. Of note, the cell-type-specific response to ageing was validated by both single-cell transcriptome and chromatin accessibility profiles (FIG. 34b).

Similar to ageing-induced changes, highly heterogeneous cell-type-specific responses to AD-associated genetic perturbations was detected in the 5×FAD mice, even though they were profiled at a relatively early stage (before 20 weeks). For example, several cell types already exhibited concordant ageing-associated changes, such as the expansion of microglia and the reduction of newborn DG neuroblasts, astrocytes, and cerebellum granule neurons (FIG. 34c), suggesting the alteration of cell-type-specific proliferation status is earlier than phenotypical observations and can be used as early markers of Alzheimer's disease.

To further validate the cell-type-specific dynamics in ageing, the newborn cells recovered from TrackerSci and the global brain cell atlas (in the companion study) were integrated for sub-clustering analysis. Indeed, the integration analysis at the sub-cluster level facilitated identifying and annotating rare progenitor cells in the brain cell atlas. These include neuronal progenitor cells (marked by Mki67, Top2a, and Egfr) and committed oligodendrocyte precursors (marked by high expression of Bmp4 and Bcas1) (FIG. 34e), both of which are remarkably down-regulated over time in both TrackerSci and the global brain cell atlas. In addition, the integration analysis revealed a reactive microglia subtype, marked by high expression of Apoe and Csf1 in both datasets. This microglia subtype has been previously reported to be enriched in both aged and AD mammalian brains (Keren-Shaul et al., Cell vol. 169 1276-1290.e17 (2017)). As expected, the proliferation of the Apoe+, Csf1+ microglia increased dramatically in both aged and 5×FAD brains, consistent with the cell-type-specific changes in the global cell population.

How ageing impacts the self-renewal and differential potential of brain progenitor cells was then quantitatively investigated. First, the self-renewal potential can be calculated as the ratio of newly generated progenitor cells divided by the ratio of total progenitor cells detected from the global population (i.e., the number of newborn cells generated per progenitor cell in a fixed time). For example, a significantly reduced self-renewal potential of neuronal progenitor cells was detected (FIG. 34h), which explained the depleted neural stem cell pool in aged brains. Meanwhile, the differentiation potential of cell types can be defined by the ratio of newly generated differentiated cells divided by all newborn cells in the same trajectory (FIG. 34g). For example, a substantial reduction of the differentiation potential in oligodendrocyte progenitor cells over time was observed, suggesting its differentiation process is severely blocked across the lifespan (FIG. 34h). This analysis represents the first quantitative measurement of cell-type-specific self-renewal and differentiation capacities in vivo.

### The Impact of Ageing on Adult Neurogenesis

Adult neurogenesis and oligodendrogenesis have been reported to decline upon ageing (Polina et al., Oncogene 30, 3105-3126 (2011); Galvan et al., Clin. Interv. Aging 2, 605-610 (2007)); however, the detailed mechanism is still unclear due to technical limitations. The impact of ageing on adult neurogenesis and oligodendrogenesis was interrogated, and the transcriptional and epigenetic controls underlying cell-type-specific proliferation and differentiation dynamics was delineated.

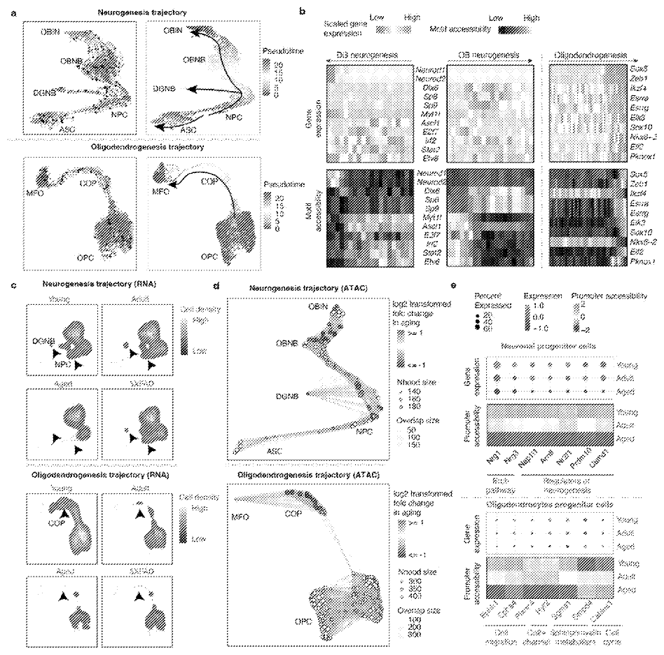

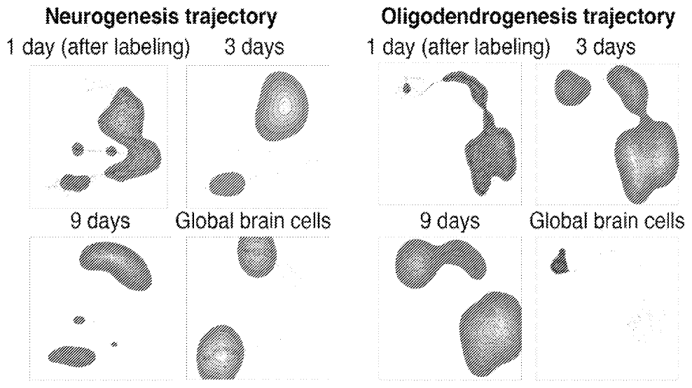

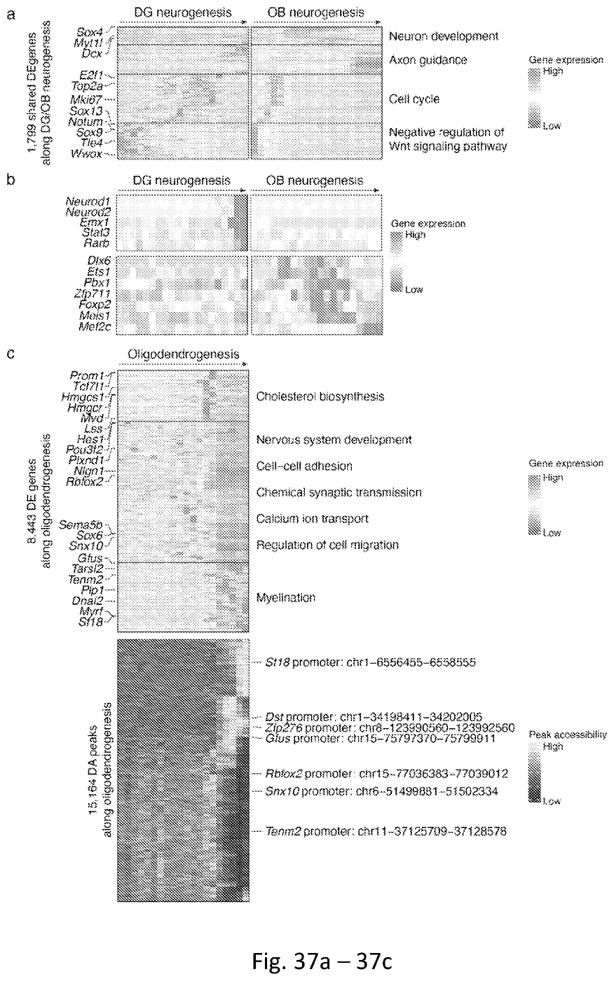

For adult neurogenesis, three main trajectories that differentiated into DG neuroblasts, OB neuroblasts, and astrocytes were identified, consistent with the cell state transition directions inferred by the RNA velocity analysis (Bergen et al., Nat. Biotechnol. 38, 1408-1414 (2020)) and prior report (Ratz et al., Nat. Neurosci. 25, 285-294 (2022)) (FIG. 35a). The trajectory was further validated through a pulse-chase experiment, where cells were harvested for TrackerSci profiling at different time points (i.e., one day, three days, and nine days post-labeling). Indeed, a gradual accumulation of more differentiated cell states with longer chasing time was observed (FIG. 36). Through differentially expressed gene analysis, 2,072 and 6,473 DE genes along the DG neurogenesis and OB neurogenesis trajectories, respectively were identified. Of all DE genes, 1,799 genes were shared between the two trajectories, including up-regulated genes (e.g., Dcx) enriched in neuron development (q-value=2.721e-8) (Chen et al., BMC Bioinformatics 14, 128 (2013)) and down-regulated genes (e.g., Notum) enriched in negative Wnt signaling regulation (q-value=0.0004) (Chen et al., BMC Bioinformatics 14, 128 (2013)) (FIG. 37a). In addition, putative trajectory- and region-specific neurogenesis programs were identified, such as transcriptional factors Neurod1, Neurod2, and Emx1 in the DG trajectory (FIG. 37b). This is consistent with previous reports about their important roles in hippocampal neurogenesis (Brulet et al., Exp. Neurol. 293, 190-198 (2017); Hong et al., Exp. Neurol. 206, 24-32 (2007); Micheli et al., Front. Cell. Neurosci. 11, 186 (2017)) (FIG. 35b).

With the chromatin accessibility profiling, 3,095 and 13,790 sites showing dynamics patterns along the DG neurogenesis and OB neurogenesis trajectories were identified, respectively, from which 20 TFs exhibiting significantly changed motif accessibility in the DG neurogenesis trajectory (FDR of 0.05, Table 8) and 318 TFs in OB neurogenesis (FDR of 0.05, Table 9) were further identified. Key TFs were further validated by strong correlations between their expression and motif accessibility dynamics. For example, the expression of the above-mentioned neurogenesis regulators, Neurod1 and Neurod2, are positively correlated with their motif accessibility. In contrast, Mytl1, a known repressor of neural differentiation (Mall et al., Nature 544, 245-249 (2017)), shows a negatively correlated gene expression and motif accessibility. Leveraging this approach, TFs shared between two neurogenesis trajectories were identified (e.g., Mytl1, Ascl1, and E2f7); many of them have been known to regulate the specification of different neuron types (e.g., Dlx6, Sp8, Sp9 uniquely enriched in OB neurogenesis (Li et al., Cereb. Cortex 28, 3278-3294 (2018); Diaz-Guerra et al., Anat. Rec. 296, 1364-1382 (2013)). Meanwhile, several TFs (e.g., Irf2, Stat2, and Etv6) that show strong enrichment of both gene expression and motif accessibility in neuronal progenitor cells were identified, but their functions in neurogenesis were less-characterized in prior studies. Interestingly, these factors have been previously identified as essential regulators of other stem cell types, such as colonic stem cells (Irf2) (Minamide et al., Sci. Rep. 10, 14639 (2020)), mesenchymal stem cells (Stat2) (Yi et al., Gene 497, 131-139 (2012)), and hematopoietic stem cells (Etv6) (Yi et al., Gene 497, 131-139 (2012); Hock et al., Genes Dev. 18, 2336-2341 (2004)). The data suggest their potential roles in maintaining the proliferation status of neuronal progenitor cells in the brain.

TABLE 8
Differential accessible TF binding motifs along
pseudotime in the DG neurogenesis trajectory.

| TF | motif_ID | pval | qval |
|---|---|---|---|
| ‘Twist2’ | ENSMUSG00000007805_LINE47_Twist2_D | 0.000237248 | 0.017433712 |
| ‘Msc’ | ENSMUSG00000025930_LINE83_Msc_D | 3.10E−09 | 2.73E−06 |
| ‘Myog’ | ENSMUSG00000026459_LINE85_Myog_D | 0.000819361 | 0.037949347 |
| ‘Neurog2’ | ENSMUSG00000027967_LINE90_Neurog2_I | 0.000483329 | 0.025019409 |
| ‘Scx’ | ENSMUSG00000034161_LINE113_Scx_I | 0.000257543 | 0.017433712 |
| ‘Atoh8’ | ENSMUSG00000037621_LINE124_Atoh8_I | 0.000303842 | 0.017825377 |
| ‘Neurod2’ | ENSMUSG00000038255_LINE127_Neurod2_I | 0.000956182 | 0.042072011 |
| ‘Olig2’ | ENSMUSG00000039830_LINE130_Olig2_I | 0.000775629 | 0.037919642 |
| ‘Neurog3’ | ENSMUSG00000044312_LINE135_Neurog3_I | 0.000483329 | 0.025019409 |
| ‘Olig1’ | ENSMUSG00000046160_LINE142_Olig1_I | 1.23E−05 | 0.002349494 |
| ‘Nhlh2’ | ENSMUSG00000048540_LINE148_Nhlh2_D | 4.16E−05 | 0.004572436 |
| ‘Tcf15’ | ENSMUSG00000068079_LINE162_Tcf15_I | 0.000257543 | 0.017433712 |
| ‘Atoh1’ | ENSMUSG00000073043_LINE166_Atoh1_D_N2 | 1.87E−05 | 0.002349494 |
| ‘Scrt1’ | ENSMUSG00000048385_LINE563_Scrt1_I | 0.000295917 | 0.017825377 |
| ‘Myt11’ | ENSMUSG00000061911_LINE940_Myt11_I | 1.84E−05 | 0.002349494 |
| ‘Pknox2’ | ENSMUSG00000035934_LINE1297_Pknox2_D_N1 | 0.000138454 | 0.013537713 |
| ‘Tal2’ | ENSMUSG00000028417_LINE214_Tal2_I_N1 | 0.000237248 | 0.017433712 |
| ‘Neurod1’ | ENSMUSG00000034701_LINE292_Neurod1_I_N7 | 1.87E−05 | 0.002349494 |
| ‘Neurod6’ | ENSMUSG00000037984_LINE330_Neurod6_I_N7 | 1.87E−05 | 0.002349494 |
| ‘Neurod4’ | ENSMUSG00000048015_LINE422_Neurod4_I_N7 | 1.87E−05 | 0.002349494 |

TABLE 9
Differential accessible TF binding motifs along pseudotime in the OB neurogenesis trajectory.

| TF | motif_ID | pval | qval |
|---|---|---|---|
| ‘Arid3b’ | ENSMUSG00000004661_LINE10_Arid3b_D | 1.75E−07 | 2.28E−06 |
| ‘Arid3a’ | ENSMUSG00000019564_LINE13_Arid3a_D_N3 | 0.001193739 | 0.005458936 |
| ‘Arid2’ | ENSMUSG00000033237_LINE27_Arid2_I | 0.000221127 | 0.00132676 |
| ‘Hmga2’ | ENSMUSG00000056758_LINE31_Hmga2_D | 0.009923832 | 0.029771496 |
| ‘Phf21a’ | ENSMUSG00000058318_LINE32_Phf21a_D | 6.45E−05 | 0.000474661 |
| ‘Ascl2’ | ENSMUSG00000009248_LINE50_Ascl2_D_N2 | 0.001919928 | 0.007962054 |
| ‘Myod1’ | ENSMUSG00000009471_LINE51_Myod1_D | 3.44E−05 | 0.000267094 |
| ‘Myc’ | ENSMUSG00000022346_LINE75_Myc_D | 4.60E−05 | 0.000350852 |
| ‘Myog’ | ENSMUSG00000026459_LINE85_Myog_D | 2.47E−06 | 2.64E−05 |
| ‘Hes2’ | ENSMUSG00000028940_LINE94_Hes2_D | 0.006030904 | 0.019548448 |
| ‘Atoh8’ | ENSMUSG00000037621_LINE124_Atoh8_I | 0.002275804 | 0.009212105 |
| ‘Hes5’ | ENSMUSG00000048001_LINE146_Hes5_D | 0.006652318 | 0.021157371 |
| ‘Max’ | ENSMUSG00000059436_LINE156_Max_D_N3 | 0.004949497 | 0.017021441 |
| ‘Atf6b’ | ENSMUSG00000015461_LINE174_Atf6b_I | 0.010335065 | 0.030678825 |
| ‘Fos' | ENSMUSG00000021250_LINE181_Fos_I | 0.000184034 | 0.001179492 |
| ‘Atf6’ | ENSMUSG00000026663_LINE196_Atf6_I | 0.010335065 | 0.030678825 |
| ‘Fosl2’ | ENSMUSG00000029135_LINE202_Fosl2_D | 0.000485873 | 0.00258521 |
| ‘Junb’ | ENSMUSG00000052837_LINE241_Junb_D | 0.011535377 | 0.033263317 |
| ‘Dbp’ | ENSMUSG00000059824_LINE252_Dbp_D_N2 | 0.002570977 | 0.010259653 |
| ‘Bcl6b’ | ENSMUSG00000000317_LINE277_Bcl6b_D | 0.00517088 | 0.017639372 |
| ‘Klf5’ | ENSMUSG00000005148_LINE292_Klf5_I | 6.33E−07 | 7.65E−06 |
| ‘Sp2’ | ENSMUSG00000018678_LINE317_Sp2_I | 8.18E−13 | 2.66E−11 |
| ‘Plagl1’ | ENSMUSG00000019817_LINE320_Plagl1_D | 0.000553122 | 0.002853301 |
| ‘Patz1’ | ENSMUSG00000020453_LINE325_Patz1_I | 0.003054142 | 0.01169142 |
| ‘Yy1’ | ENSMUSG00000021264_LINE330_Yy1_I | 0.000897737 | 0.004219362 |
| ‘Zkscan3’ | ENSMUSG00000021327_LINE332_Zkscan3_I | 0.006382467 | 0.020452905 |
| ‘Zfp369’ | ENSMUSG00000021514_LINE335_Zfp369_I | 0.005734676 | 0.01889848 |
| ‘Sp4’ | ENSMUSG00000025323_LINE369_Sp4_D_N2 | 0.003467034 | 0.013094243 |
| ‘Zfp7l1’ | ENSMUSG00000025529_LINE371_Zfp7l1_D | 5.44E−09 | 1.07E−07 |
| ‘Zfp202’ | ENSMUSG00000025602_LINE372_Zfp202_D | 0.002180448 | 0.008998337 |
| ‘Gfilb’ | ENSMUSG00000026815_LINE378_Gfilb_D | 0.00020226 | 0.00124176 |
| ‘Mecom’ | ENSMUSG00000027684_LINE385_Mecom_D | 0.001431955 | 0.006438028 |
| ‘Zfp300’ | ENSMUSG00000031079_LINE417_Zfp300_D | 0.003131419 | 0.011933244 |
| ‘Prdm1’ | ENSMUSG00000038151_LINE465_Prdm1_I | 0.004506218 | 0.016017901 |
| ‘Egr1’ | ENSMUSG00000038418_LINE467_Egr1_D_N3 | 0.008495928 | 0.026136563 |
| ‘Zfp410’ | ENSMUSG00000042472_LINE500_Zfp410_D | 0.015901061 | 0.042570562 |
| ‘Zfp3’ | ENSMUSG00000043602_LINE511_Zfp3_D | 1.76E−08 | 3.04E−07 |
| ‘Scrt1’ | ENSMUSG00000048385_LINE563_Scrt1_I | 2.60E−15 | 2.00E−13 |
| ‘Osr1’ | ENSMUSG00000048387_LINE564_Osr1_D | 0.005298076 | 0.017857259 |
| ‘Sp8’ | ENSMUSG00000048562_LINE568_Sp8_I | 0.002710296 | 0.010615324 |
| ‘Zfa’ | ENSMUSG00000049576_LINE578_Zfa_I | 6.93E−13 | 2.35E−11 |
| ‘Zfp161’ | ENSMUSG00000049672_LINE583_Zfp161_D | 0.011590856 | 0.033263317 |
| ‘Zbtb12’ | ENSMUSG00000049823_LINE587_Zbtb12_D | 0.000545838 | 0.002833 |
| ‘Hic2’ | ENSMUSG00000050240_LINE593_Hic2_I | 0.000200899 | 0.00124176 |
| ‘Zfy1’ | ENSMUSG00000053211_LINE623_Zfy1_I | 6.93E−13 | 2.35E−11 |
| ‘Zkscan4’ | ENSMUSG00000054931_LINE639_Zkscan4_I | 0.006382467 | 0.020452905 |
| ‘Zkscan5’ | ENSMUSG00000055991_LINE656_Zkscan5_D | 0.009163526 | 0.027786177 |
| ‘Zfp105’ | ENSMUSG00000057895_LINE676_Zfp105_D | 1.59E−05 | 0.000140292 |
| ‘Zfp110’ | ENSMUSG00000058638_LINE686_Zfp110_I | 0.005734676 | 0.01889848 |
| ‘Sert2’ | ENSMUSG00000060257_LINE703_Scrt2_I | 1.64E−14 | 9.89E−13 |
| ‘Sp7’ | ENSMUSG00000060284_LINE704_Sp7_I | 0.002710296 | 0.010615324 |
| ‘Zscan20’ | ENSMUSG00000061894_LINE719_Zscan20_D | 0.008925802 | 0.027162694 |
| ‘Zfp238’ | ENSMUSG00000063659_LINE743_Zfp238_I | 3.76E−07 | 4.68E−06 |
| ‘Sp9’ | ENSMUSG00000068859_LINE776_Sp9_I | 0.002710296 | 0.010615324 |
| ‘Egr4’ | ENSMUSG00000071341_LINE808_Egr4_I | 0.001020857 | 0.004771522 |
| ‘Zfx’ | ENSMUSG00000079509_LINE916_Zfx_D_N1 | 0.000281608 | 0.00160973 |
| ‘Myt1l’ | ENSMUSG00000061911_LINE940_Myt1l_I | 3.84E−28 | 3.25E−25 |
| ‘Nfya’ | ENSMUSG00000023994_LINE941_Nfya_I | 0.000202557 | 0.00124176 |
| ‘Onecut3’ | ENSMUSG00000045518_LINE965_Onecut3_I | 0.006919946 | 0.021844308 |
| ‘E2f7’ | ENSMUSG00000020185_LINE992_E2f7_I | 0.013138346 | 0.036927044 |
| ‘E2f5’ | ENSMUSG00000027552_LINE994_E2f5_I | 4.73E−05 | 0.000353752 |
| ‘E2f6’ | ENSMUSG00000057469_LINE996_E2f6_I | 0.000756822 | 0.003700992 |
| ‘Sfpi1’ | ENSMUSG00000002111_LINE997_Sfpi1_D_N2 | 0.005785705 | 0.01889848 |
| ‘Elf3’ | ENSMUSG00000003051_LINE1001_Elf3_D_N2 | 0.002385466 | 0.009610019 |
| ‘Elk3’ | ENSMUSG00000008398_LINE1009_Elk3_D_N2 | 0.000641397 | 0.003237001 |
| ‘Gabpa’ | ENSMUSG00000008976_LINE1011_Gabpa_D_N3 | 0.011638229 | 0.033263317 |
| ‘Elkl’ | ENSMUSG00000009406_LINE1014_Elk1_D | 0.001277246 | 0.005778344 |
| ‘Ehf’ | ENSMUSG00000012350_LINE1015_Ehf_D_N2 | 0.003174436 | 0.012042926 |
| ‘Elk4’ | ENSMUSG00000026436_LINE1023_Elk4_D | 0.006588712 | 0.021034153 |
| ‘Elf5’ | ENSMUSG00000027186_LINE1024_Elf5_D_N2 | 0.001131234 | 0.005201217 |
| ‘Etv6’ | ENSMUSG00000030199_LINE1026_Etv6_D | 4.66E−05 | 0.000352175 |
| ‘Elf4’ | ENSMUSG00000031103_LINE1027_Elf4_D | 0.000215233 | 0.001308792 |
| ‘Elf2’ | ENSMUSG00000037174_LINE1031_Elf2_D | 0.001746859 | 0.007426346 |
| ‘Erg’ | ENSMUSG00000040732_LINE1032_Erg_D_N1 | 0.018022373 | 0.047946314 |
| ‘Fev’ | ENSMUSG00000055197_LINE1037_Fev_I | 0.002739658 | 0.010631882 |
| ‘LINE5773’ | XP_9117244_LINE5773_Gm4881_I_N36 | 0.011638229 | 0.033263317 |
| ‘Foxj1’ | ENSMUSG00000034227_LINE1061_Foxj1_D | 3.54E−09 | 7.14E−08 |
| ‘Foxo1’ | ENSMUSG00000044167_LINE1080_Foxo1_D_N2 | 0.012637312 | 0.035637221 |
| ‘Foxi1’ | ENSMUSG00000047861_LINE1083_Foxi1_I | 0.014760252 | 0.040151683 |
| ‘Foxi2’ | ENSMUSG00000048377_LINE1084_Foxi2_I | 0.014760252 | 0.040151683 |
| ‘Foxc1’ | ENSMUSG00000050295_LINE1086_Foxc1_D_N2 | 0.001627955 | 0.006991116 |
| ‘Foxi3’ | ENSMUSG00000055874_LINE1093_Foxi3_I | 0.014760252 | 0.040151683 |
| ‘Foxd3’ | ENSMUSG00000067261_LINE1101_Foxd3_I | 0.001483943 | 0.006438028 |
| ‘Foxe1’ | ENSMUSG00000070990_LINE1102_Foxe1_I | 0.001483943 | 0.006438028 |
| ‘Foxd1’ | ENSMUSG00000078302_LINE1106_Foxd1_D | 0.004173324 | 0.015088173 |
| ‘LINE9878’ | NP_0011820571_LINE9878_Gm5294_I_N2 | 0.004173324 | 0.015088173 |
| ‘LINE9832’ | NP_0011820571_LINE9832_Gm5294_I_N1 | 3.54E−09 | 7.14E−08 |
| ‘LINE9910’ | NP_0011820571_LINE9910_Gm5294_I_N5 | 0.012176163 | 0.034451619 |
| ‘LINE9852’ | NP_0011820571_LINE9852_Gm5294_I_N1 | 0.014760252 | 0.040151683 |
| ‘LINE9851’ | NP_0011820571_LINE9851_Gm5294_I_N2 | 0.001483943 | 0.006438028 |
| ‘LINE9930’ | NP_0011820571_LINE9930_Gm5294_I_N3 | 0.010865198 | 0.03180608 |
| ‘LINE9919’ | NP_0011820571_LINE9919_Gm5294_I_N2 | 0.005275007 | 0.017850625 |
| ‘LINE9858’ | NP_0011820571_LINE9858_Gm5294_I_N1 | 0.000650461 | 0.003237001 |
| ‘LINE9950’ | NP_0320502_LINE9950_Foxl1_I_N1 | 3.54E−09 | 7.14E−08 |
| ‘LINE10003’ | NP_0320502_LINE10003_Foxl1_I_N2 | 0.004173324 | 0.015088173 |
| ‘LINE9973’ | NP_0320502_LINE9973_Foxl1_I_N1 | 0.014760252 | 0.040151683 |
| ‘LINE10033’ | NP_0320502_LINE10033_Foxl1_I_N5 | 0.012176163 | 0.034451619 |
| ‘LINE10052’ | NP_0320502_LINE10052_Foxl1_I_N5 | 7.54E−05 | 0.000535861 |
| ‘LINE9972’ | NP_0320502_LINE9972_Foxl1_I_N2 | 0.001483943 | 0.006438028 |
| ‘LINE10046’ | NP_0320502_LINE10046_Foxl1_I_N3 | 0.014958051 | 0.04055933 |
| ‘LINE10042’ | NP_0320502_LINE10042_Foxl1_I_N2 | 0.005275007 | 0.017850625 |
| ‘LINE9979’ | NP_0320502_LINE9979_Foxl1_I_N1 | 0.000650461 | 0.003237001 |
| ‘LINE9995’ | NP_0320502_LINE9995_Foxl1_I_N1 | 1.59E−06 | 1.77E−05 |
| ‘LINE10076’ | NP_0320502_LINE10076_Foxl1_I_N1 | 0.000250829 | 0.001453435 |
| ‘LINE10077’ | NP_0320502_LINE10077_Foxl1_I_N1 | 3.21E−06 | 3.35E−05 |
| ‘Gata6’ | ENSMUSG00000005836_LINE1110_Gata6_D | 0.002844155 | 0.01098701 |
| ‘Gata2’ | ENSMUSG00000015053_LINE1112_Gata2_I | 1.01E−05 | 9.31E−05 |
| ‘Gata3’ | ENSMUSG00000015619_LINE1113_Gata3_D | 1.32E−06 | 1.50E−05 |
| ‘Gata5’ | ENSMUSG00000015627_LINE1114_Gata5_D | 0.002911098 | 0.011194495 |
| ‘Gata4’ | ENSMUSG00000021944_LINE1116_Gata4_D_N1 | 8.98E−08 | 1.41E−06 |
| ‘Gata1’ | ENSMUSG00000031162_LINE1118_Gata1_D | 2.00E−06 | 2.17E−05 |
| ‘Tcfcp2l1’ | ENSMUSG00000026380_LINE1133_Tcfcp2l1_D_N2 | 0.000237672 | 0.001404282 |
| ‘LINE1139’ | A1JVI6_MOUSE_LINE1139_Dux_D | 0.00473653 | 0.016626988 |
| ‘Lhx2’ | ENSMUSG00000000247_LINE1140_Lhx2_D | 0.003509204 | 0.013194608 |
| ‘Hoxa4’ | ENSMUSG00000000942_LINE1144_Hoxa4_D | 0.001481579 | 0.006438028 |
| ‘Sebox’ | ENSMUSG00000001103_LINE1145_Sebox_D | 1.29E−07 | 1.73E−06 |
| ‘Meox1’ | ENSMUSG00000001493_LINE1146_Meox1_D | 1.81E−14 | 1.02E−12 |
| ‘Dlx3’ | ENSMUSG00000001510_LINE1149_Dlx3_D | 3.32E−07 | 4.20E−06 |
| ‘Hoxc13’ | ENSMUSG00000001655_LINE1151_Hoxc13_D | 0.000394319 | 0.002180352 |
| ‘Hoxc11’ | ENSMUSG00000001656_LINE1152_Hoxc11_D | 0.00046936 | 0.002513155 |
| ‘Hoxc8’ | ENSMUSG00000001657_LINE1153_Hoxc8_D | 5.54E−08 | 8.85E−07 |
| ‘Hoxc6’ | ENSMUSG00000001661_LINE1154_Hoxc6_D | 2.27E−20 | 3.85E−18 |
| ‘Hoxd13’ | ENSMUSG00000001819_LINE1156_Hoxd13_D_N3 | 0.000241476 | 0.001408886 |
| ‘Otx1’ | ENSMUSG00000005917_LINE1161_Otx1_D_N2 | 2.90E−05 | 0.000231314 |
| ‘Pknox1’ | ENSMUSG00000006705_LINE1167_Pknox1_D | 0.003891232 | 0.014251004 |
| ‘Pou6f1’ | ENSMUSG00000009739_LINE1170_Pou6f1_D_N2 | 0.003578326 | 0.013394972 |
| ‘Nanog’ | ENSMUSG00000012396_LINE1172_Nanog_D | 0.004662815 | 0.016505196 |
| ‘Phox2b’ | ENSMUSG00000012520_LINE1173_Phox2b_D | 6.96E−05 | 0.000499272 |
| ‘Alx3’ | ENSMUSG00000014603_LINE1174_Alx3_D | 5.10E−13 | 1.88E−11 |
| ‘Hoxa2’ | ENSMUSG00000014704_LINE1175_Hoxa2_D_N2 | 6.73E−09 | 1.27E−07 |
| ‘Lhx1’ | ENSMUSG00000018698_LINE1181_Lhx1_D | 0.000644279 | 0.003237001 |
| ‘Meis1’ | ENSMUSG00000020160_LINE1184_Meis1_D_N2 | 0.003710108 | 0.013706338 |
| ‘Hnf1b’ | ENSMUSG00000020679_LINE1186_Hnf1b_D | 1.86E−05 | 0.000157463 |
| ‘Dlx4’ | ENSMUSG00000020871_LINE1187_Dlx4_D | 2.47E−05 | 0.000202507 |
| ‘Gsc’ | ENSMUSG00000021095_LINE1189_Gsc_D | 0.000765333 | 0.003721103 |
| ‘Vsx2’ | ENSMUSG00000021239_LINE1192_Vsx2_I | 0.000459309 | 0.002475001 |
| ‘Barx1’ | ENSMUSG00000021381_LINE1193_Barx1_D | 0.017275944 | 0.046105516 |
| ‘Pitx1’ | ENSMUSG00000021506_LINE1195_Pitx1_D | 0.000141664 | 0.000990476 |
| ‘Irx4’ | ENSMUSG00000021604_LINE1196_Irx4_D | 7.89E−07 | 9.28E−06 |
| ‘Otp’ | ENSMUSG00000021685_LINE1197_Otp_D | 1.46E−20 | 3.08E−18 |
| ‘Otx2’ | ENSMUSG00000021848_LINE1198_Otx2_D | 2.90E−05 | 0.000231314 |
| ‘Hmbox1’ | ENSMUSG00000021972_LINE1199_Hmbox1_D | 1.86E−05 | 0.000157463 |
| ‘Hoxc10’ | ENSMUSG00000022484_LINE1203_Hoxc10_D_N1 | 9.21E−08 | 1.42E−06 |
| ‘Gsc2’ | ENSMUSG00000022738_LINE1207_Gsc2_I | 0.000603907 | 0.003077742 |
| ‘Dlx2’ | ENSMUSG00000023391_LINE1210_Dlx2_D_N2 | 3.03E−12 | 8.02E−11 |
| ‘Esx1’ | ENSMUSG00000023443_LINE1211_Esx1_D | 1.46E−20 | 3.08E−18 |
| ‘Rax’ | ENSMUSG00000024518_LINE1214_Rax_D | 0.000144178 | 0.000999792 |
| ‘Cdx1’ | ENSMUSG00000024619_LINE1215_Cdx1_D | 0.000151074 | 0.001022466 |
| ‘Lbx1’ | ENSMUSG00000025216_LINE1218_Lbx1_I | 3.11E−15 | 2.19E−13 |
| ‘Pitx3’ | ENSMUSG00000025229_LINE1219_Pitx3_D | 1.01E−07 | 1.45E−06 |
| ‘Msx3’ | ENSMUSG00000025469_LINE1222_Msx3_D_N2 | 0.000838914 | 0.004009725 |
| ‘Lhx4’ | ENSMUSG00000026468_LINE1223_Lhx4_D_N2 | 5.10E−13 | 1.88E−11 |
| ‘Prrx1’ | ENSMUSG00000026586_LINE1226_Prrx1_D | 1.46E−20 | 3.08E−18 |
| ‘Lmx1a’ | ENSMUSG00000026686_LINE1227_Lmx1a_D | 1.33E−06 | 1.50E−05 |
| ‘Barhl1’ | ENSMUSG00000026805_LINE1228_Barhl1_D_N3 | 0.000655022 | 0.003240634 |
| ‘Lhx3’ | ENSMUSG00000026934_LINE1233_Lhx3_D_N1 | 2.40E−12 | 7.01E−11 |
| ‘Meis2’ | ENSMUSG00000027210_LINE1237_Meis2_D_N2 | 0.001456707 | 0.006438028 |
| ‘Shox2’ | ENSMUSG00000027833_LINE1241_Shox2_D_N2 | 9.85E−06 | 9.16E−05 |
| ‘Pitx2’ | ENSMUSG00000028023_LINE1243_Pitx2_D | 0.001063538 | 0.004916685 |
| ‘Lhx8’ | ENSMUSG00000028201_LINE1244_Lhx8_D_N2 | 0.000523886 | 0.002735849 |
| ‘Dmbx1’ | ENSMUSG00000028707_LINE1248_Dmbx1_D | 1.09E−05 | 9.91E−05 |
| ‘Nkx11’ | ENSMUSG00000029112_LINE1250_Nkx11_I | 0.000162352 | 0.001056537 |
| ‘Uncx’ | ENSMUSG00000029546_LINE1251_Uncx_D_N2 | 2.56E−06 | 2.71E−05 |
| ‘Hnf1a’ | ENSMUSG00000029556_LINE1254_Hnf1a_D_N2 | 0.000200549 | 0.00124176 |
| ‘Lhx5’ | ENSMUSG00000029595_LINE1256_Lhx5_D | 3.51E−13 | 1.75E−11 |
| ‘Dlx5’ | ENSMUSG00000029755_LINE1267_Dlx5_D | 1.24E−07 | 1.69E−06 |
| ‘Hoxa1’ | ENSMUSG00000029844_LINE1268_Hoxa1_D | 1.16E−12 | 3.63E−11 |
| ‘Dbx1’ | ENSMUSG00000030507_LINE1269_Dbx1_D | 2.09E−12 | 6.31E−11 |
| ‘Cdx4’ | ENSMUSG00000031326_LINE1271_Cdx4_I | 0.000151074 | 0.001022466 |
| ‘Irx6’ | ENSMUSG00000031738_LINE1277_Irx6_D | 2.27E−05 | 0.000190192 |
| ‘Isl2’ | ENSMUSG00000032318_LINE1281_Isl2_D | 0.007602138 | 0.023472294 |
| ‘Hdx’ | ENSMUSG00000034551_LINE1289_Hdx_D | 0.003662606 | 0.013590196 |
| ‘Nkx61’ | ENSMUSG00000035187_LINE1293_Nkx61_D | 3.03E−12 | 8.02E−11 |
| ‘Arx’ | ENSMUSG00000035277_LINE1294_Arx_D_N1 | 9.85E−06 | 9.16E−05 |
| ‘Pknox2’ | ENSMUSG00000035934_LINE1297_Pknox2_D_N1 | 0.000156955 | 0.001038516 |
| ‘Gsx2’ | ENSMUSG00000035946_LINE1299_Gsx2_D | 1.49E−07 | 1.97E−06 |
| ‘Hoxc9’ | ENSMUSG00000036139_LINE1300_Hoxc9_D_N1 | 4.06E−13 | 1.81E−11 |
| ‘Meox2’ | ENSMUSG00000036144_LINE1302_Meox2_D | 5.48E−20 | 7.73E−18 |
| ‘Alx1’ | ENSMUSG00000036602_LINE1303_Alx1_D_N3 | 0.000311479 | 0.001745108 |
| ‘Hoxa13’ | ENSMUSG00000038203_LINE1307_Hoxa13_D | 1.89E−06 | 2.07E−05 |
| ‘Hoxa7’ | ENSMUSG00000038236_LINE1314_Hoxa7_D_N2 | 4.42E−10 | 9.58E−09 |
| ‘Hoxa5’ | ENSMUSG00000038253_LINE1315_Hoxa5_D | 0.01003807 | 0.030007799 |
| ‘Hoxb4’ | ENSMUSG00000038692_LINE1316_Hoxb4_D | 0.000157128 | 0.001038516 |
| ‘Pbx3’ | ENSMUSG00000038718_LINE1318_Pbx3_I | 0.005917959 | 0.01925613 |
| ‘Hoxb7’ | ENSMUSG00000038721_LINE1319_Hoxb7_D | 6.82E−05 | 0.000493196 |
| ‘Linx1b’ | ENSMUSG00000038765_LINE1320_Lmx1b_D | 4.11E−16 | 3.48E−14 |
| ‘Six3’ | ENSMUSG00000038805_LINE1321_Six3_D | 0.011904886 | 0.033910887 |
| ‘En2’ | ENSMUSG00000039095_LINE1322_En2_D_N2 | 0.001850831 | 0.007790064 |
| ‘Hlx’ | ENSMUSG00000039377_LINE1324_Hlx_D | 4.11E−16 | 3.48E−14 |
| ‘Alx4’ | ENSMUSG00000040310_LINE1330_Alx4_D_N1 | 0.000311479 | 0.001745108 |
| ‘Hesx1’ | ENSMUSG00000040726_LINE1334_Hesx1_I | 6.29E−06 | 6.34E−05 |
| ‘ENSMUSG00000040953’ | ENSMUSG00000040953_LINE1336_ENSMUSG00000040953_I | 0.014019013 | 0.038758447 |
| ‘Meis3’ | ENSMUSG00000041420_LINE1338_Meis3_D_N2 | 0.001456707 | 0.006438028 |
| ‘Crx’ | ENSMUSG00000041578_LINE1341_Crx_D_N1 | 2.90E−05 | 0.000231314 |
| ‘Obox’ | ENSMUSG00000041583_LINE1343_Obox6_D | 0.014019013 | 0.038758447 |
| ‘Dlx1’ | ENSMUSG00000041911_LINE1345_Dlx1_D_N2 | 0.000269466 | 0.001550803 |
| ‘Isl1’ | ENSMUSG00000042258_LINE1347_Isl1_I | 0.007602138 | 0.023472294 |
| ‘Hoxd11’ | ENSMUSG00000042499_LINE1350_Hoxd11_D | 0.006121383 | 0.019765991 |
| ‘Hoxa6’ | ENSMUSG00000043219_LINE1351_Hoxa6_D | 2.66E−10 | 5.98E−09 |
| ‘Hoxd9’ | ENSMUSG00000043342_LINE1352_Hoxd9_D_N1 | 0.010527 | 0.031139307 |
| ‘Gm4830’ | ENSMUSG00000044538_LINE1358_Gm4830_D | 0.000739742 | 0.003638498 |
| ‘Prop1’ | ENSMUSG00000044542_LINE1359_Prop1_D | 5.10E−13 | 1.88E−11 |
| ‘Dbx2’ | ENSMUSG00000045608_LINE1361_Dbx2_D | 1.24E−07 | 1.69E−06 |
| ‘Msx1’ | ENSMUSG00000048450_LINE1363_Msx1_D | 0.007602138 | 0.023472294 |
| ‘Nkx12’ | ENSMUSG00000048528_LINE1366_Nkx12_D | 3.01E−05 | 0.00023833 |
| ‘Hoxb13’ | ENSMUSG00000049604_LINE1368_Hoxb13_D | 6.63E−05 | 0.000483663 |
| ‘Rhox11’ | ENSMUSG00000051038_LINE1376_Rhox11_D_N4 | 0.001572542 | 0.006787607 |
| ‘Obox1’ | ENSMUSG00000054310_LINE1388_Obox1_D | 1.46E−05 | 0.000130012 |
| ‘Pou3f4’ | ENSMUSG00000056854_LINE1392_Pou3f4_D | 2.41E−05 | 0.000200248 |
| ‘En1’ | ENSMUSG00000058665_LINE1394_En1_D_N1 | 5.10E−13 | 1.88E−11 |
| ‘Irx1’ | ENSMUSG00000060969_LINE1400_Irx1_I | 0.000413591 | 0.002272067 |
| ‘Nkx63’ | ENSMUSG00000063672_LINE1405_Nkx63_I | 4.59E−08 | 7.47E−07 |
| ‘Rhox8’ | ENSMUSG00000064137_LINE1407_Rhox8_I | 0.004734791 | 0.016626988 |
| ‘Tlx2’ | ENSMUSG00000068327_LINE1414_Tlx2_D | 4.00E−07 | 4.91E−06 |
| ‘Obox5’ | ENSMUSG00000074366_LINE1427_Obox5_D | 1.79E−05 | 0.000154875 |
| ‘AC1890281’ | ENSMUSG00000074368_LINE1428_AC1890281_D | 0.011505872 | 0.033263317 |
| ‘Hoxb2’ | ENSMUSG00000075588_LINE1434_Hoxb2_I | 0.000866963 | 0.00412051 |
| ‘Hoxd3’ | ENSMUSG00000079277_LINE1439_Hoxd3_D_N2 | 8.73E−11 | 2.05E−09 |
| ‘LINE6215’ | NP_0010765961_LINE6215_NP_0010765961_I_N4 | 6.63E−06 | 6.59E−05 |
| ‘LINE6216’ | NP_0010765961_LINE6216_NP_0010765961_I_N4 | 5.41E−05 | 0.000401756 |
| ‘LINE6234’ | NP_0322962_LINE6234_NP_0322962_I_N2 | 4.06E−13 | 1.81E−11 |
| ‘LINE6255’ | NP_0322962_LINE6255_NP_0322962_I_N2 | 1.79E−07 | 2.30E−06 |
| ‘LINE6262’ | NP_0322962_LINE6262_NP_0322962_I_N2 | 0.000239027 | 0.001404282 |
| ‘LINE6275’ | NP_0323002_LINE6275_NP_0323002_I_N8 | 0.000808865 | 0.003910282 |
| ‘LINE6276’ | NP_0832781_LINE6276_NP_0832781_I_N11 | 3.03E−12 | 8.02E−11 |
| ‘LINE1462’ | NP_6637552_LINE1462_NP_6637552_I_N11 | 1.79E−05 | 0.000154875 |
| ‘LINE1463’ | Q8VHG7_MOUSE_LINE1463_Q8VHG7_MOUSE_I | 0.011505872 | 0.033263317 |
| ‘LINE1464’ | XP_0014736851_LINE1464_Nkx11_D_N7 | 0.000162352 | 0.001056537 |
| ‘Pou1f1’ | ENSMUSG00000004842_LINE1469_Pou1f1_D_N3 | 0.002547605 | 0.010214569 |
| ‘Pou2f2’ | ENSMUSG00000008496_LINE1472_Pou2f2_D_N1 | 3.37E−05 | 0.000264071 |
| ‘Pou2f1’ | ENSMUSG00000026565_LINE1482_Pou2f1_D_N1 | 3.75E−08 | 6.23E−07 |
| ‘LINE15940’ | NP_0328751_LINE15940_Pit1_I_N1 | 0.000145431 | 0.00100028 |
| ‘Hsf2’ | ENSMUSG00000019878_LINE1489_Hsf2_I | 0.013936365 | 0.038758447 |
| ‘Hsf1’ | ENSMUSG00000022556_LINE1490_Hsf1_I | 0.004839921 | 0.016781038 |
| ‘Irf9’ | ENSMUSG00000002325_LINE1495_Irf9_D | 5.52E−06 | 5.62E−05 |
| ‘Irf3’ | ENSMUSG00000003184_LINE1496_Irf3_D | 0.005108253 | 0.017496283 |
| ‘Irf1’ | ENSMUSG00000018899_LINE1497_Irf1_I | 2.75E−11 | 6.84E−10 |
| ‘Irf4’ | ENSMUSG00000021356_LINE1498_Irf4_D | 9.96E−05 | 0.000701864 |
| ‘Irf7’ | ENSMUSG00000025498_LINE1499_Irf7_D | 2.24E−08 | 3.79E−07 |
| ‘Irf5’ | ENSMUSG00000029771_LINE1501_Irf5_D | 0.013778579 | 0.038470885 |
| ‘Irf2’ | ENSMUSG00000031627_LINE1502_Irf2_D | 0.00036855 | 0.002051274 |
| ‘Mef2a’ | ENSMUSG00000030557_LINE1510_Mef2a_I | 1.38E−05 | 0.000124289 |
| ‘Cdc5l’ | ENSMUSG00000023932_LINE1532_Cdc5l_I | 0.003644491 | 0.013582551 |
| ‘Pparg’ | ENSMUSG00000000440_LINE1566_Pparg_I | 0.000170946 | 0.001103971 |
| ‘Nr1h3’ | ENSMUSG00000002108_LINE1571_Nr1h3_I | 0.0154194 | 0.041543988 |
| ‘Ppard’ | ENSMUSG00000002250_LINE1572_Ppard_I | 0.000521184 | 0.002735849 |
| ‘Nr1i3’ | ENSMUSG00000005677_LINE1576_Nr1i3_I | 3.73E−05 | 0.000287112 |
| ‘Nr2c2’ | ENSMUSG00000005893_LINE1577_Nr2c2_I | 0.002727545 | 0.010631882 |
| ‘Hnf4g’ | ENSMUSG00000017688_LINE1589_Hnf4g_I | 2.68E−10 | 5.98E−09 |
| ‘Hnf4a’ | ENSMUSG00000017950_LINE1590_Hnf4a_D_N1 | 6.68E−07 | 7.95E−06 |
| ‘Esr2’ | ENSMUSG00000021055_LINE1597_Esr2_I | 0.005409869 | 0.018161704 |
| ‘Vdr’ | ENSMUSG00000022479_LINE1604_Vdr_D | 0.005508218 | 0.018418784 |
| ‘Nr1i2’ | ENSMUSG00000022809_LINE1605_Nr1i2_I | 0.004804328 | 0.016781038 |
| ‘Nr3c1’ | ENSMUSG00000024431_LINE1607_Nr3c1_D | 2.83E−14 | 1.49E−12 |
| ‘Rorc’ | ENSMUSG00000028150_LINE1616_Rorc_I | 6.39E−09 | 1.23E−07 |
| ‘Rora’ | ENSMUSG00000032238_LINE1621_Rora_D | 0.000216585 | 0.001308792 |
| ‘Nr2e3’ | ENSMUSG00000032292_LINE1622_Nr2e3_D | 0.000295171 | 0.001675936 |
| ‘Nr1h2’ | ENSMUSG00000060601_LINE1635_Nr1h2_I | 0.0154194 | 0.041543988 |
| ‘Nr6a1’ | ENSMUSG00000063972_LINE1636_Nr6a1_I | 0.013533646 | 0.037912134 |
| ‘Rel’ | ENSMUSG00000020275_LINE1654_Rel_I | 4.13E−06 | 4.26E−05 |
| ‘Nfkb1’ | ENSMUSG00000028163_LINE1660_Nfkb1_I | 0.004302539 | 0.015358429 |
| ‘Rfx2’ | ENSMUSG00000024206_LINE1666_Rfx2_D_N1 | 9.90E−08 | 1.44E−06 |
| ‘Rfx8’ | ENSMUSG00000057173_LINE1673_Rfx8_I | 9.90E−08 | 1.44E−06 |
| ‘Runx2’ | ENSMUSG00000039153_LINE1675_Runx2_I | 0.001269727 | 0.005775211 |
| ‘Nfix’ | ENSMUSG00000001911_LINE1689_Nfix_I | 0.001674877 | 0.007156294 |
| ‘Nfic’ | ENSMUSG00000055053_LINE1700_Nfic_I | 0.006699403 | 0.021227323 |
| ‘Sox9’ | ENSMUSG00000000567_LINE1701_Sox9_I | 0.007564504 | 0.023472294 |
| ‘Hbp1’ | ENSMUSG00000002996_LINE1704_Hbp1_D | 5.30E−19 | 6.41E−17 |
| ‘Hmg20b’ | ENSMUSG00000020232_LINE1710_Hmg20b_D | 4.35E−15 | 2.83E−13 |
| ‘Bbx’ | ENSMUSG00000022641_LINE1712_Bbx_D | 0.000602098 | 0.003077742 |
| ‘Sox17’ | ENSMUSG00000025902_LINE1716_Sox17_D_N1 | 0.01062789 | 0.031304278 |
| ‘Sox3’ | ENSMUSG00000045179_LINE1741_Sox3_D_N1 | 6.85E−06 | 6.74E−05 |
| ‘Sox14’ | ENSMUSG00000053747_LINE1752_Sox14_D_N1 | 1.20E−07 | 1.69E−06 |
| ‘Tcf7l1’ | ENSMUSG00000055799_LINE1755_Tcf7l1_D | 0.015550218 | 0.041763443 |
| ‘ENSMUSG00000079994’ | ENSMUSG00000079994_LINE1775_ENSMUSG00000079994_D_N1 | 0.001869697 | 0.007830515 |
| ‘Stat6’ | ENSMUSG00000002147_LINE1780_Stat6_D | 0.001035523 | 0.004813476 |
| ‘Stat2’ | ENSMUSG00000040033_LINE1785_Stat2_I | 1.85E−16 | 1.95E−14 |
| ‘Tbp’ | ENSMUSG00000014767_LINE1805_Tbp_D | 0.00422152 | 0.015133075 |
| ‘Tbpl2’ | ENSMUSG00000061809_LINE1806_Tbpl2_I | 0.00422152 | 0.015133075 |
| ‘Prkrir’ | ENSMUSG00000030753_LINE1815_Prkrir_I | 0.001825941 | 0.007723731 |
| ‘Hmga1’ | ENSMUSG00000046711_LINE73_Hmga1_I_N2 | 0.009923832 | 0.029771496 |
| ‘Hmga1rs1’ | ENSMUSG00000078249_LINE85_Hmga1rs1_I_N2 | 0.009923832 | 0.029771496 |
| ‘Myf5’ | ENSMUSG00000000435_LINE113_Myf5_I_N8 | 1.44E−08 | 2.54E−07 |
| ‘Ascl1’ | ENSMUSG00000020052_LINE158_Ascl1_I_N2 | 0.001919928 | 0.007962054 |
| ‘Tal1’ | ENSMUSG00000028717_LINE236_Tal1_I_N4 | 0.007276594 | 0.022799994 |
| ‘Lyl1’ | ENSMUSG00000034041_LINE273_Lyl1_I_N4 | 0.007276594 | 0.022799994 |
| ‘Twist1’ | ENSMUSG00000035799_LINE296_Twist1_I_N2 | 0.003806104 | 0.013999844 |
| ‘Snai2’ | ENSMUSG00000022676_LINE952_Snai2_I_N5 | 0.000439728 | 0.002400064 |
| ‘Zfp148’ | ENSMUSG00000022811_LINE962_Zfp148_I_N2 | 5.08E−12 | 1.30E−10 |
| ‘Klf15’ | ENSMUSG00000030087_LINE1139_Klf15_I | 0.010656776 | 0.031304278 |
| ‘Maz’ | ENSMUSG00000030678_LINE1154_Maz_I | 6.94E−11 | 1.68E−09 |
| ‘Zfp219’ | ENSMUSG00000049295_LINE1480_Zfp219_I | 0.004895778 | 0.016905419 |
| ‘Ybx2’ | ENSMUSG00000018554_LINE2089_Ybx2_I_N1 | 0.005768634 | 0.01889848 |
| ‘Ybx1’ | ENSMUSG00000028639_LINE2093_Ybx1_I_N1 | 0.005768634 | 0.01889848 |
| ‘Csda’ | ENSMUSG00000030189_LINE2097_Csda_I_N1 | 0.005768634 | 0.01889848 |
| ‘Ets2’ | ENSMUSG00000022895_LINE2391_Ets2_I_N55 | 0.011638229 | 0.033263317 |
| ‘Ubp1’ | ENSMUSG00000009741_LINE5318_Ubp1_I_N2 | 0.000237672 | 0.001404282 |
| ‘Nkx62’ | ENSMUSG00000041309_LINE5799_Nkx62_I_N5 | 0.000152311 | 0.001022656 |
| ‘Pou4f2’ | ENSMUSG00000031688_LINE6328_Pou4f2_I_N4 | 9.62E−06 | 9.14E−05 |
| ‘Pou4f1’ | ENSMUSG00000048349_LINE6338_Pou4f1_I_N4 | 9.62E−06 | 9.14E−05 |
| ‘Mef2d’ | ENSMUSG00000001419_LINE6419_Mef2d_I_N14 | 0.00019202 | 0.001212305 |
| ‘Nr1d1’ | ENSMUSG00000020889_LINE6649_Nr1d1_I_N1 | 9.77E−09 | 1.76E−07 |
| ‘Nr1d2’ | ENSMUSG00000021775_LINE6674_Nr1d2_I_N1 | 9.77E−09 | 1.76E−07 |
| ‘Thrb’ | ENSMUSG00000021779_LINE6679_Thrb_I_N2 | 0.008796962 | 0.026867256 |
| ‘Nr4a1’ | ENSMUSG00000023034_LINE6710_Nr4a1_I_N5 | 0.000819831 | 0.003940777 |
| ‘Pgr’ | ENSMUSG00000031870_LINE6829_Pgr_I_N9 | 1.22E−06 | 1.41E−05 |
| ‘Thra’ | ENSMUSG00000058756_LINE6865_Thra_I_N2 | 0.008796962 | 0.026867256 |
| ‘Trp63’ | ENSMUSG00000022510_LINE6881_Trp63_I_N2 | 0.000496785 | 0.002626752 |
| ‘Relb’ | ENSMUSG00000002983_LINE7015_Relb_I | 0.00483953 | 0.016781038 |
| ‘Tbx15’ | ENSMUSG00000027868_LINE7567_Tbx15_I_N1 | 0.002255887 | 0.009175385 |
| ‘Tbx22’ | ENSMUSG00000031241_LINE7574_Tbx22_I_N1 | 0.002255887 | 0.009175385 |
| ‘Tbx18’ | ENSMUSG00000032419_LINE7580_Tbx18_I_N1 | 0.002255887 | 0.009175385 |
| ‘Tead3’ | ENSMUSG00000002249_LINE7609_Tead3_I_N1 | 0.002624079 | 0.010422397 |
| ‘Hif3a’ | ENSMUSG00000004328_LINE324_Hif3a_I_N1 | 9.69E−08 | 1.44E−06 |
| ‘Wt1’ | ENSMUSG00000016458_LINE2218_Wt1_I | 7.98E−06 | 7.76E−05 |
| ‘LINE3883’ | Q8K439_MOUSE_LINE3883_Zfp263_I_N2 | 0.000885828 | 0.004186649 |
| ‘LINE3878’ | Q8K439_MOUSE_LINE3878_Zfp263_I_N1 | 0.000448652 | 0.002433072 |
| ‘Mef2b’ | ENSMUSG00000079033_LINE16135_Mef2b_I_N14 | 0.00019202 | 0.001212305 |

To comprehensively investigate the impact of ageing on adult neurogenesis, the cellular density across different conditions along the neurogenesis trajectory were compared based on the recovered single-cell transcriptomes. Consistent with the cell type level analysis (FIG. 34c), a dramatic age-dependent reduction in the cellular density of neural progenitor cells (NPC) and DG neuroblasts (DGNB) was observed, but not in OB neuroblasts (FIG. 35c). The finding was further validated through the chromatin accessibility profile, where a recently published differential abundance testing algorithm, Milo (Dann et al., Nat. Biotechnol. (2021) doi:10.1038/s41587-021-01033-z), was applied to identify the cellular neighborhoods that are significantly altered upon ageing. Thirty-one differentially decreased cellular neighborhoods were identified (FIG. 35d, 5% FDR), mostly from the neural progenitor cells (NPC) and DG neuroblasts (DGNB). This analysis further validated that ageing affects neurogenesis by down-regulating the proliferation behaviors of its progenitor cells.

To further decipher the molecular mechanisms underlying the age-dependent changes in neuronal progenitor cells, differential gene expression analysis was performed across young, adult, and aged conditions and yielded thirty genes showing concordant changes over time, supported by both gene expression and accessibility of promoters or linked distal sites (FIG. 35e). For example, two neurotrophic factors involved in the Erbb pathway, Nrg1 and Nrg3, exhibited reduced expression and promoter accessibility upon ageing. Indeed, they have been shown to maintain neurogenesis upon administration in vivo (Mahar et al., Sci. Rep. 6, 30467 (2016)). In addition, several other known regulators of neurogenesis, such as Nr2f1 and Nap1l1 (Qiao et al., Cell Rep. 22, 2279-2293 (2018); Bertacchi et al., EMBO J. 39, e104163 (2020)), were significantly down-regulated upon ageing, suggesting they may serve as putative targets for restoring adult neurogenesis in future studies.

### The Impact of Ageing on Adult Oligodendrogenesis

Next, cell types that span multiple stages of oligodendrogenesis for pseudotime analysis were isolated in silico, yielding a simple trajectory defined by integrated transcriptome and chromatin accessibility profiles (FIG. 35a). The oligodendrogenesis trajectory was further validated by the RNA velocity analysis and the time-dependent labeling experiment mentioned above (FIG. 36). Through differential expression (DE) and differential accessibility (DA) analysis, 8,443 DE genes and 15,164 DA sites that were significantly changed along the trajectory (5% FDR) were identified. This analysis nominated known oligodendrogenesis regulators (e.g., Zfp276 (Aberle et al., Nucleic Acids Res. 50, 1951-1968 (2022)) and Myrf (Fletcher et al., Semin. Cell Dev. Biol. 118, 14-23 (2021)) and related pathways (e.g., cholesterol biosynthesis (Mathews et al., J. Neurosci. 36, 7628-7639 (2016)), as well as novel gene markers, such as Snx10, Rfbox2, and Tenm2 (FIG. 37c), that are validated by strong correlations between their expression and promoter accessibility dynamics in oligodendrogenesis but are less-characterized in previous studies. In addition, 97 TFs that exhibited significantly altered gene expression and motif accessibility were identified (FIG. 35b), including known regulators of oligodendrocyte differentiation such as Sox5, Sox10, Pknox1, and Nkx6-2 (Emery et al., Cold Spring Harb. Perspect. Biol. 7, a020461 (2015); Kato et al., PLOS One 10, e0145334 (2015); Javed et al., bioRxiv 2021.12.01.470829 (2021) doi:10.1101/2021.12.01.470829). Furthermore, novel TF markers were detected, including Ikzf4, a known regulator of Müller glia differentiation in retina (Javed et al., bioRxiv 2021.12.01.470829 (2021) doi:10.1101/2021.12.01.470829), and several potential transcriptional ‘repressors’ (e.g., Esrra, Esrrg, Elk3, Zeb1) characterized by the negative correlation between their expression and motif accessibility along the trajectory of oligodendrogenesis (FIG. 35b).

The impact of ageing on adult oligodendrogenesis was further investigated by examining cellular density across different conditions along the cellular differentiation trajectory. Unlike adult neurogenesis, a remarkable reduction in committed oligodendrocyte precursors (COPs) rather than the early progenitor cells was observed. The result is further validated through the Milo (Dann et al., Nat. Biotechnol. (2021) doi:10.1038/s41587-021-01033-z) analysis of chromatin accessibility profiles, where thirteen cellular neighborhoods that are differentially decreased upon ageing were identified, all exclusively overlapped with the committed oligodendrocyte precursors (COPs) (FIG. 35d, 5% FDR). In fact, a consistent ageing-associated depletion of newly formed oligodendrocytes was detected in the companion study (Cao et al., Science 370, 924-925 (2020)), which is in accordance with previous report (Givre et al., Journal of Neuro-Ophthalmology vol. 23 168 (2003)).

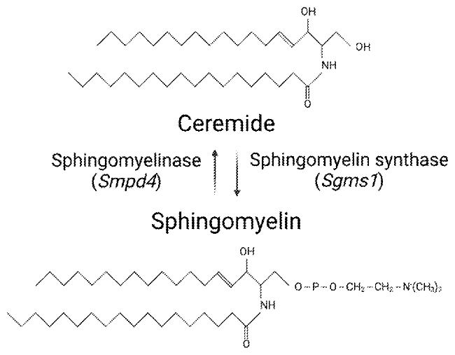

Finally, to delineate the molecular programs contributing to down-regulated oligodendrogenesis upon ageing, the significantly dysregulated genes in OPCs were examined and 242 DE genes were identified (FDR of 10%, Table 10). Many of the top DE genes are cross-validated by two independent molecular layers (i.e., both gene expression and promoter accessibility) and involved in molecular processes critical for oligodendrocyte differentiations such as cell cycle (e.g., Cables1 (He et al., Stem Cell Reports 13, 274-290 (2019)) or cell migration (e.g., Ephb1, Epha4, Plxna4) (Linneberg et al., ASN Neuro 7, (2015); Smith et al., Curr. Biol. 7, 561-570 (1997)). (FIG. 35e). For example, age-dependent down-regulation of Ryr2 (FIG. 35e) was detected, a ryanodine receptor that mediates endoplasmic reticulum Ca²⁺ release which is essential for initiating OPC differentiation (Li et al., Front. Mol. Neurosci. 11, 162 (2018)). Intriguingly, two sphingomyelin metabolism-related genes exhibited opposite dynamics between young and aged OPCs (FIG. 35e): Sgms1, a gene encoding a sphingomyelin synthase critical for converting phosphatidylcholine and ceramide to ceramide phosphocholine (sphingomyelin) and diacylglycerol at the Golgi apparatus (Tafesse et al., J. Biol. Chem. 282, 17537-17547 (2007); Huitema et al., EMBO J. 23, 33-44 (2004)), was substantially down-regulated in the aged OPCs. By contrast, Smpd4, encoding a sphingomyelin phosphodiesterase that catalyzes the reverse reaction (Krut et al., J. Biol. Chem. 281, 13784-13793 (2006)), was significantly up-regulated in OPCs upon ageing, (FIG. 38). As a result, the age-dependent changes of both Sgms1 and Smpd4 facilitate the accumulation of ceramide and depletion of sphingomyelin in OPCs, which has been reported to increase cellular susceptibility to senescence and cell death (Hannun et al., Nat. Rev. Mol. Cell Biol. 9, 139-150 (2008); Jana et al., J. Neurol. Sci. 278, 5-15 (2009)). This is consistent with a recent report that inhibiting another sphingomyelin hydrolase nSMase2 enhances myelination during the differentiation of OPCs (Yoo et al., Sci Adv 6, (2020)), suggesting a critical role of the dysregulated sphingomyelin metabolism in blocking oligodendrocyte differentiation.

TABLE 10
Differential expressed genes in oligodendrocytes progenitor cells
across aged groups supported by promoters or linked distal sites

| gene_id | gene_short_name | gene_type | pval | qval | comments |
|---|---|---|---|---|---|
| ENSMUSG00000021606.9 | ‘Ndufs6’ | PC | 5.92E−227 | 2.34E−223 | Ageing_depleted |
| ENSMUSG00000048327.7 | ‘Ckap2l’ | PC | 1.24E−67 | 6.99E−65 | Ageing_depleted |
| ENSMUSG00000042302.15 | ‘Ehbp1’ | PC | 8.21E−49 | 2.95E−46 | Ageing_depleted |
| ENSMUSG00000119584.1 | ‘Rn18s' | rRNA | 1.05E−28 | 2.87E−26 | Ageing_depleted |
| ENSMUSG00000026155.14 | ‘Smap1’ | PC | 1.52E−26 | 3.64E−24 | Ageing_depleted |
| ENSMUSG00000030990.19 | ‘Pgap2’ | PC | 2.31E−21 | 4.56E−19 | Ageing_depleted |
| ENSMUSG00000027777.16 | ‘Schip1’ | PC | 4.48E−18 | 7.72E−16 | Ageing_depleted |
| ENSMUSG00000062937.8 | ‘Mtap’ | PC | 6.40E−18 | 1.08E−15 | Ageing_depleted |
| ENSMUSG00000085456.3 | ‘Gm15398’ | IncRNA | 7.07E−17 | 1.08E−14 | Ageing_depleted |
| ENSMUSG00000029635.16 | ‘Cdk8’ | PC | 4.32E−14 | 5.60E−12 | Ageing_depleted |
| ENSMUSG00000034813.19 | ‘Grip1’ | PC | 1.55E−13 | 1.92E−11 | Ageing_depleted |
| ENSMUSG00000117441.2 | ‘Gm50021’ | IncRNA | 5.38E−13 | 6.55E−11 | Ageing_depleted |
| ENSMUSG00000069049.12 | ‘Eif2s3y’ | PC | 1.19E−12 | 1.41E−10 | Ageing_depleted |
| ENSMUSG00000024598.10 | ‘Fbn2’ | PC | 3.73E−11 | 3.79E−09 | Ageing_depleted |
| ENSMUSG00000038515.11 | ‘Grtp1’ | PC | 1.16E−10 | 1.15E−08 | Ageing_depleted |
| ENSMUSG00000021313.17 | ‘Ryr2’ | PC | 1.26E−09 | 1.14E−07 | Ageing_depleted |
| ENSMUSG00000110831.2 | ‘Gm48159’ | IncRNA | 1.60E−09 | 1.36E−07 | Ageing_depleted |
| ENSMUSG00000032537.16 | ‘Ephb1’ | PC | 2.49E−09 | 2.05E−07 | Ageing_depleted |
| ENSMUSG00000078489.3 | ‘Gm17106’ | IncRNA | 9.71E−09 | 7.54E−07 | Ageing_depleted |
| ENSMUSG00000029088.17 | ‘Kcnip4’ | PC | 1.17E−08 | 8.83E−07 | Ageing_depleted |
| ENSMUSG00000068457.15 | ‘Uty’ | PC | 7.68E−08 | 5.53E−06 | Ageing_depleted |
| ENSMUSG00000020524.17 | ‘Gria1’ | PC | 8.50E−08 | 5.91E−06 | Ageing_depleted |
| ENSMUSG00000008489.19 | ‘Elavl2’ | PC | 9.04E−08 | 6.22E−06 | Ageing_depleted |
| ENSMUSG00000046707.10 | ‘Csnk2a2 | PC | 1.86E−07 | 1.22E−05 | Ageing_depleted |
| ENSMUSG00000027238.18 | ‘Frmd5’ | PC | 2.76E−07 | 1.75E−05 | Ageing_depleted |
| ENSMUSG00000095041.8 | ‘ENSMUSG00000095041’ | PC | 7.28E−07 | 4.24E−05 | Ageing_depleted |
| ENSMUSG00000031585.14 | ‘Gtf2e2’ | PC | 1.28E−06 | 6.92E−05 | Ageing_depleted |
| ENSMUSG00000033854.11 | ‘Kcnk10’ | PC | 1.38E−06 | 7.37E−05 | Ageing_depleted |
| ENSMUSG00000029765.13 | ‘Plxna4’ | PC | 2.39E−06 | 0.000122679 | Ageing_depleted |
| ENSMUSG00000040451.19 | ‘Sgms1’ | PC | 6.71E−06 | 0.000316223 | Ageing_depleted |
| ENSMUSG00000028906.18 | ‘Epb41’ | PC | 6.85E−06 | 0.000320831 | Ageing_depleted |
| ENSMUSG00000027333.19 | ‘Smox’ | PC | 1.06E−05 | 0.000472257 | Ageing_depleted |
| ENSMUSG00000030518.18 | ‘Fam189a1’ | PC | 1.18E−05 | 0.000520296 | Ageing_depleted |
| ENSMUSG00000031790.9 | ‘Mmp15’ | PC | 1.62E−05 | 0.000691207 | Ageing_depleted |
| ENSMUSG00000026235.15 | ‘Epha4’ | PC | 1.83E−05 | 0.000764575 | Ageing_depleted |
| ENSMUSG00000074968.12 | ‘Ano3’ | PC | 1.98E−05 | 0.000823546 | Ageing_depleted |
| ENSMUSG00000067028.12 | ‘Cntnap5b’ | PC | 2.49E−05 | 0.00101514 | Ageing_depleted |
| ENSMUSG00000026914.16 | ‘Psmd14’ | PC | 3.78E−05 | 0.001490806 | Ageing_depleted |
| ENSMUSG00000034098.15 | ‘Fst15’ | PC | 4.09E−05 | 0.001596294 | Ageing_depleted |
| ENSMUSG00000028389.13 | ‘Zfp37’ | PC | 4.92E−05 | 0.001881396 | Ageing_depleted |
| ENSMUSG00000044499.12 | ‘Hs3st5’ | PC | 5.36E−05 | 0.00203401 | Ageing_depleted |
| ENSMUSG00000051323.17 | ‘Pcdh19’ | PC | 7.18E−05 | 0.002549189 | Ageing_depleted |
| ENSMUSG00000001786.15 | ‘Fbxo7’ | PC | 8.34E−05 | 0.00282182 | Ageing_depleted |
| ENSMUSG00000047213.15 | ‘Ythdf3’ | PC | 9.68E−05 | 0.003148867 | Ageing_depleted |
| ENSMUSG00000035864.15 | ‘Syt1’ | PC | 9.70E−05 | 0.003148867 | Ageing_depleted |
| ENSMUSG00000001017.16 | ‘Chtop’ | PC | 0.00010031 | 0.003202628 | Ageing_depleted |
| ENSMUSG00000025658.17 | ‘Cnksr2’ | PC | 0.000106332 | 0.003327806 | Ageing_depleted |
| ENSMUSG00000079671.9 | ‘2610203C22Rik’ | IncRNA | 0.000113831 | 0.003520758 | Ageing_depleted |
| ENSMUSG00000028949.14 | ‘Smarcd3’ | PC | 0.00012613 | 0.003826424 | Ageing_depleted |
| ENSMUSG00000042447.14 | ‘Mios' | PC | 0.000130793 | 0.003937714 | Ageing_depleted |
| ENSMUSG00000074785.6 | ‘Plxnc1’ | PC | 0.000139544 | 0.004086783 | Ageing_depleted |
| ENSMUSG00000052949.15 | ‘Rnf157’ | PC | 0.000146252 | 0.004195748 | Ageing_depleted |
| ENSMUSG00000027204.14 | ‘Fbn1’ | PC | 0.000202246 | 0.005355792 | Ageing_depleted |
| ENSMUSG00000043336.15 | ‘Filip1l’ | PC | 0.000217813 | 0.005691882 | Ageing_depleted |
| ENSMUSG00000103563.2 | ‘8030445P17Rik’ | TEC | 0.000228909 | 0.005942638 | Ageing_depleted |
| ENSMUSG00000022973.19 | ‘Synj1’ | PC | 0.000291742 | 0.007333385 | Ageing_depleted |
| ENSMUSG00000032030.17 | ‘Cul5’ | PC | 0.000331321 | 0.008076122 | Ageing_depleted |
| ENSMUSG00000011960.13 | ‘Ccnt1’ | PC | 0.000365871 | 0.008752161 | Ageing_depleted |
| ENSMUSG00000028360.11 | ‘Slc44a5’ | PC | 0.000398469 | 0.00936224 | Ageing_depleted |
| ENSMUSG00000034573.15 | ‘Ptpn13’ | PC | 0.000445318 | 0.010279966 | Ageing_depleted |
| ENSMUSG00000111842.2 | ‘Gm49318’ | PC | 0.000463107 | 0.010597926 | Ageing_depleted |
| ENSMUSG00000047261.10 | ‘Gap43’ | PC | 0.000465349 | 0.010618539 | Ageing_depleted |
| ENSMUSG00000029563.17 | ‘Foxp2’ | PC | 0.000513436 | 0.011582287 | Ageing_depleted |
| ENSMUSG00000094962.2 | ‘Gm21954’ | PC | 0.000568833 | 0.012651731 | Ageing_depleted |
| ENSMUSG00000098145.2 | ‘Gm26936’ | IncRNA | 0.000584633 | 0.01282305 | Ageing_depleted |
| ENSMUSG00000022340.16 | ‘Sybu’ | PC | 0.000583182 | 0.01282305 | Ageing_depleted |
| ENSMUSG00000026933.18 | ‘Camsap1’ | PC | 0.000681398 | 0.014464631 | Ageing_depleted |
| ENSMUSG00000021288.20 | ‘Klc1’ | PC | 9.96E−04 | 1.94E−02 | Ageing_depleted |
| ENSMUSG00000116933.2 | ‘Atp5o’ | PC | 1.02E−03 | 1.97E−02 | Ageing_depleted |
| ENSMUSG00000028698.14 | ‘Pik3r3’ | PC | 1.06E−03 | 2.03E−02 | Ageing_depleted |
| ENSMUSG00000024725.14 | ‘Ostf1’ | PC | 1.19E−03 | 2.20E−02 | Ageing_depleted |
| ENSMUSG00000024241.8 | ‘Sos1’ | PC | 1.25E−03 | 2.23E−02 | Ageing_depleted |
| ENSMUSG00000038733.15 | ‘Wdr26’ | PC | 1.26E−03 | 2.25E−02 | Ageing_depleted |
| ENSMUSG00000021676.11 | ‘Iqgap2’ | PO | 1.40E−03 | 2.42E−02 | Ageing_depleted |
| ENSMUSG00000102918.2 | ‘Pcdhgc3’ | PC | 1.45E−03 | 2.48E−02 | Ageing_depleted |
| ENSMUSG00000027339.16 | ‘Rassf2’ | PC | 1.51E−03 | 2.57E−02 | Ageing_depleted |
| ENSMUSG00000022456.19 | ‘Septin3’ | PC | 1.53E−03 | 2.60E−02 | Ageing_depleted |
| ENSMUSG00000086805.10 | ‘4932443L11Rik’ | IncRNA | 1.65E−03 | 2.75E−02 | Ageing_depleted |
| ENSMUSG00000057147.14 | ‘Dph6’ | PC | 1.69E−03 | 2.79E−02 | Ageing_depleted |
| ENSMUSG00000054976.15 | ‘Nyap2’ | PC | 1.75E−03 | 2.83E−02 | Ageing_depleted |
| ENSMUSG00000031451.7 | ‘Gas6’ | PC | 1.77E−03 | 2.85E−02 | Ageing_depleted |
| ENSMUSG00000025777.9 | ‘Gdap1’ | PC | 2.02E−03 | 3.15E−02 | Ageing_depleted |
| ENSMUSG00000041415.11 | ‘Dicer1’ | PC | 2.11E−03 | 3.26E−02 | Ageing_depleted |
| ENSMUSG00000038872.11 | ‘Zfhx3’ | PC | 2.13E−03 | 3.28E−02 | Ageing_depleted |
| ENSMUSG00000061186.16 | ‘Sfmbt2’ | PC | 2.36E−03 | 3.46E−02 | Ageing_depleted |
| ENSMUSG00000021366.9 | ‘Hivep1’ | PC | 2.38E−03 | 3.48E−02 | Ageing_depleted |
| ENSMUSG00000016933.18 | ‘Plcg1’ | PC | 2.48E−03 | 3.55E−02 | Ageing_depleted |
| ENSMUSG00000031601.17 | ‘Cnot7’ | PC | 2.74E−03 | 3.74E−02 | Ageing_depleted |
| ENSMUSG00000055214.16 | ‘Pld5’ | PC | 2.84E−03 | 3.83E−02 | Ageing_depleted |
| ENSMUSG00000028414.18 | ‘Fktn’ | PC | 3.56E−03 | 4.49E−02 | Ageing_depleted |
| ENSMUSG00000035305.6 | ‘Ror1’ | PC | 3.69E−03 | 4.61E−02 | Ageing_depleted |
| ENSMUSG00000040722.8 | ‘Scamp5’ | PC | 3.72E−03 | 0.046129142 | Ageing_depleted |
| ENSMUSG00000054752.17 | ‘Fsd1l’ | PC | 3.77E−03 | 0.04652168 | Ageing_depleted |
| ENSMUSG00000062184.12 | ‘Hs6st2’ | PC | 4.24E−03 | 0.050227065 | Ageing_depleted |
| ENSMUSG00000061950.18 | ‘Ppp4r1’ | PC | 4.97E−03 | 0.055772619 | Ageing_depleted |
| ENSMUSG00000103719.2 | ‘Gm38039’ | IncRNA | 5.42E−03 | 0.058768436 | Ageing_depleted |
| ENSMUSG00000009575.15 | ‘Cbx5’ | PC | 5.52E−03 | 0.059534429 | Ageing_depleted |
| ENSMUSG00000035517.18 | ‘Tdrd7’ | PC | 5.56E−03 | 0.059799308 | Ageing_depleted |
| ENSMUSG00000029253.13 | ‘Cenpc1’ | PC | 5.84E−03 | 0.062106203 | Ageing_depleted |
| ENSMUSG00000037013.18 | ‘Ss18’ | PC | 0.005998944 | 0.062995012 | Ageing_depleted |
| ENSMUSG00000041439.16 | ‘Mfsd6’ | PC | 0.006233812 | 0.064186372 | Ageing_depleted |
| ENSMUSG00000057880.13 | ‘Abat’ | PC | 0.006352025 | 0.064776375 | Ageing_depleted |
| ENSMUSG00000026113.18 | ‘Inpp4a’ | PC | 0.008081714 | 0.075550187 | Ageing_depleted |
| ENSMUSG00000102995.2 | ‘A330074H02Rik’ | TEC | 0.00825689 | 0.076239129 | Ageing_depleted |
| ENSMUSG00000050447.16 | ‘Lypd6’ | PC | 0.008469695 | 0.077440006 | Ageing_depleted |
| ENSMUSG00000041229.16 | ‘Phf8’ | PC | 0.008531851 | 0.077896229 | Ageing_depleted |
| ENSMUSG00000037996.18 | ‘Slc24a2’ | PC | 0.008687357 | 0.078793237 | Ageing_depleted |
| ENSMUSG00000060424.15 | ‘Pantr1’ | IncRNA | 0.009255064 | 0.081787499 | Ageing_depleted |
| ENSMUSG00000024426.18 | ‘Atat1’ | PC | 0.009279068 | 0.081816997 | Ageing_depleted |
| ENSMUSG00000049122.18 | ‘Frmd3’ | PC | 0.00955401 | 0.083405352 | Ageing_depleted |
| ENSMUSG00000002109.15 | ‘Ddb2’ | PC | 0.00963623 | 0.083938026 | Ageing_depleted |
| ENSMUSG00000037172.15 | ‘Dennd11’ | PC | 0.00977521 | 0.084497936 | Ageing_depleted |
| ENSMUSG00000101463.2 | ‘Gm28750’ | IncRNA | 0.01085931 | 0.090892192 | Ageing_depleted |
| ENSMUSG00000103831.2 | ‘Gm37608’ | TEC | 0.010991868 | 0.091325931 | Ageing_depleted |
| ENSMUSG00000028613.16 | ‘Lrp8’ | PC | 0.011595115 | 0.094603682 | Ageing_depleted |
| ENSMUSG00000066043.14 | ‘Phactr4’ | PC | 0.012068223 | 0.09695181 | Ageing_depleted |
| ENSMUSG00000033389.17 | ‘Arhgap44’ | PC | 0.012044811 | 0.09695181 | Ageing_depleted |
| ENSMUSG00000022462.8 | ‘Slc38a2’ | PC | 0.012360171 | 0.098657095 | Ageing_depleted |
| ENSMUSG00000036180.16 | ‘Gatad2a’ | PC | 4.42E−97 | 5.01E−94 | Ageing_enriched |
| ENSMUSG00000030921.18 | ‘Trim30a’ | PC | 1.94E−85 | 1.30E−82 | Ageing_enriched |
| ENSMUSG00000005534.11 | ‘Insr’ | PC | 1.97E−85 | 1.30E−82 | Ageing_enriched |
| ENSMUSG00000040265.17 | ‘Dnm3’ | PC | 1.40E−57 | 6.92E−55 | Ageing_enriched |
| ENSMUSG00000033768.18 | ‘Nrxn2’ | PC | 3.66E−55 | 1.52E−52 | Ageing_enriched |
| ENSMUSG00000112314.2 | ‘Gm49454’ | IncRNA | 1.91E−34 | 5.61E−32 | Ageing_enriched |
| ENSMUSG00000039458.16 | ‘Mtmr12’ | PC | 7.32E−34 | 2.07E−31 | Ageing_enriched |
| ENSMUSG00000091034.9 | ‘Gm17660’ | PC | 3.24E−22 | 6.93E−20 | Ageing_enriched |
| ENSMUSG00000063458.14 | ‘Lrmda’ | PC | 1.05E−21 | 2.19E−19 | Ageing_enriched |
| ENSMUSG00000101344.2 | ‘Gm29183’ | IncRNA | 2.15E−19 | 4.06E−17 | Ageing_enriched |
| ENSMUSG00000066687.6 | ‘Zbtb16’ | PC | 1.41E−18 | 2.54E−16 | Ageing_enriched |
| ENSMUSG00000022119.16 | ‘Rbm26’ | PC | 1.15E−16 | 1.72E−14 | Ageing_enriched |
| ENSMUSG00000039717.17 | ‘Ralyl’ | PC | 9.35E−16 | 1.37E−13 | Ageing_enriched |
| ENSMUSG00000062151.14 | ‘Unc13c’ | PC | 1.23E−14 | 1.65E−12 | Ageing_enriched |
| ENSMUSG00000110246.2 | ‘C130073E24Rik’ | IncRNA | 1.87E−14 | 2.47E−12 | Ageing_enriched |
| ENSMUSG00000055963.13 | ‘Triqk’ | PC | 5.43E−14 | 6.94E−12 | Ageing_enriched |
| ENSMUSG00000053279.9 | ‘Aldh1a1’ | PC | 9.97E−14 | 1.25E−11 | Ageing_enriched |
| ENSMUSG00000022123.10 | ‘Scel’ | PC | 7.93E−13 | 9.52E−11 | Ageing_enriched |
| ENSMUSG00000037921.16 | ‘Ddx60’ | PC | 7.20E−12 | 8.03E−10 | Ageing_enriched |
| ENSMUSG00000026558.14 | ‘Uck2’ | PC | 1.35E−11 | 1.40E−09 | Ageing_enriched |
| ENSMUSG00000029212.12 | ‘Gabrb1’ | PC | 2.02E−11 | 2.08E−09 | Ageing_enriched |
| ENSMUSG00000014361.6 | ‘Mertk’ | PC | 2.48E−10 | 2.43E−08 | Ageing_enriched |
| ENSMUSG00000109741.2 | ‘Gm45455’ | IncRNA | 5.80E−10 | 5.53E−08 | Ageing_enriched |
| ENSMUSG00000025314.17 | ‘Ptprj’ | PC | 1.35E−09 | 1.19E−07 | Ageing_enriched |
| ENSMUSG00000021340.14 | ‘Gpld1’ | PC | 1.48E−09 | 1.27E−07 | Ageing_enriched |
| ENSMUSG00000030075.11 | ‘Cntn3’ | PC | 2.71E−09 | 2.21E−07 | Ageing_enriched |
| ENSMUSG00000022747.18 | ‘St3gal6’ | PC | 3.77E−09 | 3.04E−07 | Ageing_enriched |
| ENSMUSG00000056966.8 | ‘Gjc3’ | PC | 8.01E−09 | 6.28E−07 | Ageing_enriched |
| ENSMUSG00000034055.17 | ‘Phka1’ | PC | 1.01E−08 | 7.74E−07 | Ageing_enriched |
| ENSMUSG00000019865.10 | ‘Nmbr’ | PC | 1.65E−08 | 1.24E−06 | Ageing_enriched |
| ENSMUSG00000040118.16 | ‘Cacna2d1’ | PC | 2.65E−08 | 1.96E−06 | Ageing_enriched |
| ENSMUSG00000115122.2 | ‘Gm49685’ | IncRNA | 7.85E−08 | 5.60E−06 | Ageing_enriched |
| ENSMUSG00000040957.16 | ‘Cables1’ | PC | 8.09E−08 | 5.72E−06 | Ageing_enriched |
| ENSMUSG00000039601.17 | ‘Rcan2’ | PC | 8.49E−08 | 5.91E−06 | Ageing_enriched |
| ENSMUSG00000028517.9 | ‘Plpp3’ | PC | 3.35E−07 | 2.07E−05 | Ageing_enriched |
| ENSMUSG00000027674.17 | ‘Pex5l’ | PC | 3.54E−07 | 2.16E−05 | Ageing_enriched |
| ENSMUSG00000019996.18 | ‘Map7’ | PC | 1.06E−06 | 5.93E−05 | Ageing_enriched |
| ENSMUSG00000007097.15 | ‘Atp1a2’ | PC | 1.16E−06 | 6.42E−05 | Ageing_enriched |
| ENSMUSG00000025474.10 | ‘Tubgcp2’ | PC | 1.23E−06 | 6.72E−05 | Ageing_enriched |
| ENSMUSG00000024998.18 | ‘Plce1’ | PC | 1.47E−06 | 7.81E−05 | Ageing_enriched |
| ENSMUSG00000037999.14 | ‘Arap2’ | PC | 2.61E−06 | 0.000132704 | Ageing_enriched |
| ENSMUSG00000033350.8 | ‘Chst2’ | PC | 2.88E−06 | 0.000145053 | Ageing_enriched |
| ENSMUSG00000100301.7 | ‘6030407O03Rik’ | IncRNA | 9.36E−06 | 0.000425995 | Ageing_enriched |
| ENSMUSG00000104785.2 | ‘Gm31121’ | IncRNA | 9.64E−06 | 0.000436219 | Ageing_enriched |
| ENSMUSG00000026888.15 | ‘Grb14’ | PC | 1.06E−05 | 0.000472257 | Ageing_enriched |
| ENSMUSG00000027864.10 | ‘Ptgfrn’ | PC | 1.36E−05 | 0.000587093 | Ageing_enriched |
| ENSMUSG00000032377.9 | ‘Plscr4’ | PC | 2.09E−05 | 0.000861721 | Ageing_enriched |
| ENSMUSG00000019820.12 | ‘Utrn’ | PC | 2.08E−05 | 0.000861721 | Ageing_enriched |
| ENSMUSG00000110723.2 | ‘Gm49353’ | PC | 3.58E−05 | 0.00142347 | Ageing_enriched |
| ENSMUSG00000020363.7 | ‘Gfpt2’ | PC | 3.61E−05 | 0.001429181 | Ageing_enriched |
| ENSMUSG00000061578.9 | ‘Ksr2’ | PC | 6.51E−05 | 0.002408377 | Ageing_enriched |
| ENSMUSG00000089941.2 | ‘Gm16168’ | IncRNA | 7.18E−05 | 0.002549189 | Ageing_enriched |
| ENSMUSG00000049420.10 | ‘Tmem200a’ | PC | 7.38E−05 | 0.002587361 | Ageing_enriched |
| ENSMUSG00000037706.18 | ‘Cd81’ | PC | 7.61E−05 | 0.002644151 | Ageing_enriched |
| ENSMUSG00000035299.17 | ‘Mid1’ | PC | 8.60E−05 | 0.002884091 | Ageing_enriched |
| ENSMUSG00000113208.2 | ‘Gm48421’ | IncRNA | 9.37E−05 | 0.003076384 | Ageing_enriched |
| ENSMUSG00000031425.16 | ‘Plp1’ | PC | 0.000102795 | 0.003255723 | Ageing_enriched |
| ENSMUSG00000030310.11 | ‘Slc6a1’ | PC | 0.000118952 | 0.003622547 | Ageing_enriched |
| ENSMUSG00000050663.9 | ‘Trhde’ | PC | 0.000133118 | 0.003964005 | Ageing_enriched |
| ENSMUSG00000026187.10 | ‘Xrcc5’ | PC | 0.000164119 | 0.004575695 | Ageing_enriched |
| ENSMUSG00000059182.9 | ‘Skap2’ | PC | 0.000187355 | 0.00506305 | Ageing_enriched |
| ENSMUSG00000109006.3 | ‘B230209E15Rik’ | IncRNA | 0.000230903 | 0.005974816 | Ageing_enriched |
| ENSMUSG00000001995.10 | ‘Sipa1l2’ | PC | 0.000251393 | 0.006400426 | Ageing_enriched |
| ENSMUSG00000052062.15 | ‘Pard3b’ | PC | 0.000309758 | 0.007712788 | Ageing_enriched |
| ENSMUSG00000054477.17 | ‘Kcnn2’ | PC | 0.000314841 | 0.007790358 | Ageing_enriched |
| ENSMUSG00000115821.2 | ‘6330576A10Rik’ | IncRNA | 0.000402382 | 0.009426209 | Ageing_enriched |
| ENSMUSG00000046768.14 | ‘Rhoj’ | PC | 0.000460485 | 0.010568454 | Ageing_enriched |
| ENSMUSG00000105068.2 | ‘Gm30835’ | IncRNA | 0.000605006 | 0.013160543 | Ageing_enriched |
| ENSMUSG00000006205.14 | ‘Htra1’ | PC | 0.000603063 | 0.013160543 | Ageing_enriched |
| ENSMUSG00000037957.15 | ‘Wdr20’ | PC | 0.000648025 | 0.013905323 | Ageing_enriched |
| ENSMUSG00000038831.17 | ‘Ralgps1’ | PC | 0.00067127 | 0.01428794 | Ageing_enriched |
| ENSMUSG00000034453.9 | ‘Polr3b’ | PC | 0.000741131 | 0.015524545 | Ageing_enriched |
| ENSMUSG00000096370.9 | ‘Gm21992’ | PC | 0.000767203 | 0.01581924 | Ageing_enriched |
| ENSMUSG00000024534.16 | ‘Sncaip’ | PC | 0.000823099 | 0.016753974 | Ageing_enriched |
| ENSMUSG00000024539.18 | ‘Ptpn2’ | PC | 0.000869798 | 0.017435598 | Ageing_enriched |
| ENSMUSG00000031027.16 | ‘Stk33’ | PC | 0.00100222 | 0.019449938 | Ageing_enriched |
| ENSMUSG00000020061.19 | ‘Mybpc1’ | PC | 0.001108769 | 0.020947576 | Ageing_enriched |
| ENSMUSG00000034235.18 | ‘Usp54’ | PC | 0.00119308 | 0.022063181 | Ageing_enriched |
| ENSMUSG00000036264.10 | ‘Fstl4’ | PC | 0.001244905 | 0.02234441 | Ageing_enriched |
| ENSMUSG00000019235.10 | ‘Rps6kl1’ | PC | 0.001231734 | 0.02234441 | Ageing_enriched |
| ENSMUSG00000057098.15 | ‘Ebf1’ | PC | 0.001357308 | 0.023620148 | Ageing_enriched |
| ENSMUSG00000037062.14 | ‘Sh3glb1’ | PC | 0.00146978 | 0.025081285 | Ageing_enriched |
| ENSMUSG00000102316.2 | ‘Gm37629’ | TEC | 0.001859806 | 0.029510906 | Ageing_enriched |
| ENSMUSG00000004360.10 | ‘9330159F19Rik’ | PC | 0.002159757 | 0.032973662 | Ageing_enriched |
| ENSMUSG00000005899.15 | ‘Smpd4’ | PC | 0.002273675 | 0.034161212 | Ageing_enriched |
| ENSMUSG00000027546.16 | ‘Atp9a’ | PC | 0.002396614 | 0.034883074 | Ageing_enriched |
| ENSMUSG00000036368.9 | ‘Rmdn2’ | PC | 0.002395621 | 0.034883074 | Ageing_enriched |
| ENSMUSG00000027695.17 | ‘Pld1’ | PC | 0.002426716 | 0.03505306 | Ageing_enriched |
| ENSMUSG00000038481.14 | ‘Cdk19’ | PC | 0.002444998 | 0.03519908 | Ageing_enriched |
| ENSMUSG00000031552.14 | ‘Adam18’ | PC | 0.002449922 | 0.035205963 | Ageing_enriched |
| ENSMUSG00000039153.18 | ‘Runx2’ | PC | 0.002527898 | 0.035615469 | Ageing_enriched |
| ENSMUSG00000070509.16 | ‘Rgma’ | PC | 0.002728281 | 0.037350515 | Ageing_enriched |
| ENSMUSG00000022788.17 | ‘Fgd4’ | PC | 0.002924409 | 0.039313188 | Ageing_enriched |
| ENSMUSG00000045100.12 | ‘Slc25a26’ | PC | 0.00307056 | 0.040588807 | Ageing_enriched |
| ENSMUSG00000073481.10 | ‘Mtarc2’ | PC | 0.003280943 | 0.042587722 | Ageing_enriched |
| ENSMUSG00000056579.19 | ‘Tug1’ | PC | 0.003692168 | 0.046111341 | Ageing_enriched |
| ENSMUSG00000102250.2 | ‘Gm38260’ | TEC | 0.003711398 | 0.046129142 | Ageing_enriched |
| ENSMUSG00000066442.18 | ‘Mthfs' | PC | 0.003875554 | 0.047079347 | Ageing_enriched |
| ENSMUSG00000024812.12 | ‘Tjp2’ | PC | 0.003888762 | 0.047081374 | Ageing_enriched |
| ENSMUSG00000040433.17 | ‘Zbtb38’ | PC | 0.004166671 | 0.049686294 | Ageing_enriched |
| ENSMUSG00000022309.10 | ‘Angpt1’ | PC | 0.005038533 | 0.05626954 | Ageing_enriched |
| ENSMUSG00000109088.2 | ‘Gm44593’ | IncRNA | 0.005099626 | 0.056552992 | Ageing_enriched |
| ENSMUSG00000042282.5 | ‘Gucy2f’ | PC | 0.00524061 | 0.057552217 | Ageing_enriched |
| ENSMUSG00000004317.15 | ‘Clcn5’ | PC | 0.005295751 | 0.057836909 | Ageing_enriched |
| ENSMUSG00000023044.3 | ‘Csad’ | PC | 0.006421775 | 0.065022525 | Ageing_enriched |
| ENSMUSG00000004040.17 | ‘Stat3’ | PC | 0.006541716 | 0.065732621 | Ageing_enriched |
| ENSMUSG00000047767.18 | ‘Atg16l2’ | PC | 0.007554165 | 0.072684641 | Ageing_enriched |
| ENSMUSG00000022469.18 | ‘Rapgef3’ | PC | 0.007591772 | 0.072951033 | Ageing_enriched |
| ENSMUSG00000030607.8 | ‘Acan’ | PC | 0.008093943 | 0.0755548 | Ageing_enriched |
| ENSMUSG00000023017.11 | ‘Asic1’ | PC | 0.008202853 | 0.076143244 | Ageing_enriched |
| ENSMUSG00000046160.7 | ‘Olig1’ | PC | 0.008252126 | 0.076239129 | Ageing_enriched |
| ENSMUSG00000030663.13 | ‘1110004F10Rik’ | PC | 0.008929043 | 0.080455684 | Ageing_enriched |
| ENSMUSG00000024513.17 | ‘Mbd2’ | PC | 0.009190469 | 0.081489511 | Ageing_enriched |
| ENSMUSG00000027287.15 | ‘Snap23’ | PC | 0.009245887 | 0.081787499 | Ageing_enriched |
| ENSMUSG00000039194.17 | ‘Rlbp1’ | PC | 0.009730984 | 0.084392042 | Ageing_enriched |
| ENSMUSG00000085631.2 | ‘9630028H03Rik’ | IncRNA | 0.009884495 | 0.085122326 | Ageing_enriched |
| ENSMUSG00000038260.11 | ‘Trpm4’ | PC | 0.010262044 | 0.087302187 | Ageing_enriched |
| ENSMUSG00000022508.6 | ‘Bcl6’ | PC | 0.010268563 | 0.087302187 | Ageing_enriched |
| ENSMUSG00000107917.2 | ‘Gm44235’ | TEC | 0.010423272 | 0.088250057 | Ageing_enriched |
| ENSMUSG00000001260.11 | ‘Gabrg1’ | PC | 0.010594864 | 0.089281937 | Ageing_enriched |
| ENSMUSG00000025931.16 | ‘Paqr8’ | PC | 0.011750938 | 0.095527646 | Ageing_enriched |
| ENSMUSG00000062234.15 | ‘Gak’ | PC | 0.01267715 | 0.099961337 | Ageing_enriched |

(PC = protein coding)

### Example 4: PerturbSci-Kinetics

The studies described here provided the first method to quantitatively characterize the genome-wide mRNA kinetic rates (e.g., synthesis and degradation rates) across hundreds of genetic perturbations in a single experiment. Furthermore, the analysis illustrates the advantages of PerturbSci-Kinetics over conventional assays that solely profile gene expression changes. By capturing three layers of readout (e.g., nascent, whole transcriptome, and sgRNA identify) at single-cell resolution, PerturbSci-Kinetics uniquely enables the dissection of the critical regulators of gene-specific transcription, splicing, and degradation in a massive-parallel manner. Finally, PerturbSci-Kinetics is built on the recently developed EasySci-RNA (Sziraki, A. et al., bioRxiv 2022.09.28.509825 (2022)) and can be easily scaled up to profiling genome-wide perturbations (e.g., 10,000 s genes or cis-regulatory elements) across tens of millions of single cells, thus enabling the systematic characterization of cell-type-specific gene regulatory network at unprecedented scale and resolution.

The Materials and Methods are now described.

### Cell Culture

The 3T3-L1-CRISPRi cell line was a gift from the Tissue Culture facility of the University of California, Berkeley, and the HEK293 cell line was a gift from the Scott Keeney Lab at Memorial Sloan Kettering Cancer Center. The HEK293T cell line was obtained from ATCC (CRL-3216). All cells were maintained at 37° C. and 5% CO2 in high glucose DMEM medium supplemented with L-Glutamine and Sodium Pyruvate (Gibco 11995065) and 10% Fetal Bovine Serum (FBS; Sigma F4135). When generating a monoclonal cell line, the medium was supplemented with 1% Penicillin-Streptomycin (Gibco 15140163). In the screening experiment, after the induction of dCas9-KRAB-MeCP2 expression by 1 ug/ml Dox (Sigma D5207), sgRNA-transduced HEK293-idCas9 cells were cultured in high glucose DMEM medium supplemented with L-Glutamine (Gibco 11965092) and 10% FBS.

### Generation of Monoclonal HEK293-idCas9 Cell Line

To generate HEK293 with Dox-inducible dCas9-KRAB-MeCP2 expression, the lentiviral plasmid Lenti-idCas9-KRAB-MeCP2-T2A-mCherry-Neo was constructed. A dCas9-KRAB-MeCP2-T2A insert was amplified from dCas9-KRAB-MeCP2 (Addgene #110821). A T2A-mCherry Gblock was synthesized by IDT. Gibson Assembly reaction (NEB E2611S) was performed at 50° C. with a mixture of Bsp119I-digested Lenti-Neo-iCas9 (Thermo FD0124; Addgene #85400), dCas9-KRAB-MeCP2-T2A amplicon, T2A-mCherry Gblock for 60 minutes to construct a dCas9-KRAB-MeCP2-T2A-mCherry plasmid. The reaction product was transformed into NEBstable competent cells (NEB C3040H), and colonies were inoculated and amplified in LB medium (Gibco 10855001) with 50 ug/ml Sodium Ampicillin (Sigma A8351) at 37° C. overnight.

After plasmid extraction (QIAGEN No. 27106) and sequencing validation, the plasmid was co-transfected with psPAX2 (Addgene #12260) and pMD2.G (Addgene #12259) into low-passage HEK293T cells in a 10 cm dish using Polyjet (SignaGen SL100688) for 24 hours. Cells were gently washed twice with PBS, then cultured in a medium with 10 mM Sodium Butyrate (Sigma TR-1008-G) for another 24 hours. The supernatant was collected, and cell debris was cleared by spinning down (5 min, 1000×g) and passed through a 0.45 μm filter. The lentivirus was concentrated 10× by the Lenti-X concentrator (TaKaRa 631231), and the virus suspension was flash frozen by Liquid Nitrogen and was stored at −80° C.

The lentivirus titer was determined by examining the ratio of mCherry+ cells after 24 hours of transduction and 48 hours of Dox induction. Polybrene (Sigma TR-1003) at a final concentration of 8 ug/ml was used to enhance the transduction efficiency. Then HEK293 cells were counted and transduced with lentivirus at MOI=0.2 for 48 hours. Cells were treated with Dox for 48 hours, and the top 10% of cells with the strongest mCherry fluorescence were sorted to each well of a 96-well plate containing 100 ul medium. After a 3-week expansion, monoclonal cells that survived were transferred to larger dishes for further expansion. The clone with inducible homogeneous strong mCherry expression and normal morphology was picked for the following experiment.

### Gene Knockdown and Efficacy Examination

To simplify the lentiviral titer measurement, CROP-seq-opti-Puro-T2A-GFP was assembled by adding a T2A-GFP downstream of Puromycin resistant protein coding sequence on the CROP-seq-opti plasmid (Addgene #106280). Flanking MluI and CsiI digestion sites were added to the GFP Gblock (IDT) by PCR. Both amplicon and CROP-seq-opti vector were digested using MluI (Thermo, FD0564) and CsiI (Thermo, FD2114) at 37° C. for 30 minutes, and were ligated at room temperature for 20 minutes using the Blunt/TA Ligase Master Mix (NEB M0367S). Transformation, clone amplification, and sequencing validation were done as stated above.

Oligos corresponding to individual guides for ligation were ordered as standard DNA oligos from IDT with the following design:

Plus strand:
5′-CACCG[20 bp sgRNA plus strand sequence]-3′
Minus strand:
5′-AAAC[20 bp sgRNA minus strand sequence]C-3′

Oligos were reconstituted into 100 μM and were mixed and phosphorylated using T4 PNK (NEB M0201S) by incubating at 37° C. for 30 minutes. The reaction was heated at 95° C. for 5 minutes and then ramped down to 25° C. by −0.1° C./second to anneal oligos into a double-stranded duplex. The CROP-seq-opti-Puro-T2A-GFP was digested by Esp3I (NEB R0734L) at 37° C. for 30 minutes, then the linearized backbone and the annealed duplex were ligated at room temperature for 20 minutes using the Blunt/TA Ligase Master Mix (NEB M0367S). Transformation, clone amplification, sequencing validation, lentivirus generation, and titer measurement were done as stated above.

For the mouse 3T3-L1-CRISPRi cells, they were counted and incubated with lentivirus inserted with either non-target control (NTC) sgRNA or sgRNA targeting an Fto gene, and 8 ug/ml of Polybrene. For the human HEK293-idCas9 cells, they were counted and incubated with NTC sgRNA or sgRNA targeting an IGF1R gene, and 8 ug/ml of Polybrene. Transduction was then performed at MOI=0.2 for 48 hours. Based on the results of the puromycin titration experiments, sgRNA-transduced 3T3-L1-CRISPRi cells were selected by 2.5 ug/ml Puromycin for 2 days and 2 ug/ml Puromycin for 3 days, and sgRNA-transduced HEK293-idCas9 cells were selected by 1.5 ug/ml Puromycin for 3 days and 1 ug/ml Puromycin for 2 days.

As dCas9-BFP-KRAB was constitutively expressed in 3T3-L1-CRISPRi cells, the target gene started being silenced once sgRNA lentivirus was introduced. For HEK293-idCas9 cells, Dox treatment for a minimum of 72 hours was required before examining the knockdown effect.

For RT-qPCR validation, primers targeting IGF1R were selected from PrimerBank (pga.mgh.harvard.edu/primerbank/) and were synthesized from IDT. Total RNA in 1e6 cells of each sample was extracted using the RNeasy Mini kit (QIAGEN 74104) and the concentration was measured by Nanodrop. 1 ug total RNA was then reverse-transcribed into the first strand cDNA by SuperScript VILO Master Mix (Thermo 11755050). PowerTrack SYBR Green Master Mix (Thermo A46109) was used for RT-qPCR following the manufacturer's instructions.

For flow cytometry validation, 1e6 cells of each sample were harvested and resuspended in 100 μl of PBS-0.1% sodium azide-2% FBS. BV421 Mouse Anti-Human CD221 (BD 565966) and BV421 Mouse IgG1 k Isotype Control (BD 562438) at the final concentration of 10 μg/ml were added, and reactions were incubated at 4° C. in the dark with rotation for 30 minutes. Cells were then washed twice using PBS-0.1% sodium azide-2% FBS, and fluorescence signals were recorded.

Construction of Pooled sgRNA Library

Genes of interest were selected manually, considering their functions and expression levels in HEK293 cells. The sgRNA sequences targeting genes of interest with the best performances were obtained from an established optimized sgRNA library (only sgRNA set A is considered) (Sanson, K. R. et al., Nat. Commun. 9, 5416 (2018)). Finally, 684 sgRNAs targeting 228 genes (3 sgRNAs/gene) and 15 additional NO-TARGET sgRNAs were included in the present study.

The single-stranded sgRNA library was synthesized in a pooled manner by IDT in the following format:

5′-GGCTTTATATATCTTGTGGAAAGGACGAAACACCG[20 bps gRNA
plus strand sequence]GTTTAAGAGCTATGCTGGAAACAGCATA
GCAAGTT-3′

100 ng of oligo pool was amplified by PCR using primers targeting 5′ homology arm (HA) and 3′ HA with limited cycles (×12) to avoid introducing amplification biases. The PCR product was purified, and double-stranded library amplicons were extracted by DNA electrophoresis and gel extraction. Then the insert was cloned into Esp3I-digested CROP-seq-opti-Puro-T2A-GFP by Gibson Assembly (50° C. for 60 minutes). In parallel, a control Gibson Assembly reaction containing only the backbone was set. Both reactions were cleaned up by 0.75× AMPURE beads (Beckman Coulter A63882) and eluted in 5 μL EB buffer (QIAGEN 19086), then were transformed into Endura Electrocompetent Cells (Lucigen, 602422) by electroporation (Gene Pulser Xcell Electroporation System, Bio-Rad, 1652662). After 1 hour of recovery at 250 rpm, 37° C., each reaction was spread onto an in-house 245 mm Square agarose plate (Corning, 431111) with 100 ug/ml of Carbenicillin (Thermo, 10177012) and was then grown at 32° C. for 13 hours to minimize potential recombination and growth biases. All colonies from each reaction were scraped from the plate and the CROP-seq-opti-Puro-T2A-GFP-sgRNA plasmid library was extracted using ZymoPURE II Plasmid Midiprep Kit (Zymo, D4200). The lentiviral library was generated as stated above with extended virus production time.

### Library Preparation for the Bulk Screen

For each replicate, 7e6 uninduced HEK293-idCas9 cells were seeded. After 12 hours, two replicates were transduced at MOI=0.1 (1000× coverage/sgRNA) and another two replicates were transduced at MOI=0.2 (2000× coverage/sgRNA) with 8 μg/ml of Polybrene for 24 hours. Then the culture medium was replaced with the virus-free medium and culture cells for another 24 hours. Transduced cells were selected by 1.5 μg/ml of Puromycin for 3 days and 1 μg/ml of Puromycin for 2 days. During the selection, cells were passaged every 2 or 3 days to ensure at least 1000× coverage. At the end of the drug selection, 1.4e6 cells were harvested in each replicate (2000× coverage/sgRNA) as day0 samples of the bulk screen and pellet down at 500×g, 4° C. for 5 minutes. Cell pellets were stored at −80° C. for genomic DNA extraction later. Then the dCas9-KRAB-MeCP2 expression was induced by adding Dox at the final concentration of 1 μg/ml, and L-glutamine+, sodium pyruvate−, high glucose DMEM was used to sensitize cells to perturbations on energy metabolism genes. Cells were cultured in this condition for additional 7 days and were passed every other day with 4000× coverage/sgRNA. On day7, 6 ml of the original media from each plate was mixed with 6 μL of 200 mM 4sU (Sigma T4509-25 MG) dissolved in DMSO (VWR 97063-136) and was put back for nascent RNA metabolic labeling. After 2 hours of treatment, 1.4e6 cells in each replicate were harvested as day7 samples of the bulk screen, and the rest of the cells were fixed and stored for single-cell Perturb-Kinetics profiling (see the next section).

Genomic DNA of bulk screen samples was extracted using Quick-DNA Miniprep Plus Kit (Zymo, D4068T) following the manufacturer's instructions and quantified by Nanodrop. All genomic DNA was used for PCR to ensure coverage. The primer targeting the U6 promoter region with P5-15-Read1 overhang and the primer targeting the sgRNA scaffold region with P7-17-Read2 overhang was used for generating the bulk screen libraries for sequencing (Tables 11 and 12).

### Library Preparation for the PerturbSci-Kinetics

After trypsinization, cells in each 10 cm dish were collected into a 15 ml falcon tube and kept on ice. Cells were spun down at 300×g for 5 minutes (4° C.) and washed once in 3 ml ice-cold PBS. Cells were fixed with 5 ml ice-cold 4% PFA in PBS (Santa Cruz Biotechnology sc-281692) for 15 minutes on ice. PFA was then quenched by adding 250 ul 2.5M Glycine (Sigma 50046-50G), and cells were pelleted at 500×g for 5 minutes (4° C.). Fixed cells were washed once with 1 ml PBSR (PBS, 0. % SUPERase In (Thermo AM2696), and 10 mM dithiothreitol (DTT; Thermo R0861)), and were then resuspended, permeabilized, and further fixed in 1 ml PBSR-triton-BS3 (PBS, 0.1% SUPERase In, 0.2% Triton-X100 (Sigma X100-500ML), 2 mM bis(sulfosuccinimidyl) suberate (BS3; Thermo, PG82083), 10 mM DTT) for 5 minutes. Additional 4 ml of PBS-BS3 (PBS, 2 mM BS3, 10 mM DTT) was then added to dilute Triton-X100 while keeping the concentration of BS3, and cells were incubated on ice for 15 minutes. Cells were pelleted at 500×g, 4° C. for 5 minutes and resuspended in 500 ul nuclease-free water (Corning 46-000-CM) supplemented with 0.1% SUPERase In and 10 mM DTT. 3 ml of 0.05N HCl (Fisher Chemical SA54-1) was added for further permeabilization. After 3 minutes of incubation on ice, 3.5 ml Tris-HCl, pH 8.0 (Thermo 15568025), and 35 ul of 10% Triton X-100 were added to each tube to neutralize the HCl. After spinning down at 4° C., 500×g for 5 minutes, cells were finally resuspended in 400 ul PSB-DTT at the concentration of ˜2e6 cells/100 ul (PBS, 1% SUPERase In, 1% Bovine Serum Albumin (BSA; NEB B90000S), 1 mM DTT), mixed with 10% DMSO, and were slow-frozen and stored in −80° C.

The chemical conversion was performed before the library preparation. Cells were thawed with shaking in the 37° C. water bath and spun down, then were washed once with 400 ul PSB without DTT. Next, cells were resuspended in 100 ul PSB, mixed with 40 ul Sodium Phosphate buffer (PH 8.0, 500 mM), 40 ul IAA (100 mM), 20 ul nuclease-free water, and 200 ul DMSO with the order. The reaction was incubated at 50° C. for 15 minutes and was quenched by adding 8 ul 1M DTT. Then cells were washed with PBS and were filtered through a 20 μm strainer (Pluriselect‡ 43-10020-60). Cells were finally resuspended in 100 μl PSB.

### Reads Processing

For bulk screen libraries, bcl files were demultiplexed into fastq files based on index 7 barcodes. Reads for each sample were further extracted by index 5 barcode matching. Then every read pair was matched against two constant sequences (Read1: 11-25 bp, Read2: 11-25 bp) to remove reads generated from the PCR by-product. For all matching steps, a maximum of 1 mismatch is allowed. Finally, sgRNA sequences were extracted from filtered read pairs (at 26-45 bp of R1), assigned to sgRNA identities with no mismatch allowed, and read counts matrices at sgRNA and gene levels were quantified.

For PerturbSci-Kinetics transcriptome reads processing and whole-transcriptome/nascent transcriptome gene counting, the pipeline was developed based on EasySci (Sziraki, A. et al., bioRxiv 2022.09.28.509825 (2022)) and Sci-fate (Cao, J., Zhou. Et al., Nat. Biotechnol. 38, 980 988 (2020)) with minor modifications. After demultiplexing on index 7, Read1 were matched against a constant sequence on the sgRNA capture primer to remove unspecific priming, and cell barcodes and UMI sequences sequenced in Read1 were added to the headers of the fastq files of Read2, which were retained for further processing. After potential poly A sequences and low-quality bases were trimmed from Read2 by Trim Galore (Krueger, F. A wrapper around Cutadapt and FastQC to consistently apply adapter and quality trimming to FastQ files, with extra functionality for RRBS data. TrimGalore), reads were aligned to a customized reference genome consisting of a complete hg38 reference genome and the dCas9-KRAB-MeCP2 sequence from Lenti-idCas9-KRAB-MECP2-T2A-mCherry-Neo using STAR (Dobin, A. et al., Bioinformatics 29, 15-21 (2013)). Unmapped reads and reads with mapping score<30 were filtered by samtools (Danecek, P. et al., Gigascience 10, (2021)). Then deduplication at the single-cell level was performed based on the UMI sequences and the alignment location, and retained reads were split into SAM files per cell. These single-cell sam files were converted into alignment tsv files using the sam2tsv function in jvarkit (Lindenbaum, P. JVarkit: java-based utilities for Bioinformatics. (2015) doi:10.6084/m9.figshare.1425030.v1). Only reads with FLAG values of 0 or 16 and high-quality mismatches with QUAL scores>45 and CIGAR of M in them were maintained. All mutations were transformed onto the plus strand and were further filtered against background SNPs called by VarScan using in-house EasySci data on HEK293 cells. Reads in which at least 30% of mutations were T to C mismatches were identified as nascent reads, and the list of reads were extracted from single-cell whole transcriptome sam files by Picard (Picard. https://broadinstitute.github.io/picard/). Finally single-cell whole transcriptome gene x cell count matrix and nascent transcriptome gene x cell count matrix were constructed by assigning reads to genes if the aligned coordinates overlapped with the gene locations on the genome. At the same time, single cell exonic/intronic read numbers were also counted by checking whether reads were mapped to the exonic or the intronic regions of genes. To quantify dCas9-KRAB-MECP2 expression, a customized gtf file consisting of the complete hg38 genomic annotations and additional annotations for dCas9 was used in this step.

Read1 and read2 of PerturbSci-Kinetics sgRNA libraries were matched against constant sequences respectively with a maximum of 1 mismatch allowed. For each filtered read pair, cell barcode, sgRNA sequence, and UMI were extracted from designed positions. Extracted sgRNA sequences with a maximum of 1 mismatch from the sgRNA library were accepted and corrected, and the corresponding UMI was used for deduplication. Duplicates were removed by collapsing identical UMI sequences of each individual corrected sgRNA under a unique cell barcode. Cells with overall sgRNA UMI counts higher than 10 were maintained and the sgRNA x cell count matrix was constructed.

sgRNA Singlets Identification and Off-Target sgRNA Removal

Cells with at least 300 whole transcriptome UMIs and 200 genes detected, and unannotated reads ratio<40% were kept. sgRNA identities of cells were assigned and doublets were removed based on the following criteria: the cell is assigned to a single sgRNA if the most abundant sgRNA in the cell took ≥60% of total sgRNA counts and is at least 3-fold of the second most abundant sgRNA. Then whole transcriptomes and sgRNA profiles of single cells were integrated with the matched nascent transcriptomes.

Target genes with the number of cells perturbed≥50 were kept for further filtering. The knockdown efficiency was calculated at the individual sgRNA level to remove potential off-target or inefficient sgRNAs: whole transcriptome counts of all cells receiving the same sgRNA were merged, normalized by the total counts, and scaled using 1e6 as the scale factor, then the fold changes of the target gene expressions were calculated by comparing the normalized expression levels between corresponding perturbations and NTC. sgRNAs with more than 40% of target gene expression reduction relative to NTC were regarded as “effective sgRNAs”, and singlets receiving these sgRNAs were kept as “on-target cells”. Downstream analyses were done at the target gene level by analyzing all cells targeting the same gene by different sgRNAs together.

### UMAP Embedding on Pseudo-Cells

Count matrix of on-target cells of which the number of cells receiving sgRNAs targeting the same gene≥50 were loaded into Seurat, and Seurat DEGs of each perturbation compared to NTC were retrieved by FindMarkers function with default parameters. Due to the relative lower sensitivity of the wilcoxon test, the “strong perturbation” was defined as groups of cells with >1 Seurat DEGs, and manually curated the filtered perturbation gene list by putting back some target genes which have overlapped functions with strong perturbations. High-fold-change (HFC) genes between perturbations and NTC were selected: the normalized expression fold change of each gene between perturbations and NTC were calculated, and were binned based on the expression level in NTC, and top 3% of genes showing highest fold changes within each bin were selected and merged. Then selected perturbations were aggregated into pseudo-cells and normalized and scaled as stated above, and merged HFC genes from all comparisons were used as features for PCA dimension reduction. Top 9 PCs were used for UMAP embedding and default parameters were used except for the following parameters: min.dist=0.3, n.neighbors=10.

### The Experimental Results are Now Described

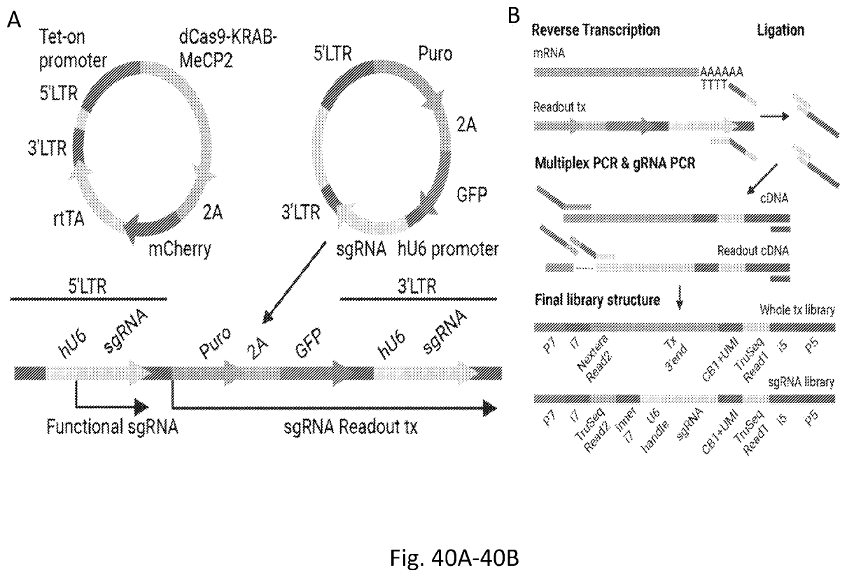

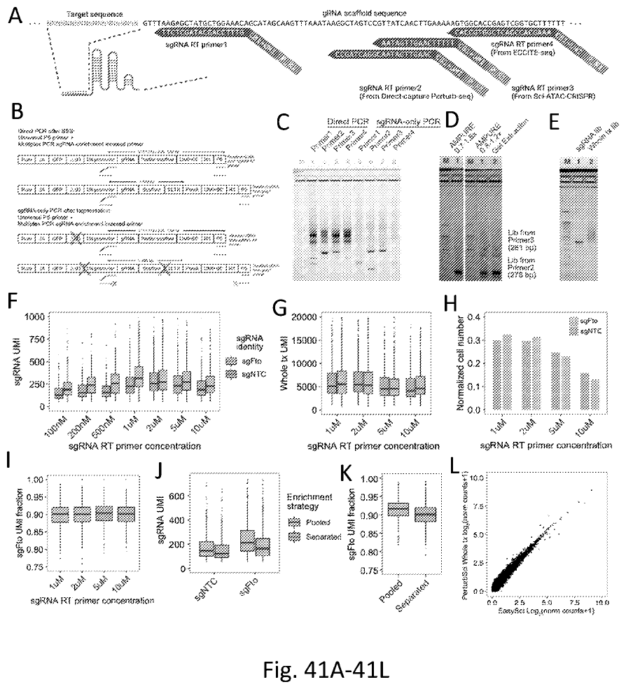

The key features of the new method include: (i) A novel combinatorial indexing strategy (referred to as ‘PerturbSci’) was developed for targeted enrichment and amplification of the sgRNA region that carries the same cellular barcode with the whole transcriptome (FIG. 39A). A modified CROP-seq vector system (Datlinger, P. et al., Nat. Methods 14, 297-301 (2017)) was adopted in PerturbSci to enable a direct capture of sgRNA sequences (FIG. 40). With the optimized sgRNA targeted enrichment strategy, as well as the extensive optimizations on primer designs, fixation, and reaction conditions, PerturbSci yields a high capture rate of sgRNA (i.e., over 97%), comparable to previous approaches for single-cell profiling of pooled CRISPR screens (FIG. 41-4) (Jaitin, D. A. et al., Cell 167, 1883-1896.e15 (2016); Adamson, B. et al., Cell 167, 1867-1882.e21 (2016); Dixit, A. et al., Cell 167, 1853-1866.e17 (2016); Xie, S. et al., Mol. Cell 66, 285-299.e5 (2017); Datlinger, P. et al., Nat. Methods 14, 297-301 (2017); Hill, A. J. et al., Nat. Methods 15, 271-274 (2018)). Furthermore, built on an extensively improved single-cell RNA-seq by three-level combinatorial indexing (i.e., EasySci-RNA (Yeo, N. C. et al., Nat. Methods 15, 611-616 (2018))), PerturbSci substantially reduced library preparation costs for single-cell RNA profiling of pooled CRISPR screens (FIG. 39B). In addition, to maximize the gene knockdown efficacy, a multimeric fusion protein dCas9-KRAB-MeCP2 (Erhard, F. et al., Nature 571, 419-423 (2019)), a highly potent transcriptional repressor that outperforms conventional dCas9 repressors, was used. (ii) By integrating PerturbSci with 4-thiouridine (4sU) labeling method, PerturbSci-Kinetics exhibited an order of magnitude higher throughput than the previous single-cell metabolic profiling approaches (e.g., scEU-seq, sci-fate, scNT-seq) (Hendriks, G.-J. et al., Nat. Commun. 10, 3138 (2019); Cao, J., Zhou. Et al., Nat. Biotechnol. 38, 980-988 (2020); Qiu, Q. et al., Nat. Methods 17, 991-1001 (2020); Cleary, M. D. et al., Nat. Biotechnol. 23, 232-237 (2005)). Following 4sU labeling and thiol (SH)-linked alkylation reaction (referred to as ‘chemical conversion’) (Dolken, L. et al., RNA 14, 1959-1972 (2008); Miller, C. et al., Mol. Syst. Biol. 7, 458-458 (2014); Duffy, E. E. et al., Mol. Cell 59, 858-866 (2015); Schwalb, B. et al., Science 352, 1225-1228 (2016); Rabani, M. et al., Nat. Biotechnol. 29, 436-442 (2011); Miller, M. R. et al., Nat. Methods 6, 439-441 (2009); Kawata, K. et al., Genome Res. 30, 1481-1491 (2020)), the nascent transcriptome and the whole transcriptome from the same cell can be distinguished by T to C conversion in reads mapping to mRNAs (Qiu, Q. et al., Nat. Methods 17, 991-1001 (2020)). The kinetic rate of mRNA dynamics (e.g., synthesis and degradation) were then calculated as a multi-layer readout for each genetic perturbation (FIG. 39A, Methods).

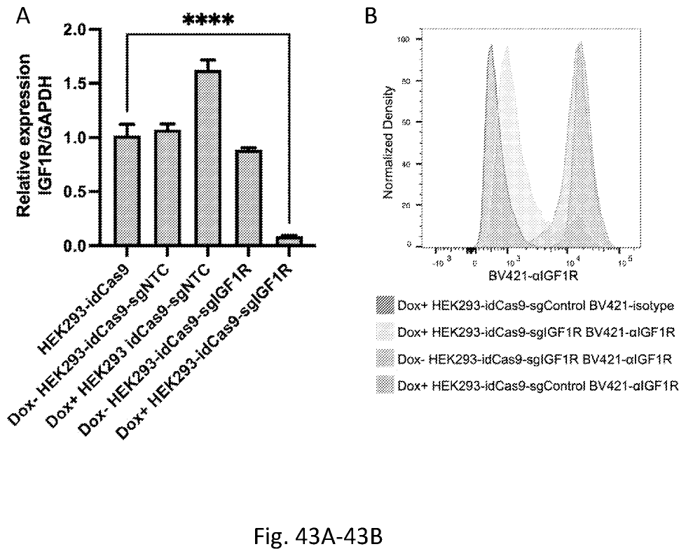

As a proof-of-concept, the approach was first tested in a mouse 3T3-L1-CRISPRi cell line transduced with a non-target control (NTC) sgRNA or sgRNA targeting an FTO gene (encoding an RNA demethylase). It was found that sgRNA expression was detected in up to 99.7% of all cells, with a median of 284 sgRNA UMI detected per cell in the optimal condition (i.e., 1 uM gRNA primer+50 uM dT primer in reverse transcription) (FIG. 41). A human HEK293 cell line with the inducible expression of dCas9-KRAB-MeCP2 (HEK293-idCas9) was then generated, and the sgRNA capture efficiency was tested using an NTC sgRNA and a sgRNA targeting the IGF-1R gene (encoding insulin-like growth factor 1 receptor). The transductions of the NTC and target sgRNAs were performed independently, such that each cell received a unique perturbation. The PerturbSci protocol was then carried out on a 1:1 mixture of cells from these two conditions. The target sgRNA expression in 97% of cells was recovered, of which 89.4% were sgRNA singlets with a median of 81 sgRNA UMIs detected per cell (FIG. 39C). Single-cell gene expression analysis confirmed the induction of dCas9 after Dox treatment and the significantly decreased IGF-1R expression in cells transduced with the target sgRNA (FIG. 39D). Strongly reduced IGF-1R mRNA and protein levels were further validated by RT-qPCR and flow cytometry (FIG. 43), indicating the high knockdown efficiency of the system.

The PerturbSci-Kinetics method was validated for capturing three-layer readout (i.e., nascent transcriptome, whole transcriptome, sgRNA identities) at the single-cell level. Following 4-thiouridine (4sU) labeling (200 uM for two hours), HEK293-idCas9 cells transduced with control or IGF1R sgRNA were mixed at a 1:1 ratio for fixation and chemical conversion. A significant enrichment of T to C mismatches was observed in mapped reads of the chemical conversion group, similar to a previous study (FIG. 39E) (Cao, J., Zhou. Et al., Nat. Biotechnol. 38, 980-988 (2020)). Also, a median of 22.1% of newly synthesized reads was recovered in labeled and chemically converted cells, compared to only 0.8% in control groups (FIG. 39F). Reassuringly, the proportion of reads mapped to exonic regions was significantly lower in newly synthesized reads compared with pre-existing reads (p-value<1e-20, Tukey's test after ANOVA) (FIG. 39G). Indeed, genes with a higher fraction of nascent reads were significantly enriched in highly dynamic biological processes such as transcription coregulator activity (q-value=5.7e-12) and protein kinase activity (q-value=2.6e-08) (FIG. 39H) (Kawata, K. et al., Genome Res. 30, 1481-1491 (2020)). By contrast, genes with a lower fraction of nascent reads were strongly enriched for processes essential for cell vitality, such as the structural constituent of ribosome (q-value=1.5e-42), unfolded protein binding (q-value=4.5e-11), and translation regulator activity (q-value=8.2e-10) (FIG. 39I). Notably, the metabolic labeling and the following chemical conversion steps are fully compatible with sgRNA detection at single-cell resolution: sgRNAs were recovered from 97% of chemically converted cells (a median of 62 sgRNA UMIs/cell), comparable to the detection efficiency in the control group (FIG. 39J-K). These analyses demonstrate the capacity of PerturbSci-Kinetics to profile both transcriptome dynamics and the associated perturbation identity at the single-cell level.

To dissect key regulators of transcriptome kinetics, a PerturbSci-Kinetics screen was performed on HEK293-idCas9 cells transduced with a library of 699 sgRNAs, containing 15 non-targeting controls (NTC) and guides targeting 228 genes involved in a variety of biological processes including mRNA transcription, processing, degradation, and others (FIG. 44A). The cloning and lentiviral packaging were performed in a pooled fashion, similar to the previous report (Joung, J. et al. Nat. Protoc. 12, 828-863 (2017)). HEK293-idCas9 cell line were then infected with the sgRNA virus library at a low multiplicity of infection (MOI) (2 repeats at MOI=0.1 and 2 repeats at MOI=0.2) to ensure most cells received only one sgRNA. After a 5-day puromycin selection to remove cells receiving no sgRNA, a fraction of cells were harvested for bulk library preparation (‘day 0’ samples). The rest of the cells were treated with Doxycycline (Dox) to induce the dCas9-KRAB-MeCP2 expression. After additional seven days for efficient gene knockdown, 4sU labeling (200 uM for two hours) was introduced and samples for both bulk and single-cell PerturbSci-Kinetics library preparation (‘day 7’ samples) were harvested. The time window for the screening period was chosen to minimize non-direct downstream transcriptional changes and population dropout (Replogle, J. M. et al., Cell 185, 2559-2575.e28 (2022)).

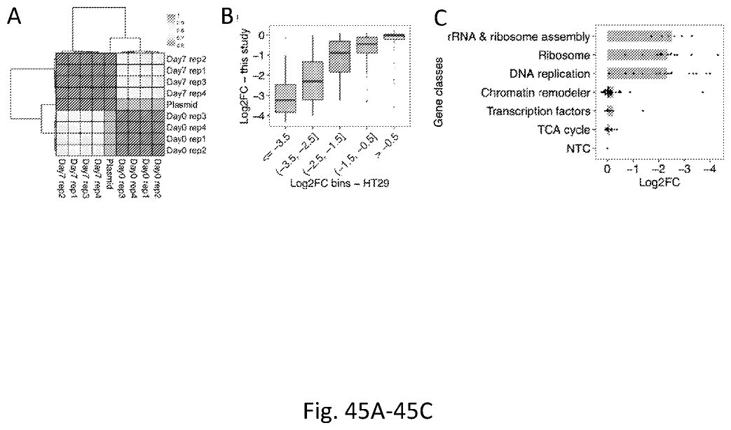

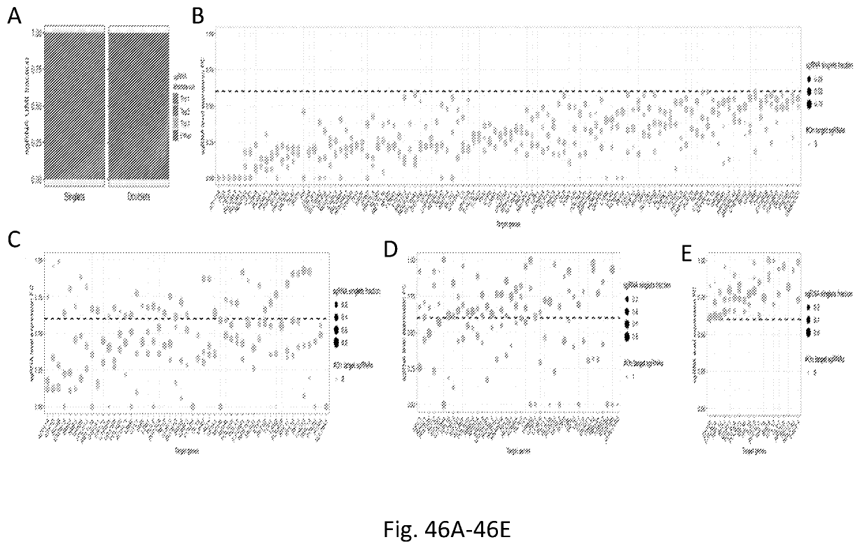

As expected, the induction of CRISPRi significantly changed the abundance of sgRNAs in the cell population, which is consistent between replicates and the previous study (FIG. 45) (Stuart, T. et al., Cell 177, 1888-1902.e21 (2019)). For example, the guides targeting genes involved in essential biological functions, such as DNA replication, ribosome assembly, and rRNA processing, were strongly depleted in the screen (FIG. 46). Reassuringly, the sgRNA abundance recovered by PerturbSci-kinetics strongly correlated with the bulk library (Pearson correlation r=0.988, p-value<2.2e-16) (FIG. 44C). After filtering out low-quality cells, 161,966 metabolic labeled cells were recovered, 88.1% of which had matched sgRNAs. Despite relatively low sequencing depth (17.9% of duplication rate), a median of 2,155 UMIs per cell was obtained. Most (698 out of 699) guide RNAs were detected, with a median of 28 sgRNA UMIs per cell. sgRNAs with low knockdown efficiencies (<=40% expression reduction of target genes compared with NTC) and cells assigned to multiple sgRNAs were further filtered out (FIG. 46). 98,315 cells were retained for downstream analysis, corresponding to a median of 484 cells per gene perturbation with a median of 67.7% knockdown efficiency of target genes (FIG. 44D). To further validate the gene perturbations, single-cell transcriptomes were aggregated to generate ‘pseudo-cells’ for each gene perturbation, followed by PCA dimension reduction and UMAP visualization (Qiu, X. et al., Cell 185, 690-711.e45 (2022)). Indeed, perturbations targeting paralogous genes (e.g., EXOSC5 and EXOSC6; CNOT2 and CNOT3) or related biological processes (e.g., RNA degradation, RNA splicing, oxidative phosphorylation (OXPHOS) and energy metabolism) were readily clustered together in the low dimension space (FIG. 44B).

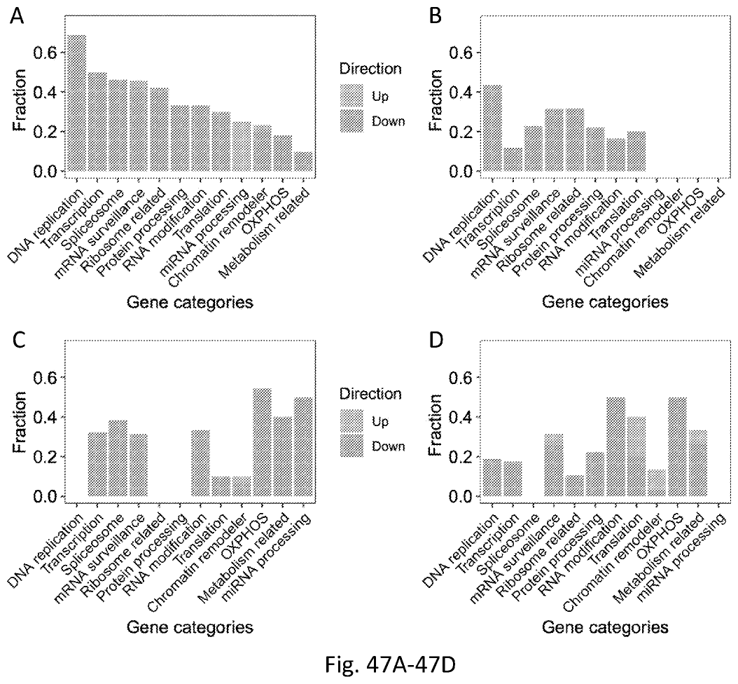

Taking advantage of PerturbSci-Kinetics for uniquely capturing multiple layers of information, gene-specific synthesis and degradation rate were quantified in each perturbation based on an ordinary differential equation (Methods) (Qiu, X. et al., Cell 185, 690-711.e45 (2022)). As a quality control, the kinetics of genes targeted by CRISPRi were examined, which were known to function through transcriptional repression (Jones, P. L. et al., Nat. Genet. 19, 187-191 (1998); Dominguez, A. et al., Nature Reviews Molecular Cell Biology vol. 17 5-15). Indeed, these genes exhibited significantly reduced synthesis rates while their degradation rates were only mildly affected (a median reduction fold in synthesis: −2.00 vs. −0.318 in degradation; FIG. 44D-F). The impact of genetic perturbations on global mRNA synthesis and degradation rates was then investigated (Methods). As expected, the knockdown of genes involved in transcription initiation (e.g., GTF2E1, TAF2, MED21, and MNAT1), mRNA synthesis (e.g., POLK2B and POLR2K), and chromatin remodeling (e.g., SMC3, RAD21, CTCF, ARID1A) significantly down-regulated the synthesis rate, but not the degradation rate, of the global transcriptome. Interestingly, perturbations targeting components of critical biological processes such as DNA replication (e.g., POLA2, POLD1), ribosome synthesis (e.g., POLR1A, POLR1B, RPLI1, RPS15A), mRNA and protein processing (e.g., CNOT2, CNOT3, CCT3, CCT4) showed a substantial defect in both global mRNA synthesis and degradation, indicating the existence of secondary signaling circuits for maintaining overall transcriptome abundance in cells (FIG. 44G-H, FIG. 47). In addition, several genes (e.g., YY1, AGO2) were identified as potential repressors of global transcription, revealing their potential non-canonical functions (Kalantari, R., et al., Nucleic Acids Res. 44, 524-537 (2016); Nishi, K. et al., RNA 19, 17-35 (2013); Gordon, S. et al., Oncogene 25, 1125-1142 (2006)).

Besides global mRNA synthesis and degradation, the regulators of mRNA processing were further investigated by examining the ratio of nascent reads mapped to exonic regions (referred to as ‘exonic reads ratio’) for each perturbation. As expected, the knockdown of genes involved in the main steps of RNA processing, including 5′ capping (e.g., NCBP1), splicing (e.g., LSM2, LSM4, PRPF38B, HNRNPK), and 3′ cleavage and polyadenylation (e.g., CPSF2, CPSF6, NUDT21, CSTF3) resulted in a significantly lower exonic reads ratio (FIG. 44I). Also, perturbing genes involved in OXPHOS & energy metabolism (e.g., GAPDH, NDUFS2, ACO2) exhibited a significant effect on exonic reads ratio (FIG. 44I, FIG. 47), consistent with the previous reports that the mRNA processing is highly energy-dependent (Kim, S. H. et al., Proc. Natl. Acad. Sci. U.S.A 90, 888-892 (1993); Colgan, D. F. et al., Genes & Development vol. 11, 2755-2766 (1997); Kikkawa, S. et al., J. Biol. Chem. 265, 21536-21540 (1990)).

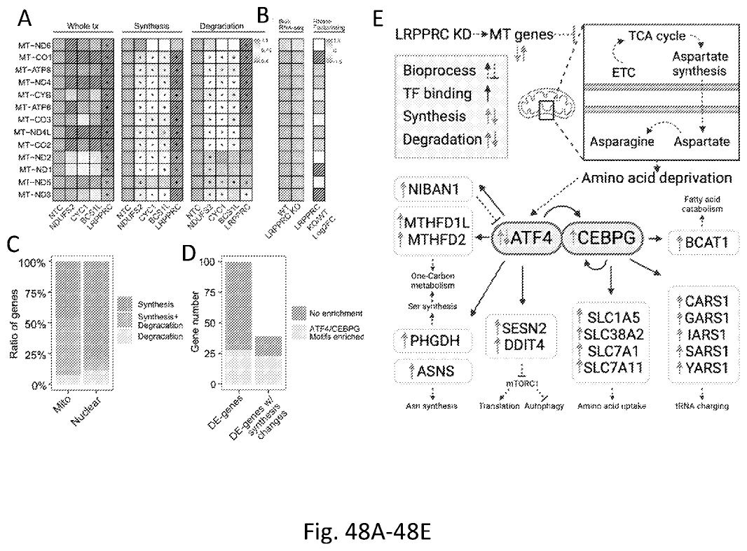

Regulators of mitochondrial mRNA turnover were then investigated by quantifying the ratio of nascent/total read counts mapped to mitochondrial genes. Notably, significantly down-regulated turnover rates of mitochondrial-specific RNA following the perturbation of multiple metabolism-related genes was observed (e.g., GAPDH, FH, PKM involved in glycolysis, ACO2 and IDH3A involved in the TCA cycle, NDUFS2 and COX6B1 involved in oxidative phosphorylation) (FIG. 44J). Furthermore, it was found that the perturbation on LRPPRC led to the most substantial defect in mitochondrial mRNA turnover (FIG. 44J) and significant expression reduction on all mitochondrial protein-coding genes (FIG. 48). Intriguingly, some mitochondrial protein-coding genes, including MT-ND6, MT-CO1, MT-ATP8, MT-ND4, MT-CYB, and MT-ATP6, are regulated at both transcription and degradation levels, consistent with the known functions of LRPPRC in regulating the life cycles of mitochondrial RNA from transcription to degradation (Colgan, D. F. et al., Genes & Development vol. 11, 2755-2766 (1997); Kikkawa, S. et al., J. Biol. Chem. 265, 21536-21540 (1990); Pajak, A. et al., PLOS Genet. 15, e1008240 (2019)). For example, 39 nuclear-encoded differentially expressed genes (DEGs) were significantly perturbed at the transcription level, while only nine were regulated by degradation following LRPPRC knockdown. Upon closer inspection of promoter regions of these genes, a significant enrichment of motifs from ATF4 and CEBPG was observed, both of which were substantially down-regulated in LRPPRC knockdown cells (FIG. 48). ATF4 and CEGPG have been reported as core transcriptional activators involved in stress sensing, suggesting their potential roles as downstream regulators of LRPPRC (Liu, L. et al., J. Biol. Chem. 286, 41253-41264 (2011))

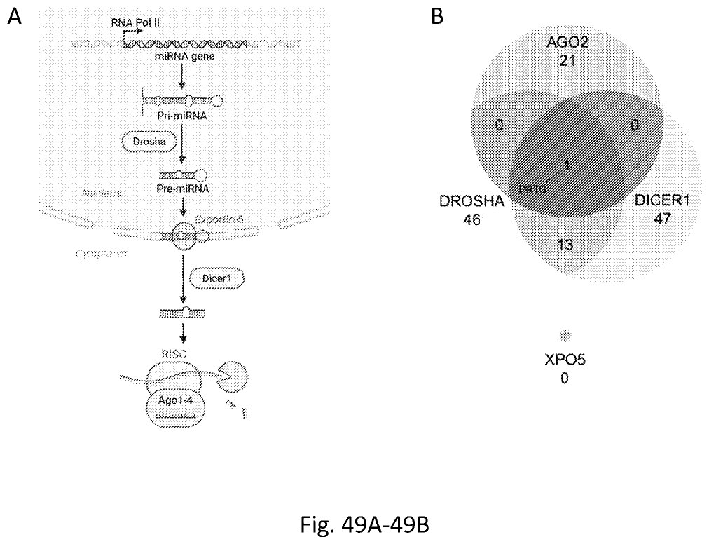

Extending on the above analysis, the gene-specific synthesis and degradation regulation across all gene perturbations was examined. Among all 14,618 DEGs identified in the study, 31.3% of DEGs exhibited significant changes in synthesis rates (19.3%), degradation rates (7.8%) or both (4.2%), suggesting complex mechanisms controlling gene expression upon genetic perturbations (Ruzzenente, B. et al., EMBO J. 31, 443-456 (2012)). For some perturbations, including genes involved in mRNA surveillance/processing (e.g., UPF1, UPF2, SMG5, SMG7 in nonsense-mediated mRNA decay pathway; EXOSC2, EXOSC5, EXOSC6 in RNA exosome; CSTF3, CPSF2, CPSF6, NUDT21, XRN2 for 3′ polyadenylation; RNMT, NCBP1 related to 5′ RNA capping) (FIG. 44L-M), their associated DEGs are mainly regulated through degradation as expected. By contrast, other perturbations may lead to more complex scenarios. For example, the knockdown of two critical regulators in the microRNA (miRNA) pathway (i.e., DROSHA and DICER1) (García-Martinez, J. et al., Nucleic Acids Res. 44, 3643-3658 (2016); Siira, S. J. et al., Nat. Commun. 8, 1532 (2017); Pakos-Zebrucka, K. et al., EMBO Rep. 17, 1374-1395 (2016)) resulted in highly overlapped DEGs that were regulated through distinct mechanisms (FIG. 44N-O, FIG. 49). Part of the up-regulated genes (FDR of 0.05, e.g., TMEM245, PRTG, TNRC6A) is regulated by significantly decreased degradation rates, while others were regulated mostly at the transcription level. These genes include known regulators of miRNA host genes (e.g., MIR181A1HG, FTX), miRNA maturation (e.g., DDX3X), and the RNA degradation machinery (e.g., TNRC6A) (Buccitelli, C. et al., Nat. Rev. Genet. 21, 630-644 (2020); Chipman, L. B. et al., Trends Genet. 35, 215-222 (2019); Treiber, T. et al., Nat. Rev. Mol. Cell Biol. 20, 5-20 (2019); Kim, Y.-K. et al., Proc. Natl. Acad. Sci. U.S.A 113, E1881-9 (2016)), suggesting a compensatory circuit for maintaining the overall miRNA/mRNA homeostasis (FIG. 44Q). To explore the underlying regulatory mechanisms, the gene-specific binding patterns of Ago2 was examined, one of the core components in miRNA-mediated silencing complex (RISC) for targeted mRNA binding and degradation (Liu, B. et al., Brief. Funct. Genomics 18, 255-266 (2018)). Indeed, Ago2 binding was strongly enriched in the first gene set with dysregulated degradation following perturbations of the miRNA pathway. The detected binding signal was primarily enriched in the 5′ and 3′ untranslated regions (UTR), consistent with prior reports (FIG. 44P) (Chureau, C. et al., Hum. Mol. Genet. 20, 705-718 (2011); Siira, S. J. et al., Nat. Commun. 8, 1532 (2017)). For comparison, there was not a detection of strong enrichment of Ago2 binding in the second gene set that exhibited up-regulated transcriptional rates upon perturbations, consistent with the result that these genes are regulated at the transcriptional level. In summary, the above analysis demonstrates the unique capacity of PerturbSci-Kinetics for inferring the underlying regulatory mechanisms associated with gene expression changes in genetic perturbations.

TABLE 11
sgRNA Capture Primers

| | | SEQ ID | | SEQ ID |
|---|---|---|---|---|
| Name | Sequence | NO: | Barcode | NO: |
| sciNEXT_RT- | /5Phos/ACGACGCTCTTCCGATCTNNNNNNNNT | 2305 | TTCTCGCATG | 193 |
| gRNA_targeted_plate1_01 | TCTCGCATGCAAGTTGATAACGGACTAGCC | | | |
| sciNEXT_RT- | /5Phos/ACGACGCTCTTCCGATCTNNNNNNNNT | 2306 | TCCTACCAGT | 194 |
| gRNA_targeted_plate1_02 | CCTACCAGTCAAGTTGATAACGGACTAGCC | | | |
| sciNEXT_RT- | /5Phos/ACGACGCTCTTCCGATCTNNNNNNNNG | 2307 | GCGTTGGAGC | 195 |
| gRNA_targeted_plate1_03 | CGTTGGAGCCAAGTTGATAACGGACTAGCC | | | |
| sciNEXT_RT- | /5Phos/ACGACGCTCTTCCGATCTNNNNNNNNG | 2308 | GATCTTACGC | 196 |
| gRNA_targeted_plate1_04 | ATCTTACGCCAAGTTGATAACGGACTAGCC | | | |
| sciNEXT_RT- | /5Phos/ACGACGCTCTTCCGATCTNNNNNNNNC | 2309 | CTGATGGTCA | 197 |
| gRNA_targeted_plate1_05 | TGATGGTCACAAGTTGATAACGGACTAGCC | | | |
| sciNEXT_RT- | /5Phos/ACGACGCTCTTCCGATCTNNNNNNNNC | 2310 | CCGAGAATCC | 198 |
| gRNA_targeted_plate1_06 | CGAGAATCCCAAGTTGATAACGGACTAGCC | | | |
| sciNEXT_RT- | /5Phos/ACGACGCTCTTCCGATCTNNNNNNNNG | 2311 | GCCGCAACGA | 199 |
| gRNA_targeted_plate1_07 | CCGCAACGACAAGTTGATAACGGACTAGCC | | | |
| sciNEXT_RT- | /5Phos/ACGACGCTCTTCCGATCTNNNNNNNNT | 2312 | TGAGTCTGGC | 200 |
| gRNA_targeted_plate1_08 | GAGTCTGGCCAAGTTGATAACGGACTAGCC | | | |
| sciNEXT_RT- | /5Phos/ACGACGCTCTTCCGATCTNNNNNNNNT | 2313 | TGCGGACCTA | 201 |
| gRNA_targeted_plate1_09 | GCGGACCTACAAGTTGATAACGGACTAGCC | | | |
| sciNEXT_RT- | /5Phos/ACGACGCTCTTCCGATCTNNNNNNNNA | 2314 | ACCTCGTTGA | 202 |
| gRNA_targeted_plate1_10 | CCTCGTTGACAAGTTGATAACGGACTAGCC | | | |
| sciNEXT_RT- | /5Phos/ACGACGCTCTTCCGATCTNNNNNNNNA | 2315 | ACGGAGGCG | 203 |
| gRNA_targeted_platel_11 | CGGAGGCGGCAAGTTGATAACGGACTAGCC | | G | |
| sciNEXT_RT- | /5Phos/ACGACGCTCTTCCGATCTNNNNNNNNT | 2316 | TAGATCTACT | 204 |
| gRNA_targeted_plate1_12 | AGATCTACTCAAGTTGATAACGGACTAGCC | | | |
| sciNEXT_RT- | /5Phos/ACGACGCTCTTCCGATCTNNNNNNNNA | 2317 | AATTAAGACT | 205 |
| gRNA_targeted_plate1_13 | ATTAAGACTCAAGTTGATAACGGACTAGCC | | | |
| sciNEXT_RT- | /5Phos/ACGACGCTCTTCCGATCTNNNNNNNNC | 2318 | CCATTGCGTT | 206 |
| gRNA_targeted_plate1_14 | CATTGCGTTCAAGTTGATAACGGACTAGCC | | | |
| sciNEXT_RT- | /5Phos/ACGACGCTCTTCCGATCTNNNNNNNNT | 2319 | TTATTCATTC | 207 |
| gRNA_targeted_platel_15 | TATTCATTCCAAGTTGATAACGGACTAGCC | | | |
| sciNEXT_RT- | /5Phos/ACGACGCTCTTCCGATCTNNNNNNNNA | 2320 | ATCTCCGAAC | 208 |
| gRNA_targeted_plate1_16 | TCTCCGAACCAAGTTGATAACGGACTAGCC | | | |
| sciNEXT_RT- | /5Phos/ACGACGCTCTTCCGATCTNNNNNNNNT | 2321 | TTGACTTCAG | 209 |
| gRNA_targeted_plate1_17 | TGACTTCAGCAAGITGATAACGGACTAGCC | | | |
| sciNEXT_RT- | /5Phos/ACGACGCTCTTCCGATCTNNNNNNNNG | 2322 | GGCAGGTATT | 210 |
| gRNA_targeted_plate1_18 | GCAGGTATTCAAGTTGATAACGGACTAGCC | | | |
| sciNEXT_RT- | /5Phos/ACGACGCTCTTCCGATCTNNNNNNNNA | 2323 | AGAGCTATAA | 211 |
| gRNA_targeted_plate1_19 | GAGCTATAACAAGTTGATAACGGACTAGCC | | | |
| sciNEXT_RT- | /5Phos/ACGACGCTCTTCCGATCTNNNNNNNNC | 2324 | CTAAGAGAAG | 212 |
| gRNA_targeted_plate1_20 | TAAGAGAAGCAAGTTGATAACGGACTAGCC | | | |
| sciNEXT_RT- | /5Phos/ACGACGCTCTTCCGATCTNNNNNNNNA | 2325 | ACTCAATAGG | 213 |
| gRNA_targeted_plate1_21 | CTCAATAGGCAAGTTGATAACGGACTAGCC | | | |
| sciNEXT_RT- | /5Phos/ACGACGCTCTTCCGATCTNNNNNNNNC | 2326 | CTTGCGCCGC | 214 |
| gRNA_targeted_platel_22 | TTGCGCCGCCAAGTTGATAACGGACTAGCC | | | |
| sciNEXT_RT- | /5Phos/ACGACGCTCTTCCGATCTNNNNNNNNA | 2327 | AATCGTAGCG | 215 |
| gRNA_targeted_plate1_23 | ATCGTAGCGCAAGTTGATAACGGACTAGCC | | | |
| sciNEXT_RT- | /5Phos/ACGACGCTCTTCCGATCTNNNNNNNNG | 2328 | GGTACTGCCT | 216 |
| gRNA_targeted_plate1_24 | GTACTGCCTCAAGTTGATAACGGACTAGCC | | | |
| sciNEXT_RT- | /5Phos/ACGACGCTCTTCCGATCTNNNNNNNNT | 2329 | TAGAATTAAC | 217 |
| gRNA_targeted_plate1_25 | AGAATTAACCAAGTTGATAACGGACTAGCC | | | |
| sciNEXT_RT- | /5Phos/ACGACGCTCTTCCGATCTNNNNNNNNG | 2330 | GCCATTCTCC | 218 |
| gRNA_targeted_plate1_26 | CCATTCTCCCAAGTTGATAACGGACTAGCC | | | |
| sciNEXT_RT- | /5Phos/ACGACGCTCTTCCGATCTNNNNNNNNT | 2331 | TGCCGGCAGA | 219 |
| gRNA_targeted_plate1_27 | GCCGGCAGACAAGTTGATAACGGACTAGCC | | | |
| sciNEXT_RT- | /5Phos/ACGACGCTCTTCCGATCTNNNNNNNNT | 2332 | TTACCGAGGC | 220 |
| gRNA_targeted_platel_28 | TACCGAGGCCAAGTTGATAACGGACTAGCC | | | |
| sciNEXT_RT- | /5Phos/ACGACGCTCTTCCGATCTNNNNNNNNA | 2333 | ATCATATTAG | 221 |
| gRNA_targeted_platel_29 | TCATATTAGCAAGTTGATAACGGACTAGCC | | | |
| sciNEXT_RT- | /5Phos/ACGACGCTCTTCCGATCTNNNNNNNNT | 2334 | TGGTCAGCCA | 222 |
| gRNA_targeted_plate1_30 | GGTCAGCCACAAGTTGATAACGGACTAGCC | | | |
| sciNEXT_RT- | /5Phos/ACGACGCTCTTCCGATCTNNNNNNNNA | 2335 | ACTATGCAAT | 223 |
| gRNA_targeted_plate1_31 | CTATGCAATCAAGTTGATAACGGACTAGCC | | | |
| sciNEXT_RT- | /5Phos/ACGACGCTCTTCCGATCTNNNNNNNNC | 2336 | CGACGCGACT | 224 |
| gRNA_targeted_plate1_32 | GACGCGACTCAAGTTGATAACGGACTAGCC | | | |
| sciNEXT_RT- | /5Phos/ACGACGCTCTTCCGATCTNNNNNNNNG | 2337 | GATACGGAAC | 225 |
| gRNA_targeted_plate1_33 | ATACGGAACCAAGTTGATAACGGACTAGCC | | | |
| sciNEXT_RT- | /5Phos/ACGACGCTCTTCCGATCTNNNNNNNNT | 2338 | TTATCCGGAT | 226 |
| gRNA_targeted_plate1_34 | TATCCGGATCAAGTTGATAACGGACTAGCC | | | |
| sciNEXT_RT- | /5Phos/ACGACGCTCTTCCGATCTNNNNNNNNT | 2339 | TAGAGTAATA | 227 |
| gRNA_targeted_plate1_35 | AGAGTAATACAAGTTGATAACGGACTAGCC | | | |
| sciNEXT_RT- | /5Phos/ACGACGCTCTTCCGATCTNNNNNNNNG | 2340 | GCAGGTCCGT | 228 |
| gRNA_targeted_plate1_36 | CAGGTCCGTCAAGTTGATAACGGACTAGCC | | | |
| sciNEXT_RT- | /5Phos/ACGACGCTCTTCCGATCTNNNNNNNNT | 2341 | TCGGCCTTAC | 229 |
| gRNA_targeted_plate1_37 | CGGCCTTACCAAGTTGATAACGGACTAGCC | | | |
| sciNEXT_RT- | /5Phos/ACGACGCTCTTCCGATCTNNNNNNNNA | 2342 | AGAACGTCTC | 230 |
| gRNA_targeted_plate1_38 | GAACGTCTCCAAGTTGATAACGGACTAGCC | | | |
| sciNEXT_RT- | /5Phos/ACGACGCTCTTCCGATCTNNNNNNNNC | 2343 | CCAGTTCCAA | 231 |
| gRNA_targeted_plate1_39 | CAGTTCCAACAAGTTGATAACGGACTAGCC | | | |
| sciNEXT_RT- | /5Phos/ACGACGCTCTTCCGATCTNNNNNNNNG | 2344 | GGCGTTAAGG | 232 |
| gRNA_targeted_platel_40 | GCGTTAAGGCAAGTTGATAACGGACTAGCC | | | |
| sciNEXT_RT- | /5Phos/ACGACGCTCTTCCGATCTNNNNNNNNA | 2345 | ACTTAACCTT | 233 |
| gRNA_targeted_plate1_41 | CTTAACCTTCAAGTTGATAACGGACTAGCC | | | |
| sciNEXT_RT- | /5Phos/ACGACGCTCTTCCGATCTNNNNNNNNC | 2346 | CAACCGCTAA | 234 |
| gRNA_targeted_plate1_42 | AACCGCTAACAAGTTGATAACGGACTAGCC | | | |
| sciNEXT_RT- | /5Phos/ACGACGCTCTTCCGATCTNNNNNNNNG | 2347 | GACCTTGATA | 235 |
| gRNA_targeted_plate1_43 | ACCTTGATACAAGTTGATAACGGACTAGCC | | | |
| sciNEXT_RT- | /5Phos/ACGACGCTCTTCCGATCTNNNNNNNNT | 2348 | TCTGATACCA | 236 |
| gRNA_targeted_plate1_44 | CTGATACCACAAGTTGATAACGGACTAGCC | | | |
| sciNEXT_RT- | /5Phos/ACGACGCTCTTCCGATCTNNNNNNNNG | 2349 | GAAGATCGAG | 237 |
| gRNA_targeted_plate1_45 | AAGATCGAGCAAGTTGATAACGGACTAGCC | | | |
| sciNEXT_RT- | /5Phos/ACGACGCTCTTCCGATCTNNNNNNNNA | 2350 | AGGAGCGGTA | 238 |
| gRNA_targeted_plate1_46 | GGAGCGGTACAAGTTGATAACGGACTAGCC | | | |
| sciNEXT_RT- | /5Phos/ACGACGCTCTTCCGATCTNNNNNNNNA | 2351 | AAGAAGCTAG | 239 |
| gRNA_targeted_plate1_47 | AGAAGCTAGCAAGTTGATAACGGACTAGCC | | | |
| sciNEXT_RT- | /5Phos/ACGACGCTCTTCCGATCTNNNNNNNNT | 2352 | TCCGGCCTCG | 240 |
| gRNA_targeted_plate1_48 | CCGGCCTCGCAAGTTGATAACGGACTAGCC | | | |
| sciNEXT_RT- | /5Phos/ACGACGCTCTTCCGATCTNNNNNNNNA | 2353 | AGAGAAGGTT | 241 |
| gRNA_targeted_plate1_49 | GAGAAGGTTCAAGTTGATAACGGACTAGCC | | | |
| sciNEXT_RT- | /5Phos/ACGACGCTCTTCCGATCTNNNNNNNNC | 2354 | CATACTCCGA | 242 |
| gRNA_targeted_plate1_50 | ATACTCCGACAAGTTGATAACGGACTAGCC | | | |
| sciNEXT_RT- | /5Phos/ACGACGCTCTTCCGATCTNNNNNNNNG | 2355 | GCTAACTTGC | 243 |
| gRNA_targeted_plate1_51 | CTAACTTGCCAAGTTGATAACGGACTAGCC | | | |
| sciNEXT_RT- | /5Phos/ACGACGCTCTTCCGATCTNNNNNNNNA | 2356 | AATCCATCTT | 244 |
| gRNA_targeted_plate1_52 | ATCCATCTTCAAGTTGATAACGGACTAGCC | | | |
| sciNEXT_RT- | /5Phos/ACGACGCTCTTCCGATCTNNNNNNNNG | 2357 | GGCTGAGCTC | 245 |
| gRNA_targeted_plate1_53 | GCTGAGCTCCAAGTTGATAACGGACTAGCC | | | |
| sciNEXT_RT- | /5Phos/ACGACGCTCTTCCGATCTNNNNNNNNC | 2358 | CCGATTCCTG | 246 |
| gRNA_targeted_plate1_54 | CGATTCCTGCAAGTTGATAACGGACTAGCC | | | |
| sciNEXT_RT- | /5Phos/ACGACGCTCTTCCGATCTNNNNNNNNA | 2359 | ACCGCCAACC | 247 |
| gRNA_targeted_plate1_55 | CCGCCAACCCAAGTTGATAACGGACTAGCC | | | |
| sciNEXT_RT- | /5Phos/ACGACGCTCTTCCGATCTNNNNNNNNT | 2360 | TGGCCTGAAG | 248 |
| gRNA_targeted_plate1_56 | GGCCTGAAGCAAGTTGATAACGGACTAGCC | | | |
| sciNEXT_RT- | /5Phos/ACGACGCTCTTCCGATCTNNNNNNNNA | 2361 | AACCTCATTC | 249 |
| gRNA_targeted_plate1_57 | ACCTCATTCCAAGTTGATAACGGACTAGCC | | | |
| sciNEXT_RT- | /5Phos/ACGACGCTCTTCCGATCTNNNNNNNNA | 2362 | ATAAGGAGCA | 250 |
| gRNA_targeted_plate1_58 | TAAGGAGCACAAGTTGATAACGGACTAGCC | | | |
| sciNEXT_RT- | /5Phos/ACGACGCTCTTCCGATCTNNNNNNNNC | 2363 | CGAACGCCGG | 251 |
| gRNA_targeted_plate1_59 | GAACGCCGGCAAGTTGATAACGGACTAGCC | | | |
| sciNEXT_RT- | /5Phos/ACGACGCTCTTCCGATCTNNNNNNNNG | 2364 | GGTATGCTTG | 252 |
| gRNA_targeted_plate1_60 | GTATGCTTGCAAGTTGATAACGGACTAGCC | | | |
| sciNEXT_RT- | /5Phos/ACGACGCTCTTCCGATCTNNNNNNNNA | 2365 | AACCTGCGTA | 253 |
| gRNA_targeted_plate1_61 | ACCTGCGTACAAGTTGATAACGGACTAGCC | | | |
| sciNEXT_RT- | /5Phos/ACGACGCTCTTCCGATCTNNNNNNNNG | 2366 | GGCAGACGCC | 254 |
| gRNA_targeted_plate1_62 | GCAGACGCCCAAGTTGATAACGGACTAGCC | | | |
| sciNEXT_RT- | /5Phos/ACGACGCTCTTCCGATCTNNNNNNNNT | 2367 | TAGCCGTCAT | 255 |
| gRNA_targeted_plate1_63 | AGCCGTCATCAAGTTGATAACGGACTAGCC | | | |
| sciNEXT_RT- | /5Phos/ACGACGCTCTTCCGATCTNNNNNNNNC | 2368 | CCTGGAAGAG | 256 |
| gRNA_targeted_plate1_64 | CTGGAAGAGCAAGTTGATAACGGACTAGCC | | | |
| sciNEXT_RT- | /5Phos/ACGACGCTCTTCCGATCTNNNNNNNNG | 2369 | GGAGGTTCTA | 257 |
| gRNA_targeted_plate1_65 | GAGGTTCTACAAGTTGATAACGGACTAGCC | | | |
| sciNEXT_RT- | /5Phos/ACGACGCTCTTCCGATCTNNNNNNNNC | 2370 | CTAGTAGTCT | 258 |
| gRNA_targeted_plate1_66 | TAGTAGTCTCAAGTTGATAACGGACTAGCC | | | |
| sciNEXT_RT- | /5Phos/ACGACGCTCTTCCGATCTNNNNNNNNA | 2371 | ATCATCAACG | 259 |
| gRNA_targeted_plate1_67 | TCATCAACGCAAGTTGATAACGGACTAGCC | | | |
| sciNEXT_RT- | /5Phos/ACGACGCTCTTCCGATCTNNNNNNNNA | 2372 | ACGCGAGATT | 260 |
| gRNA_targeted_plate1_68 | CGCGAGATTCAAGTTGATAACGGACTAGCC | | | |
| sciNEXT_RT- | /5Phos/ACGACGCTCTTCCGATCTNNNNNNNNG | 2373 | GAAGAGGCAT | 261 |
| gRNA_targeted_plate1_69 | AAGAGGCATCAAGTTGATAACGGACTAGCC | | | |
| sciNEXT_RT- | /5Phos/ACGACGCTCTTCCGATCTNNNNNNNNG | 2374 | GGTATCCGCC | 262 |
| gRNA_targeted_plate1_70 | GTATCCGCCCAAGTTGATAACGGACTAGCC | | | |
| sciNEXT_RT- | /5Phos/ACGACGCTCTTCCGATCTNNNNNNNNA | 2375 | AACTAGGCGC | 263 |
| gRNA_targeted_plate1_71 | ACTAGGCGCCAAGTTGATAACGGACTAGCC | | | |
| sciNEXT_RT- | /5Phos/ACGACGCTCTTCCGATCTNNNNNNNNT | 2376 | TCGCTAAGCA | 264 |
| gRNA_targeted_plate1_72 | CGCTAAGCACAAGTTGATAACGGACTAGCC | | | |
| sciNEXT_RT- | /5Phos/ACGACGCTCTTCCGATCTNNNNNNNNT | 2377 | TATATACTAA | 265 |
| gRNA_targeted_plate1_73 | ATATACTAACAAGTTGATAACGGACTAGCC | | | |
| sciNEXT_RT- | /5Phos/ACGACGCTCTTCCGATCTNNNNNNNNA | 2378 | ACTTGCTAGA | 266 |
| gRNA_targeted_plate1_74 | CTTGCTAGACAAGTTGATAACGGACTAGCC | | | |
| sciNEXT_RT- | /5Phos/ACGACGCTCTTCCGATCTNNNNNNNNA | 2379 | AACCATTGGA | 267 |
| gRNA_targeted_plate1_75 | ACCATTGGACAAGTTGATAACGGACTAGCC | | | |
| sciNEXT_RT- | /5Phos/ACGACGCTCTTCCGATCTNNNNNNNNT | 2380 | TCGCGGTTGG | 268 |
| gRNA_targeted_plate1_76 | CGCGGTTGGCAAGTTGATAACGGACTAGCC | | | |
| sciNEXT_RT- | /5Phos/ACGACGCTCTTCCGATCTNNNNNNNNC | 2381 | CGTAGTTACC | 269 |
| gRNA_targeted_plate1_77 | GTAGTTACCCAAGTTGATAACGGACTAGCC | | | |
| sciNEXT_RT- | /5Phos/ACGACGCTCTTCCGATCTNNNNNNNNT | 2382 | TCCAATCATC | 270 |
| gRNA_targeted_plate1_78 | CCAATCATCCAAGTTGATAACGGACTAGCC | | | |
| sciNEXT_RT- | /5Phos/ACGACGCTCTTCCGATCTNNNNNNNNA | 2383 | AATCGATAAT | 271 |
| gRNA_targeted_plate1_79 | ATCGATAATCAAGTTGATAACGGACTAGCC | | | |
| sciNEXT_RT- | /5Phos/ACGACGCTCTTCCGATCTNNNNNNNNC | 2384 | CCATTATCTA | 272 |
| gRNA_targeted_plate1_80 | CATTATCTACAAGTTGATAACGGACTAGCC | | | |
| sciNEXT_RT- | /5Phos/ACGACGCTCTTCCGATCTNNNNNNNNT | 2385 | TCAACGTAAG | 273 |
| gRNA_targeted_plate1_81 | CAACGTAAGCAAGTTGATAACGGACTAGCC | | | |
| sciNEXT_RT- | /5Phos/ACGACGCTCTTCCGATCTNNNNNNNNT | 2386 | TCTAATAGTA | 274 |
| gRNA_targeted_plate1_82 | CTAATAGTACAAGTTGATAACGGACTAGCC | | | |
| sciNEXT_RT- | /5Phos/ACGACGCTCTTCCGATCTNNNNNNNNA | 2387 | AACCGCTGGT | 275 |
| gRNA_targeted_plate1_83 | ACCGCTGGTCAAGTTGATAACGGACTAGCC | | | |
| sciNEXT_RT- | /5Phos/ACGACGCTCTTCCGATCTNNNNNNNNG | 2388 | GATCGCTTCT | 276 |
| gRNA_targeted_plate1_84 | ATCGCTTCTCAAGTTGATAACGGACTAGCC | | | |
| sciNEXT_RT- | /5Phos/ACGACGCTCTTCCGATCTNNNNNNNNC | 2389 | CTAACTAGAT | 277 |
| gRNA_targeted_plate1_85 | TAACTAGATCAAGTTGATAACGGACTAGCC | | | |
| sciNEXT_RT- | /5Phos/ACGACGCTCTTCCGATCTNNNNNNNNG | 2390 | GCTGGAACTT | 278 |
| gRNA_targeted_platel_86 | CTGGAACTTCAAGTTGATAACGGACTAGCC | | | |
| sciNEXT_RT- | /5Phos/ACGACGCTCTTCCGATCTNNNNNNNNA | 2391 | AGGTTAGTTC | 279 |
| gRNA_targeted_plate1_87 | GGTTAGTTCCAAGTTGATAACGGACTAGCC | | | |
| sciNEXT_RT- | /5Phos/ACGACGCTCTTCCGATCTNNNNNNNNC | 2392 | CATTCGACGG | 280 |
| gRNA_targeted_plate1_88 | ATTCGACGGCAAGTTGATAACGGACTAGCC | | | |
| sciNEXT_RT- | /5Phos/ACGACGCTCTTCCGATCTNNNNNNNNC | 2393 | CATTCAATCA | 281 |
| gRNA_targeted_plate1_89 | ATTCAATCACAAGTTGATAACGGACTAGCC | | | |
| sciNEXT_RT- | /5Phos/ACGACGCTCTTCCGATCTNNNNNNNNC | 2394 | CGGATTAGAA | 282 |
| gRNA_targeted_plate1_90 | GGATTAGAACAAGTTGATAACGGACTAGCC | | | |
| sciNEXT_RT- | /5Phos/ACGACGCTCTTCCGATCTNNNNNNNNA | 2395 | ATCGGCTATC | 283 |
| gRNA_targeted_plate1_91 | TCGGCTATCCAAGTTGATAACGGACTAGCC | | | |
| sciNEXT_RT- | /5Phos/ACGACGCTCTTCCGATCTNNNNNNNNC | 2396 | CCTTGATCGT | 284 |
| gRNA_targeted_plate1_92 | CTTGATCGTCAAGTTGATAACGGACTAGCC | | | |
| sciNEXT_RT- | /5Phos/ACGACGCTCTTCCGATCTNNNNNNNNA | 2397 | ACGAAGTCAA | 285 |
| gRNA_targeted_plate1_93 | CGAAGTCAACAAGTTGATAACGGACTAGCC | | | |
| sciNEXT_RT- | /5Phos/ACGACGCTCTTCCGATCTNNNNNNNNT | 2398 | TTACCTCGAC | 286 |
| gRNA_targeted_plate1_94 | TACCTCGACCAAGTTGATAACGGACTAGCC | | | |
| sciNEXT_RT- | /5Phos/ACGACGCTCTTCCGATCTNNNNNNNNG | 2399 | GGAGGATAGC | 287 |
| gRNA_targeted_plate1_95 | GAGGATAGCCAAGTTGATAACGGACTAGCC | | | |
| sciNEXT_RT- | /5Phos/ACGACGCTCTTCCGATCTNNNNNNNNG | 2400 | GGCTCTCTAT | 288 |
| gRNA_targeted_plate1_96 | GCTCTCTATCAAGTTGATAACGGACTAGCC | | | |

TABLE 12
sgRNA inner i7 primer

| | SEQ ID | | SEQ ID |
|---|---|---|---|
| Sequence | NO: | Barcode | NO: |
| CGTGTGCTCTTCCGATCTTCGGATTCGGatcttgtggaaaggacgaaaCACCG | 2401 | TCGGATTCGG | 1932 |
| CGTGTGCTCTTCCGATCTCTAAGCCTTGatcttgtggaaaggacgaaaCACCG | 2402 | CTAAGCCTTG | 1933 |
| CGTGTGCTCTTCCGATCTCTAACTAGGTatcttgtggaaaggacgaaaCACCG | 2403 | CTAACTAGGT | 1934 |
| CGTGTGCTCTTCCGATCTGCAAGACCGTatcttgtggaaaggacgaaaCACCG | 2404 | GCAAGACCGT | 1935 |
| CGTGTGCTCTTCCGATCTATGGAACGAAatcttgtggaaaggacgaaaCACCG | 2405 | ATGGAACGAA | 1936 |
| CGTGTGCTCTTCCGATCTTAGAGGCGTTatcttgtggaaaggacgaaaCACCG | 2406 | TAGAGGCGTT | 1937 |
| CGTGTGCTCTTCCGATCTGCATCGTATGatcttgtggaaaggacgaaaCACCG | 2407 | GCATCGTATG | 1938 |
| CGTGTGCTCTTCCGATCTTGGACGACTAatcttgtggaaaggacgaaaCACCG | 2408 | TGGACGACTA | 1939 |

### Example 5: Design

Single stranded sgRNA oligo for synthesis
5′-(SEQ ID NO: 2409)GGCTTTATATATCTTGTGGAAAGGACGAAACACCG
[20 bp sgRNA plus strand sequence]GTTTAAGAGCTATGCTGGAAACAGCATAGCAAGTT
(SEQ ID NO: 2410)-3′
Single gene knockdown cloning oligos for synthesis
plus strand
5′-CACCG[20 bp sgRNA plus strand sequence]-3′
minus strand
5′-AAAC[20 bp sgRNA minus strand sequence]C-3′
sgRNA readout capture RT primer
5′-(SEQ ID NO: 2411)/5Phos/ACGACGCTCTTCCGATCT[8 bp UMI][10 bp RT
barcode]CAAGTTGATAACGGACTAGCC-(SEQ ID NO: 2412)-3′
EasySci shortdT RT primer
-(SEQ ID NO: 2413)5′-/5Phos/ACGACGCTCTTCCGATCT[8 bp UMI][10bp RT
barcode]TTTTTTTTTTTTTTT-3′-(SEQ ID NO: 2414)
EasySci indexed ligation oligos
5′-(SEQ ID NO: 2415)AATGATACGGCGACCACCGAGATCTACAC[10 bp ligation
barcode]ACACTCTTTCCCTAC-3′ (SEQ ID NO: 2416)
Easy Sci indexed P7 primers
5′-(SEQ ID NO: 2417)CAAGCAGAAGACGGCATACGAGAT[10 bp index 7]
GTCTCGTGGGCTCGG-3′(SEQ ID NO: 2418)
sgRNA indexed P7 primers
5′-(SEQ ID NO: 2419)CAAGCAGAAGACGGCATACGAGAT[10 bp index
7]GTGACTGGAGTTCAGACGTGTGCTCTTCCGATCT-3′(SEQ ID NO: 2420)
Multiplex PCR sgRNA enrichment indexed primer
5′-(SEQ ID NO: 2421)CGTGTGCTCTTCCGATCT[10 bp inner
index7]ATCTTGTGGAAAGGACGAAACACCG (SEQ ID NO: 2422)-3′
Bulk screen genomic DNA amplification primers
P5 primer
5′-(SEQ ID NO: 2423)AATGATACGGCGACCACCGAGATCTACAC[10 bp index 5]
ACACTCTTTCCCTACACGACGCTCTTCCGATCTATCTTGTGGAAAGGACGAA
ACACCG-3′-(SEQ ID NO: 2424)
P7 primer
5′-(SEQ ID NO: 2425)CAAGCAGAAGACGGCATACGAGAT[10 bp index 7]
GTGACTGGAGTTCAGACGTGTGCTCTTCCGATCTCCGACTCGGTGCCACTTT
TTCAA-3′ (SEQ ID NO: 2426)

### Oligo List Sequences

KD cloning oligos
Mouse sgFto KD plus strand oligo
(SEQ ID NO: 2427)
CACCGGAAGCGCGTCCAGACCGCGG
Mouse sgFto KD minus strand oligo
(SEQ ID NO: 2428)
AAACCCGCGGTCTGGACGCGCTTCC
Mouse sgNTC KD plus strand oligo
(SEQ ID NO: 2429)
CACCGGGGAACCACATGGAATTCGA
Mouse sgNTC KD plus strand oligo
(SEQ ID NO: 2430)
AAACTCGAATTCCATGTGGTTCCCC
Human sgIGFIR KD plus strand oligo
(SEQ ID NO: 2431)
CACCGCCAGCATTAACTCCGCTGAG
Human sgIGFIR KD minus strand oligo
(SEQ ID NO: 2432)
AAACCTCAGCGGAGTTAATGCTGGC
Human sgNTC KD plus strand oligo
(SEQ ID NO: 2433)
CACCGTTTTACCTTGTTCACATGGA
Human sgNTC KD minus strand oligo
(SEQ ID NO: 2434)
AAACTCCATGTGAACAAGGTAAAAC
qPCR primers
Hsa IGF1R qPCR Fwd
(SEQ ID NO: 2435)
TCGACATCCGCAACGACTATC
Hsa IGF1R qPCR Rev
(SEQ ID NO: 2436)
CCAGGGCGTAGTTGTAGAAGAG
Hsa GAPDH qPCR Fwd
(SEQ ID NO: 2437)
GGAGCGAGATCCCTCCAAAAT
Hsa GAPDH qPCR Rev
(SEQ ID NO: 2438)
GGCTGTTGTCATACTTCTCATGG
sgRNA library amplification
Opool amplification Fwd
(SEQ ID NO: 2439)
GGCTTTATATATCTTGTGGAAAGGACGAAACACCG
Opool amplification Rev
(SEQ ID NO: 2440)
AACTTGCTATGCTGTTTCCAGCATAGCTCTTAAAC
Bulk screen amplification primers
sgRNA lib sequencing P5 primer1
(SEQ ID NO: 2441)
AATGATACGGCGACCACCGAGATCTACACACGGTCATCAACACTCTTT
CCCTACACGACGCTCTTCCGATCTATCTTGTGGAAAGGACGAAACACCG
sgRNA lib sequencing P5 primer2
(SEQ ID NO: 2442)
AATGATACGGCGACCACCGAGATCTACACCGACCGAGAGACACTCTTT
CCCTACACGACGCTCTTCCGATCTATCTTGTGGAAAGGACGAAACACCG
sgRNA lib sequencing P7 primer1
(SEQ ID NO: 2443)
CAAGCAGAAGACGGCATACGAGATCTTCTGGTCCGTGACTGGAGTTCA
GACGTGTGCTCTTCCGATCTCCGACTCGGTGCCACTTTTTCAA
sgRNA lib sequencing P7 primer1
(SEQ ID NO: 2444)
CAAGCAGAAGACGGCATACGAGATTCCTCCATACGTGACTGGAGTTCA
GACGTGTGCTCTTCCGATCTCCGACTCGGTGCCACTTTTTCAA
Library preparation oligos
Ligation adaptor
(SEQ ID NO: 2445)
A*G*A*T*C*G*G*A*A*G*A*G*C*G*T*C*G*T*G*T*A*G*G*G*
A*A*A*G*A*G*T*G*T*/3ddC/
Universal P5 primer
(SEQ ID NO: 2446)
AATGATACGGCGACCACCGAGATCTACAC

1. A method for preparing a sequencing library comprising nucleic acids from a plurality of single nuclei or cells, the method comprising:
(a) providing a plurality of nuclei or cells in a first plurality of compartments, wherein each compartment comprises a subset of nuclei or cells;
(b) labeling and processing RNA molecules in the subsets of cells or nuclei obtained from the cells; wherein the labeling comprises adding to RNA molecules present in each subset of nuclei or cells a first compartment specific index sequence to result in indexed DNA nucleic acids present in indexed nuclei or cells, wherein the method comprises the steps of contacting the RNA molecules with a reverse transcriptase, a reverse transcription primer from a set of indexed reverse transcription primers that anneals to a polyA tail of RNA molecules, an indexed random hexamer primer from a set of indexed random hexamer primers, or a combination thereof;
(d) combining the indexed nuclei or cells to generate pooled indexed nuclei or cells;
(e) providing the plurality of nuclei or cells in a second plurality of compartments, wherein each compartment comprises a subset of nuclei or cells;
(f) labeling the indexed DNA nucleic acids in the subsets of cells or nuclei obtained from the cells; wherein the process of labeling comprises adding to the indexed DNA nucleic acids present in each subset of nuclei or cells a second compartment a specific indexed ligation primer from a set of indexed ligation primers to result in double indexed DNA molecules present in double indexed nuclei or cells, wherein the labeling comprises the steps of: contacting the indexed DNA molecules with a chemically modified DNA ligation primer/adaptor complex and a DNA ligase, and ligating the compartment specific DNA ligation primer to the indexed DNA molecules to generate double indexed single stranded DNA (ssDNA) molecules;
(g) combining the double indexed nuclei or cells to generate pooled double indexed nuclei or cells;
(h) providing the plurality of double indexed nuclei or cells in a third plurality of compartments, wherein each compartment comprises a subset of nuclei or cells;
(i) generating double indexed double stranded DNA (dsDNA) molecules by contacting the ssDNA molecules with a second-strand synthesis enzyme mix and synthesizing a second complementary DNA strand;
(j) performing bead-based purification of the double indexed dsDNA molecules;
(k) performing tagmentation on the purified dsDNA molecules;
(l) labeling the double indexed DNA nucleic acids in the subsets of cells or nuclei obtained from the cells; wherein the process of labeling comprises adding to the double indexed DNA molecules present in each subset of nuclei or cells a third compartment specific index sequence to result in triple indexed DNA nucleic acids present in triple indexed nuclei or cells, wherein the labeling comprises contacting the double indexed DNA molecules with a compartment specific indexed PCR primer (referred to as P7), a universal PCR primer (referred to as P5), and a polymerase, and performing PCR amplification of the double indexed DNA molecules to generate triple indexed DNA molecules.

2. The method of claim 1, wherein the reverse transcriptase comprises Maxima Reverse Transcriptase.

3. The method of claim 1, wherein the set of oligo-dT primers comprises a set of primers comprising sequences selected from the sequences as set forth in Table 3.

4. The method of claim 1, wherein the set of indexed random hexamer primers comprises a set of primers comprising sequences selected from the sequences as set forth in Table 4.

5. The method of claim 1 wherein the set of indexed ligation primers comprises a set of primers comprising sequences selected from the sequences as set forth in Table 5.

6. The method of claim 1, wherein the adaptor comprises SEQ ID NO: 2445.

7. The method of claim 1, wherein the ligation is performed using T4 ligase.

8. The method of claim 1, wherein the method further includes one or more steps selected from the group consisting of:
a) nuclei extraction;
b) nuclei fixation; and
c) nuclei storage
which are performed prior to step a) of claim 1.

9. The method of claim 8, wherein the step of nuclei extraction is performed using a buffer comprising 1% DEPC and 0.1% SUPREase.

10. The method of claim 8, wherein the step of nuclei fixation is performed by contacting extracted nuclei with 0.1% formaldehyde for 10 minutes.

11. The method of claim 8, wherein the method of nuclei storage comprises contacting nuclei with 10% DMSO and then freezing.

12. The method of claim 1, wherein the compartment comprises a well or a droplet.

13. The method of claim 1, wherein compartments of the first plurality of compartments comprise from 50 to 20,000 nuclei or cells.

14. The method of claim 1, wherein compartments of the second plurality of compartments comprise from 50 to 20,000 nuclei or cells.

15. The method of claim 1, wherein compartments of the third plurality of compartments comprise from 50 to 20,000 nuclei or cells.

16. The method of claim 1, further comprising pooling and collecting the triple indexed nucleic acids, thereby producing a sequencing library from the plurality of nuclei or cells.

17. A kit for use in preparing a sequencing library, the kit comprising at least one set of indexed oligonucleotides for use in a method of any one of claims 1-16.

18. The kit of claim 17 comprising a set of 192 indexed primers of claim 3.

19. The kit of claim 17 comprising a set of 192 indexed primers of claim 4.

20. The kit of claim 17 comprising a set of 382 indexed primers of claim 5.

21. A method for preparing a sequencing library for determination of transcriptome kinetics, the method comprising:
a) providing a plurality of cells comprising an expression construct for expression of a catalytically dead Cas9 protein;
b) contacting the cells of a) with an sgRNA library;
c) culturing the cells of b) in the presence of a selection agent for selection of cells containing an sgRNA library molecule;
d) splitting the cells of c) into
i) a first population of cells for generation of a first “bulk” sequencing library; and
ii) a second population of cells for subsequent culturing;
e) culturing the cells of d) ii) in the presence of at least one of:
i) an inducing agent to induce expression of the catalytically dead Cas9 protein;
ii) at least one agent for perturbing cells; and
iii) at least one agent for sensitizing cells to perturbations;
f) culturing at least a portion of the cells of e) in the presence of an RNA metabolic label to label nascent transcripts;
g) splitting the cells of f) into
i) a first population of cells for generation of a second “bulk” sequencing library; and
ii) a second population of cells for subsequent chemical conversion and indexing;
h) chemically converting the RNA metabolic label in the RNA molecules from the cells of g) ii);
i) generating one or more sequencing library from the DNA molecules, RNA molecules, or a combination thereof, from the cells of step d) i), step g) i) and step h).

22. The method of claim 21, wherein the catalytically dead Cas9 protein is under the control of an inducible promoter

23. The method of claim 22, wherein the promoter is inducible by contacting the cell with doxycycline (Dox).

24. The method of claim 23, wherein the inducing agent of step e) i) comprises doxycycline.

25. The method of any one of claims 21-24, wherein the catalytically dead Cas9 protein comprises Dox-inducible dCas9-KRAB-MeCP2.

26. The method of claim 21, wherein the method of step e) iii) comprises culturing the cells in L-glutamine+, sodium pyruvate−, high glucose DMEM.

27. The method of claim 21, wherein the cell culture medium further comprises doxycycline.

28. The method of claim 21, wherein the sgRNA library comprises a library of plasmids encoding at least 500 different sgRNA molecules.

29. The method of claim 21, wherein the RNA metabolic label comprises 4-thiouridine (4sU).

30. The method of claim 21, wherein the method of step i) includes the steps of:
a) providing a plurality of nuclei or cells in a first plurality of compartments, wherein each compartment comprises a subset of nuclei or cells;
b) labeling and processing RNA molecules obtained from the cells; wherein the labeling comprises adding to RNA molecules present in each subset of nuclei or cells a first compartment specific index sequence to result in indexed DNA nucleic acids present in indexed nuclei or cells, wherein the method comprises the steps of contacting the RNA molecules with a reverse transcriptase, a reverse transcription primer from a set of indexed reverse transcription primers that anneals to a polyA tail of RNA molecules, an indexed random hexamer primer from a set of indexed random hexamer primers, or a combination thereof;
c) combining the indexed nuclei or cells to generate pooled indexed nuclei or cells;
d) providing the plurality of nuclei or cells in a second plurality of compartments, wherein each compartment comprises a subset of nuclei or cells;
e) labeling the indexed DNA nucleic acids in the subsets of cells or nuclei obtained from the cells; wherein the process of labeling comprises adding to the indexed DNA nucleic acids present in each subset of nuclei or cells a second compartment specific indexed ligation primer sequence to result in double indexed DNA molecules present in double indexed nuclei or cells, wherein the labeling comprises the steps of: contacting the indexed DNA molecules with a chemically modified DNA ligation primer/adaptor complex and a DNA ligase, and ligating the compartment specific DNA ligation primer to the indexed DNA molecules to generate double indexed single stranded DNA (ssDNA) molecules;
f) combining the double indexed nuclei or cells to generate pooled double indexed nuclei or cells;
g) providing the plurality of double indexed nuclei or cells in a third plurality of compartments, wherein each compartment comprises a subset of nuclei or cells;
h) generating double indexed double stranded DNA (dsDNA) molecules by contacting the ssDNA molecules with a second-strand synthesis enzyme mix and synthesizing a second complementary DNA strand;
i) performing bead-based purification of the double indexed dsDNA molecules;
j) performing tagmentation on the purified dsDNA molecules; and
k) labeling the double indexed DNA nucleic acids in the subsets of cells or nuclei obtained from the cells; wherein the process of labeling comprises adding to the double indexed DNA molecules present in each subset of nuclei or cells a third compartment specific index sequence to result in triple indexed DNA nucleic acids present in triple indexed nuclei or cells, wherein the labeling comprises contacting the double indexed DNA molecules with a compartment specific indexed PCR primer (referred to as P7), a universal PCR primer (referred to as P5), and a polymerase, and performing PCR amplification of the double indexed DNA molecules to generate triple indexed DNA molecules.

31. The method of claim 30, wherein the set of oligo-dT primers comprises a set of primers comprising sequences selected from the sequences as set forth in Table 3.

32. The method of claim 30, wherein the set of indexed random hexamer primers comprises a set of primers comprising sequences selected from the sequences as set forth in Table 4.

33. The method of claim 30, wherein the set of indexed ligation primers comprises a set of primers comprising sequences selected from the sequences as set forth in Table 5.

34. The method of claim 30, wherein the adaptor comprises SEQ ID NO: 2445.

35. The method of claim 30, wherein the ligation is performed using T4 ligase.

36. The method of claim 30, wherein the method further includes one or more steps selected from the group consisting of:
a) nuclei extraction;
b) nuclei fixation; and
c) nuclei storage
which are performed prior to step a) of claim 2.

37. The method of claim 36, wherein the step of nuclei extraction is performed using a buffer comprising 1% DEPC and 0.1% SUPREase.

38. The method of claim 36, wherein the step of nuclei fixation is performed by contacting extracted nuclei with 0.1% formaldehyde for 10 minutes.

39. The method of claim 36, wherein the method of nuclei storage comprises contacting nuclei with 10% DMSO and then freezing.

40. The method of claim 30, wherein the compartment comprises a well or a droplet.

41. The method of claim 30, wherein compartments of the first plurality of compartments comprise from 50 to 20,000 nuclei or cells.

42. The method of claim 30, wherein compartments of the second plurality of compartments comprise from 50 to 20,000 nuclei or cells.

43. The method of claim 30, wherein compartments of the third plurality of compartments comprise from 50 to 20,000 nuclei or cells.

44. The method of claim 30, further comprising pooling and collecting the triple indexed nucleic acids, thereby producing a sequencing library from the plurality of nuclei or cells.

45. A kit for use in preparing a sequencing library of any one of claims 21-44.

46. A method for preparing a sequencing library comprising nucleic acids from a plurality of single nuclei or cells, the method comprising:
(a) contacting a plurality of nuclei or cells with 5-Ethynyl-2-deoxyuridine (EdU);
(b) contacting the plurality of nuclei or cells with reagents for Click chemistry ligation to an azide-containing fluorophore;
(c) sorting the nuclei in a first plurality of compartments, wherein each compartment comprises a subset of nuclei or cells, wherein the sorting enriches for EdU+ nuclei or cells;
(d) labeling and processing RNA molecules in the subsets of cells or nuclei obtained from the cells; wherein the labeling comprises adding to RNA molecules present in each subset of nuclei or cells a first compartment-specific index sequence to result in indexed DNA nucleic acids present in indexed nuclei or cells, wherein the method comprises the steps of contacting the RNA molecules with a reverse transcriptase, an Oligo-dT primer that anneals to a poly A tail of RNA molecules and an indexed random primer;
(e) combining the indexed nuclei or cells to generate pooled indexed nuclei or cells;
(f) sorting the plurality of nuclei or cells into a second plurality of compartments, wherein each compartment comprises a subset of nuclei or cells;
(g) generating double stranded DNA (dsDNA) molecules by contacting the ssDNA molecules with a second-strand synthesis enzyme mix and synthesizing a second complementary DNA strand;
(h) performing tagmentation on the dsDNA molecules; and
(i) labeling the DNA nucleic acids in the subsets of cells or nuclei obtained from the cells; wherein the process of labeling comprises adding to the indexed DNA molecules present in each subset of nuclei or cells an additional compartment specific-index sequence to result in multi-indexed DNA nucleic acids present in multi-indexed nuclei or cells, wherein the labeling comprises contacting the indexed DNA molecules with a compartment specific indexed PCR primer (referred to as P7), a universal PCR primer (referred to as P5), and a polymerase, and performing PCR amplification of the double indexed DNA molecules to generate multi-indexed DNA molecules.

47. The method of claim 46, wherein the sorting in steps (c) and (f) is performed using FACS sorting gated for fluorophore and DAPI positive nuclei.

48. The method of claim 46, wherein the oligo-dT primer comprises a 5′ end as set forth in SEQ ID NO:2447 and a 3′ end as set forth in SEQ ID NO:2448 flanking a barcode sequence, wherein the barcode sequence comprises any nucleotide sequence from 5 to 20 nucleotides in length.

48. The method of claim 46, wherein compartments of the first plurality of compartments comprise from about 250 to 500 nuclei or cells.

49. The method of claim 46, wherein compartments of the second plurality of compartments comprise about 25 nuclei or cells.

50. The method of claim 46, further comprising pooling and collecting the multi-indexed nucleic acids, thereby producing a sequencing library from the plurality of nuclei or cells.

51. A method for preparing a sequencing library comprising nucleic acids from a plurality of single nuclei or cells, the method comprising:
(a) contacting a plurality of nuclei or cells with 5-Ethynyl-2-deoxyuridine (EdU);
(b) contacting the plurality of nuclei or cells with reagents for Click chemistry ligation to an azide-containing fluorophore;
(c) permeabilizing the nuclei or cells;
(d) sorting the nuclei in a first plurality of compartments, wherein each compartment comprises a subset of nuclei or cells, wherein the sorting enriches for EdU+ nuclei or cells;
(e) performing tagmentation on the nucleic acid molecules using a barcoded transposase;
(f) combining the indexed nuclei or cells to generate pooled indexed nuclei or cells;
(g) sorting the plurality of nuclei or cells into a second plurality of compartments, wherein each compartment comprises a subset of nuclei or cells; and
(h) labeling the DNA nucleic acids in the subsets of cells or nuclei obtained from the cells; wherein the process of labeling comprises adding to the indexed DNA molecules present in each subset of nuclei or cells an additional compartment specific-index sequence to result in multi-indexed DNA nucleic acids present in multi-indexed nuclei or cells, wherein the labeling comprises contacting the indexed DNA molecules with a compartment specific indexed PCR primer (referred to as P7), a universal PCR primer (referred to as P5), and a polymerase, and performing PCR amplification of the double indexed DNA molecules to generate multi-indexed DNA molecules.

52. The method of claim 51, wherein the sorting in steps (d) and (g) is performed using FACS sorting gated for fluorophore and DAPI positive nuclei.

53. The method of claim 51, wherein compartments of the first plurality of compartments comprise from about 250 to 500 nuclei or cells.

54. The method of claim 51, wherein compartments of the second plurality of compartments comprise about 25 nuclei or cells.

55. The method of claim 46, further comprising pooling and collecting the multi-indexed nucleic acids, thereby producing a sequencing library from the plurality of nuclei or cells.
